SEQUENCE LISTING (1144 sequences)

SEQ ID NO: 1 - DNA, 21 bp, Artificial
  oligonucleotide
gtttgaccagattctccgctc

SEQ ID NO: 2 - DNA, 22 bp, Artificial
  oligonucleotide
gtttgaccagattttccgctct

SEQ ID NO: 3 - DNA, 22 bp, Artificial
  oligonucleotide
gtttgaccaaattttccgctct

SEQ ID NO: 4 - DNA, 22 bp, Artificial
  oligonucleotide
gtttgtccaaattctccgctct

SEQ ID NO: 5 - DNA, 18 bp, Artificial
  oligonucleotide
cccggtcgaattaaaacc

SEQ ID NO: 6 - DNA, 18 bp, Artificial
  oligonucleotide
gcccggtcgaattaaaac

SEQ ID NO: 7 - DNA, 18 bp, Artificial
  oligonucleotide
ggcccggtcgaattaaaa

SEQ ID NO: 8 - DNA, 18 bp, Artificial
  oligonucleotide
aggcccggtcgaattaaa

SEQ ID NO: 9 - DNA, 18 bp, Artificial
  oligonucleotide
aaggcccggtcgaattaa

SEQ ID NO: 10 - DNA, 18 bp, Artificial
  oligonucleotide
atattcgagcgaaacgcc

SEQ ID NO: 11 - DNA, 18 bp, Artificial
  oligonucleotide
aaagatccggaccggccg

SEQ ID NO: 12 - DNA, 18 bp, Artificial
  oligonucleotide
ggaaagatccggaccggc

SEQ ID NO: 13 - DNA, 18 bp, Artificial
  oligonucleotide
gaaagatccggaccggcc

SEQ ID NO: 14 - DNA, 18 bp, Artificial
  oligonucleotide
gatccggaccggccgacc

SEQ ID NO: 15 - DNA, 18 bp, Artificial
  oligonucleotide
agatccggaccggccgac

SEQ ID NO: 16 - DNA, 18 bp, Artificial
  oligonucleotide
aagatccggaccggccga

SEQ ID NO: 17 - DNA, 18 bp, Artificial
  oligonucleotide
gaaaggcccggtcgaatt

SEQ ID NO: 18 - DNA, 18 bp, Artificial
  oligonucleotide
aaaggcccggtcgaatta

SEQ ID NO: 19 - DNA, 18 bp, Artificial
  oligonucleotide
ggaaaggcccggtcgaat

SEQ ID NO: 20 - DNA, 18 bp, Artificial
  oligonucleotide
aggaaaggcccggtcgaa

SEQ ID NO: 21 - DNA, 18 bp, Artificial
  oligonucleotide
aaggaaaggcccggtcga

SEQ ID NO: 22 - DNA, 20 bp, Artificial
  oligonucleotide
atagcactgggatcctcgcc

SEQ ID NO: 23 - DNA, 20 bp, Artificial
  oligonucleotide
ccagccccaaagttaccttc

SEQ ID NO: 24 - DNA, 20 bp, Artificial
  oligonucleotide
tccttgacgtaaagtcgcag

SEQ ID NO: 25 - DNA, 18 bp, Artificial
  oligonucleotide
ggaagaaaaccagtacgc

SEQ ID NO: 26 - DNA, 18 bp, Artificial
  oligonucleotide
ccggtcggaagaaaacca

SEQ ID NO: 27 - DNA, 18 bp, Artificial
  oligonucleotide
gaagaaaaccagtacgcg

SEQ ID NO: 28 - DNA, 18 bp, Artificial
  oligonucleotide
cccggtcggaagaaaacc

SEQ ID NO: 29 - DNA, 18 bp, Artificial
  oligonucleotide
cggtcggaagaaaaccag

SEQ ID NO: 30 - DNA, 18 bp, Artificial
  oligonucleotide
ggtcggaagaaaaccagt

SEQ ID NO: 31 - DNA, 18 bp, Artificial
  oligonucleotide
aagaaaaccagtacgcgg

SEQ ID NO: 32 - DNA, 18 bp, Artificial
  oligonucleotide
gtacgcggaaaaatccgg

SEQ ID NO: 33 - DNA, 18 bp, Artificial
  oligonucleotide
agtacgcggaaaaatccg

SEQ ID NO: 34 - DNA, 18 bp, Artificial
  oligonucleotide
gcggaaaaatccggaccg

SEQ ID NO: 35 - DNA, 18 bp, Artificial
  oligonucleotide
cggaagaaaaccagtacg

SEQ ID NO: 36 - DNA, 18 bp, Artificial
  oligonucleotide
gcccggtcggaagaaaac

SEQ ID NO: 37 - DNA, 18 bp, Artificial
  oligonucleotide
cgcggaaaaatccggacc

SEQ ID NO: 38 - DNA, 18 bp, Artificial
  oligonucleotide
cagtacgcggaaaaatcc

SEQ ID NO: 39 - DNA, 18 bp, Artificial
  oligonucleotide
agaaaaccagtacgcgga

SEQ ID NO: 40 - DNA, 18 bp, Artificial
  oligonucleotide
ggcccggtcggaagaaaa

SEQ ID NO: 41 - DNA, 18 bp, Artificial
  oligonucleotide
ataaacaccacccgatcc

SEQ ID NO: 42 - DNA, 18 bp, Artificial
  oligonucleotide
acgcggaaaaatccggac

SEQ ID NO: 43 - DNA, 18 bp, Artificial
  oligonucleotide
gagaggcccggtcggaag

SEQ ID NO: 44 - DNA, 18 bp, Artificial
  oligonucleotide
agaggcccggtcggaaga

SEQ ID NO: 45 - DNA, 18 bp, Artificial
  oligonucleotide
gaggcccggtcggaagaa

SEQ ID NO: 46 - DNA, 18 bp, Artificial
  oligonucleotide
aggcccggtcggaagaaa

SEQ ID NO: 47 - DNA, 18 bp, Artificial
  oligonucleotide
ccgagtgggtcagtaaat

SEQ ID NO: 48 - DNA, 18 bp, Artificial
  oligonucleotide
ccagtacgcggaaaaatc

SEQ ID NO: 49 - DNA, 18 bp, Artificial
  oligonucleotide
taaacaccacccgatccc

SEQ ID NO: 50 - DNA, 18 bp, Artificial
  oligonucleotide
ggagaggcccggtcggaa

SEQ ID NO: 51 - DNA, 18 bp, Artificial
  oligonucleotide
gaaaaccagtacgcggaa

SEQ ID NO: 52 - DNA, 18 bp, Artificial
  oligonucleotide
tacgcggaaaaatccgga

SEQ ID NO: 53 - DNA, 18 bp, Artificial
  oligonucleotide
ggccacagggacccaggg

SEQ ID NO: 54 - DNA, 18 bp, Artificial
  oligonucleotide
tcaccaagggccacaggg

SEQ ID NO: 55 - DNA, 18 bp, Artificial
  oligonucleotide
gggccacagggacccagg

SEQ ID NO: 56 - DNA, 18 bp, Artificial
  oligonucleotide
ttcaccaagggccacagg

SEQ ID NO: 57 - DNA, 18 bp, Artificial
  oligonucleotide
acagggacccagggctag

SEQ ID NO: 58 - DNA, 18 bp, Artificial
  oligonucleotide
agggccacagggacccag

SEQ ID NO: 59 - DNA, 18 bp, Artificial
  oligonucleotide
gttcaccaagggccacag

SEQ ID NO: 60 - DNA, 18 bp, Artificial
  oligonucleotide
gccacagggacccagggc

SEQ ID NO: 61 - DNA, 18 bp, Artificial
  oligonucleotide
cagggacccagggctagc

SEQ ID NO: 62 - DNA, 18 bp, Artificial
  oligonucleotide
agggacccagggctagcc

SEQ ID NO: 63 - DNA, 18 bp, Artificial
  oligonucleotide
accaagggccacagggac

SEQ ID NO: 64 - DNA, 18 bp, Artificial
  oligonucleotide
ccacagggacccagggct

SEQ ID NO: 65 - DNA, 18 bp, Artificial
  oligonucleotide
cacagggacccagggcta

SEQ ID NO: 66 - DNA, 18 bp, Artificial
  oligonucleotide
caccaagggccacaggga

SEQ ID NO: 67 - DNA, 18 bp, Artificial
  oligonucleotide
gggacccagggctagcca

SEQ ID NO: 68 - DNA, 18 bp, Artificial
  oligonucleotide
aggagaggcccggtcgga

SEQ ID NO: 69 - DNA, 18 bp, Artificial
  oligonucleotide
aaggagaggcccggtcgg

SEQ ID NO: 70 - DNA, 18 bp, Artificial
  oligonucleotide
gaaggagaggcccggtcg

SEQ ID NO: 71 - DNA, 18 bp, Artificial
  oligonucleotide
agggctagccagaaggag

SEQ ID NO: 72 - DNA, 18 bp, Artificial
  oligonucleotide
gggctagccagaaggaga

SEQ ID NO: 73 - DNA, 18 bp, Artificial
  oligonucleotide
agaaggagaggcccggtc

SEQ ID NO: 74 - DNA, 18 bp, Artificial
  oligonucleotide
caagggccacagggaccc

SEQ ID NO: 75 - DNA, 18 bp, Artificial
  oligonucleotide
ccaagggccacagggacc

SEQ ID NO: 76 - DNA, 18 bp, Artificial
  oligonucleotide
gtcggaaaaaccagtacg

SEQ ID NO: 77 - DNA, 18 bp, Artificial
  oligonucleotide
gcccggtcggaaaaacca

SEQ ID NO: 78 - DNA, 18 bp, Artificial
  oligonucleotide
ccggtcggaaaaaccagt

SEQ ID NO: 79 - DNA, 18 bp, Artificial
  oligonucleotide
cccggtcggaaaaaccag

SEQ ID NO: 80 - DNA, 18 bp, Artificial
  oligonucleotide
tcggaaaaaccagtacgc

SEQ ID NO: 81 - DNA, 18 bp, Artificial
  oligonucleotide
cggaaaaaccagtacgcg

SEQ ID NO: 82 - DNA, 18 bp, Artificial
  oligonucleotide
ggaaaaaccagtacgcgg

SEQ ID NO: 83 - DNA, 18 bp, Artificial
  oligonucleotide
gtacgcggaaaaatccgg

SEQ ID NO: 84 - DNA, 18 bp, Artificial
  oligonucleotide
agtacgcggaaaaatccg

SEQ ID NO: 85 - DNA, 18 bp, Artificial
  oligonucleotide
gcggaaaaatccggaccg

SEQ ID NO: 86 - DNA, 18 bp, Artificial
  oligonucleotide
ggtcggaaaaaccagtac

SEQ ID NO: 87 - DNA, 18 bp, Artificial
  oligonucleotide
actcctagtggtgccctt

SEQ ID NO: 88 - DNA, 18 bp, Artificial
  oligonucleotide
gctccactcctagtggtg

SEQ ID NO: 89 - DNA, 18 bp, Artificial
  oligonucleotide
cactcctagtggtgccct

SEQ ID NO: 90 - DNA, 18 bp, Artificial
  oligonucleotide
ctccactcctagtggtgc

SEQ ID NO: 91 - DNA, 18 bp, Artificial
  oligonucleotide
tccactcctagtggtgcc

SEQ ID NO: 92 - DNA, 18 bp, Artificial
  oligonucleotide
ccactcctagtggtgccc

SEQ ID NO: 93 - DNA, 18 bp, Artificial
  oligonucleotide
ggctccactcctagtggt

SEQ ID NO: 94 - DNA, 18 bp, Artificial
  oligonucleotide
aggctccactcctagtgg

SEQ ID NO: 95 - DNA, 18 bp, Artificial
  oligonucleotide
ggcccggtcggaaaaacc

SEQ ID NO: 96 - DNA, 18 bp, Artificial
  oligonucleotide
gaaaaaccagtacgcgga

SEQ ID NO: 97 - DNA, 18 bp, Artificial
  oligonucleotide
cgcggaaaaatccggacc

SEQ ID NO: 98 - DNA, 18 bp, Artificial
  oligonucleotide
cagtacgcggaaaaatcc

SEQ ID NO: 99 - DNA, 18 bp, Artificial
  oligonucleotide
cggtcggaaaaaccagta

SEQ ID NO: 100 - DNA, 18 bp, Artificial
  oligonucleotide
aaggcccggtcggaaaaa

SEQ ID NO: 101 - DNA, 18 bp, Artificial
  oligonucleotide
caggctccactcctagtg

SEQ ID NO: 102 - DNA, 18 bp, Artificial
  oligonucleotide
ctcctagtggtgcccttc

SEQ ID NO: 103 - DNA, 18 bp, Artificial
  oligonucleotide
tcctagtggtgcccttcc

SEQ ID NO: 104 - DNA, 18 bp, Artificial
  oligonucleotide
gcaggctccactcctagt

SEQ ID NO: 105 - DNA, 18 bp, Artificial
  oligonucleotide
aggcccggtcggaaaaac

SEQ ID NO: 106 - DNA, 18 bp, Artificial
  oligonucleotide
acgcggaaaaatccggac

SEQ ID NO: 107 - DNA, 18 bp, Artificial
  oligonucleotide
ccagtacgcggaaaaatc

SEQ ID NO: 108 - DNA, 18 bp, Artificial
  oligonucleotide
ctagtggtgcccttccgt

SEQ ID NO: 109 - DNA, 18 bp, Artificial
  oligonucleotide
gaaaggcccggtcggaaa

SEQ ID NO: 110 - DNA, 18 bp, Artificial
  oligonucleotide
aaaggcccggtcggaaaa

SEQ ID NO: 111 - DNA, 18 bp, Artificial
  oligonucleotide
tacgcggaaaaatccgga

SEQ ID NO: 112 - DNA, 18 bp, Artificial
  oligonucleotide
ggaaaggcccggtcggaa

SEQ ID NO: 113 - DNA, 18 bp, Artificial
  oligonucleotide
atctcttccgaaaggtcg

SEQ ID NO: 114 - DNA, 18 bp, Artificial
  oligonucleotide
catctcttccgaaaggtc

SEQ ID NO: 115 - DNA, 18 bp, Artificial
  oligonucleotide
ctcttccgaaaggtcgag

SEQ ID NO: 116 - DNA, 18 bp, Artificial
  oligonucleotide
cttccgaaaggtcgagat

SEQ ID NO: 117 - DNA, 18 bp, Artificial
  oligonucleotide
tctcttccgaaaggtcga

SEQ ID NO: 118 - DNA, 18 bp, Artificial
  oligonucleotide
tcttccgaaaggtcgaga

SEQ ID NO: 119 - DNA, 18 bp, Artificial
  oligonucleotide
cctagtggtgcccttccg

SEQ ID NO: 120 - DNA, 18 bp, Artificial
  oligonucleotide
tagtggtgcccttccgtc

SEQ ID NO: 121 - DNA, 18 bp, Artificial
  oligonucleotide
agtggtgcccttccgtca

SEQ ID NO: 122 - DNA, 18 bp, Artificial
  oligonucleotide
gccaaggttagactcgtt

SEQ ID NO: 123 - DNA, 18 bp, Artificial
  oligonucleotide
ggccaaggttagactcgt

SEQ ID NO: 124 - DNA, 18 bp, Artificial
  oligonucleotide
ccaaggttagactcgttg

SEQ ID NO: 125 - DNA, 18 bp, Artificial
  oligonucleotide
caaggttagactcgttgg

SEQ ID NO: 126 - DNA, 18 bp, Artificial
  oligonucleotide
aaggttagactcgttggc

SEQ ID NO: 127 - DNA, 20 bp, Artificial
  oligonucleotide
ctcgcctcacggggttctca

SEQ ID NO: 128 - DNA, 18 bp, Artificial
  oligonucleotide
ggcccggtcgaaattaaa

SEQ ID NO: 129 - DNA, 18 bp, Artificial
  oligonucleotide
aggcccggtcgaaattaa

SEQ ID NO: 130 - DNA, 18 bp, Artificial
  oligonucleotide
aaggcccggtcgaaatta

SEQ ID NO: 131 - DNA, 18 bp, Artificial
  oligonucleotide
aaaggcccggtcgaaatt

SEQ ID NO: 132 - DNA, 18 bp, Artificial
  oligonucleotide
gaaaggcccggtcgaaat

SEQ ID NO: 133 - DNA, 18 bp, Artificial
  oligonucleotide
atattcgagcgaaacgcc

SEQ ID NO: 134 - DNA, 18 bp, Artificial
  oligonucleotide
ggaaaggcccggtcgaaa

SEQ ID NO: 135 - DNA, 18 bp, Artificial
  oligonucleotide
aaagatccggaccggccg

SEQ ID NO: 136 - DNA, 18 bp, Artificial
  oligonucleotide
ggaaagatccggaccggc

SEQ ID NO: 137 - DNA, 18 bp, Artificial
  oligonucleotide
gaaagatccggaccggcc

SEQ ID NO: 138 - DNA, 18 bp, Artificial
  oligonucleotide
gatccggaccggccgacc

SEQ ID NO: 139 - DNA, 18 bp, Artificial
  oligonucleotide
agatccggaccggccgac

SEQ ID NO: 140 - DNA, 18 bp, Artificial
  oligonucleotide
aagatccggaccggccga

SEQ ID NO: 141 - DNA, 18 bp, Artificial
  oligonucleotide
aggaaaggcccggtcgaa

SEQ ID NO: 142 - DNA, 18 bp, Artificial
  oligonucleotide
aaggaaaggcccggtcga

SEQ ID NO: 143 - DNA, 20 bp, Artificial
  oligonucleotide
cgagcaaaacgcctgctttg

SEQ ID NO: 144 - DNA, 20 bp, Artificial
  oligonucleotide
cgctctgaaagagagttgcc

SEQ ID NO: 145 - DNA, 20 bp, Artificial
  oligonucleotide
agttgccccctacactagac

SEQ ID NO: 146 - DNA, 19 bp, Artificial
  oligonucleotide
gcttctccgtcccgcgccg

SEQ ID NO: 147 - DNA, 21 bp, Artificial
  oligonucleotide
agattytccgctctgagatgg

SEQ ID NO: 148 - DNA, 19 bp, Artificial
  oligonucleotide
cctggttcgccaaaaaggc

SEQ ID NO: 149 - DNA, 18 bp, Artificial
  oligonucleotide
gattctcggccccatggg

SEQ ID NO: 150 - DNA, 20 bp, Artificial
  oligonucleotide
accctctacggcagcctgtt

SEQ ID NO: 151 - DNA, 20 bp, Artificial
  oligonucleotide
gatcggtctccagcgattca

SEQ ID NO: 152 - DNA, 20 bp, Artificial
  oligonucleotide
accctccacggcggcctgtt

SEQ ID NO: 153 - DNA, 18 bp, Artificial
  oligonucleotide
gattctccgcgccatggg

SEQ ID NO: 154 - DNA, 20 bp, Artificial
  oligonucleotide
tcatcagacgggattctcac

SEQ ID NO: 155 - DNA, 22 bp, Artificial
  oligonucleotide
ctcatcgcacgggattctcacc

SEQ ID NO: 156 - DNA, 22 bp, Artificial
  oligonucleotide
ctcgccacacgggattctcacc

SEQ ID NO: 157 - DNA, 20 bp, Artificial
  oligonucleotide
agttgccccctcctctaagc

SEQ ID NO: 158 - DNA, 20 bp, Artificial
  oligonucleotide
ctgccacaaggacaaatggt

SEQ ID NO: 159 - DNA, 21 bp, Artificial
  oligonucleotide
tgccccctcttctaagcaaat

SEQ ID NO: 160 - DNA, 18 bp, Artificial
  oligonucleotide
ccccaaagttgccctctc

SEQ ID NO: 161 - DNA, 23 bp, Artificial
  oligonucleotide
gccgccccaaagtcgccctctac

SEQ ID NO: 162 - DNA, 20 bp, Artificial
  oligonucleotide
gccccagagtcgccttctac

SEQ ID NO: 163 - DNA, 18 bp, Artificial
  oligonucleotide
aagaccaggccacctcat

SEQ ID NO: 164 - DNA, 18 bp, Artificial
  oligonucleotide
catcatagaacaccgtcc

SEQ ID NO: 165 - DNA, 20 bp, Artificial
  oligonucleotide
ccttccgaagtcgaggtttt

SEQ ID NO: 166 - DNA, 17 bp, Artificial
  oligonucleotide
gggagtgttgccaactc

SEQ ID NO: 167 - DNA, 19 bp, Artificial
  oligonucleotide
agcggtcgttcgcaaccct

SEQ ID NO: 168 - DNA, 20 bp, Artificial
  oligonucleotide
ccgaagtcggggttttgcgg

SEQ ID NO: 169 - DNA, 20 bp, Artificial
  oligonucleotide
gatagccgaaaccacctttc

SEQ ID NO: 170 - DNA, 20 bp, Artificial
  oligonucleotide
gccgaaaccacctttcaaac

SEQ ID NO: 171 - DNA, 20 bp, Artificial
  oligonucleotide
gtgatagccgaaaccacctt

SEQ ID NO: 172 - DNA, 20 bp, Artificial
  oligonucleotide
agtgatagccgaaaccacct

SEQ ID NO: 173 - DNA, 20 bp, Artificial
  oligonucleotide
tttaacgggatgcgttcgac

SEQ ID NO: 174 - DNA, 20 bp, Artificial
  oligonucleotide
aagtgatagccgaaaccacc

SEQ ID NO: 175 - DNA, 20 bp, Artificial
  oligonucleotide
ggttgaataccgtcaacgtc

SEQ ID NO: 176 - DNA, 20 bp, Artificial
  oligonucleotide
gcacagtatgtcaagacctg

SEQ ID NO: 177 - DNA, 20 bp, Artificial
  oligonucleotide
catccgatgtgcaagcactt

SEQ ID NO: 178 - DNA, 20 bp, Artificial
  oligonucleotide
tcatccgatgtgcaagcact

SEQ ID NO: 179 - DNA, 20 bp, Artificial
  oligonucleotide
ccgatgtgcaagcacttcat

SEQ ID NO: 180 - DNA, 20 bp, Artificial
  oligonucleotide
ccactcatccgatgtgcaag

SEQ ID NO: 181 - DNA, 20 bp, Artificial
  oligonucleotide
gccacagttcgccactcatc

SEQ ID NO: 182 - DNA, 20 bp, Artificial
  oligonucleotide
cctccgcgtttgtcaccggc

SEQ ID NO: 183 - DNA, 20 bp, Artificial
  oligonucleotide
accagttcgccacagttcgc

SEQ ID NO: 184 - DNA, 20 bp, Artificial
  oligonucleotide
cactcatccgatgtgcaagc

SEQ ID NO: 185 - DNA, 20 bp, Artificial
  oligonucleotide
ccagttcgccacagttcgcc

SEQ ID NO: 186 - DNA, 20 bp, Artificial
  oligonucleotide
ctcatccgatgtgcaagcac

SEQ ID NO: 187 - DNA, 20 bp, Artificial
  oligonucleotide
tccgatgtgcaagcacttca

SEQ ID NO: 188 - DNA, 20 bp, Artificial
  oligonucleotide
cgccactcatccgatgtgca

SEQ ID NO: 189 - DNA, 20 bp, Artificial
  oligonucleotide
cagttcgccacagttcgcca

SEQ ID NO: 190 - DNA, 20 bp, Artificial
  oligonucleotide
gccactcatccgatgtgcaa

SEQ ID NO: 191 - DNA, 20 bp, Artificial
  oligonucleotide
cgccacagttcgccactcat

SEQ ID NO: 192 - DNA, 20 bp, Artificial
  oligonucleotide
atccgatgtgcaagcacttc

SEQ ID NO: 193 - DNA, 20 bp, Artificial
  oligonucleotide
gttcgccacagttcgccact

SEQ ID NO: 194 - DNA, 20 bp, Artificial
  oligonucleotide
tcctccgcgtttgtcaccgg

SEQ ID NO: 195 - DNA, 20 bp, Artificial
  oligonucleotide
cgccagggttcatcctgagc

SEQ ID NO: 196 - DNA, 20 bp, Artificial
  oligonucleotide
agttcgccacagttcgccac

SEQ ID NO: 197 - DNA, 20 bp, Artificial
  oligonucleotide
tcgccacagttcgccactca

SEQ ID NO: 198 - DNA, 20 bp, Artificial
  oligonucleotide
ttaacgggatgcgttcgact

SEQ ID NO: 199 - DNA, 20 bp, Artificial
  oligonucleotide
tcgccactcatccgatgtgc

SEQ ID NO: 200 - DNA, 20 bp, Artificial
  oligonucleotide
ccacagttcgccactcatcc

SEQ ID NO: 201 - DNA, 20 bp, Artificial
  oligonucleotide
gatttaacgggatgcgttcg

SEQ ID NO: 202 - DNA, 20 bp, Artificial
  oligonucleotide
taacgggatgcgttcgactt

SEQ ID NO: 203 - DNA, 20 bp, Artificial
  oligonucleotide
aacgggatgcgttcgacttg

SEQ ID NO: 204 - DNA, 20 bp, Artificial
  oligonucleotide
cgaaggttaccgaaccgact

SEQ ID NO: 205 - DNA, 20 bp, Artificial
  oligonucleotide
ccgaaggttaccgaaccgac

SEQ ID NO: 206 - DNA, 20 bp, Artificial
  oligonucleotide
cccgaaggttaccgaaccga

SEQ ID NO: 207 - DNA, 20 bp, Artificial
  oligonucleotide
ttcctccgcgtttgtcaccg

SEQ ID NO: 208 - DNA, 20 bp, Artificial
  oligonucleotide
ccgccagggttcatcctgag

SEQ ID NO: 209 - DNA, 20 bp, Artificial
  oligonucleotide
tccttccagaagtgatagcc

SEQ ID NO: 210 - DNA, 20 bp, Artificial
  oligonucleotide
caccagttcgccacagttcg

SEQ ID NO: 211 - DNA, 20 bp, Artificial
  oligonucleotide
acgggatgcgttcgacttgc

SEQ ID NO: 212 - DNA, 20 bp, Artificial
  oligonucleotide
gtccttccagaagtgatagc

SEQ ID NO: 213 - DNA, 20 bp, Artificial
  oligonucleotide
gccagggttcatcctgagcc

SEQ ID NO: 214 - DNA, 20 bp, Artificial
  oligonucleotide
actcatccgatgtgcaagca

SEQ ID NO: 215 - DNA, 20 bp, Artificial
  oligonucleotide
atcattgccttggtgaaccg

SEQ ID NO: 216 - DNA, 20 bp, Artificial
  oligonucleotide
tccgcgtttgtcaccggcag

SEQ ID NO: 217 - DNA, 20 bp, Artificial
  oligonucleotide
tgaaccgttactccaccaac

SEQ ID NO: 218 - DNA, 20 bp, Artificial
  oligonucleotide
gaagtgatagccgaaaccac

SEQ ID NO: 219 - DNA, 20 bp, Artificial
  oligonucleotide
ccgcgtttgtcaccggcagt

SEQ ID NO: 220 - DNA, 20 bp, Artificial
  oligonucleotide
ttcgccactcatccgatgtg

SEQ ID NO: 221 - DNA, 20 bp, Artificial
  oligonucleotide
catttaacgggatgcgttcg

SEQ ID NO: 222 - DNA, 20 bp, Artificial
  oligonucleotide
cacagttcgccactcatccg

SEQ ID NO: 223 - DNA, 20 bp, Artificial
  oligonucleotide
ttcgccacagttcgccactc

SEQ ID NO: 224 - DNA, 20 bp, Artificial
  oligonucleotide
ctccgcgtttgtcaccggca

SEQ ID NO: 225 - DNA, 20 bp, Artificial
  oligonucleotide
acgccgccagggttcatcct

SEQ ID NO: 226 - DNA, 20 bp, Artificial
  oligonucleotide
ccttccagaagtgatagccg

SEQ ID NO: 227 - DNA, 20 bp, Artificial
  oligonucleotide
tcattgccttggtgaaccgt

SEQ ID NO: 228 - DNA, 20 bp, Artificial
  oligonucleotide
cacagtatgtcaagacctgg

SEQ ID NO: 229 - DNA, 20 bp, Artificial
  oligonucleotide
ttggtgaaccgttactccac

SEQ ID NO: 230 - DNA, 20 bp, Artificial
  oligonucleotide
cttggtgaaccgttactcca

SEQ ID NO: 231 - DNA, 20 bp, Artificial
  oligonucleotide
gtgaaccgttactccaccaa

SEQ ID NO: 232 - DNA, 20 bp, Artificial
  oligonucleotide
ggctcccgaaggttaccgaa

SEQ ID NO: 233 - DNA, 20 bp, Artificial
  oligonucleotide
gaaggttaccgaaccgactt

SEQ ID NO: 234 - DNA, 20 bp, Artificial
  oligonucleotide
tggctcccgaaggttaccga

SEQ ID NO: 235 - DNA, 20 bp, Artificial
  oligonucleotide
taatacgccgcgggtccttc

SEQ ID NO: 236 - DNA, 20 bp, Artificial
  oligonucleotide
gaaccgttactccaccaact

SEQ ID NO: 237 - DNA, 20 bp, Artificial
  oligonucleotide
tacgccgcgggtccttccag

SEQ ID NO: 238 - DNA, 20 bp, Artificial
  oligonucleotide
tcaccagttcgccacagttc

SEQ ID NO: 239 - DNA, 20 bp, Artificial
  oligonucleotide
ccttggtgaaccgttactcc

SEQ ID NO: 240 - DNA, 20 bp, Artificial
  oligonucleotide
ctcaccagttcgccacagtt

SEQ ID NO: 241 - DNA, 20 bp, Artificial
  oligonucleotide
cgccgccagggttcatcctg

SEQ ID NO: 242 - DNA, 20 bp, Artificial
  oligonucleotide
ccttggtgaaccattactcc

SEQ ID NO: 243 - DNA, 20 bp, Artificial
  oligonucleotide
tggtgaaccattactccacc

SEQ ID NO: 244 - DNA, 20 bp, Artificial
  oligonucleotide
gccgccagggttcatcctga

SEQ ID NO: 245 - DNA, 20 bp, Artificial
  oligonucleotide
ggtgaaccattactccacca

SEQ ID NO: 246 - DNA, 20 bp, Artificial
  oligonucleotide
ccagggttcatcctgagcca

SEQ ID NO: 247 - DNA, 20 bp, Artificial
  oligonucleotide
aatacgccgcgggtccttcc

SEQ ID NO: 248 - DNA, 20 bp, Artificial
  oligonucleotide
cacgccgccagggttcatcc

SEQ ID NO: 249 - DNA, 20 bp, Artificial
  oligonucleotide
agttcgccactcatccgatg

SEQ ID NO: 250 - DNA, 20 bp, Artificial
  oligonucleotide
cgggatgcgttcgacttgca

SEQ ID NO: 251 - DNA, 20 bp, Artificial
  oligonucleotide
cattgccttggtgaaccgtt

SEQ ID NO: 252 - DNA, 20 bp, Artificial
  oligonucleotide
gcacgccgccagggttcatc

SEQ ID NO: 253 - DNA, 20 bp, Artificial
  oligonucleotide
cttcctccgcgtttgtcacc

SEQ ID NO: 254 - DNA, 20 bp, Artificial
  oligonucleotide
tggtgaaccgttactccacc

SEQ ID NO: 255 - DNA, 20 bp, Artificial
  oligonucleotide
ccttcctccgcgtttgtcac

SEQ ID NO: 256 - DNA, 20 bp, Artificial
  oligonucleotide
acgccgcgggtccttccaga

SEQ ID NO: 257 - DNA, 20 bp, Artificial
  oligonucleotide
ggtgaaccgttactccacca

SEQ ID NO: 258 - DNA, 20 bp, Artificial
  oligonucleotide
gggtccttccagaagtgata

SEQ ID NO: 259 - DNA, 20 bp, Artificial
  oligonucleotide
cttccagaagtgatagccga

SEQ ID NO: 260 - DNA, 20 bp, Artificial
  oligonucleotide
gccttggtgaaccattactc

SEQ ID NO: 261 - DNA, 20 bp, Artificial
  oligonucleotide
acagttcgccactcatccga

SEQ ID NO: 262 - DNA, 20 bp, Artificial
  oligonucleotide
accttcctccgcgtttgtca

SEQ ID NO: 263 - DNA, 20 bp, Artificial
  oligonucleotide
cgaaccgactttgggtgttg

SEQ ID NO: 264 - DNA, 20 bp, Artificial
  oligonucleotide
gaaccgactttgggtgttgc

SEQ ID NO: 265 - DNA, 20 bp, Artificial
  oligonucleotide
aggttaccgaaccgactttg

SEQ ID NO: 266 - DNA, 20 bp, Artificial
  oligonucleotide
accgaaccgactttgggtgt

SEQ ID NO: 267 - DNA, 20 bp, Artificial
  oligonucleotide
ttaccgaaccgactttgggt

SEQ ID NO: 268 - DNA, 20 bp, Artificial
  oligonucleotide
taccgaaccgactttgggtg

SEQ ID NO: 269 - DNA, 20 bp, Artificial
  oligonucleotide
gttaccgaaccgactttggg

SEQ ID NO: 270 - DNA, 21 bp, Artificial
  oligonucleotide
cctttctggtatggtaccgtc

SEQ ID NO: 271 - DNA, 20 bp, Artificial
  oligonucleotide
tgcaccgcggayccatctct

SEQ ID NO: 272 - DNA, 20 bp, Artificial
  oligonucleotide
agttgcagtccagtaagccg

SEQ ID NO: 273 - DNA, 20 bp, Artificial
  oligonucleotide
gttgcagtccagtaagccgc

SEQ ID NO: 274 - DNA, 20 bp, Artificial
  oligonucleotide
cagttgcagtccagtaagcc

SEQ ID NO: 275 - DNA, 20 bp, Artificial
  oligonucleotide
tgcagtccagtaagccgcct

SEQ ID NO: 276 - DNA, 20 bp, Artificial
  oligonucleotide
tcagttgcagtccagtaagc

SEQ ID NO: 277 - DNA, 20 bp, Artificial
  oligonucleotide
ttgcagtccagtaagccgcc

SEQ ID NO: 278 - DNA, 20 bp, Artificial
  oligonucleotide
gcagtccagtaagccgcctt

SEQ ID NO: 279 - DNA, 20 bp, Artificial
  oligonucleotide
gtcagttgcagtccagtaag

SEQ ID NO: 280 - DNA, 20 bp, Artificial
  oligonucleotide
ctctaggtgacgccgaagcg

SEQ ID NO: 281 - DNA, 20 bp, Artificial
  oligonucleotide
atctctaggtgacgccgaag

SEQ ID NO: 282 - DNA, 20 bp, Artificial
  oligonucleotide
tctaggtgacgccgaagcgc

SEQ ID NO: 283 - DNA, 20 bp, Artificial
  oligonucleotide
tctctaggtgacgccgaagc

SEQ ID NO: 284 - DNA, 20 bp, Artificial
  oligonucleotide
ccatctctaggtgacgccga

SEQ ID NO: 285 - DNA, 20 bp, Artificial
  oligonucleotide
catctctaggtgacgccgaa

SEQ ID NO: 286 - DNA, 20 bp, Artificial
  oligonucleotide
taggtgacgccgaagcgcct

SEQ ID NO: 287 - DNA, 20 bp, Artificial
  oligonucleotide
ctaggtgacgccgaagcgcc

SEQ ID NO: 288 - DNA, 20 bp, Artificial
  oligonucleotide
cttagacggctccttcctaa

SEQ ID NO: 289 - DNA, 20 bp, Artificial
  oligonucleotide
ccttagacggctccttccta

SEQ ID NO: 290 - DNA, 20 bp, Artificial
  oligonucleotide
acgtcagttgcagtccagta

SEQ ID NO: 291 - DNA, 20 bp, Artificial
  oligonucleotide
cgtcagttgcagtccagtaa

SEQ ID NO: 292 - DNA, 20 bp, Artificial
  oligonucleotide
acgccgaagcgccttttaac

SEQ ID NO: 293 - DNA, 20 bp, Artificial
  oligonucleotide
gacgccgaagcgccttttaa

SEQ ID NO: 294 - DNA, 20 bp, Artificial
  oligonucleotide
gccgaagcgccttttaactt

SEQ ID NO: 295 - DNA, 20 bp, Artificial
  oligonucleotide
cgccgaagcgccttttaact

SEQ ID NO: 296 - DNA, 20 bp, Artificial
  oligonucleotide
gtgacgccgaagcgcctttt

SEQ ID NO: 297 - DNA, 20 bp, Artificial
  oligonucleotide
tgacgccgaagcgcctttta

SEQ ID NO: 298 - DNA, 20 bp, Artificial
  oligonucleotide
agacggctccttcctaaaag

SEQ ID NO: 299 - DNA, 20 bp, Artificial
  oligonucleotide
acggctccttcctaaaaggt

SEQ ID NO: 300 - DNA, 20 bp, Artificial
  oligonucleotide
gacggctccttcctaaaagg

SEQ ID NO: 301 - DNA, 20 bp, Artificial
  oligonucleotide
ccttcctaaaaggttaggcc

SEQ ID NO: 302 - DNA, 20 bp, Artificial
  oligonucleotide
ggtgacgccaaagcgccttt

SEQ ID NO: 303 - DNA, 20 bp, Artificial
  oligonucleotide
aggtgacgccaaagcgcctt

SEQ ID NO: 304 - DNA, 20 bp, Artificial
  oligonucleotide
taggtgacgccaaagcgcct

SEQ ID NO: 305 - DNA, 20 bp, Artificial
  oligonucleotide
ctctaggtgacgccaaagcg

SEQ ID NO: 306 - DNA, 20 bp, Artificial
  oligonucleotide
tctaggtgacgccaaagcgc

SEQ ID NO: 307 - DNA, 20 bp, Artificial
  oligonucleotide
ctaggtgacgccaaagcgcc

SEQ ID NO: 308 - DNA, 20 bp, Artificial
  oligonucleotide
acgccaaagcgccttttaac

SEQ ID NO: 309 - DNA, 20 bp, Artificial
  oligonucleotide
cgccaaagcgccttttaact

SEQ ID NO: 310 - DNA, 20 bp, Artificial
  oligonucleotide
tgacgccaaagcgcctttta

SEQ ID NO: 311 - DNA, 20 bp, Artificial
  oligonucleotide
tctctaggtgacgccaaagc

SEQ ID NO: 312 - DNA, 20 bp, Artificial
  oligonucleotide
gtgacgccaaagcgcctttt

SEQ ID NO: 313 - DNA, 20 bp, Artificial
  oligonucleotide
gacgccaaagcgccttttaa

SEQ ID NO: 314 - DNA, 20 bp, Artificial
  oligonucleotide
atctctaggtgacgccaaag

SEQ ID NO: 315 - DNA, 20 bp, Artificial
  oligonucleotide
catctctaggtgacgccaaa

SEQ ID NO: 316 - DNA, 20 bp, Artificial
  oligonucleotide
tccatctctaggtgacgcca

SEQ ID NO: 317 - DNA, 20 bp, Artificial
  oligonucleotide
ccatctctaggtgacgccaa

SEQ ID NO: 318 - DNA, 20 bp, Artificial
  oligonucleotide
ctgccttagacggctccccc

SEQ ID NO: 319 - DNA, 20 bp, Artificial
  oligonucleotide
cctgccttagacggctcccc

SEQ ID NO: 320 - DNA, 20 bp, Artificial
  oligonucleotide
gtgtcatgcgacactgagtt

SEQ ID NO: 321 - DNA, 20 bp, Artificial
  oligonucleotide
tgtgtcatgcgacactgagt

SEQ ID NO: 322 - DNA, 20 bp, Artificial
  oligonucleotide
ctttgtgtcatgcgacactg

SEQ ID NO: 323 - DNA, 20 bp, Artificial
  oligonucleotide
ttgtgtcatgcgacactgag

SEQ ID NO: 324 - DNA, 20 bp, Artificial
  oligonucleotide
tgccttagacggctccccct

SEQ ID NO: 325 - DNA, 20 bp, Artificial
  oligonucleotide
agacggctccccctaaaagg

SEQ ID NO: 326 - DNA, 20 bp, Artificial
  oligonucleotide
tagacggctccccctaaaag

SEQ ID NO: 327 - DNA, 20 bp, Artificial
  oligonucleotide
gccttagacggctcccccta

SEQ ID NO: 328 - DNA, 20 bp, Artificial
  oligonucleotide
gctccccctaaaaggttagg

SEQ ID NO: 329 - DNA, 20 bp, Artificial
  oligonucleotide
ggctccccctaaaaggttag

SEQ ID NO: 330 - DNA, 20 bp, Artificial
  oligonucleotide
ctccccctaaaaggttaggc

SEQ ID NO: 331 - DNA, 20 bp, Artificial
  oligonucleotide
tccccctaaaaggttaggcc

SEQ ID NO: 332 - DNA, 20 bp, Artificial
  oligonucleotide
ccctaaaaggttaggccacc

SEQ ID NO: 333 - DNA, 20 bp, Artificial
  oligonucleotide
cccctaaaaggttaggccac

SEQ ID NO: 334 - DNA, 20 bp, Artificial
  oligonucleotide
cggctccccctaaaaggtta

SEQ ID NO: 335 - DNA, 20 bp, Artificial
  oligonucleotide
ccccctaaaaggttaggcca

SEQ ID NO: 336 - DNA, 20 bp, Artificial
  oligonucleotide
cttagacggctccccctaaa

SEQ ID NO: 337 - DNA, 20 bp, Artificial
  oligonucleotide
ttagacggctccccctaaaa

SEQ ID NO: 338 - DNA, 20 bp, Artificial
  oligonucleotide
gggttcgcaactcgttgtat

SEQ ID NO: 339 - DNA, 20 bp, Artificial
  oligonucleotide
ccttagacggctccccctaa

SEQ ID NO: 340 - DNA, 20 bp, Artificial
  oligonucleotide
acggctccccctaaaaggtt

SEQ ID NO: 341 - DNA, 20 bp, Artificial
  oligonucleotide
gacggctccccctaaaaggt

SEQ ID NO: 342 - DNA, 20 bp, Artificial
  oligonucleotide
acgccgcaagaccatcctct

SEQ ID NO: 343 - DNA, 20 bp, Artificial
  oligonucleotide
ctaatacgccgcaagaccat

SEQ ID NO: 344 - DNA, 20 bp, Artificial
  oligonucleotide
tacgccgcaagaccatcctc

SEQ ID NO: 345 - DNA, 20 bp, Artificial
  oligonucleotide
gttacgatctagcaagccgc

SEQ ID NO: 346 - DNA, 20 bp, Artificial
  oligonucleotide
aatacgccgcaagaccatcc

SEQ ID NO: 347 - DNA, 20 bp, Artificial
  oligonucleotide
cgccgcaagaccatcctcta

SEQ ID NO: 348 - DNA, 20 bp, Artificial
  oligonucleotide
gctaatacgccgcaagacca

SEQ ID NO: 349 - DNA, 20 bp, Artificial
  oligonucleotide
accatcctctagcgatccaa

SEQ ID NO: 350 - DNA, 20 bp, Artificial
  oligonucleotide
taatacgccgcaagaccatc

SEQ ID NO: 351 - DNA, 20 bp, Artificial
  oligonucleotide
agccatccctttctggtaag

SEQ ID NO: 352 - DNA, 20 bp, Artificial
  oligonucleotide
atacgccgcaagaccatcct

SEQ ID NO: 353 - DNA, 20 bp, Artificial
  oligonucleotide
agttacgatctagcaagccg

SEQ ID NO: 354 - DNA, 20 bp, Artificial
  oligonucleotide
agctaatacgccgcaagacc

SEQ ID NO: 355 - DNA, 20 bp, Artificial
  oligonucleotide
gccgcaagaccatcctctag

SEQ ID NO: 356 - DNA, 20 bp, Artificial
  oligonucleotide
ttacgatctagcaagccgct

SEQ ID NO: 357 - DNA, 20 bp, Artificial
  oligonucleotide
gaccatcctctagcgatcca

SEQ ID NO: 358 - DNA, 20 bp, Artificial
  oligonucleotide
ttgctacgtcactaggaggc

SEQ ID NO: 359 - DNA, 20 bp, Artificial
  oligonucleotide
acgtcactaggaggcggaaa

SEQ ID NO: 360 - DNA, 20 bp, Artificial
  oligonucleotide
tttgctacgtcactaggagg

SEQ ID NO: 361 - DNA, 20 bp, Artificial
  oligonucleotide
gccatccctttctggtaagg

SEQ ID NO: 362 - DNA, 20 bp, Artificial
  oligonucleotide
tacgtcactaggaggcggaa

SEQ ID NO: 363 - DNA, 20 bp, Artificial
  oligonucleotide
cgtcactaggaggcggaaac

SEQ ID NO: 364 - DNA, 20 bp, Artificial
  oligonucleotide
aagaccatcctctagcgatc

SEQ ID NO: 365 - DNA, 20 bp, Artificial
  oligonucleotide
gcacgtatttagccatccct

SEQ ID NO: 366 - DNA, 20 bp, Artificial
  oligonucleotide
ctctagcgatccaaaaggac

SEQ ID NO: 367 - DNA, 20 bp, Artificial
  oligonucleotide
cctctagcgatccaaaagga

SEQ ID NO: 368 - DNA, 20 bp, Artificial
  oligonucleotide
ccatcctctagcgatccaaa

SEQ ID NO: 369 - DNA, 20 bp, Artificial
  oligonucleotide
ggcacgtatttagccatccc

SEQ ID NO: 370 - DNA, 20 bp, Artificial
  oligonucleotide
tacgatctagcaagccgctt

SEQ ID NO: 371 - DNA, 20 bp, Artificial
  oligonucleotide
cagttacgatctagcaagcc

SEQ ID NO: 372 - DNA, 20 bp, Artificial
  oligonucleotide
ccgcaagaccatcctctagc

SEQ ID NO: 373 - DNA, 20 bp, Artificial
  oligonucleotide
ccatccctttctggtaaggt

SEQ ID NO: 374 - DNA, 20 bp, Artificial
  oligonucleotide
agaccatcctctagcgatcc

SEQ ID NO: 375 - DNA, 20 bp, Artificial
  oligonucleotide
caagaccatcctctagcgat

SEQ ID NO: 376 - DNA, 20 bp, Artificial
  oligonucleotide
gctacgtcactaggaggcgg

SEQ ID NO: 377 - DNA, 20 bp, Artificial
  oligonucleotide
tgctacgtcactaggaggcg

SEQ ID NO: 378 - DNA, 20 bp, Artificial
  oligonucleotide
ctacgtcactaggaggcgga

SEQ ID NO: 379 - DNA, 20 bp, Artificial
  oligonucleotide
cctcaacgtcagttacgatc

SEQ ID NO: 380 - DNA, 20 bp, Artificial
  oligonucleotide
gtcactaggaggcggaaacc

SEQ ID NO: 381 - DNA, 20 bp, Artificial
  oligonucleotide
tcctctagcgatccaaaagg

SEQ ID NO: 382 - DNA, 20 bp, Artificial
  oligonucleotide
tggcacgtatttagccatcc

SEQ ID NO: 383 - DNA, 20 bp, Artificial
  oligonucleotide
acgatctagcaagccgcttt

SEQ ID NO: 384 - DNA, 20 bp, Artificial
  oligonucleotide
gccagtctctcaactcggct

SEQ ID NO: 385 - DNA, 20 bp, Artificial
  oligonucleotide
aagctaatacgccgcaagac

SEQ ID NO: 386 - DNA, 20 bp, Artificial
  oligonucleotide
gtttgctacgtcactaggag

SEQ ID NO: 387 - DNA, 20 bp, Artificial
  oligonucleotide
cgccactctagtcattgcct

SEQ ID NO: 388 - DNA, 20 bp, Artificial
  oligonucleotide
ggccagccagtctctcaact

SEQ ID NO: 389 - DNA, 20 bp, Artificial
  oligonucleotide
cagccagtctctcaactcgg

SEQ ID NO: 390 - DNA, 20 bp, Artificial
  oligonucleotide
cccgaagatcaattcagcgg

SEQ ID NO: 391 - DNA, 20 bp, Artificial
  oligonucleotide
ccggccagtctctcaactcg

SEQ ID NO: 392 - DNA, 20 bp, Artificial
  oligonucleotide
ccagccagtctctcaactcg

SEQ ID NO: 393 - DNA, 20 bp, Artificial
  oligonucleotide
tcattgcctcacttcacccg

SEQ ID NO: 394 - DNA, 20 bp, Artificial
  oligonucleotide
gccagccagtctctcaactc

SEQ ID NO: 395 - DNA, 20 bp, Artificial
  oligonucleotide
cacccgaagatcaattcagc

SEQ ID NO: 396 - DNA, 20 bp, Artificial
  oligonucleotide
gtcattgcctcacttcaccc

SEQ ID NO: 397 - DNA, 20 bp, Artificial
  oligonucleotide
cattgcctcacttcacccga

SEQ ID NO: 398 - DNA, 20 bp, Artificial
  oligonucleotide
attgcctcacttcacccgaa

SEQ ID NO: 399 - DNA, 20 bp, Artificial
  oligonucleotide
cgaagatcaattcagcggct

SEQ ID NO: 400 - DNA, 20 bp, Artificial
  oligonucleotide
agtcattgcctcacttcacc

SEQ ID NO: 401 - DNA, 20 bp, Artificial
  oligonucleotide
tcgccactctagtcattgcc

SEQ ID NO: 402 - DNA, 20 bp, Artificial
  oligonucleotide
ttgcctcacttcacccgaag

SEQ ID NO: 403 - DNA, 20 bp, Artificial
  oligonucleotide
cggccagtctctcaactcgg

SEQ ID NO: 404 - DNA, 20 bp, Artificial
  oligonucleotide
ctggcacgtatttagccatc

SEQ ID NO: 405 - DNA, 20 bp, Artificial
  oligonucleotide
acccgaagatcaattcagcg

SEQ ID NO: 406 - DNA, 20 bp, Artificial
  oligonucleotide
tctagcgatccaaaaggacc

SEQ ID NO: 407 - DNA, 20 bp, Artificial
  oligonucleotide
ctagcgatccaaaaggacct

SEQ ID NO: 408 - DNA, 20 bp, Artificial
  oligonucleotide
gcacccatcgtttacggtat

SEQ ID NO: 409 - DNA, 20 bp, Artificial
  oligonucleotide
cacccatcgtttacggtatg

SEQ ID NO: 410 - DNA, 20 bp, Artificial
  oligonucleotide
gccactctagtcattgcctc

SEQ ID NO: 411 - DNA, 20 bp, Artificial
  oligonucleotide
cgtttgctacgtcactagga

SEQ ID NO: 412 - DNA, 20 bp, Artificial
  oligonucleotide
gcctcaacgtcagttacgat

SEQ ID NO: 413 - DNA, 20 bp, Artificial
  oligonucleotide
gccggccagtctctcaactc

SEQ ID NO: 414 - DNA, 20 bp, Artificial
  oligonucleotide
tcactaggaggcggaaacct

SEQ ID NO: 415 - DNA, 20 bp, Artificial
  oligonucleotide
agcctcaacgtcagttacga

SEQ ID NO: 416 - DNA, 20 bp, Artificial
  oligonucleotide
agccagtctctcaactcggc

SEQ ID NO: 417 - DNA, 20 bp, Artificial
  oligonucleotide
ggccagtctctcaactcggc

SEQ ID NO: 418 - DNA, 20 bp, Artificial
  oligonucleotide
caagctaatacgccgcaaga

SEQ ID NO: 419 - DNA, 20 bp, Artificial
  oligonucleotide
ttcgccactctagtcattgc

SEQ ID NO: 420 - DNA, 20 bp, Artificial
  oligonucleotide
ccgaagatcaattcagcggc

SEQ ID NO: 421 - DNA, 20 bp, Artificial
  oligonucleotide
cgcaagaccatcctctagcg

SEQ ID NO: 422 - DNA, 20 bp, Artificial
  oligonucleotide
gcaagaccatcctctagcga

SEQ ID NO: 423 - DNA, 20 bp, Artificial
  oligonucleotide
gcgtttgctacgtcactagg

SEQ ID NO: 424 - DNA, 20 bp, Artificial
  oligonucleotide
ccactctagtcattgcctca

SEQ ID NO: 425 - DNA, 20 bp, Artificial
  oligonucleotide
cactctagtcattgcctcac

SEQ ID NO: 426 - DNA, 20 bp, Artificial
  oligonucleotide
ccagtctctcaactcggcta

SEQ ID NO: 427 - DNA, 20 bp, Artificial
  oligonucleotide
ttaccttaggcaccggcctc

SEQ ID NO: 428 - DNA, 20 bp, Artificial
  oligonucleotide
acaagctaatacgccgcaag

SEQ ID NO: 429 - DNA, 20 bp, Artificial
  oligonucleotide
tttaccttaggcaccggcct

SEQ ID NO: 430 - DNA, 20 bp, Artificial
  oligonucleotide
ttttaccttaggcaccggcc

SEQ ID NO: 431 - DNA, 20 bp, Artificial
  oligonucleotide
attttaccttaggcaccggc

SEQ ID NO: 432 - DNA, 20 bp, Artificial
  oligonucleotide
gattttaccttaggcaccgg

SEQ ID NO: 433 - DNA, 20 bp, Artificial
  oligonucleotide
ctcacttcacccgaagatca

SEQ ID NO: 434 - DNA, 20 bp, Artificial
  oligonucleotide
acgccaccagcgttcatcct

SEQ ID NO: 435 - DNA, 20 bp, Artificial
  oligonucleotide
gccaagcgactttgggtact

SEQ ID NO: 436 - DNA, 20 bp, Artificial
  oligonucleotide
cggaaaattccctactgcag

SEQ ID NO: 437 - DNA, 20 bp, Artificial
  oligonucleotide
cgatctagcaagccgctttc

SEQ ID NO: 438 - DNA, 20 bp, Artificial
  oligonucleotide
ggtaccgtcaagctgaaaac

SEQ ID NO: 439 - DNA, 20 bp, Artificial
  oligonucleotide
tgcctcacttcacccgaaga

SEQ ID NO: 440 - DNA, 20 bp, Artificial
  oligonucleotide
ggccggccagtctctcaact

SEQ ID NO: 441 - DNA, 20 bp, Artificial
  oligonucleotide
ggtaaggtaccgtcaagctg

SEQ ID NO: 442 - DNA, 20 bp, Artificial
  oligonucleotide
gtaaggtaccgtcaagctga

SEQ ID NO: 443 - DNA, 20 bp, Artificial
  oligonucleotide
ccgcaagaccatcctctagg

SEQ ID NO: 444 - DNA, 20 bp, Artificial
  oligonucleotide
atttagccatccctttctgg

SEQ ID NO: 445 - DNA, 18 bp, Artificial
  oligonucleotide
aacccttcatcacacacg

SEQ ID NO: 446 - DNA, 18 bp, Artificial
  oligonucleotide
cgaaacccttcatcacac

SEQ ID NO: 447 - DNA, 18 bp, Artificial
  oligonucleotide
acccttcatcacacacgc

SEQ ID NO: 448 - DNA, 18 bp, Artificial
  oligonucleotide
taccgtcacacactgaac

SEQ ID NO: 449 - DNA, 18 bp, Artificial
  oligonucleotide
agataccgtcacacactg

SEQ ID NO: 450 - DNA, 18 bp, Artificial
  oligonucleotide
cactcaagggcggaaacc

SEQ ID NO: 451 - DNA, 18 bp, Artificial
  oligonucleotide
accgtcacacactgaaca

SEQ ID NO: 452 - DNA, 18 bp, Artificial
  oligonucleotide
cgtcacacactgaacagt

SEQ ID NO: 453 - DNA, 18 bp, Artificial
  oligonucleotide
ccgaaacccttcatcaca

SEQ ID NO: 454 - DNA, 18 bp, Artificial
  oligonucleotide
ccgtcacacactgaacag

SEQ ID NO: 455 - DNA, 18 bp, Artificial
  oligonucleotide
gataccgtcacacactga

SEQ ID NO: 456 - DNA, 18 bp, Artificial
  oligonucleotide
ggtaagataccgtcacac

SEQ ID NO: 457 - DNA, 18 bp, Artificial
  oligonucleotide
cccttcatcacacacgcg

SEQ ID NO: 458 - DNA, 18 bp, Artificial
  oligonucleotide
acagtgttttacgagccg

SEQ ID NO: 459 - DNA, 18 bp, Artificial
  oligonucleotide
cagtgttttacgagccga

SEQ ID NO: 460 - DNA, 18 bp, Artificial
  oligonucleotide
acaaagcgttcgacttgc

SEQ ID NO: 461 - DNA, 18 bp, Artificial
  oligonucleotide
cggataacgcttggaaca

SEQ ID NO: 462 - DNA, 18 bp, Artificial
  oligonucleotide
agggcggaaaccctcgaa

SEQ ID NO: 463 - DNA, 18 bp, Artificial
  oligonucleotide
gggcggaaaccctcgaac

SEQ ID NO: 464 - DNA, 18 bp, Artificial
  oligonucleotide
ggcggaaaccctcgaaca

SEQ ID NO: 465 - DNA, 18 bp, Artificial
  oligonucleotide
tgagggctttcacttcag

SEQ ID NO: 466 - DNA, 18 bp, Artificial
  oligonucleotide
agggctttcacttcagac

SEQ ID NO: 467 - DNA, 18 bp, Artificial
  oligonucleotide
gagggctttcacttcaga

SEQ ID NO: 468 - DNA, 18 bp, Artificial
  oligonucleotide
actgcactcaagtcatcc

SEQ ID NO: 469 - DNA, 18 bp, Artificial
  oligonucleotide
ccggataacgcttggaac

SEQ ID NO: 470 - DNA, 18 bp, Artificial
  oligonucleotide
tccggataacgcttggaa

SEQ ID NO: 471 - DNA, 18 bp, Artificial
  oligonucleotide
tatcccctgctaagaggt

SEQ ID NO: 472 - DNA, 18 bp, Artificial
  oligonucleotide
cctgctaagaggtaggtt

SEQ ID NO: 473 - DNA, 18 bp, Artificial
  oligonucleotide
ccctgctaagaggtaggt

SEQ ID NO: 474 - DNA, 18 bp, Artificial
  oligonucleotide
cccctgctaagaggtagg

SEQ ID NO: 475 - DNA, 18 bp, Artificial
  oligonucleotide
tcccctgctaagaggtag

SEQ ID NO: 476 - DNA, 18 bp, Artificial
  oligonucleotide
atcccctgctaagaggta

SEQ ID NO: 477 - DNA, 18 bp, Artificial
  oligonucleotide
ccgttcctttctggtaag

SEQ ID NO: 478 - DNA, 18 bp, Artificial
  oligonucleotide
gccgttcctttctggtaa

SEQ ID NO: 479 - DNA, 18 bp, Artificial
  oligonucleotide
agccgttcctttctggta

SEQ ID NO: 480 - DNA, 18 bp, Artificial
  oligonucleotide
gcacgtatttagccgttc

SEQ ID NO: 481 - DNA, 18 bp, Artificial
  oligonucleotide
cacgtatttagccgttcc

SEQ ID NO: 482 - DNA, 18 bp, Artificial
  oligonucleotide
ggcacgtatttagccgtt

SEQ ID NO: 483 - DNA, 18 bp, Artificial
  oligonucleotide
cactttcctctactgcac

SEQ ID NO: 484 - DNA, 18 bp, Artificial
  oligonucleotide
ccactttcctctactgca

SEQ ID NO: 485 - DNA, 18 bp, Artificial
  oligonucleotide
tccactttcctctactgc

SEQ ID NO: 486 - DNA, 18 bp, Artificial
  oligonucleotide
ctttcctctactgcactc

SEQ ID NO: 487 - DNA, 18 bp, Artificial
  oligonucleotide
tagccgttcctttctggt

SEQ ID NO: 488 - DNA, 18 bp, Artificial
  oligonucleotide
ttagccgttcctttctgg

SEQ ID NO: 489 - DNA, 18 bp, Artificial
  oligonucleotide
ttatcccctgctaagagg

SEQ ID NO: 490 - DNA, 18 bp, Artificial
  oligonucleotide
gttatcccctgctaagag

SEQ ID NO: 491 - DNA, 18 bp, Artificial
  oligonucleotide
cccgttcgccactctttg

SEQ ID NO: 492 - DNA, 18 bp, Artificial
  oligonucleotide
agctgagggctttcactt

SEQ ID NO: 493 - DNA, 18 bp, Artificial
  oligonucleotide
gagctgagggctttcact

SEQ ID NO: 494 - DNA, 18 bp, Artificial
  oligonucleotide
gctgagggctttcacttc

SEQ ID NO: 495 - DNA, 18 bp, Artificial
  oligonucleotide
ctgagggctttcacttca

SEQ ID NO: 496 - DNA, 18 bp, Artificial
  oligonucleotide
cccgtgtcccgaaggaac

SEQ ID NO: 497 - DNA, 18 bp, Artificial
  oligonucleotide
gcacgagtatgtcaagac

SEQ ID NO: 498 - DNA, 18 bp, Artificial
  oligonucleotide
gtatcccgtgtcccgaag

SEQ ID NO: 499 - DNA, 18 bp, Artificial
  oligonucleotide
tcccgtgtcccgaaggaa

SEQ ID NO: 500 - DNA, 18 bp, Artificial
  oligonucleotide
atcccgtgtcccgaagga

SEQ ID NO: 501 - DNA, 18 bp, Artificial
  oligonucleotide
tatcccgtgtcccgaagg

SEQ ID NO: 502 - DNA, 18 bp, Artificial
  oligonucleotide
cttaccttaggaagcgcc

SEQ ID NO: 503 - DNA, 18 bp, Artificial
  oligonucleotide
ttaccttaggaagcgccc

SEQ ID NO: 504 - DNA, 18 bp, Artificial
  oligonucleotide
cctgtatcccgtgtcccg

SEQ ID NO: 505 - DNA, 18 bp, Artificial
  oligonucleotide
ccacctgtatcccgtgtc

SEQ ID NO: 506 - DNA, 18 bp, Artificial
  oligonucleotide
cacctgtatcccgtgtcc

SEQ ID NO: 507 - DNA, 18 bp, Artificial
  oligonucleotide
acctgtatcccgtgtccc

SEQ ID NO: 508 - DNA, 18 bp, Artificial
  oligonucleotide
ctgtatcccgtgtcccga

SEQ ID NO: 509 - DNA, 18 bp, Artificial
  oligonucleotide
tgtatcccgtgtcccgaa

SEQ ID NO: 510 - DNA, 18 bp, Artificial
  oligonucleotide
cacgagtatgtcaagacc

SEQ ID NO: 511 - DNA, 18 bp, Artificial
  oligonucleotide
cggtcttaccttaggaag

SEQ ID NO: 512 - DNA, 18 bp, Artificial
  oligonucleotide
taggaagcgccctccttg

SEQ ID NO: 513 - DNA, 18 bp, Artificial
  oligonucleotide
aggaagcgccctccttgc

SEQ ID NO: 514 - DNA, 18 bp, Artificial
  oligonucleotide
ttaggaagcgccctcctt

SEQ ID NO: 515 - DNA, 18 bp, Artificial
  oligonucleotide
cttaggaagcgccctcct

SEQ ID NO: 516 - DNA, 18 bp, Artificial
  oligonucleotide
ccttaggaagcgccctcc

SEQ ID NO: 517 - DNA, 18 bp, Artificial
  oligonucleotide
accttaggaagcgccctc

SEQ ID NO: 518 - DNA, 18 bp, Artificial
  oligonucleotide
tgcacacaatggttgagc

SEQ ID NO: 519 - DNA, 18 bp, Artificial
  oligonucleotide
taccttaggaagcgccct

SEQ ID NO: 520 - DNA, 18 bp, Artificial
  oligonucleotide
accacctgtatcccgtgt

SEQ ID NO: 521 - DNA, 18 bp, Artificial
  oligonucleotide
gcaccacctgtatcccgt

SEQ ID NO: 522 - DNA, 18 bp, Artificial
  oligonucleotide
caccacctgtatcccgtg

SEQ ID NO: 523 - DNA, 18 bp, Artificial
  oligonucleotide
gcggttaggcaacctact

SEQ ID NO: 524 - DNA, 18 bp, Artificial
  oligonucleotide
tgcggttaggcaacctac

SEQ ID NO: 525 - DNA, 18 bp, Artificial
  oligonucleotide
ttgcggttaggcaaccta

SEQ ID NO: 526 - DNA, 18 bp, Artificial
  oligonucleotide
ggtcttaccttaggaagc

SEQ ID NO: 527 - DNA, 18 bp, Artificial
  oligonucleotide
gctaatacaacgcgggat

SEQ ID NO: 528 - DNA, 18 bp, Artificial
  oligonucleotide
ctaatacaacgcgggatc

SEQ ID NO: 529 - DNA, 18 bp, Artificial
  oligonucleotide
atacaacgcgggatcatc

SEQ ID NO: 530 - DNA, 18 bp, Artificial
  oligonucleotide
cggttaggcaacctactt

SEQ ID NO: 531 - DNA, 18 bp, Artificial
  oligonucleotide
tgcaccacctgtatcccg

SEQ ID NO: 532 - DNA, 18 bp, Artificial
  oligonucleotide
gaagcgccctccttgcgg

SEQ ID NO: 533 - DNA, 18 bp, Artificial
  oligonucleotide
ggaagcgccctccttgcg

SEQ ID NO: 534 - DNA, 18 bp, Artificial
  oligonucleotide
cgtccctttctggttaga

SEQ ID NO: 535 - DNA, 18 bp, Artificial
  oligonucleotide
agctaatacaacgcggga

SEQ ID NO: 536 - DNA, 18 bp, Artificial
  oligonucleotide
tagctaatacaacgcggg

SEQ ID NO: 537 - DNA, 18 bp, Artificial
  oligonucleotide
ctagctaatacaacgcgg

SEQ ID NO: 538 - DNA, 18 bp, Artificial
  oligonucleotide
ggctatgtatcatcgcct

SEQ ID NO: 539 - DNA, 18 bp, Artificial
  oligonucleotide
gagccactgccttttaca

SEQ ID NO: 540 - DNA, 18 bp, Artificial
  oligonucleotide
gtcggctatgtatcatcg

SEQ ID NO: 541 - DNA, 18 bp, Artificial
  oligonucleotide
ggtcggctatgtatcatc

SEQ ID NO: 542 - DNA, 18 bp, Artificial
  oligonucleotide
caggtcggctatgtatca

SEQ ID NO: 543 - DNA, 18 bp, Artificial
  oligonucleotide
cggctatgtatcatcgcc

SEQ ID NO: 544 - DNA, 18 bp, Artificial
  oligonucleotide
tcggctatgtatcatcgc

SEQ ID NO: 545 - DNA, 18 bp, Artificial
  oligonucleotide
gtcttaccttaggaagcg

SEQ ID NO: 546 - DNA, 18 bp, Artificial
  oligonucleotide
tcttaccttaggaagcgc

SEQ ID NO: 547 - DNA, 20 bp, Artificial
  oligonucleotide
gtacaaaccgcctacacgcc

SEQ ID NO: 548 - DNA, 20 bp, Artificial
  oligonucleotide
tgtacaaaccgcctacacgc

SEQ ID NO: 549 - DNA, 20 bp, Artificial
  oligonucleotide
gatcagcacgatgtcgccat

SEQ ID NO: 550 - DNA, 20 bp, Artificial
  oligonucleotide
ctgtacaaaccgcctacacg

SEQ ID NO: 551 - DNA, 20 bp, Artificial
  oligonucleotide
gagatcagcacgatgtcgcc

SEQ ID NO: 552 - DNA, 20 bp, Artificial
  oligonucleotide
agatcagcacgatgtcgcca

SEQ ID NO: 553 - DNA, 20 bp, Artificial
  oligonucleotide
atcagcacgatgtcgccatc

SEQ ID NO: 554 - DNA, 20 bp, Artificial
  oligonucleotide
tcagcacgatgtcgccatct

SEQ ID NO: 555 - DNA, 20 bp, Artificial
  oligonucleotide
actgtacaaaccgcctacac

SEQ ID NO: 556 - DNA, 20 bp, Artificial
  oligonucleotide
ccgccactaaggccgaaacc

SEQ ID NO: 557 - DNA, 20 bp, Artificial
  oligonucleotide
cagcacgatgtcgccatcta

SEQ ID NO: 558 - DNA, 20 bp, Artificial
  oligonucleotide
tacaaaccgcctacacgccc

SEQ ID NO: 559 - DNA, 20 bp, Artificial
  oligonucleotide
agcacgatgtcgccatctag

SEQ ID NO: 560 - DNA, 20 bp, Artificial
  oligonucleotide
cggcttttagagatcagcac

SEQ ID NO: 561 - DNA, 20 bp, Artificial
  oligonucleotide
tccgccactaaggccgaaac

SEQ ID NO: 562 - DNA, 20 bp, Artificial
  oligonucleotide
gactgtacaaaccgcctaca

SEQ ID NO: 563 - DNA, 20 bp, Artificial
  oligonucleotide
gtccgccactaaggccgaaa

SEQ ID NO: 564 - DNA, 20 bp, Artificial
  oligonucleotide
ggggatttcacatctgactg

SEQ ID NO: 565 - DNA, 20 bp, Artificial
  oligonucleotide
catacaagccctggtaaggt

SEQ ID NO: 566 - DNA, 20 bp, Artificial
  oligonucleotide
acaagccctggtaaggttct

SEQ ID NO: 567 - DNA, 20 bp, Artificial
  oligonucleotide
acaaaccgcctacacgccct

SEQ ID NO: 568 - DNA, 20 bp, Artificial
  oligonucleotide
ctgactgtacaaaccgccta

SEQ ID NO: 569 - DNA, 20 bp, Artificial
  oligonucleotide
tgactgtacaaaccgcctac

SEQ ID NO: 570 - DNA, 20 bp, Artificial
  oligonucleotide
acgatgtcgccatctagctt

SEQ ID NO: 571 - DNA, 20 bp, Artificial
  oligonucleotide
cacgatgtcgccatctagct

SEQ ID NO: 572 - DNA, 20 bp, Artificial
  oligonucleotide
cgatgtcgccatctagcttc

SEQ ID NO: 573 - DNA, 20 bp, Artificial
  oligonucleotide
gcacgatgtcgccatctagc

SEQ ID NO: 574 - DNA, 20 bp, Artificial
  oligonucleotide
gatgtcgccatctagcttcc

SEQ ID NO: 575 - DNA, 20 bp, Artificial
  oligonucleotide
atgtcgccatctagcttccc

SEQ ID NO: 576 - DNA, 20 bp, Artificial
  oligonucleotide
tgtcgccatctagcttccca

SEQ ID NO: 577 - DNA, 20 bp, Artificial
  oligonucleotide
gccatctagcttcccactgt

SEQ ID NO: 578 - DNA, 20 bp, Artificial
  oligonucleotide
tcgccatctagcttcccact

SEQ ID NO: 579 - DNA, 20 bp, Artificial
  oligonucleotide
cgccatctagcttcccactg

SEQ ID NO: 580 - DNA, 20 bp, Artificial
  oligonucleotide
gtcgccatctagcttcccac

SEQ ID NO: 581 - DNA, 20 bp, Artificial
  oligonucleotide
tacaagccctggtaaggttc

SEQ ID NO: 582 - DNA, 20 bp, Artificial
  oligonucleotide
gccactaaggccgaaacctt

SEQ ID NO: 583 - DNA, 20 bp, Artificial
  oligonucleotide
actaaggccgaaaccttcgt

SEQ ID NO: 584 - DNA, 20 bp, Artificial
  oligonucleotide
ctaaggccgaaaccttcgtg

SEQ ID NO: 585 - DNA, 20 bp, Artificial
  oligonucleotide
cactaaggccgaaaccttcg

SEQ ID NO: 586 - DNA, 20 bp, Artificial
  oligonucleotide
aaggccgaaaccttcgtgcg

SEQ ID NO: 587 - DNA, 20 bp, Artificial
  oligonucleotide
ccactaaggccgaaaccttc

SEQ ID NO: 588 - DNA, 20 bp, Artificial
  oligonucleotide
taaggccgaaaccttcgtgc

SEQ ID NO: 589 - DNA, 20 bp, Artificial
  oligonucleotide
aggccgaaaccttcgtgcga

SEQ ID NO: 590 - DNA, 20 bp, Artificial
  oligonucleotide
tctgactgtacaaaccgcct

SEQ ID NO: 591 - DNA, 20 bp, Artificial
  oligonucleotide
catctgactgtacaaaccgc

SEQ ID NO: 592 - DNA, 20 bp, Artificial
  oligonucleotide
atctgactgtacaaaccgcc

SEQ ID NO: 593 - DNA, 20 bp, Artificial
  oligonucleotide
cttcgtgcgacttgcatgtg

SEQ ID NO: 594 - DNA, 20 bp, Artificial
  oligonucleotide
ccttcgtgcgacttgcatgt

SEQ ID NO: 595 - DNA, 20 bp, Artificial
  oligonucleotide
ctctctagagtgcccaccca

SEQ ID NO: 596 - DNA, 20 bp, Artificial
  oligonucleotide
tctctagagtgcccacccaa

SEQ ID NO: 597 - DNA, 20 bp, Artificial
  oligonucleotide
acgtatcaaatgcagctccc

SEQ ID NO: 598 - DNA, 20 bp, Artificial
  oligonucleotide
cgtatcaaatgcagctccca

SEQ ID NO: 599 - DNA, 20 bp, Artificial
  oligonucleotide
cgccactaaggccgaaacct

SEQ ID NO: 600 - DNA, 20 bp, Artificial
  oligonucleotide
ccgaaaccttcgtgcgactt

SEQ ID NO: 601 - DNA, 20 bp, Artificial
  oligonucleotide
gccgaaaccttcgtgcgact

SEQ ID NO: 602 - DNA, 20 bp, Artificial
  oligonucleotide
aaccttcgtgcgacttgcat

SEQ ID NO: 603 - DNA, 20 bp, Artificial
  oligonucleotide
cgaaaccttcgtgcgacttg

SEQ ID NO: 604 - DNA, 20 bp, Artificial
  oligonucleotide
accttcgtgcgacttgcatg

SEQ ID NO: 605 - DNA, 20 bp, Artificial
  oligonucleotide
gaaaccttcgtgcgacttgc

SEQ ID NO: 606 - DNA, 20 bp, Artificial
  oligonucleotide
ggccgaaaccttcgtgcgac

SEQ ID NO: 607 - DNA, 20 bp, Artificial
  oligonucleotide
aaaccttcgtgcgacttgca

SEQ ID NO: 608 - DNA, 20 bp, Artificial
  oligonucleotide
cacgtatcaaatgcagctcc

SEQ ID NO: 609 - DNA, 20 bp, Artificial
  oligonucleotide
gctcaccggcttaaggtcaa

SEQ ID NO: 610 - DNA, 20 bp, Artificial
  oligonucleotide
cgctcaccggcttaaggtca

SEQ ID NO: 611 - DNA, 20 bp, Artificial
  oligonucleotide
tcgctcaccggcttaaggtc

SEQ ID NO: 612 - DNA, 20 bp, Artificial
  oligonucleotide
ctcaccggcttaaggtcaaa

SEQ ID NO: 613 - DNA, 20 bp, Artificial
  oligonucleotide
cccgaccgtggtcggctgcg

SEQ ID NO: 614 - DNA, 20 bp, Artificial
  oligonucleotide
gctcaccggcttaaggtcaa

SEQ ID NO: 615 - DNA, 20 bp, Artificial
  oligonucleotide
cgctcaccggcttaaggtca

SEQ ID NO: 616 - DNA, 20 bp, Artificial
  oligonucleotide
tcgctcaccggcttaaggtc

SEQ ID NO: 617 - DNA, 20 bp, Artificial
  oligonucleotide
ctcaccggcttaaggtcaaa

SEQ ID NO: 618 - DNA, 20 bp, Artificial
  oligonucleotide
cccgaccgtggtcggctgcg

SEQ ID NO: 619 - DNA, 20 bp, Artificial
  oligonucleotide
tcaccggcttaaggtcaaac

SEQ ID NO: 620 - DNA, 20 bp, Artificial
  oligonucleotide
caaccctctctcacactcta

SEQ ID NO: 621 - DNA, 20 bp, Artificial
  oligonucleotide
acaaccctctctcacactct

SEQ ID NO: 622 - DNA, 20 bp, Artificial
  oligonucleotide
ccacaaccctctctcacact

SEQ ID NO: 623 - DNA, 20 bp, Artificial
  oligonucleotide
aaccctctctcacactctag

SEQ ID NO: 624 - DNA, 20 bp, Artificial
  oligonucleotide
cacaaccctctctcacactc

SEQ ID NO: 625 - DNA, 20 bp, Artificial
  oligonucleotide
tccacaaccctctctcacac

SEQ ID NO: 626 - DNA, 20 bp, Artificial
  oligonucleotide
ttccacaaccctctctcaca

SEQ ID NO: 627 - DNA, 20 bp, Artificial
  oligonucleotide
accctctctcacactctagt

SEQ ID NO: 628 - DNA, 20 bp, Artificial
  oligonucleotide
gagccaggttgccgccttcg

SEQ ID NO: 629 - DNA, 20 bp, Artificial
  oligonucleotide
aggtcaaaccaactcccatg

SEQ ID NO: 630 - DNA, 20 bp, Artificial
  oligonucleotide
atgagccaggttgccgcctt

SEQ ID NO: 631 - DNA, 20 bp, Artificial
  oligonucleotide
tgagccaggttgccgccttc

SEQ ID NO: 632 - DNA, 20 bp, Artificial
  oligonucleotide
aggctcctccacaggcgact

SEQ ID NO: 633 - DNA, 20 bp, Artificial
  oligonucleotide
caggctcctccacaggcgac

SEQ ID NO: 634 - DNA, 20 bp, Artificial
  oligonucleotide
gcaggctcctccacaggcga

SEQ ID NO: 635 - DNA, 20 bp, Artificial
  oligonucleotide
ttcgctcaccggcttaaggt

SEQ ID NO: 636 - DNA, 20 bp, Artificial
  oligonucleotide
gttcgctcaccggcttaagg

SEQ ID NO: 637 - DNA, 20 bp, Artificial
  oligonucleotide
ggttcgctcaccggcttaag

SEQ ID NO: 638 - DNA, 20 bp, Artificial
  oligonucleotide
attccacaaccctctctcac

SEQ ID NO: 639 - DNA, 20 bp, Artificial
  oligonucleotide
tgacccgaccgtggtcggct

SEQ ID NO: 640 - DNA, 20 bp, Artificial
  oligonucleotide
ccctctctcacactctagtc

SEQ ID NO: 641 - DNA, 20 bp, Artificial
  oligonucleotide
gaattccacaaccctctctc

SEQ ID NO: 642 - DNA, 20 bp, Artificial
  oligonucleotide
agccaggttgccgccttcgc

SEQ ID NO: 643 - DNA, 20 bp, Artificial
  oligonucleotide
gccaggttgccgccttcgcc

SEQ ID NO: 644 - DNA, 20 bp, Artificial
  oligonucleotide
ggaattccacaaccctctct

SEQ ID NO: 645 - DNA, 20 bp, Artificial
  oligonucleotide
gggaattccacaaccctctc

SEQ ID NO: 646 - DNA, 20 bp, Artificial
  oligonucleotide
aacgcaggctcctccacagg

SEQ ID NO: 647 - DNA, 20 bp, Artificial
  oligonucleotide
cggcttaaggtcaaaccaac

SEQ ID NO: 648 - DNA, 20 bp, Artificial
  oligonucleotide
ccggcttaaggtcaaaccaa

SEQ ID NO: 649 - DNA, 20 bp, Artificial
  oligonucleotide
caccggcttaaggtcaaacc

SEQ ID NO: 650 - DNA, 20 bp, Artificial
  oligonucleotide
accggcttaaggtcaaacca

SEQ ID NO: 651 - DNA, 20 bp, Artificial
  oligonucleotide
acccaacatccagcacacat

SEQ ID NO: 652 - DNA, 20 bp, Artificial
  oligonucleotide
tcgctgacccgaccgtggtc

SEQ ID NO: 653 - DNA, 20 bp, Artificial
  oligonucleotide
cgctgacccgaccgtggtcg

SEQ ID NO: 654 - DNA, 20 bp, Artificial
  oligonucleotide
gacccgaccgtggtcggctg

SEQ ID NO: 655 - DNA, 20 bp, Artificial
  oligonucleotide
gctgacccgaccgtggtcgg

SEQ ID NO: 656 - DNA, 20 bp, Artificial
  oligonucleotide
ctgacccgaccgtggtcggc

SEQ ID NO: 657 - DNA, 20 bp, Artificial
  oligonucleotide
caggcgacttgcgcctttga

SEQ ID NO: 658 - DNA, 20 bp, Artificial
  oligonucleotide
tcatgcggtattagctccag

SEQ ID NO: 659 - DNA, 20 bp, Artificial
  oligonucleotide
actagctaatcgaacgcagg

SEQ ID NO: 660 - DNA, 20 bp, Artificial
  oligonucleotide
catgcggtattagctccagt

SEQ ID NO: 661 - DNA, 20 bp, Artificial
  oligonucleotide
cgcaggctcctccacaggcg

SEQ ID NO: 662 - DNA, 20 bp, Artificial
  oligonucleotide
acgcaggctcctccacaggc

SEQ ID NO: 663 - DNA, 20 bp, Artificial
  oligonucleotide
ctcaggtgtcatgcggtatt

SEQ ID NO: 664 - DNA, 20 bp, Artificial
  oligonucleotide
cgcctttgaccctcaggtgt

SEQ ID NO: 665 - DNA, 20 bp, Artificial
  oligonucleotide
accctcaggtgtcatgcggt

SEQ ID NO: 666 - DNA, 20 bp, Artificial
  oligonucleotide
cctcaggtgtcatgcggtat

SEQ ID NO: 667 - DNA, 20 bp, Artificial
  oligonucleotide
tttgaccctcaggtgtcatg

SEQ ID NO: 668 - DNA, 20 bp, Artificial
  oligonucleotide
gaccctcaggtgtcatgcgg

SEQ ID NO: 669 - DNA, 20 bp, Artificial
  oligonucleotide
tgaccctcaggtgtcatgcg

SEQ ID NO: 670 - DNA, 20 bp, Artificial
  oligonucleotide
gcctttgaccctcaggtgtc

SEQ ID NO: 671 - DNA, 20 bp, Artificial
  oligonucleotide
ttgaccctcaggtgtcatgc

SEQ ID NO: 672 - DNA, 20 bp, Artificial
  oligonucleotide
ccctcaggtgtcatgcggta

SEQ ID NO: 673 - DNA, 20 bp, Artificial
  oligonucleotide
cctttgaccctcaggtgtca

SEQ ID NO: 674 - DNA, 20 bp, Artificial
  oligonucleotide
ctttgaccctcaggtgtcat

SEQ ID NO: 675 - DNA, 20 bp, Artificial
  oligonucleotide
agttatcccccacccatgga

SEQ ID NO: 676 - DNA, 20 bp, Artificial
  oligonucleotide
ccagctatcgatcatcgcct

SEQ ID NO: 677 - DNA, 20 bp, Artificial
  oligonucleotide
accagctatcgatcatcgcc

SEQ ID NO: 678 - DNA, 20 bp, Artificial
  oligonucleotide
cagctatcgatcatcgcctt

SEQ ID NO: 679 - DNA, 20 bp, Artificial
  oligonucleotide
agctatcgatcatcgccttg

SEQ ID NO: 680 - DNA, 20 bp, Artificial
  oligonucleotide
gctatcgatcatcgccttgg

SEQ ID NO: 681 - DNA, 20 bp, Artificial
  oligonucleotide
ctatcgatcatcgccttggt

SEQ ID NO: 682 - DNA, 20 bp, Artificial
  oligonucleotide
ttcgtgcgacttgcatgtgt

SEQ ID NO: 683 - DNA, 20 bp, Artificial
  oligonucleotide
tcgatcatcgccttggtagg

SEQ ID NO: 684 - DNA, 20 bp, Artificial
  oligonucleotide
atcgatcatcgccttggtag

SEQ ID NO: 685 - DNA, 20 bp, Artificial
  oligonucleotide
cacaggcgacttgcgccttt

SEQ ID NO: 686 - DNA, 20 bp, Artificial
  oligonucleotide
ccacaggcgacttgcgcctt

SEQ ID NO: 687 - DNA, 20 bp, Artificial
  oligonucleotide
tccacaggcgacttgcgcct

SEQ ID NO: 688 - DNA, 20 bp, Artificial
  oligonucleotide
tcctccacaggcgacttgcg

SEQ ID NO: 689 - DNA, 20 bp, Artificial
  oligonucleotide
cctccacaggcgacttgcgc

SEQ ID NO: 690 - DNA, 20 bp, Artificial
  oligonucleotide
ctccacaggcgacttgcgcc

SEQ ID NO: 691 - DNA, 20 bp, Artificial
  oligonucleotide
acaggcgacttgcgcctttg

SEQ ID NO: 692 - DNA, 20 bp, Artificial
  oligonucleotide
gctcaccggcttaaggtcaa

SEQ ID NO: 693 - DNA, 20 bp, Artificial
  oligonucleotide
cgctcaccggcttaaggtca

SEQ ID NO: 694 - DNA, 20 bp, Artificial
  oligonucleotide
tcgctcaccggcttaaggtc

SEQ ID NO: 695 - DNA, 20 bp, Artificial
  oligonucleotide
ctcaccggcttaaggtcaaa

SEQ ID NO: 696 - DNA, 20 bp, Artificial
  oligonucleotide
cccgaccgtggtcggctgcg

SEQ ID NO: 697 - DNA, 20 bp, Artificial
  oligonucleotide
tcaccggcttaaggtcaaac

SEQ ID NO: 698 - DNA, 20 bp, Artificial
  oligonucleotide
caaccctctctcacactcta

SEQ ID NO: 699 - DNA, 20 bp, Artificial
  oligonucleotide
acaaccctctctcacactct

SEQ ID NO: 700 - DNA, 20 bp, Artificial
  oligonucleotide
ccacaaccctctctcacact

SEQ ID NO: 701 - DNA, 20 bp, Artificial
  oligonucleotide
aaccctctctcacactctag

SEQ ID NO: 702 - DNA, 20 bp, Artificial
  oligonucleotide
cacaaccctctctcacactc

SEQ ID NO: 703 - DNA, 20 bp, Artificial
  oligonucleotide
tccacaaccctctctcacac

SEQ ID NO: 704 - DNA, 20 bp, Artificial
  oligonucleotide
ttccacaaccctctctcaca

SEQ ID NO: 705 - DNA, 20 bp, Artificial
  oligonucleotide
accctctctcacactctagt

SEQ ID NO: 706 - DNA, 20 bp, Artificial
  oligonucleotide
gagccaggttgccgccttcg

SEQ ID NO: 707 - DNA, 20 bp, Artificial
  oligonucleotide
aggtcaaaccaactcccatg

SEQ ID NO: 708 - DNA, 20 bp, Artificial
  oligonucleotide
atgagccaggttgccgcctt

SEQ ID NO: 709 - DNA, 20 bp, Artificial
  oligonucleotide
tgagccaggttgccgccttc

SEQ ID NO: 710 - DNA, 20 bp, Artificial
  oligonucleotide
aggctcctccacaggcgact

SEQ ID NO: 711 - DNA, 20 bp, Artificial
  oligonucleotide
caggctcctccacaggcgac

SEQ ID NO: 712 - DNA, 20 bp, Artificial
  oligonucleotide
gcaggctcctccacaggcga

SEQ ID NO: 713 - DNA, 20 bp, Artificial
  oligonucleotide
ttcgctcaccggcttaaggt

SEQ ID NO: 714 - DNA, 20 bp, Artificial
  oligonucleotide
gttcgctcaccggcttaagg

SEQ ID NO: 715 - DNA, 20 bp, Artificial
  oligonucleotide
ggttcgctcaccggcttaag

SEQ ID NO: 716 - DNA, 20 bp, Artificial
  oligonucleotide
attccacaaccctctctcac

SEQ ID NO: 717 - DNA, 20 bp, Artificial
  oligonucleotide
tgacccgaccgtggtcggct

SEQ ID NO: 718 - DNA, 20 bp, Artificial
  oligonucleotide
ccctctctcacactctagtc

SEQ ID NO: 719 - DNA, 20 bp, Artificial
  oligonucleotide
gaattccacaaccctctctc

SEQ ID NO: 720 - DNA, 20 bp, Artificial
  oligonucleotide
agccaggttgccgccttcgc

SEQ ID NO: 721 - DNA, 20 bp, Artificial
  oligonucleotide
gccaggttgccgccttcgcc

SEQ ID NO: 722 - DNA, 20 bp, Artificial
  oligonucleotide
ggaattccacaaccctctct

SEQ ID NO: 723 - DNA, 20 bp, Artificial
  oligonucleotide
gggaattccacaaccctctc

SEQ ID NO: 724 - DNA, 20 bp, Artificial
  oligonucleotide
aacgcaggctcctccacagg

SEQ ID NO: 725 - DNA, 20 bp, Artificial
  oligonucleotide
cggcttaaggtcaaaccaac

SEQ ID NO: 726 - DNA, 20 bp, Artificial
  oligonucleotide
ccggcttaaggtcaaaccaa

SEQ ID NO: 727 - DNA, 20 bp, Artificial
  oligonucleotide
caccggcttaaggtcaaacc

SEQ ID NO: 728 - DNA, 20 bp, Artificial
  oligonucleotide
accggcttaaggtcaaacca

SEQ ID NO: 729 - DNA, 20 bp, Artificial
  oligonucleotide
acccaacatccagcacacat

SEQ ID NO: 730 - DNA, 20 bp, Artificial
  oligonucleotide
tcgctgacccgaccgtggtc

SEQ ID NO: 731 - DNA, 20 bp, Artificial
  oligonucleotide
cgctgacccgaccgtggtcg

SEQ ID NO: 732 - DNA, 20 bp, Artificial
  oligonucleotide
gacccgaccgtggtcggctg

SEQ ID NO: 733 - DNA, 20 bp, Artificial
  oligonucleotide
gctgacccgaccgtggtcgg

SEQ ID NO: 734 - DNA, 20 bp, Artificial
  oligonucleotide
ctgacccgaccgtggtcggc

SEQ ID NO: 735 - DNA, 20 bp, Artificial
  oligonucleotide
caggcgacttgcgcctttga

SEQ ID NO: 736 - DNA, 20 bp, Artificial
  oligonucleotide
tcatgcggtattagctccag

SEQ ID NO: 737 - DNA, 20 bp, Artificial
  oligonucleotide
actagctaatcgaacgcagg

SEQ ID NO: 738 - DNA, 20 bp, Artificial
  oligonucleotide
catgcggtattagctccagt

SEQ ID NO: 739 - DNA, 20 bp, Artificial
  oligonucleotide
cgcaggctcctccacaggcg

SEQ ID NO: 740 - DNA, 20 bp, Artificial
  oligonucleotide
acgcaggctcctccacaggc

SEQ ID NO: 741 - DNA, 20 bp, Artificial
  oligonucleotide
ctcaggtgtcatgcggtatt

SEQ ID NO: 742 - DNA, 20 bp, Artificial
  oligonucleotide
cgcctttgaccctcaggtgt

SEQ ID NO: 743 - DNA, 20 bp, Artificial
  oligonucleotide
accctcaggtgtcatgcggt

SEQ ID NO: 744 - DNA, 20 bp, Artificial
  oligonucleotide
cctcaggtgtcatgcggtat

SEQ ID NO: 745 - DNA, 20 bp, Artificial
  oligonucleotide
tttgaccctcaggtgtcatg

SEQ ID NO: 746 - DNA, 20 bp, Artificial
  oligonucleotide
gaccctcaggtgtcatgcgg

SEQ ID NO: 747 - DNA, 20 bp, Artificial
  oligonucleotide
tgaccctcaggtgtcatgcg

SEQ ID NO: 748 - DNA, 20 bp, Artificial
  oligonucleotide
gcctttgaccctcaggtgtc

SEQ ID NO: 749 - DNA, 20 bp, Artificial
  oligonucleotide
ttgaccctcaggtgtcatgc

SEQ ID NO: 750 - DNA, 20 bp, Artificial
  oligonucleotide
ccctcaggtgtcatgcggta

SEQ ID NO: 751 - DNA, 20 bp, Artificial
  oligonucleotide
cctttgaccctcaggtgtca

SEQ ID NO: 752 - DNA, 20 bp, Artificial
  oligonucleotide
ctttgaccctcaggtgtcat

SEQ ID NO: 753 - DNA, 20 bp, Artificial
  oligonucleotide
agttatcccccacccatgga

SEQ ID NO: 754 - DNA, 20 bp, Artificial
  oligonucleotide
ccagctatcgatcatcgcct

SEQ ID NO: 755 - DNA, 20 bp, Artificial
  oligonucleotide
accagctatcgatcatcgcc

SEQ ID NO: 756 - DNA, 20 bp, Artificial
  oligonucleotide
cagctatcgatcatcgcctt

SEQ ID NO: 757 - DNA, 20 bp, Artificial
  oligonucleotide
agctatcgatcatcgccttg

SEQ ID NO: 758 - DNA, 20 bp, Artificial
  oligonucleotide
gctatcgatcatcgccttgg

SEQ ID NO: 759 - DNA, 20 bp, Artificial
  oligonucleotide
ctatcgatcatcgccttggt

SEQ ID NO: 760 - DNA, 20 bp, Artificial
  oligonucleotide
ttcgtgcgacttgcatgtgt

SEQ ID NO: 761 - DNA, 20 bp, Artificial
  oligonucleotide
tcgatcatcgccttggtagg

SEQ ID NO: 762 - DNA, 20 bp, Artificial
  oligonucleotide
atcgatcatcgccttggtag

SEQ ID NO: 763 - DNA, 20 bp, Artificial
  oligonucleotide
cacaggcgacttgcgccttt

SEQ ID NO: 764 - DNA, 20 bp, Artificial
  oligonucleotide
ccacaggcgacttgcgcctt

SEQ ID NO: 765 - DNA, 20 bp, Artificial
  oligonucleotide
tccacaggcgacttgcgcct

SEQ ID NO: 766 - DNA, 20 bp, Artificial
  oligonucleotide
tcctccacaggcgacttgcg

SEQ ID NO: 767 - DNA, 20 bp, Artificial
  oligonucleotide
cctccacaggcgacttgcgc

SEQ ID NO: 768 - DNA, 20 bp, Artificial
  oligonucleotide
ctccacaggcgacttgcgcc

SEQ ID NO: 769 - DNA, 20 bp, Artificial
  oligonucleotide
acaggcgacttgcgcctttg

SEQ ID NO: 770 - DNA, 20 bp, Artificial
  oligonucleotide
tcaccggcttaaggtcaaac

SEQ ID NO: 771 - DNA, 20 bp, Artificial
  oligonucleotide
caaccctctctcacactcta

SEQ ID NO: 772 - DNA, 20 bp, Artificial
  oligonucleotide
acaaccctctctcacactct

SEQ ID NO: 773 - DNA, 20 bp, Artificial
  oligonucleotide
ccacaaccctctctcacact

SEQ ID NO: 774 - DNA, 20 bp, Artificial
  oligonucleotide
aaccctctctcacactctag

SEQ ID NO: 775 - DNA, 20 bp, Artificial
  oligonucleotide
cacaaccctctctcacactc

SEQ ID NO: 776 - DNA, 20 bp, Artificial
  oligonucleotide
tccacaaccctctctcacac

SEQ ID NO: 777 - DNA, 20 bp, Artificial
  oligonucleotide
ttccacaaccctctctcaca

SEQ ID NO: 778 - DNA, 20 bp, Artificial
  oligonucleotide
accctctctcacactctagt

SEQ ID NO: 779 - DNA, 20 bp, Artificial
  oligonucleotide
gagccaggttgccgccttcg

SEQ ID NO: 780 - DNA, 20 bp, Artificial
  oligonucleotide
aggtcaaaccaactcccatg

SEQ ID NO: 781 - DNA, 20 bp, Artificial
  oligonucleotide
atgagccaggttgccgcctt

SEQ ID NO: 782 - DNA, 20 bp, Artificial
  oligonucleotide
tgagccaggttgccgccttc

SEQ ID NO: 783 - DNA, 20 bp, Artificial
  oligonucleotide
aggctcctccacaggcgact

SEQ ID NO: 784 - DNA, 20 bp, Artificial
  oligonucleotide
caggctcctccacaggcgac

SEQ ID NO: 785 - DNA, 20 bp, Artificial
  oligonucleotide
gcaggctcctccacaggcga

SEQ ID NO: 786 - DNA, 20 bp, Artificial
  oligonucleotide
ttcgctcaccggcttaaggt

SEQ ID NO: 787 - DNA, 20 bp, Artificial
  oligonucleotide
gttcgctcaccggcttaagg

SEQ ID NO: 788 - DNA, 20 bp, Artificial
  oligonucleotide
ggttcgctcaccggcttaag

SEQ ID NO: 789 - DNA, 20 bp, Artificial
  oligonucleotide
attccacaaccctctctcac

SEQ ID NO: 790 - DNA, 20 bp, Artificial
  oligonucleotide
tgacccgaccgtggtcggct

SEQ ID NO: 791 - DNA, 20 bp, Artificial
  oligonucleotide
ccctctctcacactctagtc

SEQ ID NO: 792 - DNA, 20 bp, Artificial
  oligonucleotide
gaattccacaaccctctctc

SEQ ID NO: 793 - DNA, 20 bp, Artificial
  oligonucleotide
agccaggttgccgccttcgc

SEQ ID NO: 794 - DNA, 20 bp, Artificial
  oligonucleotide
gccaggttgccgccttcgcc

SEQ ID NO: 795 - DNA, 20 bp, Artificial
  oligonucleotide
ggaattccacaaccctctct

SEQ ID NO: 796 - DNA, 20 bp, Artificial
  oligonucleotide
gggaattccacaaccctctc

SEQ ID NO: 797 - DNA, 20 bp, Artificial
  oligonucleotide
aacgcaggctcctccacagg

SEQ ID NO: 798 - DNA, 20 bp, Artificial
  oligonucleotide
cggcttaaggtcaaaccaac

SEQ ID NO: 799 - DNA, 20 bp, Artificial
  oligonucleotide
ccggcttaaggtcaaaccaa

SEQ ID NO: 800 - DNA, 20 bp, Artificial
  oligonucleotide
caccggcttaaggtcaaacc

SEQ ID NO: 801 - DNA, 20 bp, Artificial
  oligonucleotide
accggcttaaggtcaaacca

SEQ ID NO: 802 - DNA, 20 bp, Artificial
  oligonucleotide
acccaacatccagcacacat

SEQ ID NO: 803 - DNA, 20 bp, Artificial
  oligonucleotide
tcgctgacccgaccgtggtc

SEQ ID NO: 804 - DNA, 20 bp, Artificial
  oligonucleotide
cgctgacccgaccgtggtcg

SEQ ID NO: 805 - DNA, 20 bp, Artificial
  oligonucleotide
gacccgaccgtggtcggctg

SEQ ID NO: 806 - DNA, 20 bp, Artificial
  oligonucleotide
gctgacccgaccgtggtcgg

SEQ ID NO: 807 - DNA, 20 bp, Artificial
  oligonucleotide
ctgacccgaccgtggtcggc

SEQ ID NO: 808 - DNA, 20 bp, Artificial
  oligonucleotide
caggcgacttgcgcctttga

SEQ ID NO: 809 - DNA, 20 bp, Artificial
  oligonucleotide
tcatgcggtattagctccag

SEQ ID NO: 810 - DNA, 20 bp, Artificial
  oligonucleotide
actagctaatcgaacgcagg

SEQ ID NO: 811 - DNA, 20 bp, Artificial
  oligonucleotide
catgcggtattagctccagt

SEQ ID NO: 812 - DNA, 20 bp, Artificial
  oligonucleotide
cgcaggctcctccacaggcg

SEQ ID NO: 813 - DNA, 20 bp, Artificial
  oligonucleotide
acgcaggctcctccacaggc

SEQ ID NO: 814 - DNA, 20 bp, Artificial
  oligonucleotide
ctcaggtgtcatgcggtatt

SEQ ID NO: 815 - DNA, 20 bp, Artificial
  oligonucleotide
cgcctttgaccctcaggtgt

SEQ ID NO: 816 - DNA, 20 bp, Artificial
  oligonucleotide
accctcaggtgtcatgcggt

SEQ ID NO: 817 - DNA, 20 bp, Artificial
  oligonucleotide
cctcaggtgtcatgcggtat

SEQ ID NO: 818 - DNA, 20 bp, Artificial
  oligonucleotide
tttgaccctcaggtgtcatg

SEQ ID NO: 819 - DNA, 20 bp, Artificial
  oligonucleotide
gaccctcaggtgtcatgcgg

SEQ ID NO: 820 - DNA, 20 bp, Artificial
  oligonucleotide
tgaccctcaggtgtcatgcg

SEQ ID NO: 821 - DNA, 20 bp, Artificial
  oligonucleotide
gcctttgaccctcaggtgtc

SEQ ID NO: 822 - DNA, 20 bp, Artificial
  oligonucleotide
ttgaccctcaggtgtcatgc

SEQ ID NO: 823 - DNA, 20 bp, Artificial
  oligonucleotide
ccctcaggtgtcatgcggta

SEQ ID NO: 824 - DNA, 20 bp, Artificial
  oligonucleotide
cctttgaccctcaggtgtca

SEQ ID NO: 825 - DNA, 20 bp, Artificial
  oligonucleotide
ctttgaccctcaggtgtcat

SEQ ID NO: 826 - DNA, 20 bp, Artificial
  oligonucleotide
agttatcccccacccatgga

SEQ ID NO: 827 - DNA, 20 bp, Artificial
  oligonucleotide
ccagctatcgatcatcgcct

SEQ ID NO: 828 - DNA, 20 bp, Artificial
  oligonucleotide
accagctatcgatcatcgcc

SEQ ID NO: 829 - DNA, 20 bp, Artificial
  oligonucleotide
cagctatcgatcatcgcctt

SEQ ID NO: 830 - DNA, 20 bp, Artificial
  oligonucleotide
agctatcgatcatcgccttg

SEQ ID NO: 831 - DNA, 20 bp, Artificial
  oligonucleotide
gctatcgatcatcgccttgg

SEQ ID NO: 832 - DNA, 20 bp, Artificial
  oligonucleotide
ctatcgatcatcgccttggt

SEQ ID NO: 833 - DNA, 20 bp, Artificial
  oligonucleotide
ttcgtgcgacttgcatgtgt

SEQ ID NO: 834 - DNA, 20 bp, Artificial
  oligonucleotide
tcgatcatcgccttggtagg

SEQ ID NO: 835 - DNA, 20 bp, Artificial
  oligonucleotide
atcgatcatcgccttggtag

SEQ ID NO: 836 - DNA, 20 bp, Artificial
  oligonucleotide
cacaggcgacttgcgccttt

SEQ ID NO: 837 - DNA, 20 bp, Artificial
  oligonucleotide
ccacaggcgacttgcgcctt

SEQ ID NO: 838 - DNA, 20 bp, Artificial
  oligonucleotide
tccacaggcgacttgcgcct

SEQ ID NO: 839 - DNA, 20 bp, Artificial
  oligonucleotide
tcctccacaggcgacttgcg

SEQ ID NO: 840 - DNA, 20 bp, Artificial
  oligonucleotide
cctccacaggcgacttgcgc

SEQ ID NO: 841 - DNA, 20 bp, Artificial
  oligonucleotide
ctccacaggcgacttgcgcc

SEQ ID NO: 842 - DNA, 20 bp, Artificial
  oligonucleotide
acaggcgacttgcgcctttg

SEQ ID NO: 843 - DNA, 20 bp, Artificial
  oligonucleotide
agccccggtttcccggcgtt

SEQ ID NO: 844 - DNA, 20 bp, Artificial
  oligonucleotide
cgcctttcctttttcctcca

SEQ ID NO: 845 - DNA, 20 bp, Artificial
  oligonucleotide
gccccggtttcccggcgtta

SEQ ID NO: 846 - DNA, 20 bp, Artificial
  oligonucleotide
gccgcctttcctttttcctc

SEQ ID NO: 847 - DNA, 20 bp, Artificial
  oligonucleotide
tagccccggtttcccggcgt

SEQ ID NO: 848 - DNA, 20 bp, Artificial
  oligonucleotide
ccgggtaccgtcaaggcgcc

SEQ ID NO: 849 - DNA, 20 bp, Artificial
  oligonucleotide
aagccgcctttcctttttcc

SEQ ID NO: 850 - DNA, 20 bp, Artificial
  oligonucleotide
ccccggtttcccggcgttat

SEQ ID NO: 851 - DNA, 20 bp, Artificial
  oligonucleotide
ccggcgttatcccagtctta

SEQ ID NO: 852 - DNA, 20 bp, Artificial
  oligonucleotide
agccgcctttcctttttcct

SEQ ID NO: 853 - DNA, 20 bp, Artificial
  oligonucleotide
ccgcctttcctttttcctcc

SEQ ID NO: 854 - DNA, 20 bp, Artificial
  oligonucleotide
ttagccccggtttcccggcg

SEQ ID NO: 855 - DNA, 20 bp, Artificial
  oligonucleotide
cccggcgttatcccagtctt

SEQ ID NO: 856 - DNA, 20 bp, Artificial
  oligonucleotide
gccgggtaccgtcaaggcgc

SEQ ID NO: 857 - DNA, 20 bp, Artificial
  oligonucleotide
ggccgggtaccgtcaaggcg

SEQ ID NO: 858 - DNA, 20 bp, Artificial
  oligonucleotide
tcccggcgttatcccagtct

SEQ ID NO: 859 - DNA, 20 bp, Artificial
  oligonucleotide
tggccgggtaccgtcaaggc

SEQ ID NO: 860 - DNA, 20 bp, Artificial
  oligonucleotide
gaagccgcctttcctttttc

SEQ ID NO: 861 - DNA, 20 bp, Artificial
  oligonucleotide
cccggtttcccggcgttatc

SEQ ID NO: 862 - DNA, 20 bp, Artificial
  oligonucleotide
cggcgttatcccagtcttac

SEQ ID NO: 863 - DNA, 20 bp, Artificial
  oligonucleotide
ggcgttatcccagtcttaca

SEQ ID NO: 864 - DNA, 20 bp, Artificial
  oligonucleotide
gcgttatcccagtcttacag

SEQ ID NO: 865 - DNA, 20 bp, Artificial
  oligonucleotide
cgggtaccgtcaaggcgccg

SEQ ID NO: 866 - DNA, 20 bp, Artificial
  oligonucleotide
attagccccggtttcccggc

SEQ ID NO: 867 - DNA, 20 bp, Artificial
  oligonucleotide
aaggggaaggccctgtctcc

SEQ ID NO: 868 - DNA, 20 bp, Artificial
  oligonucleotide
ggccctgtctccagggaggt

SEQ ID NO: 869 - DNA, 20 bp, Artificial
  oligonucleotide
aggccctgtctccagggagg

SEQ ID NO: 870 - DNA, 20 bp, Artificial
  oligonucleotide
aaggccctgtctccagggag

SEQ ID NO: 871 - DNA, 20 bp, Artificial
  oligonucleotide
gccctgtctccagggaggtc

SEQ ID NO: 872 - DNA, 20 bp, Artificial
  oligonucleotide
cgttatcccagtcttacagg

SEQ ID NO: 873 - DNA, 20 bp, Artificial
  oligonucleotide
gggtaccgtcaaggcgccgc

SEQ ID NO: 874 - DNA, 20 bp, Artificial
  oligonucleotide
cggcaacagagttttacgac

SEQ ID NO: 875 - DNA, 20 bp, Artificial
  oligonucleotide
ggggaaggccctgtctccag

SEQ ID NO: 876 - DNA, 20 bp, Artificial
  oligonucleotide
aggggaaggccctgtctcca

SEQ ID NO: 877 - DNA, 20 bp, Artificial
  oligonucleotide
gcagccgaagccgcctttcc

SEQ ID NO: 878 - DNA, 20 bp, Artificial
  oligonucleotide
ttcttccccggcaacagagt

SEQ ID NO: 879 - DNA, 20 bp, Artificial
  oligonucleotide
cggcacttgttcttccccgg

SEQ ID NO: 880 - DNA, 20 bp, Artificial
  oligonucleotide
gttcttccccggcaacagag

SEQ ID NO: 881 - DNA, 20 bp, Artificial
  oligonucleotide
ggcacttgttcttccccggc

SEQ ID NO: 882 - DNA, 20 bp, Artificial
  oligonucleotide
gcacttgttcttccccggca

SEQ ID NO: 883 - DNA, 20 bp, Artificial
  oligonucleotide
cacttgttcttccccggcaa

SEQ ID NO: 884 - DNA, 20 bp, Artificial
  oligonucleotide
tcttccccggcaacagagtt

SEQ ID NO: 885 - DNA, 20 bp, Artificial
  oligonucleotide
ttgttcttccccggcaacag

SEQ ID NO: 886 - DNA, 20 bp, Artificial
  oligonucleotide
acttgttcttccccggcaac

SEQ ID NO: 887 - DNA, 20 bp, Artificial
  oligonucleotide
tgttcttccccggcaacaga

SEQ ID NO: 888 - DNA, 20 bp, Artificial
  oligonucleotide
cttgttcttccccggcaaca

SEQ ID NO: 889 - DNA, 20 bp, Artificial
  oligonucleotide
acggcacttgttcttccccg

SEQ ID NO: 890 - DNA, 20 bp, Artificial
  oligonucleotide
gtccgccgctaaccttttaa

SEQ ID NO: 891 - DNA, 20 bp, Artificial
  oligonucleotide
ctggccgggtaccgtcaagg

SEQ ID NO: 892 - DNA, 20 bp, Artificial
  oligonucleotide
tctggccgggtaccgtcaag

SEQ ID NO: 893 - DNA, 20 bp, Artificial
  oligonucleotide
ttctggccgggtaccgtcaa

SEQ ID NO: 894 - DNA, 20 bp, Artificial
  oligonucleotide
caatgctggcaactaaggtc

SEQ ID NO: 895 - DNA, 20 bp, Artificial
  oligonucleotide
cgtccgccgctaacctttta

SEQ ID NO: 896 - DNA, 20 bp, Artificial
  oligonucleotide
cgaagccgcctttccttttt

SEQ ID NO: 897 - DNA, 20 bp, Artificial
  oligonucleotide
ccgaagccgcctttcctttt

SEQ ID NO: 898 - DNA, 20 bp, Artificial
  oligonucleotide
gccgaagccgcctttccttt

SEQ ID NO: 899 - DNA, 20 bp, Artificial
  oligonucleotide
agccgaagccgcctttcctt

SEQ ID NO: 900 - DNA, 20 bp, Artificial
  oligonucleotide
accgtcaaggcgccgccctg

SEQ ID NO: 901 - DNA, 20 bp, Artificial
  oligonucleotide
ccgtggctttctggccgggt

SEQ ID NO: 902 - DNA, 20 bp, Artificial
  oligonucleotide
gctttctggccgggtaccgt

SEQ ID NO: 903 - DNA, 20 bp, Artificial
  oligonucleotide
gccgtggctttctggccggg

SEQ ID NO: 904 - DNA, 20 bp, Artificial
  oligonucleotide
ggctttctggccgggtaccg

SEQ ID NO: 905 - DNA, 20 bp, Artificial
  oligonucleotide
ctttctggccgggtaccgtc

SEQ ID NO: 906 - DNA, 20 bp, Artificial
  oligonucleotide
tggctttctggccgggtacc

SEQ ID NO: 907 - DNA, 20 bp, Artificial
  oligonucleotide
gtggctttctggccgggtac

SEQ ID NO: 908 - DNA, 20 bp, Artificial
  oligonucleotide
cgtggctttctggccgggta

SEQ ID NO: 909 - DNA, 20 bp, Artificial
  oligonucleotide
tttctggccgggtaccgtca

SEQ ID NO: 910 - DNA, 20 bp, Artificial
  oligonucleotide
gggaaggccctgtctccagg

SEQ ID NO: 911 - DNA, 20 bp, Artificial
  oligonucleotide
cgaaggggaaggccctgtct

SEQ ID NO: 912 - DNA, 20 bp, Artificial
  oligonucleotide
ccgaaggggaaggccctgtc

SEQ ID NO: 913 - DNA, 20 bp, Artificial
  oligonucleotide
gaaggggaaggccctgtctc

SEQ ID NO: 914 - DNA, 20 bp, Artificial
  oligonucleotide
ggcgccgccctgttcgaacg

SEQ ID NO: 915 - DNA, 20 bp, Artificial
  oligonucleotide
aggcgccgccctgttcgaac

SEQ ID NO: 916 - DNA, 20 bp, Artificial
  oligonucleotide
aaggcgccgccctgttcgaa

SEQ ID NO: 917 - DNA, 20 bp, Artificial
  oligonucleotide
cccggcaacagagttttacg

SEQ ID NO: 918 - DNA, 20 bp, Artificial
  oligonucleotide
ccccggcaacagagttttac

SEQ ID NO: 919 - DNA, 20 bp, Artificial
  oligonucleotide
ccatctgtaagtggcagccg

SEQ ID NO: 920 - DNA, 20 bp, Artificial
  oligonucleotide
tctgtaagtggcagccgaag

SEQ ID NO: 921 - DNA, 20 bp, Artificial
  oligonucleotide
ctgtaagtggcagccgaagc

SEQ ID NO: 922 - DNA, 20 bp, Artificial
  oligonucleotide
cccatctgtaagtggcagcc

SEQ ID NO: 923 - DNA, 20 bp, Artificial
  oligonucleotide
tgtaagtggcagccgaagcc

SEQ ID NO: 924 - DNA, 20 bp, Artificial
  oligonucleotide
catctgtaagtggcagccga

SEQ ID NO: 925 - DNA, 20 bp, Artificial
  oligonucleotide
atctgtaagtggcagccgaa

SEQ ID NO: 926 - DNA, 20 bp, Artificial
  oligonucleotide
cagccgaagccgcctttcct

SEQ ID NO: 927 - DNA, 20 bp, Artificial
  oligonucleotide
ggcaacagagttttacgacc

SEQ ID NO: 928 - DNA, 20 bp, Artificial
  oligonucleotide
ccggcaacagagttttacga

SEQ ID NO: 929 - DNA, 20 bp, Artificial
  oligonucleotide
ttccccggcaacagagtttt

SEQ ID NO: 930 - DNA, 20 bp, Artificial
  oligonucleotide
cttccccggcaacagagttt

SEQ ID NO: 931 - DNA, 20 bp, Artificial
  oligonucleotide
tccccggcaacagagtttta

SEQ ID NO: 932 - DNA, 20 bp, Artificial
  oligonucleotide
ccgtccgccgctaacctttt

SEQ ID NO: 933 - DNA, 20 bp, Artificial
  oligonucleotide
cttcctccgacttacgccgg

SEQ ID NO: 934 - DNA, 20 bp, Artificial
  oligonucleotide
cctccgacttacgccggcag

SEQ ID NO: 935 - DNA, 20 bp, Artificial
  oligonucleotide
ttcctccgacttacgccggc

SEQ ID NO: 936 - DNA, 20 bp, Artificial
  oligonucleotide
tcctccgacttacgccggca

SEQ ID NO: 937 - DNA, 20 bp, Artificial
  oligonucleotide
tccgacttacgccggcagtc

SEQ ID NO: 938 - DNA, 20 bp, Artificial
  oligonucleotide
ccgacttacgccggcagtca

SEQ ID NO: 939 - DNA, 20 bp, Artificial
  oligonucleotide
gccttcctccgacttacgcc

SEQ ID NO: 940 - DNA, 20 bp, Artificial
  oligonucleotide
ccttcctccgacttacgccg

SEQ ID NO: 941 - DNA, 20 bp, Artificial
  oligonucleotide
gctctccccgagcaacagag

SEQ ID NO: 942 - DNA, 20 bp, Artificial
  oligonucleotide
ctctccccgagcaacagagc

SEQ ID NO: 943 - DNA, 20 bp, Artificial
  oligonucleotide
cgctctccccgagcaacaga

SEQ ID NO: 944 - DNA, 20 bp, Artificial
  oligonucleotide
ctccgacttacgccggcagt

SEQ ID NO: 945 - DNA, 20 bp, Artificial
  oligonucleotide
tctccccgagcaacagagct

SEQ ID NO: 946 - DNA, 20 bp, Artificial
  oligonucleotide
cgacttacgccggcagtcac

SEQ ID NO: 947 - DNA, 20 bp, Artificial
  oligonucleotide
tcggcactggggtgtgtccc

SEQ ID NO: 948 - DNA, 20 bp, Artificial
  oligonucleotide
ggcactggggtgtgtccccc

SEQ ID NO: 949 - DNA, 20 bp, Artificial
  oligonucleotide
ctggggtgtgtccccccaac

SEQ ID NO: 950 - DNA, 20 bp, Artificial
  oligonucleotide
cactggggtgtgtcccccca

SEQ ID NO: 951 - DNA, 20 bp, Artificial
  oligonucleotide
actggggtgtgtccccccaa

SEQ ID NO: 952 - DNA, 20 bp, Artificial
  oligonucleotide
gcactggggtgtgtcccccc

SEQ ID NO: 953 - DNA, 20 bp, Artificial
  oligonucleotide
tggggtgtgtccccccaaca

SEQ ID NO: 954 - DNA, 20 bp, Artificial
  oligonucleotide
cactccagacttgctcgacc

SEQ ID NO: 955 - DNA, 20 bp, Artificial
  oligonucleotide
tcactccagacttgctcgac

SEQ ID NO: 956 - DNA, 20 bp, Artificial
  oligonucleotide
cggcactggggtgtgtcccc

SEQ ID NO: 957 - DNA, 20 bp, Artificial
  oligonucleotide
cgccttcctccgacttacgc

SEQ ID NO: 958 - DNA, 20 bp, Artificial
  oligonucleotide
ctccccgagcaacagagctt

SEQ ID NO: 959 - DNA, 20 bp, Artificial
  oligonucleotide
actccagacttgctcgaccg

SEQ ID NO: 960 - DNA, 20 bp, Artificial
  oligonucleotide
cccatgccgctctccccgag

SEQ ID NO: 961 - DNA, 20 bp, Artificial
  oligonucleotide
ccatgccgctctccccgagc

SEQ ID NO: 962 - DNA, 20 bp, Artificial
  oligonucleotide
ccccatgccgctctccccga

SEQ ID NO: 963 - DNA, 20 bp, Artificial
  oligonucleotide
tcactcggtaccgtctcgca

SEQ ID NO: 964 - DNA, 20 bp, Artificial
  oligonucleotide
catgccgctctccccgagca

SEQ ID NO: 965 - DNA, 20 bp, Artificial
  oligonucleotide
atgccgctctccccgagcaa

SEQ ID NO: 966 - DNA, 20 bp, Artificial
  oligonucleotide
ttcggcactggggtgtgtcc

SEQ ID NO: 967 - DNA, 20 bp, Artificial
  oligonucleotide
tgccgctctccccgagcaac

SEQ ID NO: 968 - DNA, 20 bp, Artificial
  oligonucleotide
ttcactccagacttgctcga

SEQ ID NO: 969 - DNA, 20 bp, Artificial
  oligonucleotide
cccgcaagaagatgcctcct

SEQ ID NO: 970 - DNA, 20 bp, Artificial
  oligonucleotide
agaagatgcctcctcgcggg

SEQ ID NO: 971 - DNA, 20 bp, Artificial
  oligonucleotide
aagaagatgcctcctcgcgg

SEQ ID NO: 972 - DNA, 20 bp, Artificial
  oligonucleotide
cgcaagaagatgcctcctcg

SEQ ID NO: 973 - DNA, 20 bp, Artificial
  oligonucleotide
aagatgcctcctcgcgggcg

SEQ ID NO: 974 - DNA, 20 bp, Artificial
  oligonucleotide
ccgcaagaagatgcctcctc

SEQ ID NO: 975 - DNA, 20 bp, Artificial
  oligonucleotide
gaagatgcctcctcgcgggc

SEQ ID NO: 976 - DNA, 20 bp, Artificial
  oligonucleotide
ccccgcaagaagatgcctcc

SEQ ID NO: 977 - DNA, 20 bp, Artificial
  oligonucleotide
caagaagatgcctcctcgcg

SEQ ID NO: 978 - DNA, 20 bp, Artificial
  oligonucleotide
tccttcggcactggggtgtg

SEQ ID NO: 979 - DNA, 20 bp, Artificial
  oligonucleotide
ccgctctccccgagcaacag

SEQ ID NO: 980 - DNA, 20 bp, Artificial
  oligonucleotide
tgcctcctcgcgggcgtatc

SEQ ID NO: 981 - DNA, 20 bp, Artificial
  oligonucleotide
gacttacgccggcagtcacc

SEQ ID NO: 982 - DNA, 20 bp, Artificial
  oligonucleotide
ggctcctctctcagcggccc

SEQ ID NO: 983 - DNA, 20 bp, Artificial
  oligonucleotide
ccttcggcactggggtgtgt

SEQ ID NO: 984 - DNA, 20 bp, Artificial
  oligonucleotide
ggggtgtgtccccccaacac

SEQ ID NO: 985 - DNA, 20 bp, Artificial
  oligonucleotide
gccgctctccccgagcaaca

SEQ ID NO: 986 - DNA, 20 bp, Artificial
  oligonucleotide
agatgcctcctcgcgggcgt

SEQ ID NO: 987 - DNA, 20 bp, Artificial
  oligonucleotide
cactcggtaccgtctcgcat

SEQ ID NO: 988 - DNA, 20 bp, Artificial
  oligonucleotide
ctcactcggtaccgtctcgc

SEQ ID NO: 989 - DNA, 20 bp, Artificial
  oligonucleotide
gcaagaagatgcctcctcgc

SEQ ID NO: 990 - DNA, 20 bp, Artificial
  oligonucleotide
ctccagacttgctcgaccgc

SEQ ID NO: 991 - DNA, 20 bp, Artificial
  oligonucleotide
ttacgccggcagtcacctgt

SEQ ID NO: 992 - DNA, 20 bp, Artificial
  oligonucleotide
cttcggcactggggtgtgtc

SEQ ID NO: 993 - DNA, 20 bp, Artificial
  oligonucleotide
ctcgcgggcgtatccggcat

SEQ ID NO: 994 - DNA, 20 bp, Artificial
  oligonucleotide
gcctcctcgcgggcgtatcc

SEQ ID NO: 995 - DNA, 20 bp, Artificial
  oligonucleotide
actcggtaccgtctcgcatg

SEQ ID NO: 996 - DNA, 20 bp, Artificial
  oligonucleotide
gatgcctcctcgcgggcgta

SEQ ID NO: 997 - DNA, 20 bp, Artificial
  oligonucleotide
gggtgtgtccccccaacacc

SEQ ID NO: 998 - DNA, 20 bp, Artificial
  oligonucleotide
acttacgccggcagtcacct

SEQ ID NO: 999 - DNA, 20 bp, Artificial
  oligonucleotide
cttacgccggcagtcacctg

SEQ ID NO: 1000 - DNA, 20 bp, Artificial
  oligonucleotide
atgcctcctcgcgggcgtat

SEQ ID NO: 1001 - DNA, 20 bp, Artificial
  oligonucleotide
gcgccgcgggctcctctctc

SEQ ID NO: 1002 - DNA, 20 bp, Artificial
  oligonucleotide
ggtgtgtccccccaacacct

SEQ ID NO: 1003 - DNA, 20 bp, Artificial
  oligonucleotide
gtgtgtccccccaacaccta

SEQ ID NO: 1004 - DNA, 20 bp, Artificial
  oligonucleotide
cctcgcgggcgtatccggca

SEQ ID NO: 1005 - DNA, 20 bp, Artificial
  oligonucleotide
cctcactcggtaccgtctcg

SEQ ID NO: 1006 - DNA, 20 bp, Artificial
  oligonucleotide
tcctcactcggtaccgtctc

SEQ ID NO: 1007 - DNA, 20 bp, Artificial
  oligonucleotide
tcgcgggcgtatccggcatt

SEQ ID NO: 1008 - DNA, 20 bp, Artificial
  oligonucleotide
tttcactccagacttgctcg

SEQ ID NO: 1009 - DNA, 20 bp, Artificial
  oligonucleotide
tacgccggcagtcacctgtg

SEQ ID NO: 1010 - DNA, 20 bp, Artificial
  oligonucleotide
tccagacttgctcgaccgcc

SEQ ID NO: 1011 - DNA, 20 bp, Artificial
  oligonucleotide
ctcggtaccgtctcgcatgg

SEQ ID NO: 1012 - DNA, 20 bp, Artificial
  oligonucleotide
cgcgggcgtatccggcatta

SEQ ID NO: 1013 - DNA, 20 bp, Artificial
  oligonucleotide
gcgtatccggcattagcgcc

SEQ ID NO: 1014 - DNA, 20 bp, Artificial
  oligonucleotide
gggctcctctctcagcggcc

SEQ ID NO: 1015 - DNA, 20 bp, Artificial
  oligonucleotide
tccccgagcaacagagcttt

SEQ ID NO: 1016 - DNA, 20 bp, Artificial
  oligonucleotide
ccccgagcaacagagcttta

SEQ ID NO: 1017 - DNA, 20 bp, Artificial
  oligonucleotide
ccgagcaacagagctttaca

SEQ ID NO: 1018 - DNA, 20 bp, Artificial
  oligonucleotide
ccatcccatggttgagccat

SEQ ID NO: 1019 - DNA, 20 bp, Artificial
  oligonucleotide
gtgtccccccaacacctagc

SEQ ID NO: 1020 - DNA, 20 bp, Artificial
  oligonucleotide
gcgggcgtatccggcattag

SEQ ID NO: 1021 - DNA, 20 bp, Artificial
  oligonucleotide
cgagcggctttttgggtttc

SEQ ID NO: 1022 - DNA, 20 bp, Artificial
  oligonucleotide
ctttcactccagacttgctc

SEQ ID NO: 1023 - DNA, 20 bp, Artificial
  oligonucleotide
ttccttcggcactggggtgt

SEQ ID NO: 1024 - DNA, 20 bp, Artificial
  oligonucleotide
ccgccttcctccgacttacg

SEQ ID NO: 1025 - DNA, 20 bp, Artificial
  oligonucleotide
cccgccttcctccgacttac

SEQ ID NO: 1026 - DNA, 20 bp, Artificial
  oligonucleotide
cctcctcgcgggcgtatccg

SEQ ID NO: 1027 - DNA, 20 bp, Artificial
  oligonucleotide
tcctcgcgggcgtatccggc

SEQ ID NO: 1028 - DNA, 20 bp, Artificial
  oligonucleotide
cattagcgcccgtttccggg

SEQ ID NO: 1029 - DNA, 20 bp, Artificial
  oligonucleotide
gcattagcgcccgtttccgg

SEQ ID NO: 1030 - DNA, 20 bp, Artificial
  oligonucleotide
ggcattagcgcccgtttccg

SEQ ID NO: 1031 - DNA, 20 bp, Artificial
  oligonucleotide
gtctcgcatggggctttcca

SEQ ID NO: 1032 - DNA, 20 bp, Artificial
  oligonucleotide
gccatggactttcactccag

SEQ ID NO: 1033 - DNA, 20 bp, Artificial
  oligonucleotide
catggactttcactccagac

SEQ ID NO: 1034 - DNA, 22 bp, Artificial
  oligonucleotide
ccttcctccggcttacgccggc

SEQ ID NO: 1035 - DNA, 22 bp, Artificial
  oligonucleotide
ccttcctccgacttgcgccggc

SEQ ID NO: 1036 - DNA, 22 bp, Artificial
  oligonucleotide
ccttcctccgactttcaccggc

SEQ ID NO: 1037 - DNA, 20 bp, Artificial
  oligonucleotide
accgtctcacaaggagcttt

SEQ ID NO: 1038 - DNA, 20 bp, Artificial
  oligonucleotide
taccgtctcacaaggagctt

SEQ ID NO: 1039 - DNA, 20 bp, Artificial
  oligonucleotide
gtaccgtctcacaaggagct

SEQ ID NO: 1040 - DNA, 20 bp, Artificial
  oligonucleotide
gcctacccgtgtattatccg

SEQ ID NO: 1041 - DNA, 20 bp, Artificial
  oligonucleotide
ccgtctcacaaggagctttc

SEQ ID NO: 1042 - DNA, 20 bp, Artificial
  oligonucleotide
ctacccgtgtattatccggc

SEQ ID NO: 1043 - DNA, 20 bp, Artificial
  oligonucleotide
ggtaccgtctcacaaggagc

SEQ ID NO: 1044 - DNA, 20 bp, Artificial
  oligonucleotide
cgtctcacaaggagctttcc

SEQ ID NO: 1045 - DNA, 20 bp, Artificial
  oligonucleotide
tctcacaaggagctttccac

SEQ ID NO: 1046 - DNA, 20 bp, Artificial
  oligonucleotide
tacccgtgtattatccggca

SEQ ID NO: 1047 - DNA, 20 bp, Artificial
  oligonucleotide
gtctcacaaggagctttcca

SEQ ID NO: 1048 - DNA, 20 bp, Artificial
  oligonucleotide
acccgtgtattatccggcat

SEQ ID NO: 1049 - DNA, 20 bp, Artificial
  oligonucleotide
ctcggtaccgtctcacaagg

SEQ ID NO: 1050 - DNA, 20 bp, Artificial
  oligonucleotide
cggtaccgtctcacaaggag

SEQ ID NO: 1051 - DNA, 20 bp, Artificial
  oligonucleotide
actcggtaccgtctcacaag

SEQ ID NO: 1052 - DNA, 20 bp, Artificial
  oligonucleotide
cggctggctccataacggtt

SEQ ID NO: 1053 - DNA, 20 bp, Artificial
  oligonucleotide
acaagtagatgcctacccgt

SEQ ID NO: 1054 - DNA, 20 bp, Artificial
  oligonucleotide
tggctccataacggttacct

SEQ ID NO: 1055 - DNA, 20 bp, Artificial
  oligonucleotide
caagtagatgcctacccgtg

SEQ ID NO: 1056 - DNA, 20 bp, Artificial
  oligonucleotide
cacaagtagatgcctacccg

SEQ ID NO: 1057 - DNA, 20 bp, Artificial
  oligonucleotide
ggctccataacggttacctc

SEQ ID NO: 1058 - DNA, 20 bp, Artificial
  oligonucleotide
acacaagtagatgcctaccc

SEQ ID NO: 1059 - DNA, 20 bp, Artificial
  oligonucleotide
ctggctccataacggttacc

SEQ ID NO: 1060 - DNA, 20 bp, Artificial
  oligonucleotide
gctggctccataacggttac

SEQ ID NO: 1061 - DNA, 20 bp, Artificial
  oligonucleotide
ggctggctccataacggtta

SEQ ID NO: 1062 - DNA, 20 bp, Artificial
  oligonucleotide
gctccataacggttacctca

SEQ ID NO: 1063 - DNA, 20 bp, Artificial
  oligonucleotide
aagtagatgcctacccgtgt

SEQ ID NO: 1064 - DNA, 20 bp, Artificial
  oligonucleotide
ctccataacggttacctcac

SEQ ID NO: 1065 - DNA, 20 bp, Artificial
  oligonucleotide
tgcctacccgtgtattatcc

SEQ ID NO: 1066 - DNA, 20 bp, Artificial
  oligonucleotide
tcggtaccgtctcacaagga

SEQ ID NO: 1067 - DNA, 20 bp, Artificial
  oligonucleotide
ctcacaaggagctttccact

SEQ ID NO: 1068 - DNA, 20 bp, Artificial
  oligonucleotide
gtagatgcctacccgtgtat

SEQ ID NO: 1069 - DNA, 20 bp, Artificial
  oligonucleotide
cctacccgtgtattatccgg

SEQ ID NO: 1070 - DNA, 20 bp, Artificial
  oligonucleotide
cactcggtaccgtctcacaa

SEQ ID NO: 1071 - DNA, 20 bp, Artificial
  oligonucleotide
ctcagcgatgcagttgcatc

SEQ ID NO: 1072 - DNA, 20 bp, Artificial
  oligonucleotide
agtagatgcctacccgtgta

SEQ ID NO: 1073 - DNA, 20 bp, Artificial
  oligonucleotide
gcggctggctccataacggt

SEQ ID NO: 1074 - DNA, 20 bp, Artificial
  oligonucleotide
ccaaagcaatcccaaggttg

SEQ ID NO: 1075 - DNA, 20 bp, Artificial
  oligonucleotide
tccataacggttacctcacc

SEQ ID NO: 1076 - DNA, 20 bp, Artificial
  oligonucleotide
cccgtgtattatccggcatt

SEQ ID NO: 1077 - DNA, 20 bp, Artificial
  oligonucleotide
tctcagcgatgcagttgcat

SEQ ID NO: 1078 - DNA, 20 bp, Artificial
  oligonucleotide
ccataacggttacctcaccg

SEQ ID NO: 1079 - DNA, 20 bp, Artificial
  oligonucleotide
tcagcgatgcagttgcatct

SEQ ID NO: 1080 - DNA, 20 bp, Artificial
  oligonucleotide
ggcggctggctccataacgg

SEQ ID NO: 1081 - DNA, 20 bp, Artificial
  oligonucleotide
aagcaatcccaaggttgagc

SEQ ID NO: 1082 - DNA, 20 bp, Artificial
  oligonucleotide
tcactcggtaccgtctcaca

SEQ ID NO: 1083 - DNA, 20 bp, Artificial
  oligonucleotide
ccgagtgttattccagtctg

SEQ ID NO: 1084 - DNA, 20 bp, Artificial
  oligonucleotide
cacaaggagctttccactct

SEQ ID NO: 1085 - DNA, 20 bp, Artificial
  oligonucleotide
acaaggagctttccactctc

SEQ ID NO: 1086 - DNA, 20 bp, Artificial
  oligonucleotide
tcacaaggagctttccactc

SEQ ID NO: 1087 - DNA, 20 bp, Artificial
  oligonucleotide
cagcgatgcagttgcatctt

SEQ ID NO: 1088 - DNA, 20 bp, Artificial
  oligonucleotide
caaggagctttccactctcc

SEQ ID NO: 1089 - DNA, 20 bp, Artificial
  oligonucleotide
ccagtctgaaaggcagattg

SEQ ID NO: 1090 - DNA, 20 bp, Artificial
  oligonucleotide
cagtctgaaaggcagattgc

SEQ ID NO: 1091 - DNA, 20 bp, Artificial
  oligonucleotide
cggcggctggctccataacg

SEQ ID NO: 1092 - DNA, 20 bp, Artificial
  oligonucleotide
cctctctcagcgatgcagtt

SEQ ID NO: 1093 - DNA, 20 bp, Artificial
  oligonucleotide
ctctctcagcgatgcagttg

SEQ ID NO: 1094 - DNA, 20 bp, Artificial
  oligonucleotide
tctctcagcgatgcagttgc

SEQ ID NO: 1095 - DNA, 20 bp, Artificial
  oligonucleotide
ctctcagcgatgcagttgca

SEQ ID NO: 1096 - DNA, 20 bp, Artificial
  oligonucleotide
caatcccaaggttgagcctt

SEQ ID NO: 1097 - DNA, 20 bp, Artificial
  oligonucleotide
aatcccaaggttgagccttg

SEQ ID NO: 1098 - DNA, 20 bp, Artificial
  oligonucleotide
agcaatcccaaggttgagcc

SEQ ID NO: 1099 - DNA, 20 bp, Artificial
  oligonucleotide
ctcactcggtaccgtctcac

SEQ ID NO: 1100 - DNA, 20 bp, Artificial
  oligonucleotide
gcaatcccaaggttgagcct

SEQ ID NO: 1101 - DNA, 20 bp, Artificial
  oligonucleotide
gccttggactttcacttcag

SEQ ID NO: 1102 - DNA, 20 bp, Artificial
  oligonucleotide
cataacggttacctcaccga

SEQ ID NO: 1103 - DNA, 20 bp, Artificial
  oligonucleotide
ctcctctctcagcgatgcag

SEQ ID NO: 1104 - DNA, 20 bp, Artificial
  oligonucleotide
tcggcggctggctccataac

SEQ ID NO: 1105 - DNA, 20 bp, Artificial
  oligonucleotide
agtctgaaaggcagattgcc

SEQ ID NO: 1106 - DNA, 20 bp, Artificial
  oligonucleotide
tcctctctcagcgatgcagt

SEQ ID NO: 1107 - DNA, 20 bp, Artificial
  oligonucleotide
cccaaggttgagccttggac

SEQ ID NO: 1108 - DNA, 20 bp, Artificial
  oligonucleotide
ataacggttacctcaccgac

SEQ ID NO: 1109 - DNA, 20 bp, Artificial
  oligonucleotide
tcccaaggttgagccttgga

SEQ ID NO: 1110 - DNA, 20 bp, Artificial
  oligonucleotide
attatccggcattagcaccc

SEQ ID NO: 1111 - DNA, 20 bp, Artificial
  oligonucleotide
ctacgtgctggtaacacaga

SEQ ID NO: 1112 - DNA, 20 bp, Artificial
  oligonucleotide
gccgctagccccgaagggct

SEQ ID NO: 1113 - DNA, 20 bp, Artificial
  oligonucleotide
ctagccccgaagggctcgct

SEQ ID NO: 1114 - DNA, 20 bp, Artificial
  oligonucleotide
cgctagccccgaagggctcg

SEQ ID NO: 1115 - DNA, 20 bp, Artificial
  oligonucleotide
agccccgaagggctcgctcg

SEQ ID NO: 1116 - DNA, 20 bp, Artificial
  oligonucleotide
ccgctagccccgaagggctc

SEQ ID NO: 1117 - DNA, 20 bp, Artificial
  oligonucleotide
tagccccgaagggctcgctc

SEQ ID NO: 1118 - DNA, 20 bp, Artificial
  oligonucleotide
gctagccccgaagggctcgc

SEQ ID NO: 1119 - DNA, 20 bp, Artificial
  oligonucleotide
gccccgaagggctcgctcga

SEQ ID NO: 1120 - DNA, 20 bp, Artificial
  oligonucleotide
atcccaaggttgagccttgg

SEQ ID NO: 1121 - DNA, 20 bp, Artificial
  oligonucleotide
gagccttggactttcacttc

SEQ ID NO: 1122 - DNA, 20 bp, Artificial
  oligonucleotide
caaggttgagccttggactt

SEQ ID NO: 1123 - DNA, 20 bp, Artificial
  oligonucleotide
gagctttccactctccttgt

SEQ ID NO: 1124 - DNA, 20 bp, Artificial
  oligonucleotide
ccaaggttgagccttggact

SEQ ID NO: 1125 - DNA, 20 bp, Artificial
  oligonucleotide
cgggctcctctctcagcgat

SEQ ID NO: 1126 - DNA, 20 bp, Artificial
  oligonucleotide
ggagctttccactctccttg

SEQ ID NO: 1127 - DNA, 20 bp, Artificial
  oligonucleotide
gggctcctctctcagcgatg

SEQ ID NO: 1128 - DNA, 20 bp, Artificial
  oligonucleotide
tctccttgtcgctctccccg

SEQ ID NO: 1129 - DNA, 20 bp, Artificial
  oligonucleotide
tccttgtcgctctccccgag

SEQ ID NO: 1130 - DNA, 20 bp, Artificial
  oligonucleotide
agctttccactctccttgtc

SEQ ID NO: 1131 - DNA, 20 bp, Artificial
  oligonucleotide
ccactctccttgtcgctctc

SEQ ID NO: 1132 - DNA, 20 bp, Artificial
  oligonucleotide
ggctcctctctcagcgatgc

SEQ ID NO: 1133 - DNA, 20 bp, Artificial
  oligonucleotide
ccttgtcgctctccccgagc

SEQ ID NO: 1134 - DNA, 20 bp, Artificial
  oligonucleotide
cactctccttgtcgctctcc

SEQ ID NO: 1135 - DNA, 20 bp, Artificial
  oligonucleotide
actctccttgtcgctctccc

SEQ ID NO: 1136 - DNA, 20 bp, Artificial
  oligonucleotide
ctctccttgtcgctctcccc

SEQ ID NO: 1137 - DNA, 20 bp, Artificial
  oligonucleotide
gcgggctcctctctcagcga

SEQ ID NO: 1138 - DNA, 20 bp, Artificial
  oligonucleotide
ggctccatcatggttacctc

SEQ ID NO: 1139 - DNA, 22 bp, Artificial
  oligonucleotide
ccgtctcctaaggagctttcca

SEQ ID NO: 1140 - DNA, 22 bp, Artificial
  oligonucleotide
tccctccttaacggttacctca

SEQ ID NO: 1141 - DNA, 22 bp, Artificial
  oligonucleotide
tggctccataawggttacctca

SEQ ID NO: 1142 - DNA, 20 bp, Artificial
  oligonucleotide
cttcctccggcttgcgccgg

SEQ ID NO: 1143 - DNA, 20 bp, Artificial
  oligonucleotide
cgctcttcccgaktgactga

SEQ ID NO: 1144 - DNA, 20 bp, Artificial
  oligonucleotide
cctcgggctcctccatcwgc

(19) United States
(12) Patent Application Publication
(10) Pub. No.: US 2008/0026368 A1
(43) Pub. Date: Jan. 31, 2008

(54) Method for the Specific Rapid Detection of Beverage-Spoiling Microorganisms

(75) Inventors: Jiri Snaidr, Munchen (DE); Claudia Beimfohr, Munchen (DE); Karin Thelen, Grafelfing (DE); Angelika Lehner, Zurich (CH)

Correspondence Address: KNOBBE MARTENS OLSON & BEAR LLP, 2040 MAIN STREET, FOURTEENTH FLOOR, IRVINE, CA, 92614, US

(73) Assignee: VERMICON AG, Munchen (DE)

(21) Appl. No.: 10/574,717

(22) PCT Filed: Sep. 23, 2004
(86) PCT No.: PCT/EP04/10695

§ 371 Date: Aug. 30, 2007

(30) Foreign Application Priority Data
Sep. 23, 2003 (DE) 103 44 057.7

(51) Int. Cl. C12Q 1/68

(52) U.S. Cl. 435/6

(57) ABSTRACT

The invention relates to a method for the specific rapid detection of beverage-spoiling micro-organisms by means of in situ hybridisation. The invention also relates to specific oligonucleotide probes that are used in the detection method, and to kits containing said oligonucleotide probes.

The invention is related to a method for the specific fast detection of drink-spoiling microorganisms by in situ-hybridization. Moreover, the invention is related to specific oligonucleotide probes which are used in the course of the method for detection as well as kits which contain these oligonucleotide probes.

Under the generic clause “non-alcoholic drinks” groups of beverages are summarized like fruit juices, fruit nectars, fruit concentrates, mashed fruits, soft drinks and waters.

Basically non-alcoholic drinks can, due to their diverse/varying composition of nutrients and growth stimulating substances, be classified as potentially endangered by the growth of a large variety of microorganisms.

According to present knowledge mainly yeasts, molds, lactic acid bacteria, acetic acid bacteria, bacilli and alicyclobacilli are found in non-alcoholic drinks and are thus described as “drink-spoiling” microorganisms.

In general contaminations with these microorganisms do not lead to health defects of the consumer but are associated with turbidity, changes of taste and smell within the endproduct and cause high economic losses for the producing industry by image damage based thereon.

Based on the naturally high conccentrations of fruit acids and a corresponding low pH-value (a pH range from 2.5 to 4.5) in fruit juices and fruit nectars only acidophilic or acidotolerant microorganisms (such as lactic acid bacteria, alicyclobacilli, acid tolerant yeast and mold species) can grow and subsequently lead to a deterioration of these beverages.

A measure for restricting spoilage due to microorganisms is carbonisation of beverages. This method is commonly used for the production of soft drinks. By the addition of CO₂ almost anaerobic conditions are created in the product and only micro-aerophilic, facultatively anaerobic and anaerobic microorganisms (such as lactic acid bacteria, acetic acid bacteria and yeasts) are able to tolerate this environment.

Non-carbonated beverages are in most cases pasteurised in order to assure a long stability and quality of these products. By pasteurisation all vegetative microorganisms should be killed in a manner as comprehensive as possible. However, spores formed by bacilli or alicyclobacilli are not eliminated by this measure. Furthermore, some mold species are able to sustain this process without damage and subsequently create product damages.

A crucial factor for guaranteeing the biological quality of the beverages is the search for the cause of contamination in order to finally eliminate the same. In general, two ways of contamination are distinguished: contaminations are characterised as primary contaminations when microorganisms are introduced into the process by the raw material or by contamination within the process.

Secondary contaminations are those which appear in the filling area after the actual production of the beverage.

The challenge which arises by these different factors for the microbiological quality control, resides in the comprehensive and fast identification of all cells present in the product in order to be able to initiate corresponding counter measures as fast as possible.

Until now conventional detection of drink-spoiling microorganisms is performed by a several days lasting enrichment of the sample in a selective culture medium followed by light microscopy. Furthermore, for the accurate identification of the drink-spoiling microorganism further physiological tests (like Gram-staining, sugar consumption tests) need to be carried out.

The disadvantages of this solely cultivation-based method are the long duration of the analysis, which cause significant logistic costs in beverage-producing companies. Furthermore, there is the threat of significant image loss for said company, if, after the delivery of products whose microbiological findings had not yet been inequivocally stated, contaminationen are realised and draw-back actions of the spoiled product batches are required.

In the following the drink-spoiling microorganisms and their state of the art detection is described in detail.

Yeasts and Molds:

Microorganisms which can survive heat treatment and cause subsequently problems in the beverages are mainly the molds Byssochlamys fulva and B. nivea, Neosartorya fischeri and Talaromyces flavus as well as some yeasts. In carbonated drinks mainly the acid-tolerant, fermentative members of yeasts (Saccharomyces spp., Dekkera spp. and Zygosaccharomyces bailii) are dominating. Besides the threat of product damage based on taste alterations and turbidity caused by these “fermentative yeasts” there is a potential danger of occasional bursts of the filled bottles.

The detection of yeasts and molds is currently performed by cultivation on corresponding culture media (e.g. SSL-bouillon, OFS-medium, malt-dextrose-medium, wort-agar) and needs between 2 and 7 days. A detection on genus or even species level is very time-consuming and is normally not performed.

Lactic Acid Bacteria

The members of lactic acid bacteria are Gram-positive, non spore-forming, catalase-negative rods and cocci which are characterised by a very high nutrient demand (above all vitamines, amino acids, purines and pyrimidines). As indicated by the name all lactic acid bacteria are able to produce lactic acid as fermentation product.

Due to their anaerobic growth and for anaerobic microorganisms atypical high tolerance and insensitivity against oxygen they are described as aerotolerant anaerobics.

Up until now the genera Lactobacillus, Lactococcus, Leuconostoc, Oenococcus, Carnobacterium, Bifidobacterium, Enterococcus, Pediococcus, Weissella and Streptococcus are referred to as “lactic acid bacteria”.

Lactic acid bacteria play an ambivalent role in the food industry. On the one side their presence is wished and indispensable in some processes such as, e.g., the production of sauerkraut. On the other side their presence in beer or fruit juices can lead to a deterioration of the products. The growth of these bacteria is manifested mainly by turbidity, acidification and formation of gas and slime.

In the non-alcoholic drinks industry mainly the bacterial genera Leuconostoc, Lactococcus, Lactobacillus, Oenococcus, Weissella and Pediococcus are relevant as contaminants. Lactic acid bacteria are detected by a 5 to 7 days incubation at 25° C. on MRS agar (pH 5.7).

Acetic Acid Bacteria

Bacteria of the genera Acetobacter, Gluconobacter, Gluconoacetobacter and Acidomonas are described with the trivial name “acetic acid bacteria”. Bacteria of these genera are gram-negative, obligate aerobic, oxidase-negative rods whose optimum growth temperature is at 30° C. Acetic acid bacteria are able to grow also at pH values of 2.2 to 3.0 and, therefore, can produce product damages in beverages having this pH value.

Phylogenetically, bacteria of this genus are members of the Alphaproteobacteria.

The product damages mainly goes along with turbidity and alteration of the taste by the formation of acetic acid and gluconic acid. For the detection of acetic acid bacteria mainly ACM-agar (incubation time: 14 days) and DSM-agar (incubation time: 3 to 5 days) have proved themselves.

Bacilli:

Bacilli are Gram-positive aerobic, partly facultatively anaerobic, mostly catalase-positive spore-forming rods. Up until now Bacillus coagulans was mainly identified as spoilage microorganism in the non-alcoholic beverage industry.

The detection is performed by plating the sample on dextrose-caseine-peptone agar or yeast extract-peptone dextrose starch agar and subsequent incubation at 55° C. (incubation time: 3 days). In order to activate the spores and to achieve a germination of the spores of B. coagulans, respectively, a heat treatment of the sample is recommended at 80° C. for 10 min. before the actual incubation.

Alicyclobacilli:

Alicyclobacilli are Gram-positive, aerobic, thermophilic and catalase-positive spore-forming rods. Members of this genus produce ?-alicyclic fatty acids as main fatty acids. Up until now Alicyclobacillus acidoterrestris was mainly identified in the non-alcoholic beverage industry as spoilage organism. In rare cases also A. acidocaldarius and A. acidiphilus were identified in spoiled beverages.

The optimum range of the growth temperature for Alicyclobacillus spp. is between 26 and 55° C. The pH range where bacteria of this genus can grow, is between 2.2 and 5.8.

The growth of A. acidoterrestris leads to spoilage in fruit juices, which is manifested as alteration of the smell and taste due to the formation of guiacol and di-bromophenol. A contamination with this organism proceeds mostly in a non-apparent way, which means that only in rare cases a turbidity is seen in infected beverages.

Alicyclobacilli can be detected by cultivation for several days at 44-46° C. on orange serum agar, potato dextrose agar, K-agar, YSG-agar or BAM-agar. Furthermore, for the exact confirmation of the finding a set of physiological tests is necessary. In order to activate the spores and to achieve a germination of the spores of Alicyclobacillus ssp., respectively, heat treatment of the sample is recommended at 80° C. for 10 min. before the actual incubation.

The routine detection methods for drink-spoiling microorgansims used so far, are very protracted and are partly too inaccurate and thus prevent fast and effective counter measures in order to save the contaminated product. The inaccuracy of the detection arises from a missing differentiation up to genus and/or species level.

As a logical consequence of the difficulties presented by traditional cultivation methods for the detection of drink-spoiling microorganisms, detection methods on the basis of nucleic acids are suitable for the fast, safe and specific identification of spoilage microorganims in non-alcoholic beverages.

In PCR, which is polymerase chain reaction, a characteristic piece of the respective bacterial genome is amplified with specific primers. If the primer finds its target site, a million-fold amplification of a piece of the inherited material occurs. In the following analysis, for example by an agarose gel separating DNA fragments, a qualitative evaluation can take place. In the most simple case this leads to the conclusion that target sites for the primers used were present in the tested sample. Further conclusions are not possible; these target sites can originate from both a living bacterium and a dead bacterium, or from naked DNA. Since the PCR reaction is positive also in the presence of a dead bacterium or naked DNA, this often leads to false-positive results. A further refinement of this technique is the quantitative PCR which aims at establishing a correlation between the amount of bacteria present and the amount of amplified DNA. Advantages of the PCR are its high specificity, its ease of application and its low expenditure of time. Its main disadvantages are its high susceptibility to contamination and therefore false-positive results, as well as the aforementioned lacking possibility to discriminate between viable and dead cells, and naked DNA, respectively.

A unique approach to combine the specificity of molecularbiological methods such as PCR and the possibility of visualizing bacteria, which is provided by the antibody methods, is the method of fluorescence in situ hybridization (FISH; R. I. Amann, W. Ludwig and K.-H. Schleifer, 1995. Phylogenetic identification and in situ detection of individual microbial cells without cultivation. Microbiol. Rev. 59, p. 143-169). Using this method bacteria species, genera or groups can be identified and visualized with high specificity.

The FISH technique is based on the fact that in cells of microorganism there are certain molecules which have been mutated only to a small extent in the course of evolution because of their essential function. These are the 16S and the 23S ribosomal ribonucleic acid (rRNA). Both are components of the ribosomes, the sites of protein biosynthesis, and can serve as specific markers on account of their ubiquitous distribution, their size and their structural and functional constancy (Woese, C. R., 1987. Bacterial evolution. Microbiol. Rev. 51, p. 221-271). Based on a comparative sequence analysis, phylogenetic relationships can be established based on these data alone. For this purpose, the sequence data have to be brought into an alignment. In the alignment, which is based on the knowledge about the secondary structure and tertiary structure of these macromolecules, the homologous positions of the ribosomal nucleic acids are brought in line with each other.

Based on these data, phylogenetic calculations can be made. The use of the most modern computer technology allows to performe even large-scale calculations fast and effectively, as well as to set up large databases which contain the alignment sequences of the 16S, 18S, 23S and 26S rRNA. Due to the fast access to this data material, newly acquired sequences can be phylogenetically analyzed within a short time. These rRNA databases can be used to design species-specific and genus-specific gene probes. Hereby all available rRNA sequences are compared with each other and probes are designed for specific sequence sites, which specifically target a specific species, genus or group of bacteria.

In the FISH (fluorescence in situ hybridization) technique, these gene probes which are complementary to a certain region on the ribosomal target sequence, are intoduced into the cell. The gene probes are generally small, 16-20 bases long, single-stranded deoxyribonucleic acid pieces and are directed against a target region which is characteristic for a bacterial species or a bacterial group. If a fluorescencently labeled gene probe finds its target sequence in a cell of a microorganisms, it binds to it and the cells can be detected by means of a fluorescence microscope because of their fluorescence.

The FISH analysis is always performed on a slide, as for the evaluation the bacteria are visualized, i. e. rendered visible, by irradiation with high-energy light. But herein lies one of the disadvantages of the classical FISH analysis: because by definition only comparatively small volumina can be analyzed on a slide, the sensitivity of the method is not satisfying and not sufficient for a reliable analysis.

The present invention thus combines the advantages of the classical FISH analysis with those of cultivation. A comparatively short cultivation step ensures that the bacteria to be detected are present in sufficient numbers before the bacteria are detected using specific FISH.

The practising of the methods described in the present application for the specific detection of drink-spoiling yeasts of the genera Zygosaccharomyces, Hanseniaspora, Candida, Brettanomyces, Dekkera, Pichia, Saccharomyces and Saccharomycodes in particular the species Zygosaccharomyces bailii, Z. mellis, Z. rouxii, Z. bisporus, Z. fermentati, Z. microellipsoides, Hanseniaspora uvarum, Candida intermedia, C. crusei (Issatchenkia orientalis), C. parapsilosis, Brettanomyces bruxellensis, B. naardenensis, Dekkera anomala, Pichia membranaefaciens, P. minuta, P. anomala, Saccharomyces exiguus, S. cerevisiae, Saccharomycodes ludwigii or for the specific detection of drink-spoiling molds of the genera Mucor, Byssochlamys, Neosartorya, Aspergillus and Talaromyces in particular species of Mucor racemosus, Byssochlamys nivea, Neosartorya fischeri, Aspergillus fumigatus and A. fischeri, Talaromyces flavus, T. bacillisporus and T. flavus or for the specific detection of drink-spoiling bacteria of the genera Lactobacillus, Leuconostoc, Oenococcus, Weissella, Lactococcus, Acetobacter, Gluconobacter, Gluconoacetobacter, Bacillusand Alicyclobacillus, in particular species of Lactobacillus collinoides, Leuconostoc mesenteroides, L. pseudomesenteroides, Oenococcus oeni, Bacillus coagulans, Alicyclobacillus ssp., A. acidoterrestris, A. cycloheptanicus and A. herbarius thus comprises the following steps:
cultivating the drink-spoiling microorganisms present in the sample to be analysed
fixing the drink-spoiling microorganisms present in the sample
incubating the fixed drink-spoiling microorganisms with at least one nucleic acid probe and optionally in combination with a competitor probe, in order to achieve hybridization,
removing or washing off the non-hybridized nucleic acid probe and
detecting the drink-spoiling microorganisms hybridized to the nucleic acid probe molecules.

Within the present invention “cultivation” is understood to mean the propagation of the microorganisms present in the sample in a suitable cultivation medium.

For the detection of yeasts and molds the cultivation may occur, for example, in SSL-bouillon for 24 hours at 25° C. For the detection of lactic acid bacteria the cultivation may occur, for example, in MRS-bouillon for 48 hours at 30° C. For the detection of acetic acid bacteria the cultivation may occur, for example, on DSM-agar for 48 hours at 28° C. For the detection of bacilli, in particular B. coagulans, the cultivation may occur, for example, on dextrose-caseine-peptone agar for 48 hours at 55° C. For the detection of alicyclobacilli the cultivation may occur, for example, in BAM-bouillon for 48 hours at 44° C.

In any case, the person skilled in the art can find suitable cultivation methods in the prior art for each microorganism and each group of microorganisms to be analysed, respectively.

Within the present invention “fixing” of the microorganism is understood as a treatment with which the envelope of the microorganism is made permeable for nucleic acid probes. For fixation, usually ethanol is used. If the cell wall cannot be penetrated by the nucleic acid probes despite of using these techniques, the person skilled in the art will know enough further techniques which lead to the same result. These include, for example, methanol, mixtures of alcohols, low percentage paraformaldehyde solution or a diluted formaldehyde solution, enzymatic treatments or the like.

In a particularly preferred embodiment of the method of the present invention an enzymatic step may follow in order to cause complete cell disintegration of the microoganisms. Enzymes which can accordingly be used for this step, are, for instance, lysozyme, proteinase K, and mutanolysine. The one skilled in the art will know sufficient suitable techniques and will easily find out which means is particularly suitable for cell disintegration of a certain microorganism.

Within the present invention the fixed microorganisms are incubated with fluorescencently labeled nucleic acid probes for the “hybridization”. These nucleic acid probes can, after the fixing, penetrate the cell wall and bind to the target sequence in the cell corresponding to the nucleic acid probe. Binding is to be understood as formation of hydrogen bonds between complementary nucleic acid pieces.

In such case the nucleic acid probe can be complementary to a chromosomal or episomal DNA, but can also be complementary to an mRNA or rRNA of the microorganism to be detected. It is advantageous to select a nucleic acid probe which is complementary to a region present in copies of more than 1 in the microorganism to be detected. The sequence to be detected is preferably present in 500-100,000 copies per cell, especially preferred 1,000-50,000 copies. For this reason the sequence of the rRNA is preferably used as a target site, since the ribosomes as sites of protein biosynthesis are present many thousandfold in each active cell.

The nucleic acid probe within the meaning of the invention may be a DNA or RNA probe comprising usually between 12 and 100 nucleotides, preferably between 15 and 50, more between 17 and 25 nucleotides. The selection of the nucleic acid probes is performed taking into consideration whether a complementary sequence is present in the microorganism to be detected. By this selection of a defined sequence, a species of a microorganism, a genus of a microorganism or an entire microorganism group may be detected. In a probe consisting of 15 nucleotides, the sequences should be 100% complementary. In case of oligonucleotides of more than 15 nucleotides, depending on the length of the oligonucleotide, one or more mismatches are allowed.

To increase the specificity of nucleic acid probes competitor probes can be used. Within the present invention competitor probes are understood to mean in particular oligonucleotides which block possibly undesired bindings of the nucleic acid probes and thereby show a higher sequence similarity to the non-target genera and species of microorganisms, respectively, than to the target genera and species of microorganisms, respectively. By using competitor probes the binding of the nucleic acid probe to the nucleic acid sequence of non-target genera or species of microorganisms can be prevented and thus does not lead to false signals. The non-labelled competitor probe is always used in combination with the labelled oligonucleotide probe.

The competitor probe should be complementary to a nucleic acid sequence having high sequence similarity to the nucleic acid sequence of the genera and species of microorganism, respectively, to be detected. In a particularly preferred embodiment the competitor probe is complementary to the rRNA of non-target genera and species of microorganism, respectively.

Within the meaning of the invention the competitor probe is a DNA or RNA sequence usually comprising between 12 and 100 nucleotides, preferably between 15 and 50, particularly preferably between 17 and 25 nucleotides. By selecting a defined sequence, a bacterial species, a bacterial genus or an entire bacterial group may be blocked. A probe consisting of 15 nucleotides should be 100% complementary to the nucleic acid sequence to be blocked. In case of oligonucleotides consisting of more than 15 nucleotides, depending on the length of the oligonucleotide, one or more mismatches are allowed.

Within the methods of the present invention the nucleic acid probe molecules of the present invention have the following lengths and sequences (all nucleic acid probe molecules are noted in 5′-3′ direction).

The nucleic acid probe molecules of the present invention are useful for the specific detection of drink-spoiling yeasts of the genera Zygosaccharomyces, Hanseniaspora, Candida, Brettanomyces, Dekkera, Pichia, Saccharomyces and Saccharomycodes in particular the species Zygosaccharomyces bailii, Z. mellis, Z. rouxii, Z. bisporus, Z. fermentati, Z. microellipsoides, Hanseniaspora uvarum, Candida intermedia, C. crusei (Issatchenkia orientalis), C. parapsilosis, Brettanomyces bruxellensis, B. naardenensis, Dekkera anomala, Pichia membranaefaciens, P. minuta, P. anomala, Saccharomyces exiguus, S. cerevisiae, Saccharomycodes ludwigii or for the specific detection of drink-spoiling molds of the genera Mucor, Byssochlamys, Neosartorya, Aspergillus and Talaromyces in particular species of Mucor racemosus, Byssochlamys nivea, Neosartorya fischeri, Aspergillus fumigatus and A. fischeri, Talaromyces flavus, T bacillisporus and T. flavus or for the specific detection of drink-spoiling bacteria of the genera Lactobacillus, Leuconostoc, Oenococcus, Weissella, Lactococcus, Acetobacter, Gluconobacter, Gluconoacetobacter, Bacillus and Alicyclobacillus, in particular species of Lactobacillus collinoides, Leuconostoc mesenteroides, L. pseudomesenteroides, Oenococcus oeni, Bacillus coagulans, Alicyclobacillus ssp., A. acidoterrestris, A. cycloheptanicus and A. herbarius and are used correspondingly in the detection method according to the invention.

Within the present invention probes that detect different species of microorganims can be used in combination, in order to enable the simultaneous detection of different microoganisms. This leads likewise to an increase of speed of the detection method.

a) Nucleic Acid Molecules Which Specifically Detect Drink-Spoiling Yeasts:

| | |
|---|---|
| SEQ ID No. 1: | 5′- GTTTGACCAGATTCTCCGCTC |

The sequence SEQ ID No. 1 is particularly useful for the detection of microorganisms of the genus Zygosaccharomyces.

| | |
|---|---|
| SEQ ID No. 2: | 5′- GTTTGACCAGATTTTCCGCTCT |
| SEQ ID No. 3: | 5′- GTTTGACCAAATTTTCCGCTCT |
| SEQ ID No. 4: | 5′- GTTTGTCCAAATTCTCCGCTCT |

The nucleic acid molecules according to SEQ ID No. 2 to SEQ ID No. 4 are used as unlabelled competitor probes for the detection of microorganisms of the genus Zygosaccharomyces in combination with the nucleic acid probe according to SEQ ID No. 1 in order to prevent the binding of the labelled nucleic acid probe specific for members of the genus Zygosaccharomyces to nucleic acid sequences, which are not specific for members of the genus Zygosaccharomyces.

| | |
|---|---|
| SEQ ID No. 5: | 5′- CCCGGTCGAATTAAAACC |
| SEQ ID No. 6: | 5′- GCCCGGTCGAATTAAAAC |
| SEQ ID No. 7: | 5′- GGCCCGGTCGAATTAAAA |
| SEQ ID No. 8: | 5′- AGGCCCGGTCGAATTAAA |
| SEQ ID No. 9: | 5′- AAGGCCCGGTCGAATTAA |
| SEQ ID No. 10: | 5′- ATATTCGAGCGAAACGCC |
| SEQ ID No. 11: | 5′- AAAGATCCGGACCGGCCG |
| SEQ ID No. 12 | 5′- GGAAAGATCCGGACCGGC |
| SEQ ID No. 13 | 5′- GAAAGATCCGGACCGGCC |
| SEQ ID No. 14 | 5′- GATCCGGACCGGCCGACC |
| SEQ ID No. 15 | 5′- AGATCCGGACCGGCCGAC |
| SEQ ID No. 16 | 5′- AAGATCCGGACCGGCCGA |
| SEQ ID No. 17 | 5′- GAAAGGCCCGGTCGAATT |
| SEQ ID No. 18 | 5′- AAAGGCCCGGTCGAATTA |
| SEQ ID No. 19 | 5′- GGAAAGGCCCGGTCGAAT |
| SEQ ID No. 20 | 5′- AGGAAAGGCCCGGTCGAA |
| SEQ ID No. 21 | 5′- AAGGAAAGGCCCGGTCGA |

The sequences SEQ ID No. 5 to SEQ ID No. 21 are particularly suitable for the detection of Zygosaccharomyces bailii.

| | |
|---|---|
| SEQ ID No. 22: | 5′- ATAGCACTGGGATCCTCGCC |

The sequence SEQ ID No. 22 is particularly suitable for the detection of Zygosaccharomyces fermentati.

| | |
|---|---|
| SEQ ID No. 23: | 5′- CCAGCCCCAAAGTTACCTTC |
| SEQ ID No. 24: | 5′- TCCTTGACGTAAAGTCGCAG |

The sequences SEQ ID No. 23 to SEQ ID No. 24 are particularly suitable for the detection of Zygosaccharomyces microellipsoides.

| | |
|---|---|
| SEQ ID No. 25: | 5′- GGAAGAAAACCAGTACGC |
| SEQ ID No. 26: | 5′- CCGGTCGGAAGAAAACCA |
| SEQ ID No. 27: | 5′- GAAGAAAACCAGTACGCG |
| SEQ ID No. 28: | 5′- CCCGGTCGGAAGAAAACC |
| SEQ ID No. 29: | 5′- CGGTCGGAAGAAAACCAG |
| SEQ ID No. 30: | 5′- GGTCGGAAGAAAACCAGT |
| SEQ ID No. 31: | 5′- AAGAAAACCAGTACGCGG |
| SEQ ID No. 32: | 5′- GTACGCGGAAAAATCCGG |
| SEQ ID No. 33: | 5′- AGTACGCGGAAAAATCCG |
| SEQ ID No. 34: | 5′- GCGGAAAAATCCGGACCG |
| SEQ ID No. 35: | 5′- CGGAAGAAAACCAGTACG |
| SEQ ID No. 36: | 5′- GCCCGGTCGGAAGAAAAC |
| SEQ ID No. 37: | 5′- CGCGGAAAAATCCGGACC |
| SEQ ID No. 38: | 5′- CAGTACGCGGAAAAATCC |
| SEQ ID No. 39: | 5′- AGAAAACCAGTACGCGGA |
| SEQ ID No. 40: | 5′- GGCCCGGTCGGAAGAAAA |
| SEQ ID No. 41: | 5′- ATAAACACCACCCGATCC |
| SEQ ID No. 42: | 5′- ACGCGGAAAAATCCGGAC |
| SEQ ID No. 43: | 5′- GAGAGGCCCGGTCGGAAG |
| SEQ ID No. 44: | 5′- AGAGGCCCGGTCGGAAGA |
| SEQ ID No. 45: | 5′- GAGGCCCGGTCGGAAGAA |
| SEQ ID No. 46: | 5′- AGGCCCGGTCGGAAGAAA |
| SEQ ID No. 47: | 5′- CCGAGTGGGTCAGTAAAT |
| SEQ ID No. 48: | 5′- CCAGTACGCGGAAAAATC |
| SEQ ID No. 49: | 5′- TAAACACCACCCGATCCC |
| SEQ ID No. 50: | 5′- GGAGAGGCCCGGTCGGAA |
| SEQ ID No. 51: | 5′- GAAAACCAGTACGCGGAA |
| SEQ ID No. 52: | 5′- TACGCGGAAAAATCCGGA |
| SEQ ID No. 53: | 5′- GGCCACAGGGACCCAGGG |
| SEQ ID No. 54: | 5′- TCACCAAGGGCCACAGGG |
| SEQ ID No. 55: | 5′- GGGCCACAGGGACCCAGG |
| SEQ ID No. 56: | 5′- TTCACCAAGGGCCACAGG |
| SEQ ID No. 57: | 5′- ACAGGGACCCAGGGCTAG |
| SEQ ID No. 58: | 5′- AGGGCCACAGGGACCCAG |
| SEQ ID No. 59: | 5′- GTTCACCAAGGGCCACAG |
| SEQ ID No. 60: | 5′- GCCACAGGGACCCAGGGC |
| SEQ ID No. 61: | 5′- CAGGGACCCAGGGCTAGC |
| SEQ ID No. 62: | 5′- AGGGACCCAGGGCTAGCC |
| SEQ ID No. 63: | 5′- ACCAAGGGCCACAGGGAC |
| SEQ ID No. 64: | 5′- CCACAGGGACCCAGGGCT |
| SEQ ID No. 65: | 5′- CACAGGGACCCAGGGCTA |
| SEQ ID No. 66: | 5′- CACCAAGGGCCACAGGGA |
| SEQ ID No. 67: | 5′- GGGACCCAGGGCTAGCCA |
| SEQ ID No. 68: | 5′- AGGAGAGGCCCGGTCGGA |
| SEQ ID No. 69: | 5′- AAGGAGAGGCCCGGTCGG |
| SEQ ID No. 70: | 5′- GAAGGAGAGGCCCGGTCG |
| SEQ ID No. 71: | 5′- AGGGCTAGCCAGAAGGAG |
| SEQ ID No. 72: | 5′- GGGCTAGCCAGAAGGAGA |
| SEQ ID No. 73: | 5′- AGAAGGAGAGGCCCGGTC |
| SEQ ID No. 74: | 5′- CAAGGGCCACAGGGACCC |
| SEQ ID No. 75: | 5′- CCAAGGGCCACAGGGACC |

The sequences SEQ ID No. 25 to SEQ ID No. 75 are particularly suitable for the detection of Zygosaccharomyces mellis.

| | |
|---|---|
| SEQ ID No. 76: | 5′- GTCGGAAAAACCAGTACG |
| SEQ ID No. 77: | 5′- GCCCGGTCGGAAAAACCA |
| SEQ ID No. 78: | 5′- CCGGTCGGAAAAACCAGT |
| SEQ ID No. 79: | 5′- CCCGGTCGGAAAAACCAG |
| SEQ ID No. 80: | 5′- TCGGAAAAACCAGTACGC |
| SEQ ID No. 81: | 5′- CGGAAAAACCAGTACGCG |
| SEQ ID No. 82: | 5′- GGAAAAACCAGTACGCGG |
| SEQ ID No. 83: | 5′- GTACGCGGAAAAATCCGG |
| SEQ ID No. 84: | 5′- AGTACGCGGAAAAATCCG |
| SEQ ID No. 85: | 5′- GCGGAAAAATCCGGACCG |
| SEQ ID No. 86: | 5′- GGTCGGAAAAACCAGTAC |
| SEQ ID No. 87: | 5′- ACTCCTAGTGGTGCCCTT |
| SEQ ID No. 88: | 5′- GCTCCACTCCTAGTGGTG |
| SEQ ID No. 89: | 5′- CACTCCTAGTGGTGCCCT |
| SEQ ID No. 90: | 5′- CTCCACTCCTAGTGGTGC |
| SEQ ID No. 91: | 5′- TCCACTCCTAGTGGTGCC |
| SEQ ID No. 92: | 5′- CCACTCCTAGTGGTGCCC |
| SEQ ID No. 93: | 5′- GGCTCCACTCCTAGTGGT |
| SEQ ID No. 94: | 5′- AGGCTCCACTCCTAGTGG |
| SEQ ID No. 95: | 5′- GGCCCGGTCGGAAAAACC |
| SEQ ID No. 96: | 5′- GAAAAACCAGTACGCGGA |
| SEQ ID No. 97: | 5′- CGCGGAAAAATCCGGACC |
| SEQ ID No. 98: | 5′- CAGTACGCGGAAAAATCC |
| SEQ ID No. 99: | 5′- CGGTCGGAAAAACCAGTA |
| SEQ ID No. 100: | 5′- AAGGCCCGGTCGGAAAAA |
| SEQ ID No. 101: | 5′- CAGGCTCCACTCCTAGTG |
| SEQ ID No. 102: | 5′- CTCCTAGTGGTGCCCTTC |
| SEQ ID No. 103: | 5′- TCCTAGTGGTGCCCTTCC |
| SEQ ID No. 104: | 5′- GCAGGCTCCACTCCTAGT |
| SEQ ID No. 105: | 5′- AGGCCCGGTCGGAAAAAC |
| SEQ ID No. 106: | 5′- ACGCGGAAAAATCCGGAC |
| SEQ ID No. 107: | 5′- CCAGTACGCGGAAAAATC |
| SEQ ID No. 108: | 5′- CTAGTGGTGCCCTTCCGT |
| SEQ ID No. 109: | 5′- GAAAGGCCCGGTCGGAAA |
| SEQ ID No. 110: | 5′- AAAGGCCCGGTCGGAAAA |
| SEQ ID No. 111: | 5′- TACGCGGAAAAATCCGGA |
| SEQ ID No. 112: | 5′- GGAAAGGCCCGGTCGGAA |
| SEQ ID No. 113: | 5′- ATCTCTTCCGAAAGGTCG |
| SEQ ID No. 114: | 5′- CATCTCTTCCGAAAGGTC |
| SEQ ID No. 115: | 5′- CTCTTCCGAAAGGTCGAG |
| SEQ ID No. 116: | 5′- CTTCCGAAAGGTCGAGAT |
| SEQ ID No. 117: | 5′- TCTCTTCCGAAAGGTCGA |
| SEQ ID No. 118: | 5′- TCTTCCGAAAGGTCGAGA |
| SEQ ID No. 119: | 5′- CCTAGTGGTGCCCTTCCG |
| SEQ ID No. 120: | 5′- TAGTGGTGCCCTTCCGTC |
| SEQ ID No. 121: | 5′- AGTGGTGCCCTTCCGTCA |
| SEQ ID No. 122: | 5′- GCCAAGGTTAGACTCGTT |
| SEQ ID No. 123: | 5′- GGCCAAGGTTAGACTCGT |
| SEQ ID No. 124: | 5′- CCAAGGTTAGACTCGTTG |
| SEQ ID No. 125: | 5′- CAAGGTTAGACTCGTTGG |
| SEQ ID No. 126: | 5′- AAGGTTAGACTCGTTGGC |

The sequences SEQ ID No. 76 to SEQ ID No. 126 are particularly suitable for the detection of Zygosaccharomyces rouxii.

| | |
|---|---|
| SEQ ID No. 127: | 5′- CTCGCCTCACGGGGTTCTCA |

The sequence SEQ ID No. 127 is particularly suitable for the simultanous detection of Zygosaccharomyces mellis and Zygosaccharomyces rouxii.

| | |
|---|---|
| SEQ ID No. 128: | 5′-GGCCCGGTCGAAATTAAA |
| SEQ ID No. 129: | 5′-AGGCCCGGTCGAAATTAA |
| SEQ ID No. 130: | 5′-AAGGCCCGGTCGAAATTA |
| SEQ ID No. 131: | 5′-AAAGGCCCGGTCGAAATT |
| SEQ ID No. 132: | 5′-GAAAGGCCCGGTCGAAAT |
| SEQ ID No. 133: | 5′-ATATTCGAGCGAAACGCC |
| SEQ ID No. 134: | 5′-GGAAAGGCCCGGTCGAAA |
| SEQ ID No. 135: | 5′-AAAGATCCGGACCGGCCG |
| SEQ ID No. 136: | 5′-GGAAAGATCCGGACCGGC |
| SEQ ID No. 137: | 5′-GAAAGATCCGGACCGGCC |
| SEQ ID No. 138: | 5′-GATCCGGACCGGCCGACC |
| SEQ ID No. 139: | 5′-AGATCCGGACCGGCCGAC |
| SEQ ID No. 140: | 5′-AAGATCCGGACCGGCCGA |
| SEQ ID No. 141: | 5′-AGGAAAGGCCCGGTCGAA |
| SEQ ID No. 142: | 5′-AAGGAAAGGCCCGGTCGA |

The sequences SEQ ID No. 128 to SEQ ID No. 142 are particularly suitable for the detection of Zygosaccharomyces bisporus.

| | |
|---|---|
| SEQ ID No. 143: | 5′-CGAGCAAAACGCCTGCTTTG |
| SEQ ID No. 144: | 5′-CGCTCTGAAAGAGAGTTGCC |

The sequences SEQ ID No. 143 and SEQ ID No. 144 are particularly suitable for the detection of Hanseniaspora uvarum.

| | |
|---|---|
| SEQ ID No. 145: | 5′-AGTTGCCCCCTACACTAGAC |
| SEQ ID No. 146: | 5′-GCTTCTCCGTCCCGCGCCG |

The sequences SEQ ID No. 145 and SEQ ID No. 146 are particularly suitable for the detection of Candida intermedia.

| | |
|---|---|
| SEQ ID No. 147: | 5′-AGATTYTCCGCTCTGAGATGG |

The nucleic acid probe molecule according to SEQ ID No. 147 is used as unlabelled competitor probe for the detection of Candida intermedia in combination with the oligonucleotide probe according to SEQ ID No. 146, in order to prevent the binding of the labelled oligonucleotide probe specific for Candida intermedia to nucleic acid sequences which are not specific for Candida intermedia.

| | |
|---|---|
| SEQ ID No. 148: | 5′-CCTGGTTCGCCAAAAAGGC |

The sequence SEQ ID No. 148 is particularly suitable for the detection of Candida parapsilosis.

| | |
|---|---|
| SEQ ID No. 149: | 5′-GATTCTCGGCCCCATGGG |

The sequence SEQ ID No. 149 is particularly suitable for the detection of Candida crusei (Issatchenkia orientalis).

| | |
|---|---|
| SEQ ID No. 150: | 5′-ACCCTCTACGGCAGCCTGTT |

The sequence SEQ ID No. 150 is particularly suitable for the detection of Dekkera anomala and Brettanomyces (Dekkera) bruxellensis.

| | |
|---|---|
| SEQ ID No. 151: | 5′-GATCGGTCTCCAGCGATTCA |

The sequence SEQ ID No. 151 is particularly suitable for the detection of Brettanomyces (Dekkera) bruxellensis.

| | |
|---|---|
| SEQ ID No. 152: | 5′-ACCCTCCACGGCGGCCTGTT |

The sequence SEQ ID No. 152 is particularly suitable for the detection of Brettanomyces (Dekkera) naardenensis.

| | |
|---|---|
| SEQ ID No. 153: | 5′-GATTCTCCGCGCCATGGG |

The sequence SEQ ID No. 153 is particularly suitable for the detection of Pichia membranaefaciens.

| | |
|---|---|
| SEQ ID No. 154: | 5′-TCATCAGACGGGATTCTCAC |

The sequence SEQ ID No. 154 is particularly suitable for the simultanous detection of Pichia minuta and Pichia anomala.

| | |
|---|---|
| SEQ ID No. 155: | 5′-CTCATCGCACGGGATTCTCACC |
| SEQ ID No. 156: | 5′-CTCGCCACACGGGATTCTCACC |

The nucleic acid probe molecules according to SEQ ID No. 155 and SEQ ID No. 156 are used as unlabelled competitor probes for the simultanous detection of Pichia minuta and Pichia anomala in combination with the oligonucleotide probe according to SEQ ID No. 154, in order to prevent the binding of the labelled oligonucleotide probe specific for Pichia minuta and Pichia anomala, to nucleic acid sequences which are not specific for Pichia minuta and Pichia anomala.

| | |
|---|---|
| SEQ ID No. 157: | 5′-AGTTGCCCCCTCCTCTAAGC |

The sequence SEQ ID No. 157 is particularly suitable for the detection of Saccharomyces exiguus.

| | |
|---|---|
| SEQ ID No. 158: | 5′-CTGCCACAAGGACAAATGGT |
| SEQ ID No. 159: | 5′-TGCCCCCTCTTCTAAGCAAAT |

The sequences SEQ ID No. 158 and SEQ ID No. 159 are particularly suitable for the detection of Saccharomyces ludwigii.

| | |
|---|---|
| SEQ ID No. 160: | 5′-CCCCAAAGTTGCCCTCTC |

The sequence SEQ ID No. 160 is particularly suitable for the detection of Saccharomyces cerevisiae.

| | |
|---|---|
| SEQ ID No. 161: | 5′-GCCGCCCCAAAGTCGCCCTCTAC |
| SEQ ID No. 162: | 5′-GCCCCAGAGTCGCCTTCTAC |

The nucleic acid probe molecules according to SEQ ID No. 161 and SEQ ID No. 162 are used as unlabelled competitor probes for the detection of Saccharomyces cerevisiae in combination with the oligonucleotide probe according to SEQ ID No. 160, in order to prevent the binding of the labelled oligonucleotide probe specific for Saccharomyces cerevisiae, to nucleic acid sequences which are not specific for Saccharomyces cerevisiae.

b) Nucleic Acid Probe Molecules Which Specifically Detect Drink-Spoiling Molds:

| | |
|---|---|
| SEQ ID No. 163: | 5′-AAGACCAGGCCACCTCAT |

The sequence SEQ ID No. 163 is particularly suitable for the detection of Mucor racemosus.

| | |
|---|---|
| SEQ ID No. 164: | 5′-CATCATAGAACACCGTCC |

The sequence SEQ ID No. 164 is particularly suitable for the detection of Byssochlamys nivea.

| | |
|---|---|
| SEQ ID No. 165: | 5′-CCTTCCGAAGTCGAGGTTTT |

The sequence SEQ ID No. 165 is particularly suitable for the detection of Neosartorya fischeri.

| | |
|---|---|
| SEQ ID No. 166: | 5′-GGGAGTGTTGCCAACTC |

The sequence SEQ ID No. 166 is particularly suitable for the simultaneous detection of Aspergillus fumigatus and A. fischeri.

| | |
|---|---|
| SEQ ID No. 167: | 5′-AGCGGTCGTTCGCAACCCT |

The sequence SEQ ID No. 167 is particularly suitable for the detection of Talaromyces flavus.

| | |
|---|---|
| SEQ ID No. 168: | 5′-CCGAAGTCGGGGTTTTGCGG |

The sequence SEQ ID No. 168 is particularly suitable for the simultaneous detection of Talaromyces bacillisporus and T flavus.

c) Nucleic Acid Probe Molecules, Which Specifically Detect Drink-Spoiling Lactic Acid Bacteria

| | |
|---|---|
| SEQ ID No. 169: | 5′-GATAGCCGAAACCACCTTTC |
| SEQ ID No. 170: | 5′-GCCGAAACCACCTTTCAAAC |
| SEQ ID No. 171: | 5′-GTGATAGCCGAAACCACCTT |
| SEQ ID No. 172: | 5′-AGTGATAGCCGAAACCACCT |
| SEQ ID No. 173: | 5′-TTTAACGGGATGCGTTCGAC |
| SEQ ID No. 174: | 5′-AAGTGATAGCCGAAACCACC |
| SEQ ID No. 175: | 5′-GGTTGAATACCGTCAACGTC |
| SEQ ID No. 176: | 5′-GCACAGTATGTCAAGACCTG |
| SEQ ID No. 177: | 5′-CATCCGATGTGCAAGCACTT |
| SEQ ID No. 178: | 5′-TCATCCGATGTGCAAGCACT |
| SEQ ID No. 179: | 5′-CCGATGTGCAAGCACTTCAT |
| SEQ ID No. 180: | 5′-CCACTCATCCGATGTGCAAG |
| SEQ ID No. 181: | 5′-GCCACAGTTCGCCACTCATC |
| SEQ ID No. 182: | 5′-CCTCCGCGTTTGTCACCGGC |
| SEQ ID No. 183: | 5′-ACCAGTTCGCCACAGTTCGC |
| SEQ ID No. 184: | 5′-CACTCATCCGATGTGCAAGC |
| SEQ ID No. 185: | 5′-CCAGTTCGCCACAGTTCGCC |
| SEQ ID No. 186: | 5′-CTCATCCGATGTGCAAGCAC |
| SEQ ID No. 187: | 5′-TCCGATGTGCAAGCACTTCA |
| SEQ ID No. 188: | 5′-CGCCACTCATCCGATGTGCA |
| SEQ ID No. 189: | 5′-CAGTTCGCCACAGTTCGCCA |
| SEQ ID No. 190: | 5′-GCCACTCATCCGATGTGCAA |
| SEQ ID No. 191: | 5′-CGCCACAGTTCGCCACTCAT |
| SEQ ID No. 192: | 5′-ATCCGATGTGCAAGCACTTC |
| SEQ ID No. 193: | 5′-GTTCGCCACAGTTCGCCACT |
| SEQ ID No. 194: | 5′-TCCTCCGCGTTTGTCACCGG |
| SEQ ID No. 195: | 5′-CGCCAGGGTTCATCCTGAGC |
| SEQ ID No. 196: | 5′-AGTTCGCCACAGTTCGCCAC |
| SEQ ID No. 197: | 5′-TCGCCACAGTTCGCCACTCA |
| SEQ ID No. 198: | 5′-TTAACGGGATGCGTTCGACT |
| SEQ ID No. 199: | 5′-TCGCCACTCATCCGATGTGC |
| SEQ ID No. 200: | 5′-CCACAGTTCGCCACTCATCC |
| SEQ ID No. 201: | 5′-GATTTAACGGGATGCGTTCG |
| SEQ ID No. 202: | 5′-TAACGGGATGCGTTCGACTT |
| SEQ ID No. 203: | 5′-AACGGGATGCGTTCGACTTG |
| SEQ ID No. 204: | 5′-CGAAGGTTACCGAACCGACT |
| SEQ ID No. 205: | 5′-CCGAAGGTTACCGAACCGAC |
| SEQ ID No. 206: | 5′-CCCGAAGGTTACCGAACCGA |
| SEQ ID No. 207: | 5′-TTCCTCCGCGTTTGTCACCG |
| SEQ ID No. 208: | 5′-CCGCCAGGGTTCATCCTGAG |
| SEQ ID No. 209: | 5′-TCCTTCCAGAAGTGATAGCC |
| SEQ ID No. 210: | 5′-CACCAGTTCGCCACAGTTCG |
| SEQ ID No. 211: | 5′-ACGGGATGCGTTCGACTTGC |
| SEQ ID No. 212: | 5′-GTCCTTCCAGAAGTGATAGC |
| SEQ ID No. 213: | 5′-GCCAGGGTTCATCCTGAGCC |
| SEQ ID No. 214: | 5′-ACTCATCCGATGTGCAAGCA |
| SEQ ID No. 215: | 5′-ATCATTGCCTTGGTGAACCG |
| SEQ ID No. 216: | 5′-TCCGCGTTTGTCACCGGCAG |
| SEQ ID No. 217: | 5′-TGAACCGTTACTCCACCAAC |
| SEQ ID No. 218: | 5′-GAAGTGATAGCCGAAACCAC |
| SEQ ID No. 219: | 5′-CCGCGTTTGTCACCGGCAGT |
| SEQ ID No. 220: | 5′-TTCGCCACTCATCCGATGTG |
| SEQ ID No. 221: | 5′-CATTTAACGGGATGCGTTCG |
| SEQ ID No. 222: | 5′-CACAGTTCGCCACTCATCCG |
| SEQ ID No. 223: | 5′-TTCGCCACAGTTCGCCACTC |
| SEQ ID No. 224: | 5′-CTCCGCGTTTGTCACCGGCA |
| SEQ ID No. 225: | 5′-ACGCCGCCAGGGTTCATCCT |
| SEQ ID No. 226: | 5′-CCTTCCAGAAGTGATAGCCG |
| SEQ ID No. 227: | 5′-TCATTGCCTTGGTGAACCGT |
| SEQ ID No. 228: | 5′-CACAGTATGTCAAGACCTGG |
| SEQ ID No. 229: | 5′-TTGGTGAACCGTTACTCCAC |
| SEQ ID No. 230: | 5′-CTTGGTGAACCGTTACTCCA |
| SEQ ID No. 231: | 5′-GTGAACCGTTACTCCACCAA |
| SEQ ID No. 232: | 5′-GGCTCCCGAAGGTTACCGAA |
| SEQ ID No. 233: | 5′-GAAGGTTACCGAACCGACTT |
| SEQ ID No. 234: | 5′-TGGCTCCCGAAGGTTACCGA |
| SEQ ID No. 235: | 5′-TAATACGCCGCGGGTCCTTC |
| SEQ ID No. 236: | 5′-GAACCGTTACTCCACCAACT |
| SEQ ID No. 237: | 5′-TACGCCGCGGGTCCTTCCAG |
| SEQ ID No. 238: | 5′-TCACCAGTTCGCCACAGTTC |
| SEQ ID No. 239: | 5′-CCTTGGTGAACCGTTACTCC |
| SEQ ID No. 240: | 5′-CTCACCAGTTCGCCACAGTT |
| SEQ ID No. 241: | 5′-CGCCGCCAGGGTTCATCCTG |
| SEQ ID No. 242: | 5′-CCTTGGTGAACCATTACTCC |
| SEQ ID No. 243: | 5′-TGGTGAACCATTACTCCACC |
| SEQ ID No. 244: | 5′-GCCGCCAGGGTTCATCCTGA |
| SEQ ID No. 245: | 5′-GGTGAACCATTACTCCACCA |
| SEQ ID No. 246: | 5′-CCAGGGTTCATCCTGAGCCA |
| SEQ ID No. 247: | 5′-AATACGCCGCGGGTCCTTCC |
| SEQ ID No. 248: | 5′-CACGCCGCCAGGGTTCATCC |
| SEQ ID No. 249: | 5′-AGTTCGCCACTCATCCGATG |
| SEQ ID No. 250: | 5′-CGGGATGCGTTCGACTTGCA |
| SEQ ID No. 251: | 5′-CATTGCCTTGGTGAACCGTT |
| SEQ ID No. 252: | 5′-GCACGCCGCCAGGGTTCATC |
| SEQ ID No. 253: | 5′-CTTCCTCCGCGTTTGTCACC |
| SEQ ID No. 254: | 5′-TGGTGAACCGTTACTCCACC |
| SEQ ID No. 255: | 5′-CCTTCCTCCGCGTTTGTCAC |
| SEQ ID No. 256: | 5′-ACGCCGCGGGTCCTTCCAGA |
| SEQ ID No. 257: | 5′-GGTGAACCGTTACTCCACCA |
| SEQ ID No. 258: | 5′-GGGTCCTTCCAGAAGTGATA |
| SEQ ID No. 259: | 5′-CTTCCAGAAGTGATAGCCGA |
| SEQ ID No. 260: | 5′-GCCTTGGTGAACCATTACTC |
| SEQ ID No. 261: | 5′-ACAGTTCGCCACTCATCCGA |
| SEQ ID No. 262: | 5′-ACCTTCCTCCGCGTTTGTCA |
| SEQ ID No. 263: | 5′-CGAACCGACTTTGGGTGTTG |
| SEQ ID No. 264: | 5′-GAACCGACTTTGGGTGTTGC |
| SEQ ID No. 265: | 5′-AGGTTACCGAACCGACTTTG |
| SEQ ID No. 266: | 5′-ACCGAACCGACTTTGGGTGT |
| SEQ ID No. 267: | 5′-TTACCGAACCGACTTTGGGT |
| SEQ ID No. 268: | 5′-TACCGAACCGACTTTGGGTG |
| SEQ ID No. 269: | 5′-GTTACCGAACCGACTTTGGG |

The sequences SEQ ID No. 169 to SEQ ID No. 269 are particularly suitable for the detection of Lactobacillus collinoides.

| | |
|---|---|
| SEQ ID No. 270: | 5′-CCTTTCTGGTATGGTACCGTC |
| SEQ ID No: 271: | 5′-TGCACCGCGGAYCCATCTCT |

The sequences SEQ ID No. 270 to SEQ ID No. 271 are particularly suitable for the detection of members of the genus Leuconostoc.

| | |
|---|---|
| SEQ ID No. 272: | 5′-AGTTGCAGTCCAGTAAGCCG |
| SEQ ID No. 273: | 5′-GTTGCAGTCCAGTAAGCCGC |
| SEQ ID No. 274: | 5′-CAGTTGCAGTCCAGTAAGCC |
| SEQ ID No. 275: | 5′-TGCAGTCCAGTAAGCCGCCT |
| SEQ ID No. 276: | 5′-TCAGTTGCAGTCCAGTAAGC |
| SEQ ID No. 277: | 5′-TTGCAGTCCAGTAAGCCGCC |
| SEQ ID No. 278: | 5′-GCAGTCCAGTAAGCCGCCTT |
| SEQ ID No. 279: | 5′-GTCAGTTGCAGTCCAGTAAG |
| SEQ ID No. 280: | 5′-CTCTAGGTGACGCCGAAGCG |
| SEQ ID No. 281: | 5′-ATCTCTAGGTGACGCCGAAG |
| SEQ ID No. 282: | 5′-TCTAGGTGACGCCGAAGCGC |
| SEQ ID No. 283: | 5′-TCTCTAGGTGACGCCGAAGC |
| SEQ ID No. 284: | 5′-CCATCTCTAGGTGACGCCGA |
| SEQ ID No. 285: | 5′-CATCTCTAGGTGACGCCGAA |
| SEQ ID No. 286: | 5′-TAGGTGACGCCGAAGCGCCT |
| SEQ ID No. 287: | 5′-CTAGGTGACGCCGAAGCGCC |
| SEQ ID No. 288: | 5′-CTTAGACGGCTCCTTCCTAA |
| SEQ ID No. 289: | 5′-CCTTAGACGGCTCCTTCCTA |
| SEQ ID No. 290: | 5′-ACGTCAGTTGCAGTCCAGTA |
| SEQ ID No. 291: | 5′-CGTCAGTTGCAGTCCAGTAA |
| SEQ ID No. 292: | 5′-ACGCCGAAGCGCCTTTTAAC |
| SEQ ID No. 293: | 5′-GACGCCGAAGCGCCTTTTAA |
| SEQ ID No. 294: | 5′-GCCGAAGCGCCTTTTAACTT |
| SEQ ID No. 295: | 5′-CGCCGAAGCGCCTTTTAACT |
| SEQ ID No. 296: | 5′-GTGACGCCGAAGCGCCTTTT |
| SEQ ID No. 297: | 5′-TGACGCCGAAGCGCCTTTTA |
| SEQ ID No. 298: | 5′-AGACGGCTCCTTCCTAAAAG |
| SEQ ID No. 299: | 5′-ACGGCTCCTTCCTAAAAGGT |
| SEQ ID No. 300: | 5′-GACGGCTCCTTCCTAAAAGG |
| SEQ ID No. 301: | 5′-CCTTCCTAAAAGGTTAGGCC |

The sequences SEQ ID No. 272 to SEQ ID No. 301 are particularly suitable for the simultanous detection of Leuconostoc mesenteroides and Leuconostoc pseudomesenteroides.

| | |
|---|---|
| SEQ ID No. 302: | 5′-GGTGACGCCAAAGCGCCTTT |
| SEQ ID No. 303: | 5′-AGGTGACGCCAAAGCGCCTT |
| SEQ ID No. 304: | 5′-TAGGTGACGCCAAAGCGCCT |
| SEQ ID No. 305: | 5′-CTCTAGGTGACGCCAAAGCG |
| SEQ ID No. 306: | 5′-TCTAGGTGACGCCAAAGCGC |
| SEQ ID No. 307: | 5′-CTAGGTGACGCCAAAGCGCC |
| SEQ ID No. 308: | 5′-ACGCCAAAGCGCCTTTTAAC |
| SEQ ID No. 309: | 5′-CGCCAAAGCGCCTTTTAACT |
| SEQ ID No. 310: | 5′-TGACGCCAAAGCGCCTTTTA |
| SEQ ID No. 311: | 5′-TCTCTAGGTGACGCCAAAGC |
| SEQ ID No. 312: | 5′-GTGACGCCAAAGCGCCTTTT |
| SEQ ID No. 313: | 5′-GACGCCAAAGCGCCTTTTAA |
| SEQ ID No. 314: | 5′-ATCTCTAGGTGACGCCAAAG |
| SEQ ID No. 315: | 5′-CATCTCTAGGTGACGCCAAA |
| SEQ ID No. 316: | 5′-TCCATCTCTAGGTGACGCCA |
| SEQ ID No. 317: | 5′-CCATCTCTAGGTGACGCCAA |
| SEQ ID No. 318: | 5′-CTGCCTTAGACGGCTCCCCC |
| SEQ ID No. 319: | 5′-CCTGCCTTAGACGGCTCCCC |
| SEQ ID No. 320: | 5′-GTGTCATGCGACACTGAGTT |
| SEQ ID No. 321: | 5′-TGTGTCATGCGACACTGAGT |
| SEQ ID No. 322: | 5′-CTTTGTGTCATGCGACACTG |
| SEQ ID No. 323: | 5′-TTGTGTCATGCGACACTGAG |
| SEQ ID No. 324: | 5′-TGCCTTAGACGGCTCCCCCT |
| SEQ ID No. 325: | 5′-AGACGGCTCCCCCTAAAAGG |
| SEQ ID No. 326: | 5′-TAGACGGCTCCCCCTAAAAG |
| SEQ ID No. 327: | 5′-GCCTTAGACGGCTCCCCCTA |
| SEQ ID No. 328: | 5′-GCTCCCCCTAAAAGGTTAGG |
| SEQ ID No. 329: | 5′-GGCTCCCCCTAAAAGGTTAG |
| SEQ ID No. 330: | 5′-CTCCCCCTAAAAGGTTAGGC |
| SEQ ID No. 331: | 5′-TCCCCCTAAAAGGTTAGGCC |
| SEQ ID No. 332: | 5′-CCCTAAAAGGTTAGGCCACC |
| SEQ ID No. 333: | 5′-CCCCTAAAAGGTTAGGCCAC |
| SEQ ID No. 334: | 5′-CGGCTCCCCCTAAAAGGTTA |
| SEQ ID No. 335: | 5′-CCCCCTAAAAGGTTAGGCCA |
| SEQ ID No. 336: | 5′-CTTAGACGGCTCCCCCTAAA |
| SEQ ID No. 337: | 5′-TTAGACGGCTCCCCCTAAAA |
| SEQ ID No. 338: | 5′-GGGTTCGCAACTCGTTGTAT |
| SEQ ID No. 339: | 5′-CCTTAGACGGCTCCCCCTAA |
| SEQ ID No. 340: | 5′-ACGGCTCCCCCTAAAAGGTT |
| SEQ ID No. 341: | 5′-GACGGCTCCCCCTAAAAGGT |

The sequences SEQ ID No. 302 to SEQ ID No. 341 are particularly suitable for the detection of Leuconostoc pseudomesenteroides.

| | |
|---|---|
| SEQ ID No. 342: | 5′-ACGCCGCAAGACCATCCTCT |
| SEQ ID No. 343: | 5′-CTAATACGCCGCAAGACCAT |
| SEQ ID No. 344: | 5′-TACGCCGCAAGACCATCCTC |
| SEQ ID No. 345: | 5′-GTTACGATCTAGCAAGCCGC |
| SEQ ID No. 346: | 5′-AATACGCCGCAAGACCATCC |
| SEQ ID No. 347: | 5′-CGCCGCAAGACCATCCTCTA |
| SEQ ID No. 348: | 5′-GCTAATACGCCGCAAGACCA |
| SEQ ID No. 349: | 5′-ACCATCCTCTAGCGATCCAA |
| SEQ ID No. 350: | 5′-TAATACGCCGCAAGACCATC |
| SEQ ID No. 351: | 5′-AGCCATCCCTTTCTGGTAAG |
| SEQ ID No. 352: | 5′-ATACGCCGCAAGACCATCCT |
| SEQ ID No. 353: | 5′-AGTTACGATCTAGCAAGCCG |
| SEQ ID No. 354: | 5′-AGCTAATACGCCGCAAGACC |
| SEQ ID No. 355: | 5′-GCCGCAAGACCATCCTCTAG |
| SEQ ID No. 356: | 5′-TTACGATCTAGCAAGCCGCT |
| SEQ ID No. 357: | 5′-GACCATCCTCTAGCGATCCA |
| SEQ ID No. 358: | 5′-TTGCTACGTCACTAGGAGGC |
| SEQ ID No. 359: | 5′-ACGTCACTAGGAGGCGGAAA |
| SEQ ID No. 360: | 5′-TTTGCTACGTCACTAGGAGG |
| SEQ ID No. 361: | 5′-GCCATCCCTTTCTGGTAAGG |
| SEQ ID No. 362: | 5′-TACGTCACTAGGAGGCGGAA |
| SEQ ID No. 363: | 5′-CGTCACTAGGAGGCGGAAAC |
| SEQ ID No. 364: | 5′-AAGACCATCCTCTAGCGATC |
| SEQ ID No. 365: | 5′-GCACGTATTTAGCCATCCCT |
| SEQ ID No. 366: | 5′-CTCTAGCGATCCAAAAGGAC |
| SEQ ID No. 367: | 5′-CCTCTAGCGATCCAAAAGGA |
| SEQ ID No. 368: | 5′-CCATCCTCTAGCGATCCAAA |
| SEQ ID No. 369: | 5′-GGCACGTATTTAGCCATCCC |
| SEQ ID No. 370: | 5′-TACGATCTAGCAAGCCGCTT |
| SEQ ID No. 371: | 5′-CAGTTACGATCTAGCAAGCC |
| SEQ ID No. 372: | 5′-CCGCAAGACCATCCTCTAGC |
| SEQ ID No. 373: | 5′-CCATCCCTTTCTGGTAAGGT |
| SEQ ID No. 374: | 5′-AGACCATCCTCTAGCGATCC |
| SEQ ID No. 375: | 5′-CAAGACCATCCTCTAGCGAT |
| SEQ ID No. 376: | 5′-GCTACGTCACTAGGAGGCGG |
| SEQ ID No. 377: | 5′-TGCTACGTCACTAGGAGGCG |
| SEQ ID No. 378: | 5′-CTACGTCACTAGGAGGCGGA |
| SEQ ID No. 379: | 5′-CCTCAACGTCAGTTACGATC |
| SEQ ID No. 380: | 5′-GTCACTAGGAGGCGGAAACC |
| SEQ ID No. 381: | 5′-TCCTCTAGCGATCCAAAAGG |
| SEQ ID No. 382: | 5′-TGGCACGTATTTAGCCATCC |
| SEQ ID No. 383: | 5′-ACGATCTAGCAAGCCGCTTT |
| SEQ ID No. 384: | 5′-GCCAGTCTCTCAACTCGGCT |
| SEQ ID No. 385: | 5′-AAGCTAATACGCCGCAAGAC |
| SEQ ID No. 386: | 5′-GTTTGCTACGTCACTAGGAG |
| SEQ ID No. 387: | 5′-CGCCACTCTAGTCATTGCCT |
| SEQ ID No. 388: | 5′-GGCCAGCCAGTCTCTCAACT |
| SEQ ID No. 389: | 5′-CAGCCAGTCTCTCAACTCGG |
| SEQ ID No. 390: | 5′-CCCGAAGATCAATTCAGCGG |
| SEQ ID No. 391: | 5′-CCGGCCAGTCTCTCAACTCG |
| SEQ ID No. 392: | 5′-CCAGCCAGTCTCTCAACTCG |
| SEQ ID No. 393: | 5′-TCATTGCCTCACTTCACCCG |
| SEQ ID No. 394: | 5′-GCCAGCCAGTCTCTCAACTC |
| SEQ ID No. 395: | 5′-CACCCGAAGATCAATTCAGC |
| SEQ ID No. 396: | 5′-GTCATTGCCTCACTTCACCC |
| SEQ ID No. 397: | 5′-CATTGCCTCACTTCACCCGA |
| SEQ ID No. 398: | 5′-ATTGCCTCACTTCACCCGAA |
| SEQ ID No. 399: | 5′-CGAAGATCAATTCAGCGGCT |
| SEQ ID No. 400: | 5′-AGTCATTGCCTCACTTCACC |
| SEQ ID No. 401: | 5′-TCGCCACTCTAGTCATTGCC |
| SEQ ID No. 402: | 5′-TTGCCTCACTTCACCCGAAG |
| SEQ ID No. 403: | 5′-CGGCCAGTCTCTCAACTCGG |
| SEQ ID No. 404: | 5′-CTGGCACGTATTTAGCCATC |
| SEQ ID No. 405: | 5′-ACCCGAAGATCAATTCAGCG |
| SEQ ID No. 406: | 5′-TCTAGCGATCCAAAAGGACC |
| SEQ ID No. 407: | 5′-CTAGCGATCCAAAAGGACCT |
| SEQ ID No. 408: | 5′-GCACCCATCGTTTACGGTAT |
| SEQ ID No. 409: | 5′-CACCCATCGTTTACGGTATG |
| SEQ ID No. 410: | 5′-GCCACTCTAGTCATTGCCTC |
| SEQ ID No. 411: | 5′-CGTTTGCTACGTCACTAGGA |
| SEQ ID No. 412: | 5′-GCCTCAACGTCAGTTACGAT |
| SEQ ID No. 413: | 5′-GCCGGCCAGTCTCTCAACTC |
| SEQ ID No. 414: | 5′-TCACTAGGAGGCGGAAACCT |
| SEQ ID No. 415: | 5′-AGCCTCAACGTCAGTTACGA |
| SEQ ID No. 416: | 5′-AGCCAGTCTCTCAACTCGGC |
| SEQ ID No. 417: | 5′-GGCCAGTCTCTCAACTCGGC |
| SEQ ID No. 418: | 5′-CAAGCTAATACGCCGCAAGA |
| SEQ ID No. 419: | 5′-TTCGCCACTCTAGTCATTGC |
| SEQ ID No. 420: | 5′-CCGAAGATCAATTCAGCGGC |
| SEQ ID No. 421: | 5′-CGCAAGACCATCCTCTAGCG |
| SEQ ID No. 422: | 5′-GCAAGACCATCCTCTAGCGA |
| SEQ ID No. 423: | 5′-GCGTTTGCTACGTCACTAGG |
| SEQ ID No. 424: | 5′-CCACTCTAGTCATTGCCTCA |
| SEQ ID No. 425: | 5′-CACTCTAGTCATTGCCTCAC |
| SEQ ID No. 426: | 5′-CCAGTCTCTCAACTCGGCTA |
| SEQ ID No. 427: | 5′-TTACCTTAGGCACCGGCCTC |
| SEQ ID No. 428: | 5′-ACAAGCTAATACGCCGCAAG |
| SEQ ID No. 429: | 5′-TTTACCTTAGGCACCGGCCT |
| SEQ ID No. 430: | 5′-TTTTACCTTAGGCACCGGCC |
| SEQ ID No. 431: | 5′-ATTTTACCTTAGGCACCGGC |
| SEQ ID No. 432: | 5′-GATTTTACCTTAGGCACCGG |
| SEQ ID No. 433: | 5′-CTCACTTCACCCGAAGATCA |
| SEQ ID No. 434: | 5′-ACGCCACCAGCGTTCATCCT |
| SEQ ID No. 435: | 5′-GCCAAGCGACTTTGGGTACT |
| SEQ ID No. 436: | 5′-CGGAAAATTCCCTACTGCAG |
| SEQ ID No. 437: | 5′-CGATCTAGCAAGCCGCTTTC |
| SEQ ID No. 438: | 5′-GGTACCGTCAAGCTGAAAAC |
| SEQ ID No. 439: | 5′-TGCCTCACTTCACCCGAAGA |
| SEQ ID No. 440: | 5′-GGCCGGCCAGTCTCTCAACT |
| SEQ ID No. 441: | 5′-GGTAAGGTACCGTCAAGCTG |
| SEQ ID No. 442: | 5′-GTAAGGTACCGTCAAGCTGA |
| SEQ ID No. 443: | 5′-CCGCAAGACCATCCTCTAGG |
| SEQ ID No. 444: | 5′-ATTTAGCCATCCCTTTCTGG |

The sequences SEQ ID No. 342 to SEQ ID No. 444 are particularly suitable for the detection of Oenococcus oeni.

| | |
|---|---|
| SEQ ID No. 445: | 5′-AACCCTTCATCACACACG |
| SEQ ID No. 446: | 5′-CGAAACCCTTCATCACAC |
| SEQ ID No. 447: | 5′-ACCCTTCATCACACACGC |
| SEQ ID No. 448: | 5′-TACCGTCACACACTGAAC |
| SEQ ID No. 449: | 5′-AGATACCGTCACACACTG |
| SEQ ID No. 450: | 5′-CACTCAAGGGCGGAAACC |
| SEQ ID No. 451: | 5′-ACCGTCACACACTGAACA |
| SEQ ID No. 452: | 5′-CGTCACACACTGAACAGT |
| SEQ ID No. 453: | 5′-CCGAAACCCTTCATCACA |
| SEQ ID No. 454: | 5′-CCGTCACACACTGAACAG |
| SEQ ID No. 455: | 5′-GATACCGTCACACACTGA |
| SEQ ID No. 456: | 5′-GGTAAGATACCGTCACAC |
| SEQ ID No. 457: | 5′-CCCTTCATCACACACGCG |
| SEQ ID No. 458: | 5′-ACAGTGTTTTACGAGCCG |
| SEQ ID No. 459: | 5′-CAGTGTTTTACGAGCCGA |
| SEQ ID No. 460: | 5′-ACAAAGCGTTCGACTTGC |
| SEQ ID No. 461: | 5′-CGGATAACGCTTGGAACA |
| SEQ ID No. 462: | 5′-AGGGCGGAAACCCTCGAA |
| SEQ ID No. 463: | 5′-GGGCGGAAACCCTCGAAC |
| SEQ ID No. 464: | 5′-GGCGGAAACCCTCGAACA |
| SEQ ID No. 465: | 5′-TGAGGGCTTTCACTTCAG |
| SEQ ID No. 466: | 5′-AGGGCTTTCACTTCAGAC |
| SEQ ID No. 467: | 5′-GAGGGCTTTCACTTCAGA |
| SEQ ID No. 468: | 5′-ACTGCACTCAAGTCATCC |
| SEQ ID No. 469: | 5′-CCGGATAACGCTTGGAAC |
| SEQ ID No. 470: | 5′-TCCGGATAACGCTTGGAA |
| SEQ ID No. 471: | 5′-TATCCCCTGCTAAGAGGT |
| SEQ ID No. 472: | 5′-CCTGCTAAGAGGTAGGTT |
| SEQ ID No. 473: | 5′-CCCTGCTAAGAGGTAGGT |
| SEQ ID No. 474: | 5′-CCCCTGCTAAGAGGTAGG |
| SEQ ID No. 475: | 5′-TCCCCTGCTAAGAGGTAG |
| SEQ ID No. 476: | 5′-ATCCCCTGCTAAGAGGTA |
| SEQ ID No. 477: | 5′-CCGTTCCTTTCTGGTAAG |
| SEQ ID No. 478: | 5′-GCCGTTCCTTTCTGGTAA |
| SEQ ID No. 479: | 5′-AGCCGTTCCTTTCTGGTA |
| SEQ ID No. 480: | 5′-GCACGTATTTAGCCGTTC |
| SEQ ID No. 481: | 5′-CACGTATTTAGCCGTTCC |
| SEQ ID No. 482: | 5′-GGCACGTATTTAGCCGTT |
| SEQ ID No. 483: | 5′-CACTTTCCTCTACTGCAC |
| SEQ ID No. 484: | 5′-CCACTTTCCTCTACTGCA |
| SEQ ID No. 485: | 5′-TCCACTTTCCTCTACTGC |
| SEQ ID No. 486: | 5′-CTTTCCTCTACTGCACTC |
| SEQ ID No. 487: | 5′-TAGCCGTTCCTTTCTGGT |
| SEQ ID No. 488: | 5′-TTAGCCGTTCCTTTCTGG |
| SEQ ID No. 489: | 5′-TTATCCCCTGCTAAGAGG |
| SEQ ID No. 490: | 5′-GTTATCCCCTGCTAAGAG |
| SEQ ID No. 491: | 5′-CCCGTTCGCCACTCTTTG |
| SEQ ID No. 492: | 5′-AGCTGAGGGCTTTCACTT |
| SEQ ID No. 493: | 5′-GAGCTGAGGGCTTTCACT |
| SEQ ID No. 494: | 5′-GCTGAGGGCTTTCACTTC |
| SEQ ID No. 495: | 5′-CTGAGGGCTTTCACTTCA |

The sequences SEQ ID No. 445 to SEQ ID No. 495 are particularly suitable for the detection of bacteria of the genus Weissella.

| | |
|---|---|
| SEQ ID No. 496: | 5′ CCCGTGTCCCGAAGGAAC |
| SEQ ID No. 497: | 5′ GCACGAGTATGTCAAGAC |
| SEQ ID No. 498: | 5′ GTATCCCGTGTCCCGAAG |
| SEQ ID No. 499: | 5′ TCCCGTGTCCCGAAGGAA |
| SEQ ID No. 500: | 5′ ATCCCGTGTCCCGAAGGA |
| SEQ ID No. 501: | 5′ TATCCCGTGTCCCGAAGG |
| SEQ ID No. 502: | 5′ CTTACCTTAGGAAGCGCC |
| SEQ ID No. 503: | 5′ TTACCTTAGGAAGCGCCC |
| SEQ ID No. 504: | 5′ CCTGTATCCCGTGTCCCG |
| SEQ ID No. 505: | 5′ CCACCTGTATCCCGTGTC |
| SEQ ID No. 506: | 5′ CACCTGTATCCCGTGTCC |
| SEQ ID No. 507: | 5′ ACCTGTATCCCGTGTCCC |
| SEQ ID No. 508: | 5′ CTGTATCCCGTGTCCCGA |
| SEQ ID No. 509: | 5′ TGTATCCCGTGTCCCGAA |
| SEQ ID No. 510: | 5′ CACGAGTATGTCAAGACC |
| SEQ ID No. 511: | 5′ CGGTCTTACCTTAGGAAG |
| SEQ ID No. 512: | 5′ TAGGAAGCGCCCTCCTTG |
| SEQ ID No. 513: | 5′ AGGAAGCGCCCTCCTTGC |
| SEQ ID No. 514: | 5′ TTAGGAAGCGCCCTCCTT |
| SEQ ID No. 515: | 5′ CTTAGGAAGCGCCCTCCT |
| SEQ ID No. 516: | 5′ CCTTAGGAAGCGCCCTCC |
| SEQ ID No. 517: | 5′ ACCTTAGGAAGCGCCCTC |
| SEQ ID No. 518: | 5′ TGCACACAATGGTTGAGC |
| SEQ ID No. 519: | 5′ TACCTTAGGAAGCGCCCT |
| SEQ ID No. 520: | 5′ ACCACCTGTATCCCGTGT |
| SEQ ID No. 521: | 5′ GCACCACCTGTATCCCGT |
| SEQ ID No. 522: | 5′ CACCACCTGTATCCCGTG |
| SEQ ID No. 523: | 5′ GCGGTTAGGCAACCTACT |
| SEQ ID No. 524: | 5′ TGCGGTTAGGCAACCTAC |
| SEQ ID No. 525: | 5′ TTGCGGTTAGGCAACCTA |
| SEQ ID No. 526: | 5′ GGTCTTACCTTAGGAAGC |
| SEQ ID No. 527: | 5′ GCTAATACAACGCGGGAT |
| SEQ ID No. 528: | 5′ CTAATACAACGCGGGATC |
| SEQ ID No. 529: | 5′ ATACAACGCGGGATCATC |
| SEQ ID No. 530: | 5′ CGGTTAGGCAACCTACTT |
| SEQ ID No. 531: | 5′ TGCACCACCTGTATCCCG |
| SEQ ID No. 532: | 5′ GAAGCGCCCTCCTTGCGG |
| SEQ ID No. 533: | 5′ GGAAGCGCCCTCCTTGCG |
| SEQ ID No. 534: | 5′ CGTCCCTTTCTGGTTAGA |
| SEQ ID No. 535: | 5′ AGCTAATACAACGCGGGA |
| SEQ ID No. 536: | 5′ TAGCTAATACAACGCGGG |
| SEQ ID No. 537: | 5′ CTAGCTAATACAACGCGG |
| SEQ ID No. 538: | 5′ GGCTATGTATCATCGCCT |
| SEQ ID No. 539: | 5′ GAGCCACTGCCTTTTACA |
| SEQ ID No. 540: | 5′ GTCGGCTATGTATCATCG |
| SEQ ID No. 541: | 5′ GGTCGGCTATGTATCATC |
| SEQ ID No. 542: | 5′ CAGGTCGGCTATGTATCA |
| SEQ ID No. 543: | 5′ CGGCTATGTATCATCGCC |
| SEQ ID No. 544: | 5′ TCGGCTATGTATCATCGC |
| SEQ ID No. 545: | 5′ GTCTTACCTTAGGAAGCG |
| SEQ ID No. 546: | 5′ TCTTACCTTAGGAAGCGC |

The sequences SEQ ID No. 496 to SEQ ID No. 546 are particularly suitable for the detection of bacteria of the genus Lactococcus.

d) Nucleic Acid Molecules, Which Specifically Detect Drink-Spoiling Acetic Acid Bacteria:

| | |
|---|---|
| SEQ ID No. 547: | 5′-GTACAAACCGCCTACACGCC |
| SEQ ID No. 548: | 5′-TGTACAAACCGCCTACACGC |
| SEQ ID No. 549: | 5′-GATCAGCACGATGTCGCCAT |
| SEQ ID No. 550: | 5′-CTGTACAAACCGCCTACACG |
| SEQ ID No. 551: | 5′-GAGATCAGCACGATGTCGCC |
| SEQ ID No. 552: | 5′-AGATCAGCACGATGTCGCCA |
| SEQ ID No. 553: | 5′-ATCAGCACGATGTCGCCATC |
| SEQ ID No. 554: | 5′-TCAGCACGATGTCGCCATCT |
| SEQ ID No. 555: | 5′-ACTGTACAAACCGCCTACAC |
| SEQ ID No. 556: | 5′-CCGCCACTAAGGCCGAAACC |
| SEQ ID No. 557: | 5′-CAGCACGATGTCGCCATCTA |
| SEQ ID No. 558: | 5′-TACAAACCGCCTACACGCCC |
| SEQ ID No. 559: | 5′-AGCACGATGTCGCCATCTAG |
| SEQ ID No. 560: | 5′-CGGCTTTTAGAGATCAGCAC |
| SEQ ID No. 561: | 5′-TCCGCCACTAAGGCCGAAAC |
| SEQ ID No. 562: | 5′-GACTGTACAAACCGCCTACA |
| SEQ ID No. 563: | 5′-GTCCGCCACTAAGGCCGAAA |
| SEQ ID No. 564: | 5′-GGGGATTTCACATCTGACTG |
| SEQ ID No. 565: | 5′-CATACAAGCCCTGGTAAGGT |
| SEQ ID No. 566: | 5′-ACAAGCCCTGGTAAGGTTCT |
| SEQ ID No. 567: | 5′-ACAAACCGCCTACACGCCCT |
| SEQ ID No. 568: | 5′-CTGACTGTACAAACCGCCTA |
| SEQ ID No. 569: | 5′-TGACTGTACAAACCGCCTAC |
| SEQ ID No. 570: | 5′-ACGATGTCGCCATCTAGCTT |
| SEQ ID No. 571: | 5′-CACGATGTCGCCATCTAGCT |
| SEQ ID No. 572: | 5′-CGATGTCGCCATCTAGCTTC |
| SEQ ID No. 573: | 5′-GCACGATGTCGCCATCTAGC |
| SEQ ID No. 574: | 5′-GATGTCGCCATCTAGCTTCC |
| SEQ ID No. 575: | 5′-ATGTCGCCATCTAGCTTCCC |
| SEQ ID No. 576: | 5′-TGTCGCCATCTAGCTTCCCA |
| SEQ ID No. 577: | 5′-GCCATCTAGCTTCCCACTGT |
| SEQ ID No. 578: | 5′-TCGCCATCTAGCTTCCCACT |
| SEQ ID No. 579: | 5′-CGCCATCTAGCTTCCCACTG |
| SEQ ID No. 580: | 5′-GTCGCCATCTAGCTTCCCAC |
| SEQ ID No. 581: | 5′-TACAAGCCCTGGTAAGGTTC |
| SEQ ID No. 582: | 5′-GCCACTAAGGCCGAAACCTT |
| SEQ ID No. 583: | 5′-ACTAAGGCCGAAACCTTCGT |
| SEQ ID No. 584: | 5′-CTAAGGCCGAAACCTTCGTG |
| SEQ ID No. 585: | 5′-CACTAAGGCCGAAACCTTCG |
| SEQ ID No. 586: | 5′-AAGGCCGAAACCTTCGTGCG |
| SEQ ID No. 587: | 5′-CCACTAAGGCCGAAACCTTC |
| SEQ ID No. 588: | 5′-TAAGGCCGAAACCTTCGTGC |
| SEQ ID No. 589: | 5′-AGGCCGAAACCTTCGTGCGA |
| SEQ ID No. 590: | 5′-TCTGACTGTACAAACCGCCT |
| SEQ ID No. 591: | 5′-CATCTGACTGTACAAACCGC |
| SEQ ID No. 592: | 5′-ATCTGACTGTACAAACCGCC |
| SEQ ID No. 593: | 5′-CTTCGTGCGACTTGCATGTG |
| SEQ ID No. 594: | 5′-CCTTCGTGCGACTTGCATGT |
| SEQ ID No. 595: | 5′-CTCTCTAGAGTGCCCACCCA |
| SEQ ID No. 596: | 5′-TCTCTAGAGTGCCCACCCAA |
| SEQ ID No. 597: | 5′-ACGTATCAAATGCAGCTCCC |
| SEQ ID No. 598: | 5′-CGTATCAAATGCAGCTCCCA |
| SEQ ID No. 599: | 5′-CGCCACTAAGGCCGAAACCT |
| SEQ ID No. 600: | 5′-CCGAAACCTTCGTGCGACTT |
| SEQ ID No. 601: | 5′-GCCGAAACCTTCGTGCGACT |
| SEQ ID No. 602: | 5′-AACCTTCGTGCGACTTGCAT |
| SEQ ID No. 603: | 5′-CGAAACCTTCGTGCGACTTG |
| SEQ ID No. 604: | 5′-ACCTTCGTGCGACTTGCATG |
| SEQ ID No. 605: | 5′-GAAACCTTCGTGCGACTTGC |
| SEQ ID No. 606: | 5′-GGCCGAAACCTTCGTGCGAC |
| SEQ ID No. 607: | 5′-AAACCTTCGTGCGACTTGCA |
| SEQ ID No. 608: | 5′-CACGTATCAAATGCAGCTCC |

The sequences SEQ ID No. 547 to SEQ ID No. 608 are particularly suitable for the simultanous detection of bacteria of the genera Acetobacter and Gliconobacter.

| | |
|---|---|
| SEQ ID No. 609: | 5′- GCTCACCGGCTTAAGGTCAA |
| SEQ ID No. 610: | 5′- CGCTCACCGGCTTAAGGTCA |
| SEQ ID No. 611: | 5′- TCGCTCACCGGCTTAAGGTC |
| SEQ ID No. 612: | 5′- CTCACCGGCTTAAGGTCAAA |
| SEQ ID No. 613: | 5′- CCCGACCGTGGTCGGCTGCG |
| SEQ ID No. 614: | 5′- GCTCACCGGCTTAAGGTCAA |
| SEQ ID No. 615: | 5′- CGCTCACCGGCTTAAGGTCA |
| SEQ ID No. 616: | 5′- TCGCTCACCGGCTTAAGGTC |
| SEQ ID No. 617: | 5′- CTCACCGGCTTAAGGTCAAA |
| SEQ ID No. 618: | 5′- CCCGACCGTGGTCGGCTGCG |
| SEQ ID No. 619: | 5′- TCACCGGCTTAAGGTCAAAC |
| SEQ ID No. 620: | 5′- CAACCCTCTCTCACACTCTA |
| SEQ ID No. 621: | 5′- ACAACCCTCTCTCACACTCT |
| SEQ ID No. 622: | 5′- CCACAACCCTCTCTCACACT |
| SEQ ID No. 623: | 5′- AACCCTCTCTCACACTCTAG |
| SEQ ID No. 624: | 5′- CACAACCCTCTCTCACACTC |
| SEQ ID No. 625: | 5′- TCCACAACCCTCTCTCACAC |
| SEQ ID No. 626: | 5′- TTCCACAACCCTCTCTCACA |
| SEQ ID No. 627: | 5′- ACCCTCTCTCACACTCTAGT |
| SEQ ID No. 628: | 5′- GAGCCAGGTTGCCGCCTTCG |
| SEQ ID No. 629: | 5′- AGGTCAAACCAACTCCCATG |
| SEQ ID No. 630: | 5′- ATGAGCCAGGTTGCCGCCTT |
| SEQ ID No. 631: | 5′- TGAGCCAGGTTGCCGCCTTC |
| SEQ ID No. 632: | 5′- AGGCTCCTCCACAGGCGACT |
| SEQ ID No. 633: | 5′- CAGGCTCCTCCACAGGCGAC |
| SEQ ID No. 634: | 5′- GCAGGCTCCTCCACAGGCGA |
| SEQ ID No. 635: | 5′- TTCGCTCACCGGCTTAAGGT |
| SEQ ID No. 636: | 5′- GTTCGCTCACCGGCTTAAGG |
| SEQ ID No. 637: | 5′- GGTTCGCTCACCGGCTTAAG |
| SEQ ID No. 638: | 5′- ATTCCACAACCCTCTCTCAC |
| SEQ ID No. 639: | 5′- TGACCCGACCGTGGTCGGCT |
| SEQ ID No. 640: | 5′- CCCTCTCTCACACTCTAGTC |
| SEQ ID No. 641: | 5′- GAATTCCACAACCCTCTCTC |
| SEQ ID No. 642: | 5′- AGCCAGGTTGCCGCCTTCGC |
| SEQ ID No. 643: | 5′- GCCAGGTTGCCGCCTTCGCC |
| SEQ ID No. 644: | 5′- GGAATTCCACAACCCTCTCT |
| SEQ ID No. 645: | 5′- GGGAATTCCACAACCCTCTC |
| SEQ ID No. 646: | 5′- AACGCAGGCTCCTCCACAGG |
| SEQ ID No. 647: | 5′- CGGCTTAAGGTCAAACCAAC |
| SEQ ID No. 648: | 5′- CCGGCTTAAGGTCAAACCAA |
| SEQ ID No. 649: | 5′- CACCGGCTTAAGGTCAAACC |
| SEQ ID No. 650: | 5′- ACCGGCTTAAGGTCAAACCA |
| SEQ ID No. 651: | 5′- ACCCAACATCCAGCACACAT |
| SEQ ID No. 652: | 5′- TCGCTGACCCGACCGTGGTC |
| SEQ ID No. 653: | 5′- CGCTGACCCGACCGTGGTCG |
| SEQ ID No. 654: | 5′- GACCCGACCGTGGTCGGCTG |
| SEQ ID No. 655: | 5′- GCTGACCCGACCGTGGTCGG |
| SEQ ID No. 656: | 5′- CTGACCCGACCGTGGTCGGC |
| SEQ ID No. 657: | 5′- CAGGCGACTTGCGCCTTTGA |
| SEQ ID No. 658: | 5′- TCATGCGGTATTAGCTCCAG |
| SEQ ID No. 659: | 5′- ACTAGCTAATCGAACGCAGG |
| SEQ ID No. 660: | 5′- CATGCGGTATTAGCTCCAGT |
| SEQ ID No. 661: | 5′- CGCAGGCTCCTCCACAGGCG |
| SEQ ID No. 662: | 5′- ACGCAGGCTCCTCCACAGGC |
| SEQ ID No. 663: | 5′- CTCAGGTGTCATGCGGTATT |
| SEQ ID No. 664: | 5′- CGCCTTTGACCCTCAGGTGT |
| SEQ ID No. 665: | 5′- ACCCTCAGGTGTCATGCGGT |
| SEQ ID No. 666: | 5′- CCTCAGGTGTCATGCGGTAT |
| SEQ ID No. 667: | 5′- TTTGACCCTCAGGTGTCATG |
| SEQ ID No. 668: | 5′- GACCCTCAGGTGTCATGCGG |
| SEQ ID No. 669: | 5′- TGACCCTCAGGTGTCATGCG |
| SEQ ID No. 670: | 5′- GCCTTTGACCCTCAGGTGTC |
| SEQ ID No. 671: | 5′- TTGACCCTCAGGTGTCATGC |
| SEQ ID No. 672: | 5′- CCCTCAGGTGTCATGCGGTA |
| SEQ ID No. 673: | 5′- CCTTTGACCCTCAGGTGTCA |
| SEQ ID No. 674: | 5′- CTTTGACCCTCAGGTGTCAT |
| SEQ ID No. 675: | 5′- AGTTATCCCCCACCCATGGA |
| SEQ ID No. 676: | 5′- CCAGCTATCGATCATCGCCT |
| SEQ ID No. 677: | 5′- ACCAGCTATCGATCATCGCC |
| SEQ ID No. 678: | 5′- CAGCTATCGATCATCGCCTT |
| SEQ ID No. 679: | 5′- AGCTATCGATCATCGCCTTG |
| SEQ ID No. 680: | 5′- GCTATCGATCATCGCCTTGG |
| SEQ ID No. 681: | 5′- CTATCGATCATCGCCTTGGT |
| SEQ ID No. 682: | 5′- TTCGTGCGACTTGCATGTGT |
| SEQ ID No. 683: | 5′- TCGATCATCGCCTTGGTAGG |
| SEQ ID No. 684: | 5′- ATCGATCATCGCCTTGGTAG |
| SEQ ID No. 685: | 5′- CACAGGCGACTTGCGCCTTT |
| SEQ ID No. 686: | 5′- CCACAGGCGACTTGCGCCTT |
| SEQ ID No. 687: | 5′- TCCACAGGCGACTTGCGCCT |
| SEQ ID No. 688: | 5′- TCCTCCACAGGCGACTTGCG |
| SEQ ID No. 689: | 5′- CCTCCACAGGCGACTTGCGC |
| SEQ ID No. 690: | 5′- CTCCACAGGCGACTTGCGCC |
| SEQ ID No. 691: | 5′- ACAGGCGACTTGCGCCTTTG |
| SEQ ID No. 692: | 5′- GCTCACCGGCTTAAGGTCAA |
| SEQ ID No. 693: | 5′- CGCTCACCGGCTTAAGGTCA |
| SEQ ID No. 694: | 5′- TCGCTCACCGGCTTAAGGTC |
| SEQ ID No. 695: | 5′- CTCACCGGCTTAAGGTCAAA |
| SEQ ID No. 696: | 5′- CCCGACCGTGGTCGGCTGCG |
| SEQ ID No. 697: | 5′- TCACCGGCTTAAGGTCAAAC |
| SEQ ID No. 698: | 5′- CAACCCTCTCTCACACTCTA |
| SEQ ID No. 699: | 5′- ACAACCCTCTCTCACACTCT |
| SEQ ID No. 700: | 5′- CCACAACCCTCTCTCACACT |
| SEQ ID No. 701: | 5′- AACCCTCTCTCACACTCTAG |
| SEQ ID No. 702: | 5′- CACAACCCTCTCTCACACTC |
| SEQ ID No. 703: | 5′- TCCACAACCCTCTCTCACAC |
| SEQ ID No. 704: | 5′- TTCCACAACCCTCTCTCACA |
| SEQ ID No. 705: | 5′- ACCCTCTCTCACACTCTAGT |
| SEQ ID No. 706: | 5′- GAGCCAGGTTGCCGCCTTCG |
| SEQ ID No. 707: | 5′- AGGTCAAACCAACTCCCATG |
| SEQ ID No. 708: | 5′- ATGAGCCAGGTTGCCGCCTT |
| SEQ ID No. 709: | 5′- TGAGCCAGGTTGCCGCCTTC |
| SEQ ID No. 710: | 5′- AGGCTCCTCCACAGGCGACT |
| SEQ ID No. 711: | 5′- CAGGCTCCTCCACAGGCGAC |
| SEQ ID No. 712: | 5′- GCAGGCTCCTCCACAGGCGA |
| SEQ ID No. 713: | 5′- TTCGCTCACCGGCTTAAGGT |
| SEQ ID No. 714: | 5′- GTTCGCTCACCGGCTTAAGG |
| SEQ ID No. 715: | 5′- GGTTCGCTCACCGGCTTAAG |
| SEQ ID No. 716: | 5′- ATTCCACAACCCTCTCTCAC |
| SEQ ID No. 717: | 5′- TGACCCGACCGTGGTCGGCT |
| SEQ ID No. 718: | 5′- CCCTCTCTCACACTCTAGTC |
| SEQ ID No. 719: | 5′- GAATTCCACAACCCTCTCTC |
| SEQ ID No. 720: | 5′- AGCCAGGTTGCCGCCTTCGC |
| SEQ ID No. 721: | 5′- GCCAGGTTGCCGCCTTCGCC |
| SEQ ID No. 722: | 5′- GGAATTCCACAACCCTCTCT |
| SEQ ID No. 723: | 5′- GGGAATTCCACAACCCTCTC |
| SEQ ID No. 724: | 5′- AACGCAGGCTCCTCCACAGG |
| SEQ ID No. 725: | 5′- CGGCTTAAGGTCAAACCAAC |
| SEQ ID No. 726: | 5′- CCGGCTTAAGGTCAAACCAA |
| SEQ ID No. 727: | 5′- CACCGGCTTAAGGTCAAACC |
| SEQ ID No. 728: | 5′- ACCGGCTTAAGGTCAAACCA |
| SEQ ID No. 729: | 5′- ACCCAACATCCAGCACACAT |
| SEQ ID No. 730: | 5′- TCGCTGACCCGACCGTGGTC |
| SEQ ID No. 731: | 5′- CGCTGACCCGACCGTGGTCG |
| SEQ ID No. 732: | 5′- GACCCGACCGTGGTCGGCTG |
| SEQ ID No. 733: | 5′- GCTGACCCGACCGTGGTCGG |
| SEQ ID No. 734: | 5′- CTGACCCGACCGTGGTCGGC |
| SEQ ID No. 735: | 5′- CAGGCGACTTGCGCCTTTGA |
| SEQ ID No. 736: | 5′- TCATGCGGTATTAGCTCCAG |
| SEQ ID No. 737: | 5′- ACTAGCTAATCGAACGCAGG |
| SEQ ID No. 738: | 5′- CATGCGGTATTAGCTCCAGT |
| SEQ ID No. 739: | 5′- CGCAGGCTCCTCCACAGGCG |
| SEQ ID No. 740: | 5′- ACGCAGGCTCCTCCACAGGC |
| SEQ ID No. 741: | 5′- CTCAGGTGTCATGCGGTATT |
| SEQ ID No. 742: | 5′- CGCCTTTGACCCTCAGGTGT |
| SEQ ID No. 743: | 5′- ACCCTCAGGTGTCATGCGGT |
| SEQ ID No. 744: | 5′- CCTCAGGTGTCATGCGGTAT |
| SEQ ID No. 745: | 5′- TTTGACCCTCAGGTGTCATG |
| SEQ ID No. 746: | 5′- GACCCTCAGGTGTCATGCGG |
| SEQ ID No. 747: | 5′- TGACCCTCAGGTGTCATGCG |
| SEQ ID No. 748: | 5′- GCCTTTGACCCTCAGGTGTC |
| SEQ ID No. 749: | 5′- TTGACCCTCAGGTGTCATGC |
| SEQ ID No. 750: | 5′- CCCTCAGGTGTCATGCGGTA |
| SEQ ID No. 751: | 5′- CCTTTGACCCTCAGGTGTCA |
| SEQ ID No. 752: | 5′- CTTTGACCCTCAGGTGTCAT |
| SEQ ID No. 753: | 5′- AGTTATCCCCCACCCATGGA |
| SEQ ID No. 754: | 5′- CCAGCTATCGATCATCGCCT |
| SEQ ID No. 755: | 5′- ACCAGCTATCGATCATCGCC |
| SEQ ID No. 756: | 5′- CAGCTATCGATCATCGCCTT |
| SEQ ID No. 757: | 5′- AGCTATCGATCATCGCCTTG |
| SEQ ID No. 758: | 5′- GCTATCGATCATCGCCTTGG |
| SEQ ID No. 759: | 5′- CTATCGATCATCGCCTTGGT |
| SEQ ID No. 760: | 5′- TTCGTGCGACTTGCATGTGT |
| SEQ ID No. 761: | 5′- TCGATCATCGCCTTGGTAGG |
| SEQ ID No. 762: | 5′- ATCGATCATCGCCTTGGTAG |
| SEQ ID No. 763: | 5′- CACAGGCGACTTGCGCCTTT |
| SEQ ID No. 764: | 5′- CCACAGGCGACTTGCGCCTT |
| SEQ ID No. 765: | 5′- TCCACAGGCGACTTGCGCCT |
| SEQ ID No. 766: | 5′- TCCTCCACAGGCGACTTGCG |
| SEQ ID No. 767: | 5′- CCTCCACAGGCGACTTGCGC |
| SEQ ID No. 768: | 5′- CTCCACAGGCGACTTGCGCC |
| SEQ ID No. 769: | 5′- ACAGGCGACTTGCGCCTTTG |
| SEQ ID No. 770: | 5′- TCACCGGCTTAAGGTCAAAC |
| SEQ ID No. 771: | 5′- CAACCCTCTCTCACACTCTA |
| SEQ ID No. 772: | 5′- ACAACCCTCTCTCACACTCT |
| SEQ ID No. 773: | 5′- CCACAACCCTCTCTCACACT |
| SEQ ID No. 774: | 5′- AACCCTCTCTCACACTCTAG |
| SEQ ID No. 775: | 5′- CACAACCCTCTCTCACACTC |
| SEQ ID No. 776: | 5′- TCCACAACCCTCTCTCACAC |
| SEQ ID No. 777: | 5′- TTCCACAACCCTCTCTCACA |
| SEQ ID No. 778: | 5′- ACCCTCTCTCACACTCTAGT |
| SEQ ID No. 779: | 5′- GAGCCAGGTTGCCGCCTTCG |
| SEQ ID No. 780: | 5′- AGGTCAAACCAACTCCCATG |
| SEQ ID No. 781: | 5′- ATGAGCCAGGTTGCCGCCTT |
| SEQ ID No. 782: | 5′- TGAGCCAGGTTGCCGCCTTC |
| SEQ ID No. 783: | 5′- AGGCTCCTCCACAGGCGACT |
| SEQ ID No. 784: | 5′- CAGGCTCCTCCACAGGCGAC |
| SEQ ID No. 785: | 5′- GCAGGCTCCTCCACAGGCGA |
| SEQ ID No. 786: | 5′- TTCGCTCACCGGCTTAAGGT |
| SEQ ID No. 787: | 5′- GTTCGCTCACCGGCTTAAGG |
| SEQ ID No. 788: | 5′- GGTTCGCTCACCGGCTTAAG |
| SEQ ID No. 789: | 5′- ATTCCACAACCCTCTCTCAC |
| SEQ ID No. 790: | 5′- TGACCCGACCGTGGTCGGCT |
| SEQ ID No. 791: | 5′- CCCTCTCTCACACTCTAGTC |
| SEQ ID No. 792: | 5′- GAATTCCACAACCCTCTCTC |
| SEQ ID No. 793: | 5′- AGCCAGGTTGCCGCCTTCGC |
| SEQ ID No. 794: | 5′- GCCAGGTTGCCGCCTTCGCC |
| SEQ ID No. 795: | 5′- GGAATTCCACAACCCTCTCT |
| SEQ ID No. 796: | 5′- GGGAATTCCACAACCCTCTC |
| SEQ ID No. 797: | 5′- AACGCAGGCTCCTCCACAGG |
| SEQ ID No. 798: | 5′- CGGCTTAAGGTCAAACCAAC |
| SEQ ID No. 799: | 5′- CCGGCTTAAGGTCAAACCAA |
| SEQ ID No. 800: | 5′- CACCGGCTTAAGGTCAAACC |
| SEQ ID No. 801: | 5′- ACCGGCTTAAGGTCAAACCA |
| SEQ ID No. 802: | 5′- ACCCAACATCCAGCACACAT |
| SEQ ID No. 803: | 5′- TCGCTGACCCGACCGTGGTC |
| SEQ ID No. 804: | 5′- CGCTGACCCGACCGTGGTCG |
| SEQ ID No. 805: | 5′- GACCCGACCGTGGTCGGCTG |
| SEQ ID No. 806: | 5′- GCTGACCCGACCGTGGTCGG |
| SEQ ID No. 807: | 5′- CTGACCCGACCGTGGTCGGC |
| SEQ ID No. 808: | 5′- CAGGCGACTTGCGCCTTTGA |
| SEQ ID No. 809: | 5′- TCATGCGGTATTAGCTCCAG |
| SEQ ID No. 810: | 5′- ACTAGCTAATCGAACGCAGG |
| SEQ ID No. 811: | 5′- CATGCGGTATTAGCTCCAGT |
| SEQ ID No. 812: | 5′- CGCAGGCTCCTCCACAGGCG |
| SEQ ID No. 813: | 5′- ACGCAGGCTCCTCCACAGGC |
| SEQ ID No. 814: | 5′- CTCAGGTGTCATGCGGTATT |
| SEQ ID No. 815: | 5′- CGCCTTTGACCCTCAGGTGT |
| SEQ ID No. 816: | 5′- ACCCTCAGGTGTCATGCGGT |
| SEQ ID No. 817: | 5′- CCTCAGGTGTCATGCGGTAT |
| SEQ ID No. 818: | 5′- TTTGACCCTCAGGTGTCATG |
| SEQ ID No. 819: | 5′- GACCCTCAGGTGTCATGCGG |
| SEQ ID No. 820: | 5′- TGACCCTCAGGTGTCATGCG |
| SEQ ID No. 821: | 5′- GCCTTTGACCCTCAGGTGTC |
| SEQ ID No. 822: | 5′- TTGACCCTCAGGTGTCATGC |
| SEQ ID No. 823: | 5′- CCCTCAGGTGTCATGCGGTA |
| SEQ ID No. 824: | 5′- CCTTTGACCCTCAGGTGTCA |
| SEQ ID No. 825: | 5′- CTTTGACCCTCAGGTGTCAT |
| SEQ ID No. 826: | 5′- AGTTATCCCCCACCCATGGA |
| SEQ ID No. 827: | 5′- CCAGCTATCGATCATCGCCT |
| SEQ ID No. 828: | 5′- ACCAGCTATCGATCATCGCC |
| SEQ ID No. 829: | 5′- CAGCTATCGATCATCGCCTT |
| SEQ ID No. 830: | 5′- AGCTATCGATCATCGCCTTG |
| SEQ ID No. 831: | 5′- GCTATCGATCATCGCCTTGG |
| SEQ ID No. 832: | 5′- CTATCGATCATCGCCTTGGT |
| SEQ ID No. 833: | 5′- TTCGTGCGACTTGCATGTGT |
| SEQ ID No. 834: | 5′- TCGATCATCGCCTTGGTAGG |
| SEQ ID No. 835: | 5′- ATCGATCATCGCCTTGGTAG |
| SEQ ID No. 836: | 5′- CACAGGCGACTTGCGCCTTT |
| SEQ ID No. 837: | 5′- CCACAGGCGACTTGCGCCTT |
| SEQ ID No. 838: | 5′- TCCACAGGCGACTTGCGCCT |
| SEQ ID No. 839: | 5′- TCCTCCACAGGCGACTTGCG |
| SEQ ID No. 840: | 5′- CCTCCACAGGCGACTTGCGC |
| SEQ ID No. 841: | 5′- CTCCACAGGCGACTTGCGCC |
| SEQ ID No. 842: | 5′- ACAGGCGACTTGCGCCTTTG |

The sequences SEQ ID No. 609 to SEQ ID No. 842 are particularly suitable for the simultanous detection of bacteria of the genera Acetobacter, Gluconobacter and Gluconoacetobacter.

e) Nucleic Acid Probe Molecules, Which Specifically Detect Drink-Spoiling Bacilli:

| | |
|---|---|
| SEQ ID No. 843: | 5′- AGCCCCGGTTTCCCGGCGTT |
| SEQ ID No. 844: | 5′- CGCCTTTCCTTTTTCCTCCA |
| SEQ ID No. 845: | 5′- GCCCCGGTTTCCCGGCGTTA |
| SEQ ID No. 846: | 5′- GCCGCCTTTCCTTTTTCCTC |
| SEQ ID No. 847: | 5′- TAGCCCCGGTTTCCCGGCGT |
| SEQ ID No. 848: | 5′- CCGGGTACCGTCAAGGCGCC |
| SEQ ID No. 849: | 5′- AAGCCGCCTTTCCTTTTTCC |
| SEQ ID No. 850: | 5′- CCCCCGTTTCCCGGCGTTAT |
| SEQ ID NO. 851: | 5′- CCGGCGTTATCCCAGTCTTA |
| SEQ ID No. 852: | 5′- AGCCGCCTTTCCTTTTTCCT |
| SEQ ID No. 853: | 5′- CCGCCTTTCCTTTTTCCTCC |
| SEQ ID No. 854: | 5′- TTAGCCCCGGTTTCCCGGCG |
| SEQ ID No. 855: | 5′- CCCGGCGTTATCCCAGTCTT |
| SEQ ID No. 856: | 5′- GCCGGGTACCGTCAAGGCGC |
| SEQ ID No. 857: | 5′- GGCCGGGTACCGTCAAGGCG |
| SEQ ID No. 858: | 5′- TCCCGGCGTTATCCCAGTCT |
| SEQ ID No. 859: | 5′- TGGCCGGGTACCGTCAAGGC |
| SEQ ID No. 860: | 5′- GAAGCCGCCTTTCCTTTTTC |
| SEQ ID No. 861: | 5′- CCCGGTTTCCCGGCGTTATC |
| SEQ ID No: 862: | 5′- CGGCGTTATCCCAGTCTTAC |
| SEQ ID No. 863: | 5′- GGCGTTATCCCAGTCTTACA |
| SEQ ID No. 864: | 5′- GCGTTATCCCAGTCTTACAG |
| SEQ ID No. 865: | 5′- CGGGTACCGTCAAGGCGCCG |
| SEQ ID No. 866: | 5′- ATTAGCCCCGGTTTCCCGGC |
| SEQ ID No. 867: | 5′- AAGGGGAAGGCCCTGTCTCC |
| SEQ ID No. 868: | 5′- GGCCCTGTCTCCAGGGAGGT |
| SEQ ID No. 869: | 5′- AGGCCCTGTCTCCAGGGAGG |
| SEQ ID No. 870: | 5′- AAGGCCCTGTCTCCAGGGAG |
| SEQ ID No. 871: | 5′- GCCCTGTCTCCAGGGAGGTC |
| SEQ ID No. 872: | 5′- CGTTATCCCAGTCTTACAGG |
| SEQ ID No. 873: | 5′- GGGTACCGTCAAGGCGCCGC |
| SEQ ID No. 874: | 5′- CGGCAACAGAGTTTTACGAC |
| SEQ ID No. 875: | 5′- GGGGAAGGCCCTGTCTCCAG |
| SEQ ID No. 876: | 5′- AGGGGAAGGCCCTGTCTCCA |
| SEQ ID No. 877: | 5′- GCAGCCGAAGCCGCCTTTCC |
| SEQ ID No. 878: | 5′- TTCTTCCCCGGCAACAGAGT |
| SEQ ID No. 879: | 5′- CGGCACTTGTTCTTCCCCGG |
| SEQ ID No. 880: | 5′- GTTCTTCCCCGGCAACAGAG |
| SEQ ID No. 881: | 5′- GGCACTTGTTCTTCCCCGGC |
| SEQ ID No. 882: | 5′- GCACTTGTTCTTCCCCGGCA |
| SEQ ID No. 883: | 5′- CACTTGTTCTTCCCCGGCAA |
| SEQ ID No. 884: | 5′- TCTTCCCCGGCAACAGAGTT |
| SEQ ID No. 885: | 5′- TTGTTCTTCCCCGGCAACAG |
| SEQ ID No. 886: | 5′- ACTTGTTCTTCCCCGGCAAC |
| SEQ ID No. 887: | 5′- TGTTCTTCCCCGGCAACAGA |
| SEQ ID No. 888: | 5′- CTTGTTCTTCCCCGGCAACA |
| SEQ ID No. 889: | 5′- ACGGCACTTGTTCTTCCCCG |
| SEQ ID No. 890: | 5′- GTCCGCCGCTAACCTTTTAA |
| SEQ ID No. 891: | 5′- CTGGCCGGGTACCGTCAAGG |
| SEQ ID No. 892: | 5′- TCTGGCCGGGTACCGTCAAG |
| SEQ ID No. 893: | 5′- TTCTGGCCGGGTACCGTCAA |
| SEQ ID No. 894: | 5′- CAATGCTGGCAACTAAGGTC |
| SEQ ID No. 895: | 5′- CGTCCGCCGCTAACCTTTTA |
| SEQ ID No. 896: | 5′- CGAAGCCGCCTTTCCTTTTT |
| SEQ ID No. 897: | 5′- CCGAAGCCGCCTTTCCTTTT |
| SEQ ID No. 898: | 5′- GCCGAAGCCGCCTTTCCTTT |
| SEQ ID No. 899: | 5′- AGCCGAAGCCGCCTTTCCTT |
| SEQ ID No. 900: | 5′- ACCGTCAAGGCGCCGCCCTG |
| SEQ ID No. 901: | 5′- CCGTGGCTTTCTGGCCGGGT |
| SEQ ID No. 902: | 5′- GCTTTCTGGCCGGGTACCGT |
| SEQ ID No. 903: | 5′- GCCGTGGCTTTCTGGCCGGG |
| SEQ ID No. 904: | 5′- GGCTTTCTGGCCGGGTACCG |
| SEQ ID No. 905: | 5′- CTTTCTGGCCGGGTACCGTC |
| SEQ ID No. 906: | 5′- TGGCTTTCTGGCCGGGTACC |
| SEQ ID No. 907: | 5′- GTGGCTTTCTGGCCGGGTAC |
| SEQ ID No. 908: | 5′- CGTGGCTTTCTGGCCGGGTA |
| SEQ ID No. 909: | 5′- TTTCTGGCCGGGTACCGTCA |
| SEQ ID No. 910: | 5′- GGGAAGGCCCTGTCTCCAGG |
| SEQ ID No. 911: | 5′- CGAAGGGGAAGGCCCTGTCT |
| SEQ ID No. 912: | 5′- CCGAAGGGGAAGGCCCTGTC |
| SEQ ID No. 913: | 5′- GAAGGGGAAGGCCCTGTCTC |
| SEQ ID No. 914: | 5′- GGCGCCGCCCTGTTCGAACG |
| SEQ ID No. 915: | 5′- AGGCGCCGCCCTGTTCGAAC |
| SEQ ID No. 916: | 5′- AAGGCGCCGCCCTGTTCGAA |
| SEQ ID No. 917: | 5′- CCCGGCAACAGAGTTTTACG |
| SEQ ID No. 918: | 5′- CCCCGGCAACAGAGTTTTAC |
| SEQ ID No. 919: | 5′- CCATCTGTAAGTGGCAGCCG |
| SEQ ID No. 920: | 5′- TCTGTAAGTGGCAGCCGAAG |
| SEQ ID No. 921: | 5′- CTGTAAGTGGCAGCCGAAGC |
| SEQ ID No. 922: | 5′- CCCATCTGTAAGTGGCAGCC |
| SEQ ID No. 923: | 5′- TGTAAGTGGCAGCCGAAGCC |
| SEQ ID No. 924: | 5′- CATCTGTAAGTGGCAGCCGA |
| SEQ ID No. 925: | 5′- ATCTGTAAGTGGCAGCCGAA |
| SEQ ID No. 926: | 5′- CAGCCGAAGCCGCCTTTCCT |
| SEQ ID No. 927: | 5′- GGCAACAGAGTTTTACGACC |
| SEQ ID No. 928: | 5′- CCGGCAACAGAGTTTTACGA |
| SEQ ID No. 929: | 5′- TTCCCCGGCAACAGAGTTTT |
| SEQ ID No. 930: | 5′- CTTCCCCGGCAACAGAGTTT |
| SEQ ID No. 931: | 5′- TCCCCGGCAACAGAGTTTTA |
| SEQ ID No. 932: | 5′- CCGTCCGCCGCTAACCTTTT |

The sequences SEQ ID No. 843 to SEQ ID No. 932 are particularly suitable for the detection of Bacillus coagulans.

f) Nucleic Acid Probe Molecules Which Specifically Detect Drink-Spoiling Alicyclobacilli:

| | |
|---|---|
| SEQ ID No. 933: | 5′- CTTCCTCCGACTTACGCCGG |
| SEQ ID No. 934: | 5′- CCTCCGACTTACGCCGGCAG |
| SEQ ID No. 935: | 5′- TTCCTCCGACTTACGCCGGC |
| SEQ ID No. 936: | 5′- TCCTCCGACTTACGCCGGCA |
| SEQ ID No. 937: | 5′- TCCGACTTACGCCGGCAGTC |
| SEQ ID No. 938: | 5′- CCGACTTACGCCGGCAGTCA |
| SEQ ID No. 939: | 5′- GCCTTCCTCCGACTTACGCC |
| SEQ ID No. 940: | 5′- CCTTCCTCCGACTTACGCCG |
| SEQ ID No. 941: | 5′- GCTCTCCCCGAGCAACAGAG |
| SEQ ID No. 942: | 5′- CTCTCCCCGAGCAACAGAGC |
| SEQ ID No. 943: | 5′- CGCTCTCCCCGAGCAACAGA |
| SEQ ID No. 944: | 5′- CTCCGACTTACGCCGGCAGT |
| SEQ ID No. 945: | 5′- TCTCCCCGAGCAACAGAGCT |
| SEQ ID No. 946: | 5′- CGACTTACGCCGGCAGTCAC |
| SEQ ID No. 947: | 5′- TCGGCACTGGGGTGTGTCCC |
| SEQ ID No. 948: | 5′- GGCACTGGGGTGTGTCCCCC |
| SEQ ID No. 949: | 5′- CTGGGGTGTGTCCCCCCAAC |
| SEQ ID No. 950: | 5′- CACTGGGGTGTGTCCCCCCA |
| SEQ ID No. 951: | 5′- ACTGGGGTGTGTCCCCCCAA |
| SEQ ID No. 952: | 5′- GCACTGGGGTGTGTCCCCCC |
| SEQ ID No. 953: | 5′- TGGGGTGTGTCCCCCCAACA |
| SEQ ID No. 954: | 5′- CACTCCAGACTTGCTCGACC |
| SEQ ID No. 955: | 5′- TCACTCCAGACTTGCTCGAC |
| SEQ ID No. 956: | 5′- CGGCACTGGGGTGTGTCCCC |
| SEQ ID No. 957: | 5′- CGCCTTCCTCCGACTTACGC |
| SEQ ID No. 958: | 5′- CTCCCCGAGCAACAGAGCTT |
| SEQ ID No. 959: | 5′- ACTCCAGACTTGCTCGACCG |
| SEQ ID No. 960: | 5′- CCCATGCCGCTCTCCCCGAG |
| SEQ ID No. 961: | 5′- CCATGCCGCTCTCCCCGAGC |
| SEQ ID No. 962: | 5′- CCCCATGCCGCTCTCCCCGA |
| SEQ ID No. 963: | 5′- TCACTCGGTACCGTCTCGCA |
| SEQ ID No. 964: | 5′- CATGCCGCTCTCCCCGAGCA |
| SEQ ID No. 965: | 5′- ATGCCGCTCTCCCCGAGCAA |
| SEQ ID No. 966: | 5′- TTCGGCACTGGGGTGTGTCC |
| SEQ ID No. 967: | 5′- TGCCGCTCTCCCCGAGCAAC |
| SEQ ID No. 968: | 5′- TTCACTCCAGACTTGCTCGA |
| SEQ ID No. 969: | 5′- CCCGCAAGAAGATGCCTCCT |
| SEQ ID No. 970: | 5′- AGAAGATGCCTCCTCGCGGG |
| SEQ ID No. 971: | 5′- AAGAAGATGCCTCCTCGCGG |
| SEQ ID No. 972: | 5′- CGCAAGAAGATGCCTCCTCG |
| SEQ ID No. 973: | 5′- AAGATGCCTCCTCGCGGGCG |
| SEQ ID No. 974: | 5′- CCGCAAGAAGATGCCTCCTC |
| SEQ ID No. 975: | 5′- GAAGATGCCTCCTCGCGGGC |
| SEQ ID No. 976: | 5′- CCCCGCAAGAAGATGCCTCC |
| SEQ ID No. 977: | 5′- CAAGAAGATGCCTCCTCGCG |
| SEQ ID No. 978: | 5′- TCCTTCGGCACTGGGGTGTG |
| SEQ ID No. 979: | 5′- CCGCTCTCCCCGAGCAACAG |
| SEQ ID No. 980: | 5′- TGCCTCCTCGCGGGCGTATC |
| SEQ ID No. 981: | 5′- GACTTACGCCGGCAGTCACC |
| SEQ ID No. 982: | 5′- GGCTCCTCTCTCAGCGGCCC |
| SEQ ID No. 983: | 5′- CCTTCGGCACTGGGGTGTGT |
| SEQ ID No. 984: | 5′- GGGGTGTGTCCCCCCAACAC |
| SEQ ID No. 985: | 5′- GCCGCTCTCCCCGAGCAACA |
| SEQ ID No. 986: | 5′- AGATGCCTCCTCGCGGGCGT |
| SEQ ID No. 987: | 5′- CACTCGGTACCGTCTCGCAT |
| SEQ ID No. 988: | 5′- CTCACTCGGTACCGTCTCGC |
| SEQ ID No. 989: | 5′- GCAAGAAGATGCCTCCTCGC |
| SEQ ID No. 990: | 5′- CTCCAGACTTGCTCGACCGC |
| SEQ ID No. 991: | 5′- TTACGCCGGCAGTCACCTGT |
| SEQ ID No. 992: | 5′- CTTCGGCACTGGGGTGTGTC |
| SEQ ID No. 993: | 5′- CTCGCGGGCGTATCCGGCAT |
| SEQ ID No. 994: | 5′- GCCTCCTCGCGGGCGTATCC |
| SEQ ID No. 995: | 5′- ACTCGGTACCGTCTCGCATG |
| SEQ ID No. 996: | 5′- GATGCCTCCTCGCGGGCGTA |
| SEQ ID No. 997: | 5′- GGGTGTGTCCCCCCAACACC |
| SEQ ID No. 998: | 5′- ACTTACGCCGGCAGTCACCT |
| SEQ ID No. 999: | 5′- CTTACGCCGGCAGTCACCTG |
| SEQ ID No. 1000: | 5′- ATGCCTCCTCGCGGGCGTAT |
| SEQ ID No. 1001: | 5′- GCGCCGCGGGCTCCTCTCTC |
| SEQ ID No. 1002: | 5′- GGTGTGTCCCCCCAACACCT |
| SEQ ID No. 1003: | 5′- GTGTGTCCCCCCAACACCTA |
| SEQ ID No. 1004: | 5′- CCTCGCGGGCGTATCCGGCA |
| SEQ ID No. 1005: | 5′- CCTCACTCGGTACCGTCTCG |
| SEQ ID No. 1006: | 5′- TCCTCACTCGGTACCGTCTC |
| SEQ ID No. 1007: | 5′- TCGCGGGCGTATCCGGCATT |
| SEQ ID No. 1008: | 5′- TTTCACTCCAGACTTGCTCG |
| SEQ ID No. 1009: | 5′- TACGCCGGCAGTCACCTGTG |
| SEQ ID No. 1010: | 5′- TCCAGACTTGCTCGACCGCC |
| SEQ ID No. 1011: | 5′- CTCGGTACCGTCTCGCATGG |
| SEQ ID No. 1012: | 5′- CGCGGGCGTATCCGGCATTA |
| SEQ ID No. 1013: | 5′- GCGTATCCGGCATTAGCGCC |
| SEQ ID No. 1014: | 5′- GGGCTCCTCTCTCAGCGGCC |
| SEQ ID No. 1015: | 5′- TCCCCGAGCAACAGAGCTTT |
| SEQ ID No. 1016: | 5′- CCCCGAGCAACAGAGCTTTA |
| SEQ ID No. 1017: | 5′- CCGAGCAACAGAGCTTTACA |
| SEQ ID No. 1018: | 5′- CCATCCCATGGTTGAGCCAT |
| SEQ ID No. 1019: | 5′- GTGTCCCCCCAACACCTAGC |
| SEQ ID No. 1020: | 5′- GCGGGCGTATCCGGCATTAG |
| SEQ ID No. 1021: | 5′- CGAGCGGCTTTTTGGGTTTC |
| SEQ ID No. 1022: | 5′- CTTTCACTCCAGACTTGCTC |
| SEQ ID No. 1023: | 5′- TTCCTTCGGCACTGGGGTGT |
| SEQ ID No. 1024: | 5′- CCGCCTTCCTCCGACTTACG |
| SEQ ID No. 1025: | 5′- CCCGCCTTCCTCCGACTTAC |
| SEQ ID No. 1026: | 5′- CCTCCTCGCGGGCGTATCCG |
| SEQ ID No. 1027: | 5′- TCCTCGCGGGCGTATCCGGC |
| SEQ ID No. 1028: | 5′- CATTAGCGCCCGTTTCCGGG |
| SEQ ID No. 1029: | 5′- GCATTAGCGCCCGTTTCCGG |
| SEQ ID No. 1030: | 5′- GGCATTAGCGCCCGTTTCCG |
| SEQ ID No. 1031: | 5′- GTCTCGCATGGGGCTTTCCA |
| SEQ ID No. 1032: | 5′- GCCATGGACTTTCACTCCAG |
| SEQ ID No. 1033: | 5′- CATGGACTTTCACTCCAGAC |

The sequences SEQ ID No. 933 to SEQ ID No. 1033 are particularly suitable for the detection of bacteria of the genus Alicyclobacillus.

| | |
|---|---|
| SEQ ID No. 1034: | 5′- CCTTCCTCCGGCTTACGCCGGC |
| SEQ ID No. 1035: | 5′- CCTTCCTCCGACTTGCGCCGGC |
| SEQ ID No. 1036: | 5′- CCTTCCTCCGACTTTCACCGGC |

The nucleic acid probe molecules according to SEQ ID No. 1034 to SEQ ID No. 1036 are used as unlabelled competitor probes for the detection of bacteria of the genus Alicyclobacillus in combination with the oligonucleotide probe according to SEQ ID No. 933, in order to prevent the binding of the labelled oligonucleotide probe specific for bacteria of the genus Alicyclobacillus to nucleic acid sequences which are not specific for bacteria of the genus Alicyclobacillus.

| | |
|---|---|
| SEQ ID No. 1037: | 5′- ACCGTCTCACAAGGAGCTTT |
| SEQ ID No. 1038: | 5′- TACCGTCTCACAAGGAGCTT |
| SEQ ID No. 1039: | 5′- GTACCGTCTCACAAGGAGCT |
| SEQ ID No. 1040: | 5′- GCCTACCCGTGTATTATCCG |
| SEQ ID No. 1041: | 5′- CCGTCTCACAAGGAGCTTTC |
| SEQ ID No. 1042: | 5′- CTACCCGTGTATTATCCGGC |
| SEQ ID No. 1043: | 5′- GGTACCGTCTCACAAGGAGC |
| SEQ ID No. 1044: | 5′- CGTCTCACAAGGAGCTTTCC |
| SEQ ID No. 1045: | 5′- TCTCACAAGGAGCTTTCCAC |
| SEQ ID No. 1046: | 5′- TACCCGTGTATTATCCGGCA |
| SEQ ID No. 1047: | 5′- GTCTCACAAGGAGCTTTCCA |
| SEQ ID No. 1048: | 5′- ACCCGTGTATTATCCGGCAT |
| SEQ ID No. 1049: | 5′- CTCGGTACCGTCTCACAAGG |
| SEQ ID No. 1050: | 5′- CGGTACCGTCTCACAAGGAG |
| SEQ ID No. 1051: | 5′- ACTCGGTACCGTCTCACAAG |
| SEQ ID No. 1052: | 5′- CGGCTGGCTCCATAACGGTT |
| SEQ ID No. 1053: | 5′- ACAAGTAGATGCCTACCCGT |
| SEQ ID No. 1054: | 5′- TGGCTCCATAACGGTTACCT |
| SEQ ID No. 1055: | 5′- CAAGTAGATGCCTACCCGTG |
| SEQ ID No. 1056: | 5′- CACAAGTAGATGCCTACCCG |
| SEQ ID No. 1057: | 5′- GGCTCCATAACGGTTACCTC |
| SEQ ID No. 1058: | 5′- ACACAAGTAGATGCCTACCC |
| SEQ ID No. 1059: | 5′- CTGGCTCCATAACGGTTACC |
| SEQ ID No. 1060: | 5′- GCTGGCTCCATAACGGTTAC |
| SEQ ID No. 1061: | 5′- GGCTGGCTCCATAACGGTTA |
| SEQ ID No. 1062: | 5′- GCTCCATAACGGTTACCTCA |
| SEQ ID No. 1063: | 5′- AAGTAGATGCCTACCCGTGT |
| SEQ ID No. 1064: | 5′- CTCCATAACGGTTACCTCAC |
| SEQ ID No. 1065: | 5′- TGCCTACCCGTGTATTATCC |
| SEQ ID No. 1066: | 5′- TCGGTACCGTCTCACAAGGA |
| SEQ ID No. 1067: | 5′- CTCACAAGGAGCTTTCCACT |
| SEQ ID No. 1068: | 5′- GTAGATGCCTACCCGTGTAT |
| SEQ ID No. 1069: | 5′- CCTACCCGTGTATTATCCGG |
| SEQ ID No. 1070: | 5′- CACTCGGTACCGTCTCACAA |
| SEQ ID No. 1071: | 5′- CTCAGCGATGCAGTTGCATC |
| SEQ ID No. 1072: | 5′- AGTAGATGCCTACCCGTGTA |
| SEQ ID No. 1073: | 5′- GCGGCTGGCTCCATAACGGT |
| SEQ ID No. 1074: | 5′- CCAAAGCAATCCCAAGGTTG |
| SEQ ID No. 1075: | 5′- TCCATAACGGTTACCTCACC |
| SEQ ID No. 1076: | 5′- CCCGTGTATTATCCGGCATT |
| SEQ ID No. 1077: | 5′- TCTCAGCGATGCAGTTGCAT |
| SEQ ID No. 1078: | 5′- CCATAACGGTTACCTCACCG |
| SEQ ID No. 1079: | 5′- TCAGCGATGCAGTTGCATCT |
| SEQ ID No. 1080: | 5′- GGCGGCTGGCTCCATAACGG |
| SEQ ID No. 1081: | 5′- AAGCAATCCCAAGGTTGAGC |
| SEQ ID No. 1082: | 5′- TCACTCGGTACCGTCTCACA |
| SEQ ID No. 1083: | 5′- CCGAGTGTTATTCCAGTCTG |
| SEQ ID No. 1084: | 5′- CACAAGGAGCTTTCCACTCT |
| SEQ ID No. 1085: | 5′- ACAAGGAGCTTTCCACTCTC |
| SEQ ID No. 1086: | 5′- TCACAAGGAGCTTTCCACTC |
| SEQ ID No. 1087: | 5′- CAGCGATGCAGTTGCATCTT |
| SEQ ID No. 1088: | 5′- CAAGGAGCTTTCCACTCTCC |
| SEQ ID No. 1089: | 5′- CCAGTCTGAAAGGCAGATTG |
| SEQ ID No. 1090: | 5′- CAGTCTGAAAGGCAGATTGC |
| SEQ ID No. 1091: | 5′- CGGCGGCTGGCTCCATAACG |
| SEQ ID No: 1092: | 5′- CCTCTCTCAGCGATGCAGTT |
| SEQ ID No. 1093: | 5′- CTCTCTCAGCGATGCAGTTG |
| SEQ ID No. 1094: | 5′- TCTCTCAGCGATGCAGTTGC |
| SEQ ID No. 1095: | 5′- CTCTCAGCGATGCAGTTGCA |
| SEQ ID No. 1096: | 5′- CAATCCCAAGGTTGAGCCTT |
| SEQ ID No. 1097: | 5′- AATCCCAAGGTTGAGCCTTG |
| SEQ ID No. 1098: | 5′- AGCAATCCCAAGGTTGAGCC |
| SEQ ID No. 1099: | 5′- CTCACTCGGTACCGTCTCAC |
| SEQ ID No. 1100: | 5′- GCAATCCCAAGGTTGAGCCT |
| SEQ ID No. 1101: | 5′- GCCTTGGACTTTCACTTCAG |
| SEQ ID No. 1102: | 5′- CATAACGGTTACCTCACCGA |
| SEQ ID No. 1103: | 5′- CTCCTCTCTCAGCGATGCAG |
| SEQ ID No. 1104: | 5′- TCGGCGGCTGGCTCCATAAC |
| SEQ ID No. 1105: | 5′- AGTCTGAAAGGCAGATTGCC |
| SEQ ID No. 1106: | 5′- TCCTCTCTCAGCGATGCAGT |
| SEQ ID No. 1107: | 5′- CCCAAGGTTGAGCCTTGGAC |
| SEQ ID No. 1108: | 5′- ATAACGGTTACCTCACCGAC |
| SEQ ID No. 1109: | 5′- TCCCAAGGTTGAGCCTTGGA |
| SEQ ID No. 1110: | 5′- ATTATCCGGCATTAGCACCC |
| SEQ ID No. 1111: | 5′- CTACGTGCTGGTAACACAGA |
| SEQ ID No. 1112: | 5′- GCCGCTAGCCCCGAAGGGCT |
| SEQ ID No. 1113: | 5′- CTAGCCCCGAAGGGCTCGCT |
| SEQ ID No. 1114: | 5′- CGCTAGCCCCGAAGGGCTCG |
| SEQ ID No. 1115: | 5′- AGCCCCGAAGGGCTCGCTCG |
| SEQ ID No. 1116: | 5′- CCGCTAGCCCCGAAGGGCTC |
| SEQ ID No. 1117: | 5′- TAGCCCCGAAGGGCTCGCTC |
| SEQ ID No. 1118: | 5′- GCTAGCCCCGAAGGGCTCGC |
| SEQ ID No. 1119: | 5′- GCCCCGAAGGGCTCGCTCGA |
| SEQ ID No. 1120: | 5′- ATCCCAAGGTTGAGCCTTGG |
| SEQ ID No. 1121: | 5′- GAGCCTTGGACTTTCACTTC |
| SEQ ID No. 1122: | 5′- CAAGGTTGAGCCTTGGACTT |
| SEQ ID No. 1123: | 5′- GAGCTTTCCACTCTCCTTGT |
| SEQ ID No. 1124: | 5′- CCAAGGTTGAGCCTTGGACT |
| SEQ ID No. 1125: | 5′- CGGGCTCCTCTCTCAGCGAT |
| SEQ ID No. 1126: | 5′- GGAGCTTTCCACTCTCCTTG |
| SEQ ID No. 1127: | 5′- GGGCTCCTCTCTCAGCGATG |
| SEQ ID No. 1128: | 5′- TCTCCTTGTCGCTCTCCCCG |
| SEQ ID No. 1129: | 5′- TCCTTGTCGCTCTCCCCGAG |
| SEQ ID No. 1130: | 5′- AGCTTTCCACTCTCCTTGTC |
| SEQ ID No. 1131: | 5′- CCACTCTCCTTGTCGCTCTC |
| SEQ ID No. 1132: | 5′- GGCTCCTCTCTCAGCGATGC |
| SEQ ID No. 1133: | 5′- CCTTGTCGCTCTCCCCGAGC |
| SEQ ID No. 1134: | 5′- CACTCTCCTTGTCGCTCTCC |
| SEQ ID No. 1135: | 5′- ACTCTCCTTGTCGCTCTCCC |
| SEQ ID No. 1136: | 5′- CTCTCCTTGTCGCTCTCCCC |
| SEQ ID No. 1137: | 5′- GCGGGCTCCTCTCTCAGCGA |
| SEQ ID No. 1138: | 5′- GGCTCCATCATGGTTACCTC |

The sequences SEQ ID No. 1037 to SEQ ID No. 1138 are particularly suitable for the detection of Alicyclobacillus acidoterrestris.

| | |
|---|---|
| SEQ ID No. 1139: | 5′- CCGTCTCCTAAGGAGCTTTCCA |

The nucleic acid probe molecule according to SEQ ID No. 1139 is used as unlabelled competitor probe for the detection of Alicyclobacillus acidoterrestris in combination with the oligonucleotide probe according to SEQ ID No. 1044, in order to prevent the binding of the labelled oligonucleotide probe specific for Alicyclobacillus acidoterrestris to nucleic acid sequences which are not specific for Alicyclobacillus acidoterrestris.

| | |
|---|---|
| SEQ ID No. 1140: | 5′- TCCCTCCTTAACGGTTACCTCA |
| SEQ ID No. 1141: | 5′- TGGCTCCATAA(A/T)GGTTACCTCA |

The nucleic acid probe molecules according to SEQ ID No. 1140 to SEQ ID No. 1141 are used as unlabelled competitor probe for the detection of Alicyclobacillus acidoterrestris in combination with the oligonucleotide probe according to SEQ ID No. 1057, in order to prevent the binding of the labelled oligonucleotide probe specific for Alicyclobacillus acidoterrestris, to nucleic acid sequences which are not specific for Alicyclobacillus acidoterrestris.

| | |
|---|---|
| SEQ ID No. 1142: | 5′- CTTCCTCCGGCTTGCGCCGG |
| SEQ ID No. 1143: | 5′- CGCTCTTCCCGA(G/T)TGACTGA |
| SEQ ID No. 1144: | 5′- CCTCGGGCTCCTCCATC(A/T)GC |

The sequences SEQ ID No. 1142 to SEQ ID No. 1144 are particularly suitable for the simultanous detection of Alicyclobacillus cycloheptanicus and A. herbarius.

A further subject of the invention are derivatives of the above oligonucleotide sequences, demonstrating specific hybridization with target nucleic acid sequences of the respective microorganism despite deviations in sequence and/or length, and which are therefore suitable for use in a method according to the invention and ensure the the specific detection of the respective micororganism. These derivatives especially include:
a) nucleic acid molecules which (i) are identical with respect to the bases to one of the above oligonucleotide sequences (SEQ ID No. 1, 5 to 146, 148 to 154, 157 to 160, 163 to 1033, 1037 to 1138, 1142 to 1144) to at least 80%, preferably to at least 90% particularly preferred to at least 92%, 94%, 96%, or (ii) differ from the above oligonucleotide sequences by one or more deletions and/or additions and which allow for a specific hybridization with nucleic acid sequences of drink-spoiling yeasts of the genera Zygosaccharomyces, Hanseniaspora, Candida, Brettanomyces, Dekkera, Pichia, Saccharomyces and Saccharomycodes and in particular of the species Zygosaccharomyces bailii, Z. mellis, Z. rouxii, Z. bisporus, Z. fermentati, Z. microellipsoides, Hanseniaspora uvarum, Candida intermedia, C. crusei (Issatchenkia orientalis), C. parapsilosis, Brettanomyces bruxellensis, B. naardenensis, Dekkera anomala, Pichia membranaefaciens, P. minuta, P. anomala, Saccharomyces exiguus, S. cerevisiae, Saccharomycodes ludwigii or of the drink-spoiling molds of the genera Mucor, Byssochlamys, Neosartorya, Aspergillus and Talaromyces, in particular of the species Mucor racemosus, Byssochlamys nivea, Neosartorya fischeri, Aspergillus fumigatus and A. fischeri, Talaromyces flavus, T. bacillisporus and T. flavus or of the drink-spoiling bacteria of the genera Lactobacillus, Leuconostoc, Oenococcus, Weissella, Lactococcus, Acetobacter, Gluconobacter, Gluconoacetobacter, Bacillus and Alicyclobacillus, in particular of the species Lactobacillus collinoides, Leuconostoc mesenteroides, L. pseudomesenteroides, Oenococcus oeni, Bacillus coagulans, Alicyclobacillus ssp., A. acidoterrestris, A. cycloheptanicus and A. herbarium. In this context “specific hybridization” means that under the hybridization conditions described here or those known to the person skilled in the art in relation to in situ hybridization techniques, only the ribosomal RNA of the target organisms binds to the oligonucleotide, but not the rRNA of non-target microrganisms.
b) nucleic acid molecules which specifically hybridize under stringent conditions to a sequence complementary to the nucleic acid molecules mentioned in a) or to one of the probes SEQ ID No. 1, 5 to 146, 148 to 154, 157 to 160, 163 to 1033, 1037 to 1138, 1142 to 1144.
c) Nucleic acid molecules comprising an oligonucleotide sequence of SEQ ID No. 1, 5 to 146, 148 to 154, 157 to 160, 163 to 1033, 1037 to 1138, 1142 to 1144 or the sequence of a nucleic acid molecule according to a) or b) and having at least one further nucleotide in addition to the mentioned sequences and their derivatives, respectively, according to a) or b) and allowing specific hybridization with nucleic acid sequences of target organisms.

A further subject of the invention are also derivatives of the above competitor probe sequences, showing specific hybridizations with target nucleic acid sequences of the respective non-target genrera and species, respectively, despite variations in sequence and/or length, and which therefore prevent the binding of the oligonucleotide probe to the nucleic acid sequences of the genera and species, respectively, not to be detected. They are suitable for use in a method according to the invention and ensure a specific detection of the respective microorganism. These derivatives especially include

a) nucleic acid molecules which (i) are identical in terms of bases to one of the above oligonucleotide sequences (SEQ ID No. 2 to 4, 147, 155 to 156, 161 to 162, 1034 to 1036, 1139 to 1141) to at least 80%, preferably to at least 90%, particularly preferably to at least 92%, 94%, 96%, or (ii) differ from the above oligonucleotide sequences by one or more deletions and/or additions and which inhibit a specific hybridization of a specific oligonucleotide probe to nucleic acid sequences of a microorganism not to be detected.

b) Nucleic acid molecules which specifically hybridize to a sequence complementary to the nucleic acid molecules mentioned in a) or to one of the probes SEQ ID No. 2 to 4, 147, 155 to 156, 161 to 162, 1034 to 1036, 1139 to 1141 under stringent conditions.

c) Nucleic acid molecules comprising an oligonucleotide sequence of SEQ ID No. 2 to 4, 147, 155 to 156, 161 to 162, 1034 to 1036, 1139 to 1141 or the sequence of a nucleic acid molecule according to a) or b) and having at least one further nucleotide in addition to the mentioned sequences and their derivatives, respectively, according to a) or b) and prevent the binding of a specific oligonucleotide probe to the nucleic acid sequence of a non-target microorganism.

The degree of sequence identity of a nucleic acid probe molecule to the oligonucleotide probes having SEQ ID No. 1 to SEQ ID No. 1144 can be determined using the usual algorithms. In this respect, for example, the program for determining the sequence identity available under http://www.ncbi.nlm.nih.gov/BLAST (on this page for example the link “Standard nucleotide-nucleotide BLAST [blastn]”) is suitable.

In the present invention “hybridization” can have the same meaning as “complementary”. The present invention also comprises those oligonucleotides, which hybridize to the (theoretical) antisense strand of one of the inventive oligonucleotides including the derivatives of the present invention of SEQ ID No. 1 bis SEQ ID No. 1144.

The term “stringent conditions” generally means conditions under which a nucleic acid sequence preferentially hybridizes to its target sequence and to a clearly lower extent, or not at all, to other sequences. Stringent conditions are partly sequence-dependent and will vary under different circumstances. Longer sequences hybridize specifically at higher temperatures. In general, stringent conditions are selected in such a way that the temperature is approximately 5° C. below the thermal melting point (Tₘ) for the specific sequence at a defined ionic strength, pH and nucleic acid concentration. The Tₘ is the temperature (under defined ionic strength, pH and nucleic acid concentration) at which 50% of the probe molecules complementary to the target sequence hybridize to the target sequence in the steady state.

The nucleic acid probe molecules of the present invention may be used within the detection method with various hybridization solutions. Various organic solvents may be used in concentrations of 0-80%. By keeping stringent hybridization conditions, it is guaranteed that the nucleic acid probe molecule indeed hybridizes to the target sequence. Moderate conditions within the meaning of the invention are e.g. 0% formamide in a hybridization buffer as described below. Stringent conditions within the meaning of the invention are for example 20% to 80% formamide in the hybridization buffer.

Within the method according to the invention for the specific detection of yeasts of the genera Zygosaccharomyces, Hanseniaspora, Candida, Brettanomyces, Dekkera, Pichia, Saccharomyces and Saccharomycodes, in particular of the species Zygosaccharomyces bailii, Z. mellis, Z. rouxii, Z. bisporus, Z. fermentati, Z. microellipsoides, Hanseniaspora uvarum, Candida intermedia, C. crusei (Issatchenkia orientalis), C. parapsilosis, Brettanomyces bruxellensis, B. naardenensis, Dekkera anomala, Pichia membranaefaciens, P. minuta, P. anomala, Saccharomyces exiguus, S. cerevisiae, Saccharomycodes ludwigii a typical hybridization solution contains 0%-80% formamide, preferably 20%-60% formamide, particularly preferably 40% formamide. In addition, it has a salt concentration of 0.1 mol/l-1.5 mol/l, preferably of 0.7 mol/l-1.0 mol/l, and particularly preferably of 0.9 mol/l, whereby the salt preferably being sodium chloride. Further, the hybridization solution usually comprises a detergent, such as for instance sodium dodecyl sulfate (SDS) in a concentration of 0.001%-0.2%, preferably in a concentration of 0.005%-0.05%, particularly preferably in a concentration of 0.01%. For buffering the hybridization solution; various compounds such as Tris-HCl, sodium citrate, PIPES or HEPES may be used, which are usually used in concentrations of 0.01-0.1 mol/l, preferably of 0.01 to 0.05 mol/l, in a pH range of 6.0-9.0, preferably 7.0 to 8.0. The particularly preferred embodiment of the hybridization solution in accordance with the invention contains 0.02 mol/l Tris-HCl, pH 8.0.

Within the method according to the invention for the specific detection of molds of the genera Mucor, Byssochlamys Neosartorya, Aspergillus and Talaromyces, in particular of the species Mucor racemosus, Byssochlamys nivea, Neosartorya fischeri, Aspergillus fumigatus und A. fischeri, Talaromyces flavus, T. bacillisporus and T. flavus, a typical hybridization solution contains 0%-80% formamide, preferably 10%-60% formamide, particularly preferably 20% formamide. In addition, it has a salt concentration of 0.1 mol/l-1.5 mol/l, preferably of 0.7 mol/l-1.0 mol/l, and particularly preferably of 0.9 mol/l, whereby the salt preferably being sodium chloride. Further, the hybridization solution usually comprises a detergent, such as for instance sodium dodecyl sulfate (SDS) at a concentration of 0.001%-0.2%, preferably at a concentration of 0.005-0.05%, particularly preferably at a concentration of 0.01%. For buffering the hybridization solution, various compounds such as Tris-HCl, sodium citrate, PIPES or HEPES may be used, which are usually used in concentrations of 0.01-0.1 mol/l, preferably of 0.01 to 0.05 mol/l, in a pH range of 6.0-9.0, preferably 7.0 to 8.0. The particularly preferred embodiment of the hybridization solution in accordance with the invention contains 0.02 mol/l Tris-HCl, pH 8.0.

Within the method according to the invention for the specific detection of bacteria of the genera Lactobacillus, Leuconostoc, Oenococcus, Weissella, Lactococcus, Acetobacter, Gluconobacter, Gluconoacetobacter, Bacillus and Alicyclobacillus, in particular of the species Lactobacillus collinoides, Leuconostoc mesenteroides, L. pseudomesenteroides, Oenococcus oeni, Bacillus coagulans, Alicyclobacillus ssp., A. acidoterrestris, A. cycloheptanicus and A. herbarium, a typical hybridization solution contains 0%-80% formamide, preferably 10%-60% formamide, particularly preferably 20% formamide. In addition, it has a salt concentration of 0.1 mol/l-1.5 mol/l, preferably of 0.7 mol/l-1.0 mol/l, and particularly preferably of 0.9 mol/l, whereby the salt preferably being sodium chloride. Further, the hybridization solution usually comprises a detergent, such as for instance sodium dodecyl sulfate (SDS) at a concentration of 0.001%-0.2%, preferably at a concentration of 0.005%-0.05%, particularly preferably at a concentration of 0.01%. For buffering the hybridization solution, various compounds such as Tris-HCl, sodium citrate, PIPES or HEPES may be used, which are usually used in concentrations of 0.01-0.1 mol/l, preferably of 0.01 to 0.05 mol/l, in a pH range of 6.0-9.0, preferably 7.0 to 8.0. The particularly preferred embodiment of the hybridization solution in accordance with the invention contains 0.02 mol/l Tris-HCl, pH 8.0.

It shall be understood that the one skilled in the art can select the specified concentrations of the constituents of the hybridization buffer in such a way that the desired stringency of the hybridization reaction is achieved. Particularly preferred embodiments are related from stringent to particularly stringent hybridization conditions. Using these stringent conditions the one skilled in the art can determine whether a particular nucleic acid molecule allows the specific detection of nucleic acid sequences of target organisms and may thus be reliably used within the invention.

The concentration of the nucleic acid probe in the hybridization buffer depends on the kind of label and on the number of target structures. In order to allow rapid and efficient hybridization, the number of nucleic acid probe molecules should exceed the number of target structures by several orders of magnitude. However, it has to be taken into consideration that in fluorescence in situ-hybridization (FISH) too high levels of fluorescencently labelled nucleic acid probe molecules result in increased background fluorescence. The concentration of the nucleic acid probe molecules should therefore be in the range between 0.5 and 500 ng/μl. Within the method of the present invention the preferred nucleic acid probe concentration is between 1.0 and 10 ng for each nucleic acid probe molecule used per μl of hybridization solution. The volume of hybridization solution used should be between 8 μl and 100 ml, in a particularly preferred embodiment of the method of present invention it is 30 μl.

The concentration of the competitor probe in the hybridization buffer depends on the number of target structures. In order to allow rapid and efficient hybridization, the number of competitor probes should exceed the number of target structures by several orders of magnitude. The concentration of the competitor probe molecules should therefore be in a range between 0.5 and 500 ng/μl. Within the method of the present invention the preferred concentration is between 1.0 and 10 ng for each competitor probe molecule used per μl of hybridization solution. The volume of hybridization solution used should be between 8 μl and 100 ml, in a particularly preferred embodiment of the method of present invention it is 30 μl.

The hybridization usually lasts between 10 minutes and 12 hours, preferably the hybridization lasts for about 1.5 hours. The hybridization temperature is preferably between 44° C. and 48° C., particularly preferably 46° C., whereby the parameter of the hybridization temperature as well as the concentration of salts and detergents in the hybridization solution may be optimized depending on the nucleic acid probes, especially their lengths and the degree to which they are complementary to the target sequence in the cell to be detected. The one skilled in the art is familiar with appropriate calculations.

After hybridization the non-hybridized and excess nucleic acid probe molecules should be removed or washed off, which is usually achieved by a conventional washing solution. This washing solution may, if desired, contain 0.001-0.1%, preferably 0.005-0.05%, particularly preferably 0.01% of a detergent such as SDS, as well as Tris-HCl in a concentration of 0.001-0.1 mol/l, preferably 0.01-0.05 mol/l, particularly preferably 0.02 mol/l, wherein the pH value of Tris-HCl is within the range of 6.0 to 9.0, preferably of 7.0 to 8.0, particularly preferably 8.0. A detergent may be contained, although this is not obligatorily necessary. Furthermore, the washing solution usually contains NaCl, whereby the concentration is 0.003 mol/l to 0.9 mol/l, preferably 0.01 mol/l to 0.9 mol/l, depending on the stringency required. Moreover, the washing solution may contain EDTA, whereby the concentration is preferably 0.005 mol/l. The washing solution may further contain suitable amounts of preservatives known to the expert.

In general, buffer solutions are used in the washing step which can in principle be very similar to the hybridization buffer (buffered sodium chloride solution), except that the washing step is usually performed in a buffer with a lower salt concentration and at a higher temperature, respectively. For theoretical estimation of the hybridization conditions, the following formula may be used:

Td=81.5+16.6 lg[Na⁺]+0.4×(% GC)−820/n−0.5×(% FA)

Td=dissociation temperature in ° C.

[Na⁺]=molarity of the sodium ions

% GC=percentage of guanine and cytosine nucleotides relative to the total number of bases

n=length of the hybrid

% FA=formamide content

Using this formula, the formamide content (which should be as low as possible due to the toxicity of the formamide) of the washing buffer may for example be replaced by a correspondingly lower sodium chloride content. However, the person skilled in the art is, from the extensive literature concerning in situ hybridization methods, aware of the fact that, and in which way, the mentioned contents can be varied. Concerning the stringency of the hybridization conditions, the same applies as outlined above for the hybridization buffer.

The “washing off” of the non-bound nucleic acid probe molecules is usually performed at a temperature in the range of 44° C. to 52° C., preferably of 44° C. to 50° C. and particularly preferably at 46° C. for 10 to 40 minutes, preferably for 15 minutes.

The specifically hybridized nucleic acid probe molecules can then be detected in the respective-cells, provided that the nucleic acid probe molecule is detectable, e.g., by linking the nucleic acid probe molecule to a marker by covalent binding. As detectable markers, for example, fluorescent groups, such as for example CY2 (available from Amersham Life Sciences, Inc., Arlington Heights, USA), CY3 (also available from Amersham Life Sciences), CY5 (also obtainable from Amersham Life Sciences), FITC (Molecular Probes Inc., Eugene, USA), FLUOS (available from Roche Diagnostics GmbH, Mannheim, Germany), TRITC (available from Molecular Probes Inc., Eugene, USA), 6-FAM or FLUOS-PRIME are used, which are well known to the person skilled in the art. Also chemical markers, radioactive markers or enzymatic markers, such as horseradish peroxidase, acid phosphatase, alkaline phosphatase, peroxidase may be used. For each of these enzymes a number of chromogens is known which may be converted instead of the natural substrate and may be transformed into either coloured or fluorescent products. Examples of such chromogens are listed in the following table:

TABLE

| Enzyme | Chromogen |
|---|---|
| 1. Alkaline | 4-methylumbelliferyl phosphate (*), bis(4- |
| phosphatase and | methylumbelliferyl phosphate, (*) 3-O- |
| acid phosphatase | methylfluorescein, flavone-3-diphosphate |
| | triammonium salt (*), p-nitrophenylphosphate |
| | disodium salt |
| 2. Peroxidase | tyramine hydrochloride (*), 3-(p-hydroxyphenyl)- |
| | propionate (*), p-hydroxyphenethyl alcohol (*), 2,2′- |
| | azino-di-3-ethylbenzothiazoline sulfonic acid |
| | (ABTS), ortho-phenylendiamine dihydrochloride, o- |
| | dianisidine, 5-aminosalicylic acid, p-ucresol (*), |
| | 3,3′-dimethyloxy benzidine, 3-methyl-2- |
| | benzothiazoline hydrazone, tetramethylbenzidine |
| 3. Horseradish | H₂O₂ + diammonium benzidine |
| peroxidase | H₂O₂ + tetramethylbenzidine |
| 4. β-D- | o-nitrophenyl-β-D-galactopyranoside, 4- |
| galactosidase | methylumbelliferyl-β-D-galactoside |
| 5. Glucose oxidase | ABTS, glucose and thiazolyl blue |

*fluorescence

Finally, it is possible to design the nucleic acid probe molecules in such a way that another nucleic acid sequence suitable for hybridization is present at their 5′ or 3′ ends. This nucleic acid sequence in turn comprises about 15 to 100, preferably 15-50 nucleotides. This second nucleic acid region may in turn be detected by a nucleic acid probe molecule which is detectable by one of the above-mentioned agents.

Another possibility is the coupling of the detectable nucleic acid probe molecules to a haptene which may subsequently be brought into contact with an antibody recognising the haptene. Digoxigenin may be mentioned as an example of such a haptene. Other examples in addition to those mentioned are well known to the one skilled in the art.

The final evaluation is, depending on the kind of labelling of the probe used, possible, among others, with an optical microscope, epifluorescence microscope, chemoluminometer, fluorometer.

An important advantage of the methods described in this application for the specific detection of drink-spoiling yeasts of the genera Zygosaccharomyces, Hanseniaspora, Candida, Brettanomyces, Dekkera, Pichia, Saccharomyces and Saccharomycodes, in particular of the species Zygosaccharomyces bailii, Z. mellis, Z. rouxii, Z. bisporus, Z. fermentati, Z. microellipsoides, Hanseniaspora uvarum, Candida intermedia, C. crusei (Issatchenkia orientalis), C. parapsilosis, Brettanomyces bruxellensis, B. naardenensis, Dekkera anomala, Pichia membranaefaciens, P. minuta, P. anomala, Saccharomyces exiguus, S. cerevisiae, Saccharomycodes ludwigii or for the specific detection of drink-spoiling molds of the genera Mucor, Byssochlamys, Neosartorya, Aspergillus and Talaromyces, in particular of species Mucor racemosus, Byssochlamys nivea, Neosartorya fischeri, Aspergillus fumigatus and A. fischeri, Talaromyces flavus, T bacillisporus and T. flavus, or for the specific detection of drink-spoiling bacteria of the genera Lactobacillus, Leuconostoc, Oenococcus, Weissella, Lactococcus, Acetobacter, Gluconobacter, Gluconoacetobacter, Bacillus and Alicyclobacillus, in particular of the species Lactobacillus collinoides, Leuconostoc mesenteroides, L. pseudomesenteroides, Oenococcus oeni, Bacillus coagulans, Alicyclobacillus ssp., A. acidoterrestris, A. cycloheptanicus and A. herbarius compared to the detection methods described above is the exceptional speed. In comparison to conventional cultivation methods which need up to 10 days, the result is obtained within 24 to 48 hours when the methods according to the invention are used.

Another advantage is the ability to perform an accurate differentiation of the drink-spoiling microorganims to be detected. With the methods common up to now no differentiation of the microorganisms was carried out until the genus or species level, as the differentiation was either not possible at all or was too time-consuming.

Another advantage is the specificity of these methods. With the nucleic acid probe molecules used, drink-spoiling yeasts of the genera Zygosaccharomyces, Hanseniaspora, Candida, Brettanomyces, Dekkera, Pichia, Saccharomyces and Saccharomycodes, in particular the species Zygosaccharomyces bailii, Z. mellis, Z. rouxii, Z. bisporus, Z. fermentati, Z. microellipsoides, Hanseniaspora uvarum, Candida intermedia, C. crusei (Issatchenkia orientalis), C. parapsilosis, Brettanomyces bruxellensis, B. naardensis, Dekkera anomala, Pichia membranaefaciens, P. minuta, P. anomala, Saccharomyces exiguus, S. cerevisiae, Saccharomycodes ludwigii or drink-spoiling molds of the genera Mucor, Byssochlamys, Neosartorya, Aspergillus and Talaromyces, in particular of the species Mucor racemosus, Byssochlamys nivea, Neosartorya fischeri, Aspergillus fumigatus and A. fischeri, Talaromyces flavus, T. bacillisporus and T. flavus or drink-spoiling bacteria of the genera Lactobacillus, Leuconostoc, Oenococcus, Weissella, Lactococcus, Acetobacter, Gluconobacter, Gluconoacetobacter, Bacillus and Alicyclobacillus, in particular of the species Lactobacillus collinoides, Leuconostoc mesenteroides, L. pseudomesenteroides, Oenococcus oeni, Bacillus coagulans, Alicyclobacillus ssp., A. acidoterrestris, A. cycloheptanicus and A. herbarius can be detected in a highly specific manner. By the visualisation of the microorganisms a visual control may be performed at the same time. False-positive results, such as often occurring in polymerase chain reaction, are therefore ruled out.

Another advantage of the methods according to the invention is their ease of use. Thus, using this methods, large numbers of samples can be easily tested regarding the presence of the mentioned microorganims.

Finally, an important advantage compared to the state of the art is the possible simultaneous detection of several of the mentioned microorganisms by the use of respective mixtures of probes. Following this approach all practise relevant drink-spoiling microorganisms can be detected in a few tests.

Different probes may hereby be coupled with different labels, so that the various, detected micororganisms may be discriminated in an easy and reliable way. For example, a first oligonucleotide may be specifically labelled with a green fluorescence dye and serves for the detection of a certain genus or species of microorganism. A second oligonucleotide is also specifically labelled with, for instance, a red fluorescence dye and serves for the detection of a second genus or species of microorganism. The oligonucleotides referred to as competitor probes remain non-labelled and prevent the binding of the first and/or the second oligonucleotide probe to bacteria which do not belong to the genera or species to be detected. The different labels, e.g. the green fluorescence dye on the one hand and the red fluorescence dye on the other hand may be differentiated in an easy manner, for example by using different filters in fluorescence microscopy.

The methods according to the invention may be used in various ways.

For example, non-alcoholic drinks (e.g. fruit juices, fruct nectars, fruit concentrates, mashed fruits, soft drinks and waters) may be tested for the presence of the microorganisms to be detected.

For example, also environmental samples can be tested for the presence of the micororganisms to be detected. Theses samples may be, for example, collected from soil or be parts of plants.

The method according to the invention may further be used for testing sewage samples or silage samples.

The method according to the invention may further be used for testing medicinal samples, e.g. stool samples, blood cultures, sputum, tissue samples (also sections), wound material, urine, samples from the respiratory tract, implants and catheter surfaces.

Another field of use of the method according to the invention is the control of food. In preferred embodiments the food samples are obtained from milk or milk products (yogurt, cheese, curd, butter, buttermilk), drinking water, alcoholic drinks (beer, wine, spirits), bakery products or meat products.

A further field of use of the method according to the invention is the analysis of pharmaceutical and cosmetic products, e.g. ointments, creams, tinctures, juices, solutions, drops, etc.

Furthermore, according to the invention, kits for performing the respective methods are provided. The hybridization arrangement contained in these kits is described for example in German patent application 100 61 655.0. Express reference is herewith made to the disclosure contained in this document with respect to the in situ hybridization arrangement.

Besides the described hybridization arrangement (referred to as VIT reactor), the most important component of the kits is the respective hybridization solution (referred to as VIT solution) with the nucleic acid probe molecules specific for the microorganisms to be detected, which are described above (VIT solution). Further contained are the respective hybridization buffer (Solution C) and a concentrate of the respective washing solution (Solution D). Also contained are optionally fixation solutions (Solution A and Solution B) as well as optionally an embedding solution (finisher). Optionally, solutions are contained for performing in parallel a positive control as well as of a negative control.

The following example is intended to illustrate the invention without limitation.

### EXAMPLE

Specific rapid detection of drink spoiling microorganisms in a sample

A sample is cultivated for 20 to 48 hours in a suitable manner. For the detection of yeasts and molds cultivation may be performed, for example, in SSL-bouillon for 24 hours at 25° C. For the detection of lactic acid bacteria the cultivation may be performed for example in MRS-bouillon for 48 hours at 30° C. For the detection of aceteic acid bacteria the cultivation may be performed, for example, on DSM-agar for 48 hours at 28° C. For the detection of bacilli, in particular B. coagulans, the cultivation may be performed, for example, on dextrose-casein-peptone-agar for 48 hours at 55° C. For the detection of alicyclobacilli, the cultivation may be performed, for example, in BAM-bouillon for 48 hours at 44° C.

To an aliquot of the culture the same volume of fixation solution (Solution B, ethanol absolute) is added. Alternatively, an aliquot of the culture may be centrifuged (4000 g, 5 min, room temperature) and, after discarding the supernatant, the pellet may be dissolved in 4 drops of fixation solution (Solution B).

For performing the hybridization a suitable aliquot of the fixed cells (preferably 5 μl) is applied onto a slide and dried (46° C., 30 min, or until completely dry). Alternatively, the cells may also be applied to other carrier materials (e.g. a microtiter plate or a filter). The dried cells are then completely dehydrated by again adding the fixation solution (Solution B). The slide is again dried (room temperature, 3 min, or until completely dry).

Then the hybridization solution (VIT solution, hybridization buffer containing labeled probe molecules) containing the above described nucleic acid probe molecules specific for the microorganisms to be detected, is applied to the fixed, dehydrated cells. The preferred volume is 40 μl. The slide is then incubated (46° C., 90 min) in a chamber humidified with hybridization buffer (Solution C), preferably the VIT reactor (c. f. DE 100 61 655.0).

Then the slide is removed from the chamber, the chamber is filled with washing solution (Solution D, diluted 1:10 with distilled water) and the slide is incubated in the chamber (46° C., 15 min).

Then the chamber is filled with distilled water, the slide is briefly immersed and then air-dried in lateral position (46° C., 30 min or until completely dry).

Then the slide is embedded in a suitable medium (Finisher).

Finally, the sample is analyzed with the help of a fluorescence microscope.

1. A method for the detection of drink-spoiling microorganisms in a sample, whereby the detection is carried out by using at least one oligonucleotide probe having a nucleic acid sequence selected from the group consisting of:

| | |
|---|---|
| SEQ ID No. 1: | 5′- GTTTGACCAGATTCTCCGCTC |
| SEQ ID No. 5: | 5′- CCCGGTCGAATTAAAACC |
| SEQ ID No. 6: | 5′- GCCCGGTCGAATTAAAAC |
| SEQ ID No. 7: | 5′- GGCCCGGTCGAATTAAAA |
| SEQ ID No. 8: | 5′- AGGCCCGGTCGAATTAAA |
| SEQ ID No. 9: | 5′- AAGGCCCGGTCGAATTAA |
| SEQ ID No. 10: | 5′- ATATTCGAGCGAAACGCC |
| SEQ ID No. 11: | 5′- AAAGATCCGGACCGGCCG |
| SEQ ID No. 12 | 5′- GGAAAGATCCGGACCGGC |
| SEQ ID No. 13 | 5′- GAAAGATCCGGACCGGCC |
| SEQ ID No. 14 | 5′- GATCCGGACCGGCCGACC |
| SEQ ID No. 15 | 5′- AGATCCGGACCGGCCGAC |
| SEQ ID No. 16 | 5′- AAGATCCGGACCGGCCGA |
| SEQ ID No. 17 | 5′- GAAAGGCCCGGTCGAATT |
| SEQ ID No. 18 | 5′- AAAGGCCCGGTCGAATTA |
| SEQ ID No. 19 | 5′- GGAAAGGCCCGGTCGAAT |
| SEQ ID No. 20 | 5′- AGGAAAGGCCCGGTCGAA |
| SEQ ID No. 21 | 5′- AAGGAAAGGCCCGGTCGA |
| SEQ ID No. 22: | 5′- ATAGCACTGGGATCCTCGCC |
| SEQ ID No. 23: | 5′- CCAGCCCCAAAGTTACCTTC |
| SEQ ID No. 24: | 5′- TCCTTGACGTAAAGTCGCAG |
| SEQ ID No. 25: | 5′- GGAAGAAAACCAGTACGC |
| SEQ ID No. 26: | 5′- CCGGTCGGAAGAAAACCA |
| SEQ ID No. 27: | 5′- GAAGAAAACCAGTACGCG |
| SEQ ID No. 28: | 5′- CCCGGTCGGAAGAAAACC |
| SEQ ID No. 29: | 5′- CGGTCGGAAGAAAACCAG |
| SEQ ID No. 30: | 5′- GGTCGGAAGAAAACCAGT |
| SEQ ID No. 31: | 5′- AAGAAAACCAGTACGCGG |
| SEQ ID No. 32: | 5′- GTACGCGGAAAAATCCGG |
| SEQ ID No. 33: | 5′- AGTACGCGGAAAAATCCG |
| SEQ ID No. 34: | 5′- GCGGAAAAATCCGGACCG |
| SEQ ID No. 35: | 5′- CGGAAGAAAACCAGTACG |
| SEQ ID No. 36: | 5′- GCCCGGTCGGAAGAAAAC |
| SEQ ID No. 37: | 5′- CGCGGAAAAATCCGGACC |
| SEQ ID No. 38: | 5′- CAGTACGCGGAAAAATCC |
| SEQ ID No. 39: | 5′- AGAAAACCAGTACGCGGA |
| SEQ ID No. 40: | 5′- GGCCCGGTCGGAAGAAAA |
| SEQ ID No. 41: | 5′- ATAAACACCACCCGATCC |
| SEQ ID No. 42: | 5′- ACGCGGAAAAATCCGGAC |
| SEQ ID No. 43: | 5′- GAGAGGCCCGGTCGGAAG |
| SEQ ID No. 44: | 5′- AGAGGCCCGGTCGGAAGA |
| SEQ ID No. 45: | 5′- GAGGCCCGGTCGGAAGAA |
| SEQ ID No. 46: | 5′- AGGCCCGGTCGGAAGAAA |
| SEQ ID No. 47: | 5′- CCGAGTGGGTCAGTAAAT |
| SEQ ID No. 48: | 5′- CCAGTACGCGGAAAAATC |
| SEQ ID No. 49: | 5′- TAAACACCACCCGATCCC |
| SEQ ID No. 50: | 5′- GGAGAGGCCCGGTCGGAA |
| SEQ ID No. 51: | 5′- GAAAACCAGTACGCGGAA |
| SEQ ID No. 52: | 5′- TACGCGGAAAAATCCGGA |
| SEQ ID No. 53: | 5′- GGCCACAGGGACCCAGGG |
| SEQ ID No. 54: | 5′- TCACCAAGGGCCACAGGG |
| SEQ ID No. 55: | 5′- GGGCCACAGGGACCCAGG |
| SEQ ID No. 56: | 5′- TTCACCAAGGGCCACAGG |
| SEQ ID No. 57: | 5′- ACAGGGACCCAGGGCTAG |
| SEQ ID No. 58: | 5′- AGGGCCACAGGGACCCAG |
| SEQ ID No. 59: | 5′- GTTCACCAAGGGCCACAG |
| SEQ ID No. 60: | 5′- GCCACAGGGACCCAGGGC |
| SEQ ID No. 61: | 5′- CAGGGACCCAGGGCTAGC |
| SEQ ID No. 62: | 5′- AGGGACCCAGGGCTAGCC |
| SEQ ID No. 63: | 5′- ACCAAGGGCCACAGGGAC |
| SEQ ID No. 64: | 5′- CCACAGGGACCCAGGGCT |
| SEQ ID No. 65: | 5′- CACAGGGACCCAGGGCTA |
| SEQ ID No. 66: | 5′- CACCAAGGGCCACAGGGA |
| SEQ ID No. 67: | 5′- GGGACCCAGGGCTAGCCA |
| SEQ ID No. 68: | 5′- AGGAGAGGCCCGGTCGGA |
| SEQ ID No. 69: | 5′- AAGGAGAGGCCCGGTCGG |
| SEQ ID No. 70: | 5′- GAAGGAGAGGCCCGGTCG |
| SEQ ID No. 71: | 5′- AGGGCTAGCCAGAAGGAG |
| SEQ ID No. 72: | 5′- GGGCTAGCCAGAAGGAGA |
| SEQ ID No. 73: | 5′- AGAAGGAGAGGCCCGGTC |
| SEQ ID No. 74: | 5′- CAAGGGCCACAGGGACCC |
| SEQ ID No. 75: | 5′- CCAAGGGCCACAGGGACC |
| SEQ ID No. 76: | 5′- GTCGGAAAAACCAGTACG |
| SEQ ID No. 77: | 5′- GCCCGGTCGGAAAAACCA |
| SEQ ID No. 78: | 5′- CCGGTCGGAAAAACCAGT |
| SEQ ID No. 79: | 5′- CCCGGTCGGAAAAACCAG |
| SEQ ID No. 80: | 5′- TCGGAAAAACCAGTACGC |
| SEQ ID No. 81: | 5′- CGGAAAAACCAGTACGCG |
| SEQ ID No. 82: | 5′- GGAAAAACCAGTACGCGG |
| SEQ ID No. 83: | 5′- GTACGCGGAAAAATCCGG |
| SEQ ID No. 84: | 5′- AGTACGCGGAAAAATCCG |
| SEQ ID No. 85: | 5′- GCGGAAAAATCCGGACCG |
| SEQ ID No. 86: | 5′- GGTCGGAAAAACCAGTAC |
| SEQ ID No. 87: | 5′- ACTCCTAGTGGTGCCCTT |
| SEQ ID No. 88: | 5′- GCTCCACTCCTAGTGGTG |
| SEQ ID No. 89: | 5′- CACTCCTAGTGGTGCCCT |
| SEQ ID No. 90: | 5′- CTCCACTCCTAGTGGTGC |
| SEQ ID No. 91: | 5′- TCCACTCCTAGTGGTGCC |
| SEQ ID No. 92: | 5′- CCACTCCTAGTGGTGCCC |
| SEQ ID No. 93: | 5′- GGCTCCACTCCTAGTGGT |
| SEQ ID No. 94: | 5′- AGGCTCCACTCCTAGTGG |
| SEQ ID No. 95: | 5′- GGCCCGGTCGGAAAAACC |
| SEQ ID No. 96: | 5′- GAAAAACCAGTACGCGGA |
| SEQ ID No. 97: | 5′- CGCGGAAAAATCCGGACC |
| SEQ ID No. 98: | 5′- CAGTACGCGGAAAAATCC |
| SEQ ID No. 99: | 5′- CGGTCGGAAAAACCAGTA |
| SEQ ID No. 100: | 5′- AAGGCCCGGTCGGAAAAA |
| SEQ ID No. 101: | 5′- CAGGCTCCACTCCTAGTG |
| SEQ ID No. 102: | 5′- CTCCTAGTGGTGCCCTTC |
| SEQ ID No. 103: | 5′- TCCTAGTGGTGCCCTTCC |
| SEQ ID No. 104: | 5′- GCAGGCTCCACTCCTAGT |
| SEQ ID No. 105: | 5′- AGGCCCGGTCGGAAAAAC |
| SEQ ID No. 106: | 5′- ACGCGGAAAAATCCGGAC |
| SEQ ID No. 107: | 5′- CCAGTACGCGGAAAAATC |
| SEQ ID No. 108: | 5′- CTAGTGGTGCCCTTCCGT |
| SEQ ID No. 109: | 5′- GAAAGGCCCGGTCGGAAA |
| SEQ ID No. 110: | 5′- AAAGGCCCGGTCGGAAAA |
| SEQ ID No. 111: | 5′- TACGCGGAAAAATCCGGA |
| SEQ ID No. 112: | 5′- GGAAAGGCCCGGTCGGAA |
| SEQ ID No. 113: | 5′- ATCTCTTCCGAAAGGTCG |
| SEQ ID No. 114: | 5′- CATCTCTTCCGAAAGGTC |
| SEQ ID No. 115: | 5′- CTCTTCCGAAAGGTCGAG |
| SEQ ID No. 116: | 5′- CTTCCGAAAGGTCGAGAT |
| SEQ ID No. 117: | 5′- TCTCTTCCGAAAGGTCGA |
| SEQ ID No. 118: | 5′- TCTTCCGAAAGGTCGAGA |
| SEQ ID No. 119: | 5′- CCTAGTGGTGCCCTTCCG |
| SEQ ID No. 120: | 5′- TAGTGGTGCCCTTCCGTC |
| SEQ ID No. 121: | 5′- AGTGGTGCCCTTCCGTCA |
| SEQ ID No. 122: | 5′- GCCAAGGTTAGACTCGTT |
| SEQ ID No. 123: | 5′- GGCCAAGGTTAGACTCGT |
| SEQ ID No. 124: | 5′- CCAAGGTTAGACTCGTTG |
| SEQ ID No. 125: | 5′- CAAGGTTAGACTCGTTGG |
| SEQ ID No. 126: | 5′- AAGGTTAGACTCGTTGGC |
| SEQ ID No. 127: | 5′- CTCGCCTCACGGGGTTCTCA |
| SEQ ID No. 128: | 5′- GGCCCGGTCGAAATTAAA |
| SEQ ID No. 129: | 5′- AGGCCCGGTCGAAATTAA |
| SEQ ID No. 130: | 5′- AAGGCCCGGTCGAAATTA |
| SEQ ID No. 131: | 5′- AAAGGCCCGGTCGAAATT |
| SEQ ID No. 132: | 5′- GAAAGGCCCGGTCGAAAT |
| SEQ ID No. 133: | 5′- ATATTCGAGCGAAACGCC |
| SEQ ID No. 134: | 5′- GGAAAGGCCCGGTCGAAA |
| SEQ ID No. 135: | 5′- AAAGATCCGGACCGGCCG |
| SEQ ID No. 136: | 5′- GGAAAGATCCGGACCGGC |
| SEQ ID No. 137: | 5′- GAAAGATCCGGACCGGCC |
| SEQ ID No. 138: | 5′- GATCCGGACCGGCCGACC |
| SEQ ID No. 139: | 5′- AGATCCGGACCGGCCGAC |
| SEQ ID No. 140: | 5′- AAGATCCGGACCGGCCGA |
| SEQ ID No. 141: | 5′- AGGAAAGGCCCGGTCGAA |
| SEQ ID No. 142: | 5′- AAGGAAAGGCCCGGTCGA |
| SEQ ID No. 143: | 5′- CGAGCAAAACGCCTGCTTTG |
| SEQ ID No. 144: | 5′- CGCTCTGAAAGAGAGTTGCC |
| SEQ ID No. 145: | 5′- AGTTGCCCCCTACACTAGAC |
| SEQ ID No. 146: | 5′- GCTTCTCCGTCCCGCGCCG |
| SEQ ID No. 148: | 5′- CCTGGTTCGCCAAAAAGGC |
| SEQ ID No. 149: | 5′- GATTCTCGGCCCCATGGG |
| SEQ ID No. 150: | 5′- ACCCTCTACGGCAGCCTGTT |
| SEQ ID No. 151: | 5′- GATCGGTCTCCAGCGATTCA |
| SEQ ID No. 152: | 5′- ACCCTCCACGGCGGCCTGTT |
| SEQ ID No. 153: | 5′- GATTCTCCGCGCCATGGG |
| SEQ ID No. 154: | 5′- TCATCAGACGGGATTCTCAC |
| SEQ ID No. 157: | 5′- AGTTGCCCCCTCCTCTAAGC |
| SEQ ID No. 158: | 5′- CTGCCACAAGGACAAATGGT |
| SEQ ID No. 159: | 5′- TGCCCCCTCTTCTAAGCAAAT |
| SEQ ID No. 160: | 5′- CCCCAAAGTTGCCCTCTC |
| SEQ ID No. 163: | 5′- AAGACCAGGCCACCTCAT |
| SEQ ID No. 164: | 5′- CATCATAGAACACCGTCC |
| SEQ ID No. 165: | 5′- CCTTCCGAAGTCGAGGTTTT |
| SEQ ID No. 166: | 5′- GGGAGTGTTGCCAACTC |
| SEQ ID No. 167: | 5′- AGCGGTCGTTCGCAACCCT |
| SEQ ID No. 168: | 5′- CCGAAGTCGGGGTTTTGCGG |
| SEQ ID No. 169: | 5′- GATAGCCGAAACCACCTTTC |
| SEQ ID No. 170: | 5′- GCCGAAACCACCTTTCAAAC |
| SEQ ID No. 171: | 5′- GTGATAGCCGAAACCACCTT |
| SEQ ID No. 172: | 5′- AGTGATAGCCGAAACCACCT |
| SEQ ID No. 173: | 5′- TTTAACGGGATGCGTTCGAC |
| SEQ ID No. 174: | 5′- AAGTGATAGCCGAAACCACC |
| SEQ ID No. 175: | 5′- GGTTGAATACCGTCAACGTC |
| SEQ ID No. 176: | 5′- GCACAGTATGTCAAGACCTG |
| SEQ ID No. 177: | 5′- CATCCGATGTGCAAGCACTT |
| SEQ ID No. 178: | 5′- TCATCCGATGTGCAAGCACT |
| SEQ ID No. 179: | 5′- CCGATGTGCAAGCACTTCAT |
| SEQ ID No. 180: | 5′- CCACTCATCCGATGTGCAAG |
| SEQ ID No. 181: | 5′- GCCACAGTTCGCCACTCATC |
| SEQ ID No. 182: | 5′- CCTCCGCGTTTGTCACCGGC |
| SEQ ID No. 183: | 5′- ACCAGTTCGCCACAGTTCGC |
| SEQ ID No. 184: | 5′- CACTCATCCGATGTGCAAGC |
| SEQ ID No. 185: | 5′- CCAGTTCGCCACAGTTCGCC |
| SEQ ID No. 186: | 5′- CTCATCCGATGTGCAAGCAC |
| SEQ ID No. 187: | 5′- TCCGATGTGCAAGCACTTCA |
| SEQ ID No. 188: | 5′- CGCCACTCATCCGATGTGCA |
| SEQ ID No. 189: | 5′- CAGTTCGCCACAGTTCGCCA |
| SEQ ID No. 190: | 5′- GCCACTCATCCGATGTGCAA |
| SEQ ID No. 191: | 5′- CGCCACAGTTCGCCACTCAT |
| SEQ ID No. 192: | 5′- ATCCGATGTGCAAGCACTTC |
| SEQ ID No. 193: | 5′- GTTCGCCACAGTTCGCCACT |
| SEQ ID No. 194: | 5′- TCCTCCGCGTTTGTCACCGG |
| SEQ ID No. 195: | 5′- CGCCAGGGTTCATCCTGAGC |
| SEQ ID No. 196: | 5′- AGTTCGCCACAGTTCGCCAC |
| SEQ ID No. 197: | 5′- TCGCCACAGTTCGCCACTCA |
| SEQ ID No. 198: | 5′- TTAACGGGATGCGTTCGACT |
| SEQ ID No. 199: | 5′- TCGCCACTCATCCGATGTGC |
| SEQ ID No. 200: | 5′- CCACAGTTCGCCACTCATCC |
| SEQ ID No. 201: | 5′- GATTTAACGGGATGCGTTCG |
| SEQ ID No. 202: | 5′- TAACGGGATGCGTTCGACTT |
| SEQ ID No. 203: | 5′- AACGGGATGCGTTCGACTTG |
| SEQ ID No. 204: | 5′- CGAAGGTTACCGAACCGACT |
| SEQ ID No. 205: | 5′- CCGAAGGTTACCGAACCGAC |
| SEQ ID No. 206: | 5′- CCCGAAGGTTACCGAACCGA |
| SEQ ID No. 207: | 5′- TTCCTCCGCGTTTGTCACCG |
| SEQ ID No. 208: | 5′- CCGCCAGGGTTCATCCTGAG |
| SEQ ID No. 209: | 5′- TCCTTCCAGAAGTGATAGCC |
| SEQ ID No. 210: | 5′- CACCAGTTCGCCACAGTTCG |
| SEQ ID No. 211: | 5′- ACGGGATGCGTTCGACTTGC |
| SEQ ID No. 212: | 5′- GTCCTTCCAGAAGTGATAGC |
| SEQ ID No. 213: | 5′- GCCAGGGTTCATCCTGAGCC |
| SEQ ID No. 214: | 5′- ACTCATCCGATGTGCAAGCA |
| SEQ ID No. 215: | 5′- ATCATTGCCTTGGTGAACCG |
| SEQ ID No. 216: | 5′- TCCGCGTTTGTCACCGGCAG |
| SEQ ID No. 217: | 5′- TGAACCGTTACTCCACCAAC |
| SEQ ID No. 218: | 5′- GAAGTGATAGCCGAAACCAC |
| SEQ ID No. 219: | 5′- CCGCGTTTGTCACCGGCAGT |
| SEQ ID No. 220: | 5′- TTCGCCACTCATCCGATGTG |
| SEQ ID No. 221: | 5′- CATTTAACGGGATGCGTTCG |
| SEQ ID No. 222: | 5′- CACAGTTCGCCACTCATCCG |
| SEQ ID No. 223: | 5′- TTCGCCACAGTTCGCCACTC |
| SEQ ID No. 224: | 5′- CTCCGCGTTTGTCACCGGCA |
| SEQ ID No. 225: | 5′- ACGCCGCCAGGGTTCATCCT |
| SEQ ID No. 226: | 5′- CCTTCCAGAAGTGATAGCCG |
| SEQ ID No. 227: | 5′- TCATTGCCTTGGTGAACCGT |
| SEQ ID No. 228: | 5′- CACAGTATGTCAAGACCTGG |
| SEQ ID No. 229: | 5′- TTGGTGAACCGTTACTCCAC |
| SEQ ID No. 230: | 5′- CTTGGTGAACCGTTACTCCA |
| SEQ ID No. 231: | 5′- GTGAACCGTTACTCCACCAA |
| SEQ ID No. 232: | 5′- GGCTCCCGAAGGTTACCGAA |
| SEQ ID No. 233: | 5′- GAAGGTTACCGAACCGACTT |
| SEQ ID No. 234: | 5′- TGGCTCCCGAAGGTTACCGA |
| SEQ ID No. 235: | 5′- TAATACGCCGCGGGTCCTTC |
| SEQ ID No. 236: | 5′- GAACCGTTACTCCACCAACT |
| SEQ ID No. 237: | 5′- TACGCCGCGGGTCCTTCCAG |
| SEQ ID No. 238: | 5′- TCACCAGTTCGCCACAGTTC |
| SEQ ID No. 239: | 5′- CCTTGGTGAACCGTTACTCC |
| SEQ ID No. 240: | 5′- CTCACCAGTTCGCCACAGTT |
| SEQ ID No. 241: | 5′- CGCCGCCAGGGTTCATCCTG |
| SEQ ID No. 242: | 5′- CCTTGGTGAACCATTACTCC |
| SEQ ID No. 243: | 5′- TGGTGAACCATTACTCCACC |
| SEQ ID No. 244: | 5′- GCCGCCAGGGTTCATCCTGA |
| SEQ ID No. 245: | 5′- GGTGAACCATTACTCCACCA |
| SEQ ID No. 246: | 5′- CCAGGGTTCATCCTGAGCCA |
| SEQ ID No. 247: | 5′- AATACGCCGCGGGTCCTTCC |
| SEQ ID No. 248: | 5′- CACGCCGCCAGGGTTCATCC |
| SEQ ID No. 249: | 5′- AGTTCGCCACTCATCCGATG |
| SEQ ID No. 250: | 5′- CGGGATGCGTTCGACTTGCA |
| SEQ ID No. 251: | 5′- CATTGCCTTGGTGAACCGTT |
| SEQ ID No. 252: | 5′- GCACGCCGCCAGGGTTCATC |
| SEQ ID No. 253: | 5′- CTTCCTCCGCGTTTGTCACC |
| SEQ ID No. 254: | 5′- TGGTGAACCGTTACTCCACC |
| SEQ ID No. 255: | 5′- CCTTCCTCCGCGTTTGTCAC |
| SEQ ID No. 256: | 5′- ACGCCGCGGGTCCTTCCAGA |
| SEQ ID No. 257: | 5′- GGTGAACCGTTACTCCACCA |
| SEQ ID No. 258: | 5′- GGGTCCTTCCAGAAGTGATA |
| SEQ ID No. 259: | 5′- CTTCCAGAAGTGATAGCCGA |
| SEQ ID No. 260: | 5′- GCCTTGGTGAACCATTACTC |
| SEQ ID No. 261: | 5′- ACAGTTCGCCACTCATCCGA |
| SEQ ID No. 262: | 5′- ACCTTCCTCCGCGTTTGTCA |
| SEQ ID No. 263: | 5′- CGAACCGACTTTGGGTGTTG |
| SEQ ID No. 264: | 5′- GAACCGACTTTGGGTGTTGC |
| SEQ ID No. 265: | 5′- AGGTTACCGAACCGACTTTG |
| SEQ ID No. 266: | 5′- ACCGAACCGACTTTGGGTGT |
| SEQ ID No. 267: | 5′- TTACCGAACCGACTTTGGGT |
| SEQ ID No. 268: | 5′- TACCGAACCGACTTTGGGTG |
| SEQ ID No. 269: | 5′- GTTACCGAACCGACTTTGGG |
| SEQ ID No. 270: | 5′- CCTTTCTGGTATGGTACCGTC |
| SEQ ID No. 271: | 5′- TGCACCGCGGAYCCATCTCT |
| SEQ ID No. 272: | 5′- AGTTGCAGTCCAGTAAGCCG |
| SEQ ID No. 273: | 5′- GTTGCAGTCCAGTAAGCCGC |
| SEQ ID No. 274: | 5′- CAGTTGCAGTCCAGTAAGCC |
| SEQ ID No. 275: | 5′- TGCAGTCCAGTAAGCCGCCT |
| SEQ ID No. 276: | 5′- TCAGTTGCAGTCCAGTAAGC |
| SEQ ID No. 277: | 5′- TTGCAGTCCAGTAAGCCGCC |
| SEQ ID No. 278: | 5′- GCAGTCCAGTAAGCCGCCTT |
| SEQ ID No. 279: | 5′- GTCAGTTGCAGTCCAGTAAG |
| SEQ ID No. 280: | 5′- CTCTAGGTGACGCCGAAGCG |
| SEQ ID No. 281: | 5′- ATCTCTAGGTGACGCCGAAG |
| SEQ ID No. 282: | 5′- TCTAGGTGACGCCGAAGCGC |
| SEQ ID No. 283: | 5′- TCTCTAGGTGACGCCGAAGC |
| SEQ ID No. 284: | 5′- CCATCTCTAGGTGACGCCGA |
| SEQ ID No. 285: | 5′- CATCTCTAGGTGACGCCGAA |
| SEQ ID No. 286: | 5′- TAGGTGACGCCGAAGCGCCT |
| SEQ ID No. 287: | 5′- CTAGGTGACGCCGAAGCGCC |
| SEQ ID No. 288: | 5′- CTTAGACGGCTCCTTCCTAA |
| SEQ ID No. 289: | 5′- CCTTAGACGGCTCCTTCCTA |
| SEQ ID No. 290: | 5′- ACGTCAGTTGCAGTCCAGTA |
| SEQ ID No. 291: | 5′- CGTCAGTTGCAGTCCAGTAA |
| SEQ ID No. 292: | 5′- ACGCCGAAGCGCCTTTTAAC |
| SEQ ID No. 293: | 5′- GACGCCGAAGCGCCTTTTAA |
| SEQ ID No. 294: | 5′- GCCGAAGCGCCTTTTAACTT |
| SEQ ID No. 295: | 5′- CGCCGAAGCGCCTTTTAACT |
| SEQ ID No. 296: | 5′- GTGACGCCGAAGCGCCTTTT |
| SEQ ID No. 297: | 5′- TGACGCCGAAGCGCCTTTTA |
| SEQ ID No. 298: | 5′- AGACGGCTCCTTCCTAAAAG |
| SEQ ID No. 299: | 5′- ACGGCTCCTTCCTAAAAGGT |
| SEQ ID No. 300: | 5′- GACGGCTCCTTCCTAAAAGG |
| SEQ ID No. 301: | 5′- CCTTCCTAAAAGGTTAGGCC |
| SEQ ID No. 302: | 5′- GGTGACGCCAAAGCGCCTTT |
| SEQ ID No. 303: | 5′- AGGTGACGCCAAAGCGCCTT |
| SEQ ID No. 304: | 5′- TAGGTGACGCCAAAGCGCCT |
| SEQ ID No. 305: | 5′- CTCTAGGTGACGCCAAAGCG |
| SEQ ID No. 306: | 5′- TCTAGGTGACGCCAAAGCGC |
| SEQ ID No. 307: | 5′- CTAGGTGACGCCAAAGCGCC |
| SEQ ID No. 308: | 5′- ACGCCAAAGCGCCTTTTAAC |
| SEQ ID No. 309: | 5′- CGCCAAAGCGCCTTTTAACT |
| SEQ ID No. 310: | 5′- TGACGCCAAAGCGCCTTTTA |
| SEQ ID No. 311: | 5′- TCTCTAGGTGACGCCAAAGC |
| SEQ ID No. 312: | 5′- GTGACGCCAAAGCGCCTTTT |
| SEQ ID No. 313: | 5′- GACGCCAAAGCGCCTTTTAA |
| SEQ ID No. 314: | 5′- ATCTCTAGGTGACGCCAAAG |
| SEQ ID No. 315: | 5′- CATCTCTAGGTGACGCCAAA |
| SEQ ID No. 316: | 5′- TCCATCTCTAGGTGACGCCA |
| SEQ ID No. 317: | 5′- CCATCTCTAGGTGACGCCAA |
| SEQ ID No. 318: | 5′- CTGCCTTAGACGGCTCCCCC |
| SEQ ID No. 319: | 5′- CCTGCCTTAGACGGCTCCCC |
| SEQ ID No. 320: | 5′- GTGTCATGCGACACTGAGTT |
| SEQ ID No. 321: | 5′- TGTGTCATGCGACACTGAGT |
| SEQ ID No. 322: | 5′- CTTTGTGTCATGCGACACTG |
| SEQ ID No. 323: | 5′- TTGTGTCATGCGACACTGAG |
| SEQ ID No. 324: | 5′- TGCCTTAGACGGCTCCCCCT |
| SEQ ID No. 325: | 5′- AGACGGCTCCCCCTAAAAGG |
| SEQ ID No. 326: | 5′- TAGACGGCTCCCCCTAAAAG |
| SEQ ID No. 327: | 5′- GCCTTAGACGGCTCCCCCTA |
| SEQ ID No. 328: | 5′- GCTCCCCCTAAAAGGTTAGG |
| SEQ ID No. 329: | 5′- GGCTCCCCCTAAAAGGTTAG |
| SEQ ID No. 330: | 5′- CTCCCCCTAAAAGGTTAGGC |
| SEQ ID No. 331: | 5′- TCCCCCTAAAAGGTTAGGCC |
| SEQ ID No. 332: | 5′- CCCTAAAAGGTTAGGCCACC |
| SEQ ID No. 333: | 5′- CCCCTAAAAGGTTAGGCCAC |
| SEQ ID No. 334: | 5′- CGGCTCCCCCTAAAAGGTTA |
| SEQ ID No. 335: | 5′- CCCCCTAAAAGGTTAGGCCA |
| SEQ ID No. 336: | 5′- CTTAGACGGCTCCCCCTAAA |
| SEQ ID No. 337: | 5′- TTAGACGGCTCCCCCTAAAA |
| SEQ ID No. 338: | 5′- GGGTTCGCAACTCGTTGTAT |
| SEQ ID No. 339: | 5′- CCTTAGACGGCTCCCCCTAA |
| SEQ ID No. 340: | 5′- ACGGCTCCCCCTAAAAGGTT |
| SEQ ID No. 341: | 5′- GACGGCTCCCCCTAAAAGGT |
| SEQ ID No. 342: | 5′- ACGCCGCAAGACCATCCTCT |
| SEQ ID No. 343: | 5′- CTAATACGCCGCAAGACCAT |
| SEQ ID No. 344: | 5′- TACGCCGCAAGACCATCCTC |
| SEQ ID No. 345: | 5′- GTTACGATCTAGCAAGCCGC |
| SEQ ID No. 346: | 5′- AATACGCCGCAAGACCATCC |
| SEQ ID No. 347: | 5′- CGCCGCAAGACCATCCTCTA |
| SEQ ID No. 348: | 5′- GCTAATACGCCGCAAGACCA |
| SEQ ID No. 349: | 5′- ACCATCCTCTAGCGATCCAA |
| SEQ ID No. 350: | 5′- TAATACGCCGCAAGACCATC |
| SEQ ID No. 351: | 5′- AGCCATCCCTTTCTGGTAAG |
| SEQ ID No. 352: | 5′- ATACGCCGCAAGACCATCCT |
| SEQ ID No. 353: | 5′- AGTTACGATCTAGCAAGCCG |
| SEQ ID No. 354: | 5′- AGCTAATACGCCGCAAGACC |
| SEQ ID No. 355: | 5′- GCCGCAAGACCATCCTCTAG |
| SEQ ID No. 356: | 5′- TTACGATCTAGCAAGCCGCT |
| SEQ ID No. 357: | 5′- GACCATCCTCTAGCGATCCA |
| SEQ ID No. 358: | 5′- TTGCTACGTCACTAGGAGGC |
| SEQ ID No. 359: | 5′- ACGTCACTAGGAGGCGGAAA |
| SEQ ID No. 360: | 5′- TTTGCTACGTCACTAGGAGG |
| SEQ ID No. 361: | 5′- GCCATCCCTTTCTGGTAAGG |
| SEQ ID No. 362: | 5′- TACGTCACTAGGAGGCGGAA |
| SEQ ID No. 363: | 5′- CGTCACTAGGAGGCGGAAAC |
| SEQ ID No. 364: | 5′- AAGACCATCCTCTAGCGATC |
| SEQ ID No. 365: | 5′- GCACGTATTTAGCCATCCCT |
| SEQ ID No. 366: | 5′- CTCTAGCGATCCAAAAGGAC |
| SEQ ID No. 367: | 5′- CCTCTAGCGATCCAAAAGGA |
| SEQ ID No. 368: | 5′- CCATCCTCTAGCGATCCAAA |
| SEQ ID No. 369: | 5′- GGCACGTATTTAGCCATCCC |
| SEQ ID No. 370: | 5′- TACGATCTAGCAAGCCGCTT |
| SEQ ID No. 371: | 5′- CAGTTACGATCTAGCAAGCC |
| SEQ ID No. 372: | 5′- CCGCAAGACCATCCTCTAGC |
| SEQ ID No. 373: | 5′- CCATCCCTTTCTGGTAAGGT |
| SEQ ID No. 374: | 5′- AGACCATCCTCTAGCGATCC |
| SEQ ID No. 375: | 5′- CAAGACCATCCTCTAGCGAT |
| SEQ ID No. 376: | 5′- GCTACGTCACTAGGAGGCGG |
| SEQ ID No. 377: | 5′- TGCTACGTCACTAGGAGGCG |
| SEQ ID No. 378: | 5′- CTACGTCACTAGGAGGCGGA |
| SEQ ID No. 379: | 5′- CCTCAACGTCAGTTACGATC |
| SEQ ID No. 380: | 5′- GTCACTAGGAGGCGGAAACC |
| SEQ ID No. 381: | 5′- TCCTCTAGCGATCCAAAAGG |
| SEQ ID No. 382: | 5′- TGGCACGTATTTAGCCATCC |
| SEQ ID No. 383: | 5′- ACGATCTAGCAAGCCGCTTT |
| SEQ ID No. 384: | 5′- GCCAGTCTCTCAACTCGGCT |
| SEQ ID No. 385: | 5′- AAGCTAATACGCCGCAAGAC |
| SEQ ID No. 386: | 5′- GTTTGCTACGTCACTAGGAG |
| SEQ ID No. 387: | 5′- CGCCACTCTAGTCATTGCCT |
| SEQ ID No. 388: | 5′- GGCCAGCCAGTCTCTCAACT |
| SEQ ID No. 389: | 5′- CAGCCAGTCTCTCAACTCGG |
| SEQ ID No. 390: | 5′- CCCGAAGATCAATTCAGCGG |
| SEQ ID No. 391: | 5′- CCGGCCAGTCTCTCAACTCG |
| SEQ ID No. 392: | 5′- CCAGCCAGTCTCTCAACTCG |
| SEQ ID No. 393: | 5′- TCATTGCCTCACTTCACCCG |
| SEQ ID No. 394: | 5′- GCCAGCCAGTCTCTCAACTC |
| SEQ ID No. 395: | 5′- CACCCGAAGATCAATTCAGC |
| SEQ ID No. 396: | 5′- GTCATTGCCTCACTTCACCC |
| SEQ ID No. 397: | 5′- CATTGCCTCACTTCACCCGA |
| SEQ ID No. 398: | 5′- ATTGCCTCACTTCACCCGAA |
| SEQ ID No. 399: | 5′- CGAAGATCAATTCAGCGGCT |
| SEQ ID No. 400: | 5′- AGTCATTGCCTCACTTCACC |
| SEQ ID No. 401: | 5′- TCGCCACTCTAGTCATTGCC |
| SEQ ID No. 402: | 5′- TTGCCTCACTTCACCCGAAG |
| SEQ ID No. 403: | 5′- CGGCCAGTCTCTCAACTCGG |
| SEQ ID No. 404: | 5′- CTGGCACGTATTTAGCCATC |
| SEQ ID No. 405: | 5′- ACCCGAAGATCAATTCAGCG |
| SEQ ID No. 406: | 5′- TCTAGCGATCCAAAAGGACC |
| SEQ ID No. 407: | 5′- CTAGCGATCCAAAAGGACCT |
| SEQ ID No. 408: | 5′- GCACCCATCGTTTACGGTAT |
| SEQ ID No. 409: | 5′- CACCCATCGTTTACGGTATG |
| SEQ ID No. 410: | 5′- GCCACTCTAGTCATTGCCTC |
| SEQ ID No. 411: | 5′- CGTTTGCTACGTCACTAGGA |
| SEQ ID No. 412: | 5′- GCCTCAACGTCAGTTACGAT |
| SEQ ID No. 413: | 5′- GCCGGCCAGTCTCTCAACTC |
| SEQ ID No. 414: | 5′- TCACTAGGAGGCGGAAACCT |
| SEQ ID No. 415: | 5′- AGCCTCAACGTCAGTTACGA |
| SEQ ID No. 416: | 5′- AGCCAGTCTCTCAACTCGGC |
| SEQ ID No. 417: | 5′- GGCCAGTCTCTCAACTCGGC |
| SEQ ID No. 418: | 5′- CAAGCTAATACGCCGCAAGA |
| SEQ ID No. 419: | 5′- TTCGCCACTCTAGTCATTGC |
| SEQ ID No. 420: | 5′- CCGAAGATCAATTCAGCGGC |
| SEQ ID No. 421: | 5′- CGCAAGACCATCCTCTAGCG |
| SEQ ID No. 422: | 5′- GCAAGACCATCCTCTAGCGA |
| SEQ ID No. 423: | 5′- GCGTTTGCTACGTCACTAGG |
| SEQ ID No. 424: | 5′- CCACTCTAGTCATTGCCTCA |
| SEQ ID No. 425: | 5′- CACTCTAGTCATTGCCTCAC |
| SEQ ID No. 426: | 5′- CCAGTCTCTCAACTCGGCTA |
| SEQ ID No. 427: | 5′- TTACCTTAGGCACCGGCCTC |
| SEQ ID No. 428: | 5′- ACAAGCTAATACGCCGCAAG |
| SEQ ID No. 429: | 5′- TTTACCTTAGGCACCGGCCT |
| SEQ ID No. 430: | 5′- TTTTACCTTAGGCACCGGCC |
| SEQ ID No. 431: | 5′- ATTTTACCTTAGGCACCGGC |
| SEQ ID No. 432: | 5′- GATTTTACCTTAGGCACCGG |
| SEQ ID No. 433: | 5′- CTCACTTCACCCGAAGATCA |
| SEQ ID No. 434: | 5′- ACGCCACCAGCGTTCATCCT |
| SEQ ID No. 435: | 5′- GCCAAGCGACTTTGGGTACT |
| SEQ ID No. 436: | 5′- CGGAAAATTCCCTACTGCAG |
| SEQ ID No. 437: | 5′- CGATCTAGCAAGCCGCTTTC |
| SEQ ID No. 438: | 5′- GGTACCGTCAAGCTGAAAAC |
| SEQ ID No. 439: | 5′- TGCCTCACTTCACCCGAAGA |
| SEQ ID No. 440: | 5′- GGCCGGCCAGTCTCTCAACT |
| SEQ ID No. 441: | 5′- GGTAAGGTACCGTCAAGCTG |
| SEQ ID No. 442: | 5′- GTAAGGTACCGTCAAGCTGA |
| SEQ ID No. 443: | 5′- CCGCAAGACCATCCTCTAGG |
| SEQ ID No. 444: | 5′- ATTTAGCCATCCCTTTCTGG |
| SEQ ID No. 445: | 5′- AACCCTTCATCACACACG |
| SEQ ID No. 446: | 5′- CGAAACCCTTCATCACAC |
| SEQ ID No. 447: | 5′- ACCCTTCATCACACACGC |
| SEQ ID No. 448: | 5′- TACCGTCACACACTGAAC |
| SEQ ID No. 449: | 5′- AGATACCGTCACACACTG |
| SEQ ID No. 450: | 5′- CACTCAAGGGCGGAAACC |
| SEQ ID No. 451: | 5′- ACCGTCACACACTGAACA |
| SEQ ID No. 452: | 5′- CGTCACACACTGAACAGT |
| SEQ ID No. 453: | 5′- CCGAAACCCTTCATCACA |
| SEQ ID No. 454: | 5′- CCGTCACACACTGAACAG |
| SEQ ID No. 455: | 5′- GATACCGTCACACACTGA |
| SEQ ID No. 456: | 5′- GGTAAGATACCGTCACAC |
| SEQ ID No. 457: | 5′- CCCTTCATCACACACGCG |
| SEQ ID No. 458: | 5′- ACAGTGTTTTACGAGCCG |
| SEQ ID No. 459: | 5′- CAGTGTTTTACGAGCCGA |
| SEQ ID No. 460: | 5′- ACAAAGCGTTCGACTTGC |
| SEQ ID No. 461: | 5′- CGGATAACGCTTGGAACA |
| SEQ ID No. 462: | 5′- AGGGCGGAAACCCTCGAA |
| SEQ ID No. 463: | 5′- GGGCGGAAACCCTCGAAC |
| SEQ ID No. 464: | 5′- GGCGGAAACCCTCGAACA |
| SEQ ID No. 465: | 5′- TGAGGGCTTTCACTTCAG |
| SEQ ID No. 466: | 5′- AGGGCTTTCACTTCAGAC |
| SEQ ID No. 467: | 5′- GAGGGCTTTCACTTCAGA |
| SEQ ID No. 468: | 5′- ACTGCACTCAAGTCATCC |
| SEQ ID No. 469: | 5′- CCGGATAACGCTTGGAAC |
| SEQ ID No. 470: | 5′- TCCGGATAACGCTTGGAA |
| SEQ ID No. 471: | 5′- TATCCCCTGCTAAGAGGT |
| SEQ ID No. 472: | 5′- CCTGCTAAGAGGTAGGTT |
| SEQ ID No. 473: | 5′- CCCTGCTAAGAGGTAGGT |
| SEQ ID No. 474: | 5′- CCCCTGCTAAGAGGTAGG |
| SEQ ID No. 475: | 5′- TCCCCTGCTAAGAGGTAG |
| SEQ ID No. 476: | 5′- ATCCCCTGCTAAGAGGTA |
| SEQ ID No. 477: | 5′- CCGTTCCTTTCTGGTAAG |
| SEQ ID No. 478: | 5′- GCCGTTCCTTTCTGGTAA |
| SEQ ID No. 479: | 5′- AGCCGTTCCTTTCTGGTA |
| SEQ ID No. 480: | 5′- GCACGTATTTAGCCGTTC |
| SEQ ID No. 481: | 5′- CACGTATTTAGCCGTTCC |
| SEQ ID No. 482: | 5′- GGCACGTATTTAGCCGTT |
| SEQ ID No. 483: | 5′- CACTTTCCTCTACTGCAC |
| SEQ ID No. 484: | 5′- CCACTTTCCTCTACTGCA |
| SEQ ID No. 485: | 5′- TCCACTTTCCTCTACTGC |
| SEQ ID No. 486: | 5′- CTTTCCTCTACTGCACTC |
| SEQ ID No. 487: | 5′- TAGCCGTTCCTTTCTGGT |
| SEQ ID No. 488: | 5′- TTAGCCGTTCCTTTCTGG |
| SEQ ID No. 489: | 5′- TTATCCCCTGCTAAGAGG |
| SEQ ID No. 490: | 5′- GTTATCCCCTGCTAAGAG |
| SEQ ID No. 491: | 5′- CCCGTTCGCCACTCTTTG |
| SEQ ID No. 492: | 5′- AGCTGAGGGCTTTCACTT |
| SEQ ID No. 493: | 5′- GAGCTGAGGGCTTTCACT |
| SEQ ID No. 494: | 5′- GCTGAGGGCTTTCACTTC |
| SEQ ID No. 495: | 5′- CTGAGGGCTTTCACTTCA |
| SEQ ID No. 496: | 5′ CCCGTGTCCCGAAGGAAC |
| SEQ ID No. 497: | 5′ GCACGAGTATGTCAAGAC |
| SEQ ID No. 498: | 5′ GTATCCCGTGTCCCGAAG |
| SEQ ID No. 499: | 5′ TCCCGTGTCCCGAAGGAA |
| SEQ ID No. 500: | 5′ ATCCCGTGTCCCGAAGGA |
| SEQ ID No. 501: | 5′ TATCCCGTGTCCCGAAGG |
| SEQ ID No. 502: | 5′ CTTACCTTAGGAAGCGCC |
| SEQ ID No. 503: | 5′ TTACCTTAGGAAGCGCCC |
| SEQ ID No. 504: | 5′ CCTGTATCCCGTGTCCCG |
| SEQ ID No. 505: | 5′ CCACCTGTATCCCGTGTC |
| SEQ ID No. 506: | 5′ CACCTGTATCCCGTGTCC |
| SEQ ID No. 507: | 5′ ACCTGTATCCCGTGTCCC |
| SEQ ID No. 508: | 5′ CTGTATCCCGTGTCCCGA |
| SEQ ID No. 509: | 5′ TGTATCCCGTGTCCCGAA |
| SEQ ID No. 510: | 5′ CACGAGTATGTCAAGACC |
| SEQ ID No. 511: | 5′ CGGTCTTACCTTAGGAAG |
| SEQ ID No. 512: | 5′ TAGGAAGCGCCCTCCTTG |
| SEQ ID No. 513: | 5′ AGGAAGCGCCCTCCTTGC |
| SEQ ID No. 514: | 5′ TTAGGAAGCGCCCTCCTT |
| SEQ ID No. 515: | 5′ CTTAGGAAGCGCCCTCCT |
| SEQ ID No. 516: | 5′ CCTTAGGAAGCGCCCTCC |
| SEQ ID No. 517: | 5′ ACCTTAGGAAGCGCCCTC |
| SEQ ID No. 518: | 5′ TGCACACAATGGTTGAGC |
| SEQ ID No. 519: | 5′ TACCTTAGGAAGCGCCCT |
| SEQ ID No. 520: | 5′ ACCACCTGTATCCCGTGT |
| SEQ ID No. 521: | 5′ GCACCACCTGTATCCCGT |
| SEQ ID No. 522: | 5′ CACCACCTGTATCCCGTG |
| SEQ ID No. 523: | 5′ GCGGTTAGGCAACCTACT |
| SEQ ID No. 524: | 5′ TGCGGTTAGGCAACCTAC |
| SEQ ID No. 525: | 5′ TTGCGGTTAGGCAACCTA |
| SEQ ID No. 526: | 5′ GGTCTTACCTTAGGAAGC |
| SEQ ID No. 527: | 5′ GCTAATACAACGCGGGAT |
| SEQ ID No. 528: | 5′ CTAATACAACGCGGGATC |
| SEQ ID No. 529: | 5′ ATACAACGCGGGATCATC |
| SEQ ID No. 530: | 5′ CGGTTAGGCAACCTACTT |
| SEQ ID No. 531: | 5′ TGCACCACCTGTATCCCG |
| SEQ ID No. 532: | 5′ GAAGCGCCCTCCTTGCGG |
| SEQ ID No. 533: | 5′ GGAAGCGCCCTCCTTGCG |
| SEQ ID No. 534: | 5′ CGTCCCTTTCTGGTTAGA |
| SEQ ID No. 535: | 5′ AGCTAATACAACGCGGGA |
| SEQ ID No. 536: | 5′ TAGCTAATACAACGCGGG |
| SEQ ID No. 537: | 5′ CTAGCTAATACAACGCGG |
| SEQ ID No. 538: | 5′ GGCTATGTATCATCGCCT |
| SEQ ID No. 539: | 5′ GAGCCACTGCCTTTTACA |
| SEQ ID No. 540: | 5′ GTCGGCTATGTATCATCG |
| SEQ ID No. 541: | 5′ GGTCGGCTATGTATCATC |
| SEQ ID No. 542: | 5′ CAGGTCGGCTATGTATCA |
| SEQ ID No. 543: | 5′ CGGCTATGTATCATCGCC |
| SEQ ID No. 544: | 5′ TCGGCTATGTATCATCGC |
| SEQ ID No. 545: | 5′ GTCTTACCTTAGGAAGCG |
| SEQ ID No. 546: | 5′ TCTTACCTTAGGAAGCGC |
| SEQ ID No. 547: | 5′- GTACAAACCGCCTACACGCC |
| SEQ ID No. 548: | 5′- TGTACAAACCGCCTACACGC |
| SEQ ID No. 549: | 5′- GATCAGCACGATGTCGCCAT |
| SEQ ID No. 550: | 5′- CTGTACAAACCGCCTACACG |
| SEQ ID No. 551: | 5′- GAGATCAGCACGATGTCGCC |
| SEQ ID No. 552: | 5′- AGATCAGCACGATGTCGCCA |
| SEQ ID No. 553: | 5′- ATCAGCACGATGTCGCCATC |
| SEQ ID No. 554: | 5′- TCAGCACGATGTCGCCATCT |
| SEQ ID No. 555: | 5′- ACTGTACAAACCGCCTACAC |
| SEQ ID No. 556: | 5′- CCGCCACTAAGGCCGAAACC |
| SEQ ID No. 557: | 5′- CAGCACGATGTCGCCATCTA |
| SEQ ID No. 558: | 5′- TACAAACCGCCTACACGCCC |
| SEQ ID No. 559: | 5′- AGCACGATGTCGCCATCTAG |
| SEQ ID No. 560: | 5′- CGGCTTTTAGAGATCAGCAC |
| SEQ ID No. 561: | 5′- TCCGCCACTAAGGCCGAAAC |
| SEQ ID No. 562: | 5′- GACTGTACAAACCGCCTACA |
| SEQ ID No. 563: | 5′- GTCCGCCACTAAGGCCGAAA |
| SEQ ID No. 564: | 5′- GGGGATTTCACATCTGACTG |
| SEQ ID No. 565: | 5′- CATACAAGCCCTGGTAAGGT |
| SEQ ID No. 566: | 5′- ACAAGCCCTGGTAAGGTTCT |
| SEQ ID No. 567: | 5′- ACAAACCGCCTACACGCCCT |
| SEQ ID No. 568: | 5′- CTGACTGTACAAACCGCCTA |
| SEQ ID No. 569: | 5′- TGACTGTACAAACCGCCTAC |
| SEQ ID No. 570: | 5′- ACGATGTCGCCATCTAGCTT |
| SEQ ID No. 571: | 5′- CACGATGTCGCCATCTAGCT |
| SEQ ID No. 572: | 5′- CGATGTCGCCATCTAGCTTC |
| SEQ ID No. 573: | 5′- GCACGATGTCGCCATCTAGC |
| SEQ ID No. 574: | 5′- GATGTCGCCATCTAGCTTCC |
| SEQ ID No. 575: | 5′- ATGTCGCCATCTAGCTTCCC |
| SEQ ID No. 576: | 5′- TGTCGCCATCTAGCTTCCCA |
| SEQ ID No. 577: | 5′- GCCATCTAGCTTCCCACTGT |
| SEQ ID No. 578: | 5′- TCGCCATCTAGCTTCCCACT |
| SEQ ID No. 579: | 5′- CGCCATCTAGCTTCCCACTG |
| SEQ ID No. 580: | 5′- GTCGCCATCTAGCTTCCCAC |
| SEQ ID No. 581: | 5′- TACAAGCCCTGGTAAGGTTC |
| SEQ ID No. 582: | 5′- GCCACTAAGGCCGAAACCTT |
| SEQ ID No. 583: | 5′- ACTAAGGCCGAAACCTTCGT |
| SEQ ID No. 584: | 5′- CTAAGGCCGAAACCTTCGTG |
| SEQ ID No. 585: | 5′- CACTAAGGCCGAAACCTTCG |
| SEQ ID No. 586: | 5′- AAGGCCGAAACCTTCGTGCG |
| SEQ ID No. 587: | 5′- CCACTAAGGCCGAAACCTTC |
| SEQ ID No. 588: | 5′- TAAGGCCGAAACCTTCGTGC |
| SEQ ID No. 589: | 5′- AGGCCGAAACCTTCGTGCGA |
| SEQ ID No. 590: | 5′- TCTGACTGTACAAACCGCCT |
| SEQ ID No. 591: | 5′- CATCTGACTGTACAAACCGC |
| SEQ ID No. 592: | 5′- ATCTGACTGTACAAACCGCC |
| SEQ ID No. 593: | 5′- CTTCGTGCGACTTGCATGTG |
| SEQ ID No. 594: | 5′- CCTTCGTGCGACTTGCATGT |
| SEQ ID No. 595: | 5′- CTCTCTAGAGTGCCCACCCA |
| SEQ ID No. 596: | 5′- TCTCTAGAGTGCCCACCCAA |
| SEQ ID No. 597: | 5′- ACGTATCAAATGCAGCTCCC |
| SEQ ID No. 598: | 5′- CGTATCAAATGCAGCTCCCA |
| SEQ ID No. 599: | 5′- CGCCACTAAGGCCGAAACCT |
| SEQ ID No. 600: | 5′- CCGAAACCTTCGTGCGACTT |
| SEQ ID No. 601: | 5′- GCCGAAACCTTCGTGCGACT |
| SEQ ID No. 602: | 5′- AACCTTCGTGCGACTTGCAT |
| SEQ ID No. 603: | 5′- CGAAACCTTCGTGCGACTTG |
| SEQ ID No. 604: | 5′- ACCTTCGTGCGACTTGCATG |
| SEQ ID No. 605: | 5′- GAAACCTTCGTGCGACTTGC |
| SEQ ID No. 606: | 5′- GGCCGAAACCTTCGTGCGAC |
| SEQ ID No. 607: | 5′- AAACCTTCGTGCGACTTGCA |
| SEQ ID No. 608: | 5′- CACGTATCAAATGCAGCTCC |
| SEQ ID No. 609: | 5′- GCTCACCGGCTTAAGGTCAA |
| SEQ ID No. 610: | 5′- CGCTCACCGGCTTAAGGTCA |
| SEQ ID No. 611: | 5′- TCGCTCACCGGCTTAAGGTC |
| SEQ ID No. 612: | 5′- CTCACCGGCTTAAGGTCAAA |
| SEQ ID No. 613: | 5′- CCCGACCGTGGTCGGCTGCG |
| SEQ ID No. 614: | 5′- GCTCACCGGCTTAAGGTCAA |
| SEQ ID No. 615: | 5′- CGCTCACCGGCTTAAGGTCA |
| SEQ ID No. 616: | 5′- TCGCTCACCGGCTTAAGGTC |
| SEQ ID No. 617: | 5′- CTCACCGGCTTAAGGTCAAA |
| SEQ ID No. 618: | 5′- CCCGACCGTGGTCGGCTGCG |
| SEQ ID No. 619: | 5′- TCACCGGCTTAAGGTCAAAC |
| SEQ ID No. 620: | 5′- CAACCCTCTCTCACACTCTA |
| SEQ ID No. 621: | 5′- ACAACCCTCTCTCACACTCT |
| SEQ ID No. 622: | 5′- CCACAACCCTCTCTCACACT |
| SEQ ID No. 623: | 5′- AACCCTCTCTCACACTCTAG |
| SEQ ID No. 624: | 5′- CACAACCCTCTCTCACACTC |
| SEQ ID No. 625: | 5′- TCCACAACCCTCTCTCACAC |
| SEQ ID No. 626: | 5′- TTCCACAACCCTCTCTCACA |
| SEQ ID No. 627: | 5′- ACCCTCTCTCACACTCTAGT |
| SEQ ID No. 628: | 5′- GAGCCAGGTTGCCGCCTTCG |
| SEQ ID No. 629: | 5′- AGGTCAAACCAACTCCCATG |
| SEQ ID No. 630: | 5′- ATGAGCCAGGTTGCCGCCTT |
| SEQ ID No. 631: | 5′- TGAGCCAGGTTGCCGCCTTC |
| SEQ ID No. 632: | 5′- AGGCTCCTCCACAGGCGACT |
| SEQ ID No. 633: | 5′- CAGGCTCCTCCACAGGCGAC |
| SEQ ID No. 634: | 5′- GCAGGCTCCTCCACAGGCGA |
| SEQ ID No. 635: | 5′- TTCGCTCACCGGCTTAAGGT |
| SEQ ID No. 636: | 5′- GTTCGCTCACCGGCTTAAGG |
| SEQ ID No. 637: | 5′- GGTTCGCTCACCGGCTTAAG |
| SEQ ID No. 638: | 5′- ATTCCACAACCCTCTCTCAC |
| SEQ ID No. 639: | 5′- TGACCCGACCGTGGTCGGCT |
| SEQ ID No. 640: | 5′- CCCTCTCTCACACTCTAGTC |
| SEQ ID No. 641: | 5′- GAATTCCACAACCCTCTCTC |
| SEQ ID No. 642: | 5′- AGCCAGGTTGCCGCCTTCGC |
| SEQ ID No. 643: | 5′- GCCAGGTTGCCGCCTTCGCC |
| SEQ ID No. 644: | 5′- GGAATTCCACAACCCTCTCT |
| SEQ ID No. 645: | 5′- GGGAATTCCACAACCCTCTC |
| SEQ ID No. 646: | 5′- AACGCAGGCTCCTCCACAGG |
| SEQ ID No. 647: | 5′- CGGCTTAAGGTCAAACCAAC |
| SEQ ID No. 648: | 5′- CCGGCTTAAGGTCAAACCAA |
| SEQ ID No. 649: | 5′- CACCGGCTTAAGGTCAAACC |
| SEQ ID No. 650: | 5′- ACCGGCTTAAGGTCAAACCA |
| SEQ ID No. 651: | 5′- ACCCAACATCCAGCACACAT |
| SEQ ID No. 652: | 5′- TCGCTGACCCGACCGTGGTC |
| SEQ ID No. 653: | 5′- CGCTGACCCGACCGTGGTCG |
| SEQ ID No. 654: | 5′- GACCCGACCGTGGTCGGCTG |
| SEQ ID No. 655: | 5′- GCTGACCCGACCGTGGTCGG |
| SEQ ID No. 656: | 5′- CTGACCCGACCGTGGTCGGC |
| SEQ ID No. 657: | 5′- CAGGCGACTTGCGCCTTTGA |
| SEQ ID No. 658: | 5′- TCATGCGGTATTAGCTCCAG |
| SEQ ID No. 659: | 5′- ACTAGCTAATCGAACGCAGG |
| SEQ ID No. 660: | 5′- CATGCGGTATTAGCTCCAGT |
| SEQ ID No. 661: | 5′- CGCAGGCTCCTCCACAGGCG |
| SEQ ID No. 662: | 5′- ACGCAGGCTCCTCCACAGGC |
| SEQ ID No. 663: | 5′- CTCAGGTGTCATGCGGTATT |
| SEQ ID No. 664: | 5′- CGCCTTTGACCCTCAGGTGT |
| SEQ ID No. 665: | 5′- ACCCTCAGGTGTCATGCGGT |
| SEQ ID No. 666: | 5′- CCTCAGGTGTCATGCGGTAT |
| SEQ ID No. 667: | 5′- TTTGACCCTCAGGTGTCATG |
| SEQ ID No. 668: | 5′- GACCCTCAGGTGTCATGCGG |
| SEQ ID No. 669: | 5′- TGACCCTCAGGTGTCATGCG |
| SEQ ID No. 670: | 5′- GCCTTTGACCCTCAGGTGTC |
| SEQ ID No. 671: | 5′- TTGACCCTCAGGTGTCATGC |
| SEQ ID No. 672: | 5′- CCCTCAGGTGTCATGCGGTA |
| SEQ ID No. 673: | 5′- CCTTTGACCCTCAGGTGTCA |
| SEQ ID No. 674: | 5′- CTTTGACCCTCAGGTGTCAT |
| SEQ ID No. 675: | 5′- AGTTATCCCCCACCCATGGA |
| SEQ ID No. 676: | 5′- CCAGCTATCGATCATCGCCT |
| SEQ ID No. 677: | 5′- ACCAGCTATCGATCATCGCC |
| SEQ ID No. 678: | 5′- CAGCTATCGATCATCGCCTT |
| SEQ ID No. 679: | 5′- AGCTATCGATCATCGCCTTG |
| SEQ ID No. 680: | 5′- GCTATCGATCATCGCCTTGG |
| SEQ ID No. 681: | 5′- CTATCGATCATCGCCTTGGT |
| SEQ ID No. 682: | 5′- TTCGTGCGACTTGCATGTGT |
| SEQ ID No. 683: | 5′- TCGATCATCGCCTTGGTAGG |
| SEQ ID No. 684: | 5′- ATCGATCATCGCCTTGGTAG |
| SEQ ID No. 685: | 5′- CACAGGCGACTTGCGCCTTT |
| SEQ ID No. 686: | 5′- CCACAGGCGACTTGCGCCTT |
| SEQ ID No. 687: | 5′- TCCACAGGCGACTTGCGCCT |
| SEQ ID No. 688: | 5′- TCCTCCACAGGCGACTTGCG |
| SEQ ID No. 689: | 5′- CCTCCACAGGCGACTTGCGC |
| SEQ ID No. 690: | 5′- CTCCACAGGCGACTTGCGCC |
| SEQ ID No. 691: | 5′- ACAGGCGACTTGCGCCTTTG |
| SEQ ID No. 692: | 5′- GCTCACCGGCTTAAGGTCAA |
| SEQ ID No. 693: | 5′- CGCTCACCGGCTTAAGGTCA |
| SEQ ID No. 694: | 5′- TCGCTCACCGGCTTAAGGTC |
| SEQ ID No. 695: | 5′- CTCACCGGCTTAAGGTCAAA |
| SEQ ID No. 696: | 5′- CCCGACCGTGGTCGGCTGCG |
| SEQ ID No. 697: | 5′- TCACCGGCTTAAGGTCAAAC |
| SEQ ID No. 698: | 5′- CAACCCTCTCTCACACTCTA |
| SEQ ID No. 699: | 5′- ACAACCCTCTCTCACACTCT |
| SEQ ID No. 700: | 5′- CCACAACCCTCTCTCACACT |
| SEQ ID No. 701: | 5′- AACCCTCTCTCACACTCTAG |
| SEQ ID No. 702: | 5′- CACAACCCTCTCTCACACTC |
| SEQ ID No. 703: | 5′- TCCACAACCCTCTCTCACAC |
| SEQ ID No. 704: | 5′- TTCCACAACCCTCTCTCACA |
| SEQ ID No. 705: | 5′- ACCCTCTCTCACACTCTAGT |
| SEQ ID No. 706: | 5′- GAGCCAGGTTGCCGCCTTCG |
| SEQ ID No. 707: | 5′- AGGTCAAACCAACTCCCATG |
| SEQ ID No. 708: | 5′- ATGAGCCAGGTTGCCGCCTT |
| SEQ ID No. 709: | 5′- TGAGCCAGGTTGCCGCCTTC |
| SEQ ID No. 710: | 5′- AGGCTCCTCCACAGGCGACT |
| SEQ ID No. 711: | 5′- CAGGCTCCTCCACAGGCGAC |
| SEQ ID No. 712: | 5′- GCAGGCTCCTCCACAGGCGA |
| SEQ ID No. 713: | 5′- TTCGCTCACCGGCTTAAGGT |
| SEQ ID No. 714: | 5′- GTTCGCTCACCGGCTTAAGG |
| SEQ ID No. 715: | 5′- GGTTCGCTCACCGGCTTAAG |
| SEQ ID No. 716: | 5′- ATTCCACAACCCTCTCTCAC |
| SEQ ID No. 717: | 5′- TGACCCGACCGTGGTCGGCT |
| SEQ ID No. 718: | 5′- CCCTCTCTCACACTCTAGTC |
| SEQ ID No. 719: | 5′- GAATTCCACAACCCTCTCTC |
| SEQ ID No. 720: | 5′- AGCCAGGTTGCCGCCTTCGC |
| SEQ ID No. 721: | 5′- GCCAGGTTGCCGCCTTCGCC |
| SEQ ID No. 722: | 5′- GGAATTCCACAACCCTCTCT |
| SEQ ID No. 723: | 5′- GGGAATTCCACAACCCTCTC |
| SEQ ID No. 724: | 5′- AACGCAGGCTCCTCCACAGG |
| SEQ ID No. 725: | 5′- CGGCTTAAGGTCAAACCAAC |
| SEQ ID No. 726: | 5′- CCGGCTTAAGGTCAAACCAA |
| SEQ ID No. 727: | 5′- CACCGGCTTAAGGTCAAACC |
| SEQ ID No. 728: | 5′- ACCGGCTTAAGGTCAAACCA |
| SEQ ID No. 729: | 5′- ACCCAACATCCAGCACACAT |
| SEQ ID No. 730: | 5′- TCGCTGACCCGACCGTGGTC |
| SEQ ID No. 731: | 5′- CGCTGACCCGACCGTGGTCG |
| SEQ ID No. 732: | 5′- GACCCGACCGTGGTCGGCTG |
| SEQ ID No. 733: | 5′- GCTGACCCGACCGTGGTCGG |
| SEQ ID No. 734: | 5′- CTGACCCGACCGTGGTCGGC |
| SEQ ID No. 735: | 5′- CAGGCGACTTGCGCCTTTGA |
| SEQ ID No. 736: | 5′- TCATGCGGTATTAGCTCCAG |
| SEQ ID No. 737: | 5′- ACTAGCTAATCGAACGCAGG |
| SEQ ID No. 738: | 5′- CATGCGGTATTAGCTCCAGT |
| SEQ ID No. 739: | 5′- CGCAGGCTCCTCCACAGGCG |
| SEQ ID No. 740: | 5′- ACGCAGGCTCCTCCACAGGC |
| SEQ ID No. 741: | 5′- CTCAGGTGTCATGCGGTATT |
| SEQ ID No. 742: | 5′- CGCCTTTGACCCTCAGGTGT |
| SEQ ID No. 743: | 5′- ACCCTCAGGTGTCATGCGGT |
| SEQ ID No. 744: | 5′- CCTCAGGTGTCATGCGGTAT |
| SEQ ID No. 745: | 5′- TTTGACCCTCAGGTGTCATG |
| SEQ ID No. 746: | 5′- GACCCTCAGGTGTCATGCGG |
| SEQ ID No. 747: | 5′- TGACCCTCAGGTGTCATGCG |
| SEQ ID No. 748: | 5′- GCCTTTGACCCTCAGGTGTC |
| SEQ ID No. 749: | 5′- TTGACCCTCAGGTGTCATGC |
| SEQ ID No. 750: | 5′- CCCTCAGGTGTCATGCGGTA |
| SEQ ID No. 751: | 5′- CCTTTGACCCTCAGGTGTCA |
| SEQ ID No. 752: | 5′- CTTTGACCCTCAGGTGTCAT |
| SEQ ID No. 753: | 5′- AGTTATCCCCCACCCATGGA |
| SEQ ID No. 754: | 5′- CCAGCTATCGATCATCGCCT |
| SEQ ID No. 755: | 5′- ACCAGCTATCGATCATCGCC |
| SEQ ID No. 756: | 5′- CAGCTATCGATCATCGCCTT |
| SEQ ID No. 757: | 5′- AGCTATCGATCATCGCCTTG |
| SEQ ID No. 758: | 5′- GCTATCGATCATCGCCTTGG |
| SEQ ID No. 759: | 5′- CTATCGATCATCGCCTTGGT |
| SEQ ID No. 760: | 5′- TTCGTGCGACTTGCATGTGT |
| SEQ ID No. 761: | 5′- TCGATCATCGCCTTGGTAGG |
| SEQ ID No. 762: | 5′- ATCGATCATCGCCTTGGTAG |
| SEQ ID No. 763: | 5′- CACAGGCGACTTGCGCCTTT |
| SEQ ID No. 764: | 5′- CCACAGGCGACTTGCGCCTT |
| SEQ ID No. 765: | 5′- TCCACAGGCGACTTGCGCCT |
| SEQ ID No. 766: | 5′- TCCTCCACAGGCGACTTGCG |
| SEQ ID No. 767: | 5′- CCTCCACAGGCGACTTGCGC |
| SEQ ID No. 768: | 5′- CTCCACAGGCGACTTGCGCC |
| SEQ ID No. 769: | 5′- ACAGGCGACTTGCGCCTTTG |
| SEQ ID No. 770: | 5′- TCACCGGCTTAAGGTCAAAC |
| SEQ ID No. 771: | 5′- CAACCCTCTCTCACACTCTA |
| SEQ ID No. 772: | 5′- ACAACCCTCTCTCACACTCT |
| SEQ ID No. 773: | 5′- CCACAACCCTCTCTCACACT |
| SEQ ID No. 774: | 5′- AACCCTCTCTCACACTCTAG |
| SEQ ID No. 775: | 5′- CACAACCCTCTCTCACACTC |
| SEQ ID No. 776: | 5′- TCCACAACCCTCTCTCACAC |
| SEQ ID No. 777: | 5′- TTCCACAACCCTCTCTCACA |
| SEQ ID No. 778: | 5′- ACCCTCTCTCACACTCTAGT |
| SEQ ID No. 779: | 5′- GAGCCAGGTTGCCGCCTTCG |
| SEQ ID No. 780: | 5′- AGGTCAAACCAACTCCCATG |
| SEQ ID No. 781: | 5′- ATGAGCCAGGTTGCCGCCTT |
| SEQ ID No. 782: | 5′- TGAGCCAGGTTGCCGCCTTC |
| SEQ ID No. 783: | 5′- AGGCTCCTCCACAGGCGACT |
| SEQ ID No. 784: | 5′- CAGGCTCCTCCACAGGCGAC |
| SEQ ID No. 785: | 5′- GCAGGCTCCTCCACAGGCGA |
| SEQ ID No. 786: | 5′- TTCGCTCACCGGCTTAAGGT |
| SEQ ID No. 787: | 5′- GTTCGCTCACCGGCTTAAGG |
| SEQ ID No. 788: | 5′- GGTTCGCTCACCGGCTTAAG |
| SEQ ID No. 789: | 5′- ATTCCACAACCCTCTCTCAC |
| SEQ ID No. 790: | 5′- TGACCCGACCGTGGTCGGCT |
| SEQ ID No. 791: | 5′- CCCTCTCTCACACTCTAGTC |
| SEQ ID No. 792: | 5′- GAATTCCACAACCCTCTCTC |
| SEQ ID No. 793: | 5′- AGCCAGGTTGCCGCCTTCGC |
| SEQ ID No. 794: | 5′- GCCAGGTTGCCGCCTTCGCC |
| SEQ ID No. 795: | 5′- GGAATTCCACAACCCTCTCT |
| SEQ ID No. 796: | 5′- GGGAATTCCACAACCCTCTC |
| SEQ ID No. 797: | 5′- AACGCAGGCTCCTCCACAGG |
| SEQ ID No. 798: | 5′- CGGCTTAAGGTCAAACCAAC |
| SEQ ID No. 799: | 5′- CCGGCTTAAGGTCAAACCAA |
| SEQ ID No. 800: | 5′- CACCGGCTTAAGGTCAAACC |
| SEQ ID No. 801: | 5′- ACCGGCTTAAGGTCAAACCA |
| SEQ ID No. 802: | 5′- ACCCAACATCCAGCACACAT |
| SEQ ID No. 803: | 5′- TCGCTGACCCGACCGTGGTC |
| SEQ ID No. 804: | 5′- CGCTGACCCGACCGTGGTCG |
| SEQ ID No. 805: | 5′- GACCCGACCGTGGTCGGCTG |
| SEQ ID No. 806: | 5′- GCTGACCCGACCGTGGTCGG |
| SEQ ID No. 807: | 5′- CTGACCCGACCGTGGTCGGC |
| SEQ ID No. 808: | 5′- CAGGCGACTTGCGCCTTTGA |
| SEQ ID No. 809: | 5′- TCATGCGGTATTAGCTCCAG |
| SEQ ID No. 810: | 5′- ACTAGCTAATCGAACGCAGG |
| SEQ ID No. 811: | 5′- CATGCGGTATTAGCTCCAGT |
| SEQ ID No. 812: | 5′- CGCAGGCTCCTCCACAGGCG |
| SEQ ID No. 813: | 5′- ACGCAGGCTCCTCCACAGGC |
| SEQ ID No. 814: | 5′- CTCAGGTGTCATGCGGTATT |
| SEQ ID No. 815: | 5′- CGCCTTTGACCCTCAGGTGT |
| SEQ ID No. 816: | 5′- ACCCTCAGGTGTCATGCGGT |
| SEQ ID No. 817: | 5′- CCTCAGGTGTCATGCGGTAT |
| SEQ ID No. 818: | 5′- TTTGACCCTCAGGTGTCATG |
| SEQ ID No. 819: | 5′- GACCCTCAGGTGTCATGCGG |
| SEQ ID No. 820: | 5′- TGACCCTCAGGTGTCATGCG |
| SEQ ID No. 821: | 5′- GCCTTTGACCCTCAGGTGTC |
| SEQ ID No. 822: | 5′- TTGACCCTCAGGTGTCATGC |
| SEQ ID No. 823: | 5′- CCCTCAGGTGTCATGCGGTA |
| SEQ ID No. 824: | 5′- CCTTTGACCCTCAGGTGTCA |
| SEQ ID No. 825: | 5′- CTTTGACCCTCAGGTGTCAT |
| SEQ ID No. 826: | 5′- AGTTATCCCCCACCCATGGA |
| SEQ ID No. 827: | 5′- CCAGCTATCGATCATCGCCT |
| SEQ ID No. 828: | 5′- ACCAGCTATCGATCATCGCC |
| SEQ ID No. 829: | 5′- CAGCTATCGATCATCGCCTT |
| SEQ ID No. 830: | 5′- AGCTATCGATCATCGCCTTG |
| SEQ ID No. 831: | 5′- GCTATCGATCATCGCCTTGG |
| SEQ ID No. 832: | 5′- CTATCGATCATCGCCTTGGT |
| SEQ ID No. 833: | 5′- TTCGTGCGACTTGCATGTGT |
| SEQ ID No. 834: | 5′- TCGATCATCGCCTTGGTAGG |
| SEQ ID No. 835: | 5′- ATCGATCATCGCCTTGGTAG |
| SEQ ID No. 836: | 5′- CACAGGCGACTTGCGCCTTT |
| SEQ ID No. 837: | 5′- CCACAGGCGACTTGCGCCTT |
| SEQ ID No. 838: | 5′- TCCACAGGCGACTTGCGCCT |
| SEQ ID No. 839: | 5′- TCCTCCACAGGCGACTTGCG |
| SEQ ID No. 840: | 5′- CCTCCACAGGCGACTTGCGC |
| SEQ ID No. 841: | 5′- CTCCACAGGCGACTTGCGCC |
| SEQ ID No. 842: | 5′- ACAGGCGACTTGCGCCTTTG |
| SEQ ID No. 843: | 5′- AGCCCCGGTTTCCCGGCGTT |
| SEQ ID No. 844: | 5′- CGCCTTTCCTTTTTCCTCCA |
| SEQ ID No. 845: | 5′- GCCCCGGTTTCCCGGCGTTA |
| SEQ ID No. 846: | 5′- GCCGCCTTTCCTTTTTCCTC |
| SEQ ID No. 847: | 5′- TAGCCCCGGTTTCCCGGCGT |
| SEQ ID No. 848: | 5′- CCGGGTACCGTCAAGGCGCC |
| SEQ ID No. 849: | 5′- AAGCCGCCTTTCCTTTTTCC |
| SEQ ID No. 850: | 5′- CCCCGGTTTCCCGGCGTTAT |
| SEQ ID No. 851: | 5′- CCGGCGTTATCCCAGTCTTA |
| SEQ ID No. 852: | 5′- AGCCGCCTTTCCTTTTTCCT |
| SEQ ID No. 853: | 5′- CCGCCTTTCCTTTTTCCTCC |
| SEQ ID No. 854: | 5′- TTAGCCCCGGTTTCCCGGCG |
| SEQ ID No. 855: | 5′- CCCGGCGTTATCCCAGTCTT |
| SEQ ID No. 856: | 5′- GCCGGGTACCGTCAAGGCGC |
| SEQ ID No. 857: | 5′- GGCCGGGTACCGTCAAGGCG |
| SEQ ID No. 858: | 5′- TCCCGGCGTTATCCCAGTCT |
| SEQ ID No. 859: | 5′- TGGCCGGGTACCGTCAAGGC |
| SEQ ID No. 860: | 5′- GAAGCCGCCTTTCCTTTTTC |
| SEQ ID No. 861: | 5′- CCCGGTTTCCCGGCGTTATC |
| SEQ ID No. 862: | 5′- CGGCGTTATCCCAGTCTTAC |
| SEQ ID No. 863: | 5′- GGCGTTATCCCAGTCTTACA |
| SEQ ID No. 864: | 5′- GCGTTATCCCAGTCTTACAG |
| SEQ ID No. 865: | 5′- CGGGTACCGTCAAGGCGCCG |
| SEQ ID No. 866: | 5′- ATTAGCCCCGGTTTCCCGGC |
| SEQ ID No. 867: | 5′- AAGGGGAAGGCCCTGTCTCC |
| SEQ ID No. 868: | 5′- GGCCCTGTCTCCAGGGAGGT |
| SEQ ID No. 869: | 5′- AGGCCCTGTCTCCAGGGAGG |
| SEQ ID No. 870: | 5′- AAGGCCCTGTCTCCAGGGAG |
| SEQ ID No. 871: | 5′- GCCCTGTCTCCAGGGAGGTC |
| SEQ ID No. 872: | 5′- CGTTATCCCAGTCTTACAGG |
| SEQ ID No. 873: | 5′- GGGTACCGTCAAGGCGCCGC |
| SEQ ID No. 874: | 5′- CGGCAACAGAGTTTTACGAC |
| SEQ ID No. 875: | 5′- GGGGAAGGCCCTGTCTCCAG |
| SEQ ID No. 876: | 5′- AGGGGAAGGCCCTGTCTCCA |
| SEQ ID No. 877: | 5′- GCAGCCGAAGCCGCCTTTCC |
| SEQ ID No. 878: | 5′- TTCTTCCCCGGCAACAGAGT |
| SEQ ID No. 879: | 5′- CGGCACTTGTTCTTCCCCGG |
| SEQ ID No. 880: | 5′- GTTCTTCCCCGGCAACAGAG |
| SEQ ID No. 881: | 5′- GGCACTTGTTCTTCCCCGGC |
| SEQ ID No. 882: | 5′- GCACTTGTTCTTCCCCGGCA |
| SEQ ID No. 883: | 5′- CACTTGTTCTTCCCCGGCAA |
| SEQ ID No. 884: | 5′- TCTTCCCCGGCAACAGAGTT |
| SEQ ID No. 885: | 5′- TTGTTCTTCCCCGGCAACAG |
| SEQ ID No. 886: | 5′- ACTTGTTCTTCCCCGGCAAC |
| SEQ ID No. 887: | 5′- TGTTCTTCCCCGGCAACAGA |
| SEQ ID No. 888: | 5′- CTTGTTCTTCCCCGGCAACA |
| SEQ ID No. 889: | 5′- ACGGCACTTGTTCTTCCCCG |
| SEQ ID No. 890: | 5′- GTCCGCCGCTAACCTTTTAA |
| SEQ ID No. 891: | 5′- CTGGCCGGGTACCGTCAAGG |
| SEQ ID No. 892: | 5′- TCTGGCCGGGTACCGTCAAG |
| SEQ ID No. 893: | 5′- TTCTGGCCGGGTACCGTCAA |
| SEQ ID No. 894: | 5′- CAATGCTGGCAACTAAGGTC |
| SEQ ID No. 895: | 5′- CGTCCGCCGCTAACCTTTTA |
| SEQ ID No. 896: | 5′- CGAAGCCGCCTTTCCTTTTT |
| SEQ ID No. 897: | 5′- CCGAAGCCGCCTTTCCTTTT |
| SEQ ID No. 898: | 5′- GCCGAAGCCGCCTTTCCTTT |
| SEQ ID No. 899: | 5′- AGCCGAAGCCGCCTTTCCTT |
| SEQ ID No. 900: | 5′- ACCGTCAAGGCGCCGCCCTG |
| SEQ ID No. 901: | 5′- CCGTGGCTTTCTGGCCGGGT |
| SEQ ID No. 902: | 5′- GCTTTCTGGCCGGGTACCGT |
| SEQ ID No. 903: | 5′- GCCGTGGCTTTCTGGCCGGG |
| SEQ ID No. 904: | 5′- GGCTTTCTGGCCGGGTACCG |
| SEQ ID No. 905: | 5′- CTTTCTGGCCGGGTACCGTC |
| SEQ ID No. 906: | 5′- TGGCTTTCTGGCCGGGTACC |
| SEQ ID No. 907: | 5′- GTGGCTTTCTGGCCGGGTAC |
| SEQ ID No. 908: | 5′- CGTGGCTTTCTGGCCGGGTA |
| SEQ ID No. 909: | 5′- TTTCTGGCCGGGTACCGTCA |
| SEQ ID No. 910: | 5′- GGGAAGGCCCTGTCTCCAGG |
| SEQ ID No. 911: | 5′- CGAAGGGGAAGGCCCTGTCT |
| SEQ ID No. 912: | 5′- CCGAAGGGGAAGGCCCTGTC |
| SEQ ID No. 913: | 5′- GAAGGGGAAGGCCCTGTCTC |
| SEQ ID No. 914: | 5′- GGCGCCGCCCTGTTCGAACG |
| SEQ ID No. 915: | 5′- AGGCGCCGCCCTGTTCGAAC |
| SEQ ID No. 916: | 5′- AAGGCGCCGCCCTGTTCGAA |
| SEQ ID No. 917: | 5′- CCCGGCAACAGAGTTTTACG |
| SEQ ID No. 918: | 5′- CCCCGGCAACAGAGTTTTAC |
| SEQ ID No. 919: | 5′- CCATCTGTAAGTGGCAGCCG |
| SEQ ID No. 920: | 5′- TCTGTAAGTGGCAGCCGAAG |
| SEQ ID No. 921: | 5′- CTGTAAGTGGCAGCCGAAGC |
| SEQ ID No. 922: | 5′- CCCATCTGTAAGTGGCAGCC |
| SEQ ID No. 923: | 5′- TGTAAGTGGCAGCCGAAGCC |
| SEQ ID No. 924: | 5′- CATCTGTAAGTGGCAGCCGA |
| SEQ ID No. 925: | 5′- ATCTGTAAGTGGCAGCCGAA |
| SEQ ID No. 926: | 5′- CAGCCGAAGCCGCCTTTCCT |
| SEQ ID No. 927: | 5′- GGCAACAGAGTTTTACGACC |
| SEQ ID No. 928: | 5′- CCGGCAACAGAGTTTTACGA |
| SEQ ID No. 929: | 5′- TTCCCCGGCAACAGAGTTTT |
| SEQ ID No. 930: | 5′- CTTCCCCGGCAACAGAGTTT |
| SEQ ID No. 931: | 5′- TCCCCGGCAACAGAGTTTTA |
| SEQ ID No. 932: | 5′- CCGTCCGCCGCTAACCTTTT |
| SEQ ID No. 933: | 5′- CTTCCTCCGACTTACGCCGG |
| SEQ ID No. 934: | 5′- CCTCCGACTTACGCCGGCAG |
| SEQ ID No. 935: | 5′- TTCCTCCGACTTACGCCGGC |
| SEQ ID No. 936: | 5′- TCCTCCGACTTACGCCGGCA |
| SEQ ID No. 937: | 5′- TCCGACTTACGCCGGCAGTC |
| SEQ ID No. 938: | 5′- CCGACTTACGCCGGCAGTCA |
| SEQ ID No. 939: | 5′- GCCTTCCTCCGACTTACGCC |
| SEQ ID No. 940: | 5′- CCTTCCTCCGACTTACGCCG |
| SEQ ID No. 941: | 5′- GCTCTCCCCGAGCAACAGAG |
| SEQ ID No. 942: | 5′- CTCTCCCCGAGCAACAGAGC |
| SEQ ID No. 943: | 5′- CGCTCTCCCCGAGCAACAGA |
| SEQ ID No. 944: | 5′- CTCCGACTTACGCCGGCAGT |
| SEQ ID No. 945: | 5′- TCTCCCCGAGCAACAGAGCT |
| SEQ ID No. 946: | 5′- CGACTTACGCCGGCAGTCAC |
| SEQ ID No. 947: | 5′- TCGGCACTGGGGTGTGTCCC |
| SEQ ID No. 948: | 5′- GGCACTGGGGTGTGTCCCCC |
| SEQ ID No. 949: | 5′- CTGGGGTGTGTCCCCCCAAC |
| SEQ ID No. 950: | 5′- CACTGGGGTGTGTCCCCCCA |
| SEQ ID No. 951: | 5′- ACTGGGGTGTGTCCCCCCAA |
| SEQ ID No. 952: | 5′- GCACTGGGGTGTGTCCCCCC |
| SEQ ID No. 953: | 5′- TGGGGTGTGTCCCCCCAACA |
| SEQ ID No. 954: | 5′- CACTCCAGACTTGCTCGACC |
| SEQ ID No. 955: | 5′- TCACTCCAGACTTGCTCGAC |
| SEQ ID No. 956: | 5′- CGGCACTGGGGTGTGTCCCC |
| SEQ ID No. 957: | 5′- CGCCTTCCTCCGACTTACGC |
| SEQ ID No. 958: | 5′- CTCCCCGAGCAACAGAGCTT |
| SEQ ID No. 959: | 5′- ACTCCAGACTTGCTCGACCG |
| SEQ ID No. 960: | 5′- CCCATGCCGCTCTCCCCGAG |
| SEQ ID No. 961: | 5′- CCATGCCGCTCTCCCCGAGC |
| SEQ ID No. 962: | 5′- CCCCATGCCGCTCTCCCCGA |
| SEQ ID No. 963: | 5′- TCACTCGGTACCGTCTCGCA |
| SEQ ID No. 964: | 5′- CATGCCGCTCTCCCCGAGCA |
| SEQ ID No. 965: | 5′- ATGCCGCTCTCCCCGAGCAA |
| SEQ ID No. 966: | 5′- TTCGGCACTGGGGTGTGTCC |
| SEQ ID No. 967: | 5′- TGCCGCTCTCCCCGAGCAAC |
| SEQ ID No. 968: | 5′- TTCACTCCAGACTTGCTCGA |
| SEQ ID No. 969: | 5′- CCCGCAAGAAGATGCCTCCT |
| SEQ ID No. 970: | 5′- AGAAGATGCCTCCTCGCGGG |
| SEQ ID No. 971: | 5′- AAGAAGATGCCTCCTCGCGG |
| SEQ ID No. 972: | 5′- CGCAAGAAGATGCCTCCTCG |
| SEQ ID No. 973: | 5′- AAGATGCCTCCTCGCGGGCG |
| SEQ ID No. 974: | 5′- CCGCAAGAAGATGCCTCCTC |
| SEQ ID No. 975: | 5′- GAAGATGCCTCCTCGCGGGC |
| SEQ ID No. 976: | 5′- CCCCGCAAGAAGATGCCTCC |
| SEQ ID No. 977: | 5′- CAAGAAGATGCCTCCTCGCG |
| SEQ ID No. 978: | 5′- TCCTTCGGCACTGGGGTGTG |
| SEQ ID No. 979: | 5′- CCGCTCTCCCCGAGCAACAG |
| SEQ ID No. 980: | 5′- TGCCTCCTCGCGGGCGTATC |
| SEQ ID No. 981: | 5′- GACTTACGCCGGCAGTCACC |
| SEQ ID No. 982: | 5′- GGCTCCTCTCTCAGCGGCCC |
| SEQ ID No. 983: | 5′- CCTTCGGCACTGGGGTGTGT |
| SEQ ID No. 984: | 5′- GGGGTGTGTCCCCCCAACAC |
| SEQ ID No. 985: | 5′- GCCGCTCTCCCCGAGCAACA |
| SEQ ID No. 986: | 5′- AGATGCCTCCTCGCGGGCGT |
| SEQ ID No. 987: | 5′- CACTCGGTACCGTCTCGCAT |
| SEQ ID No. 988: | 5′- CTCACTCGGTACCGTCTCGC |
| SEQ ID No. 989: | 5′- GCAAGAAGATGCCTCCTCGC |
| SEQ ID No. 990: | 5′- CTCCAGACTTGCTCGACCGC |
| SEQ ID No. 991: | 5′- TTACGCCGGCAGTCACCTGT |
| SEQ ID No. 992: | 5′- CTTCGGCACTGGGGTGTGTC |
| SEQ ID No. 993: | 5′- CTCGCGGGCGTATCCGGCAT |
| SEQ ID No. 994: | 5′- GCCTCCTCGCGGGCGTATCC |
| SEQ ID No. 995: | 5′- ACTCGGTACCGTCTCGCATG |
| SEQ ID No. 996: | 5′- GATGCCTCCTCGCGGGCGTA |
| SEQ ID No. 997: | 5′- GGGTGTGTCCCCCCAACACC |
| SEQ ID No. 998: | 5′- ACTTACGCCGGCAGTCACCT |
| SEQ ID No. 999: | 5′- CTTACGCCGGCAGTCACCTG |
| SEQ ID No. 1000: | 5′- ATGCCTCCTCGCGGGCGTAT |
| SEQ ID No. 1001: | 5′- GCGCCGCGGGCTCCTCTCTC |
| SEQ ID No. 1002: | 5′- GGTGTGTCCCCCCAACACCT |
| SEQ ID No. 1003: | 5′- GTGTGTCCCCCCAACACCTA |
| SEQ ID No. 1004: | 5′- CCTCGCGGGCGTATCCGGCA |
| SEQ ID No. 1005: | 5′- CCTCACTCGGTACCGTCTCG |
| SEQ ID No. 1006: | 5′- TCCTCACTCGGTACCGTCTC |
| SEQ ID No. 1007: | 5′- TCGCGGGCGTATCCGGCATT |
| SEQ ID No. 1008: | 5′- TTTCACTCCAGACTTGCTCG |
| SEQ ID No. 1009: | 5′- TACGCCGGCAGTCACCTGTG |
| SEQ ID No. 1010: | 5′- TCCAGACTTGCTCGACCGCC |
| SEQ ID No. 1011: | 5′- CTCGGTACCGTCTCGCATGG |
| SEQ ID No. 1012: | 5′- CGCGGGCGTATCCGGCATTA |
| SEQ ID No. 1013: | 5′- GCGTATCCGGCATTAGCGCC |
| SEQ ID No. 1014: | 5′- GGGCTCCTCTCTCAGCGGCC |
| SEQ ID No. 1015: | 5′- TCCCCGAGCAACAGAGCTTT |
| SEQ ID No. 1016: | 5′- CCCCGAGCAACAGAGCTTTA |
| SEQ ID No. 1017: | 5′- CCGAGCAACAGAGCTTTACA |
| SEQ ID No. 1018: | 5′- CCATCCCATGGTTGAGCCAT |
| SEQ ID No. 1019: | 5′- GTGTCCCCCCAACACCTAGC |
| SEQ ID No. 1020: | 5′- GCGGGCGTATCCGGCATTAG |
| SEQ ID No. 1021: | 5′- CGAGCGGCTTTTTGGGTTTC |
| SEQ ID No. 1022: | 5′- CTTTCACTCCAGACTTGCTC |
| SEQ ID No. 1023: | 5′- TTCCTTCGGCACTGGGGTGT |
| SEQ ID No. 1024: | 5′- CCGCCTTCCTCCGACTTACG |
| SEQ ID No. 1025: | 5′- CCCGCCTTCCTCCGACTTAC |
| SEQ ID No. 1026: | 5′- CCTCCTCGCGGGCGTATCCG |
| SEQ ID No. 1027: | 5′- TCCTCGCGGGCGTATCCGGC |
| SEQ ID No. 1028: | 5′- CATTAGCGCCCGTTTCCGGG |
| SEQ ID No. 1029: | 5′- GCATTAGCGCCCGTTTCCGG |
| SEQ ID No. 1030: | 5′- GGCATTAGCGCCCGTTTCCG |
| SEQ ID No. 1031: | 5′- GTCTCGCATGGGGCTTTCCA |
| SEQ ID No. 1032: | 5′- GCCATGGACTTTCACTCCAG |
| SEQ ID No. 1033: | 5′- CATGGACTTTCACTCCAGAC |
| SEQ ID No. 1037: | 5′- ACCGTCTCACAAGGAGCTTT |
| SEQ ID No. 1038: | 5′- TACCGTCTCACAAGGAGCTT |
| SEQ ID No. 1039: | 5′- GTACCGTCTCACAAGGAGCT |
| SEQ ID No. 1040: | 5′- GCCTACCCGTGTATTATCCG |
| SEQ ID No. 1041: | 5′- CCGTCTCACAAGGAGCTTTC |
| SEQ ID No. 1042: | 5′- CTACCCGTGTATTATCCGGC |
| SEQ ID No. 1043: | 5′- GGTACCGTCTCACAAGGAGC |
| SEQ ID No. 1044: | 5′- CGTCTCACAAGGAGCTTTCC |
| SEQ ID No. 1045: | 5′- TCTCACAAGGAGCTTTCCAC |
| SEQ ID No. 1046: | 5′- TACCCGTGTATTATCCGGCA |
| SEQ ID No. 1047: | 5′- GTCTCACAAGGAGCTTTCCA |
| SEQ ID No. 1048: | 5′- ACCCGTGTATTATCCGGCAT |
| SEQ ID No. 1049: | 5′- CTCGGTACCGTCTCACAAGG |
| SEQ ID No. 1050: | 5′- CGGTACCGTCTCACAAGGAG |
| SEQ ID No. 1051: | 5′- ACTCGGTACCGTCTCACAAG |
| SEQ ID No. 1052: | 5′- CGGCTGGCTCCATAACGGTT |
| SEQ ID No. 1053: | 5′- ACAAGTAGATGCCTACCCGT |
| SEQ ID No. 1054: | 5′- TGGCTCCATAACGGTTACCT |
| SEQ ID No. 1055: | 5′- CAAGTAGATGCCTACCCGTG |
| SEQ ID No. 1056: | 5′- CACAAGTAGATGCCTACCCG |
| SEQ ID No. 1057: | 5′- GGCTCCATAACGGTTACCTC |
| SEQ ID No. 1058: | 5′- ACACAAGTAGATGCCTACCC |
| SEQ ID No. 1059: | 5′- CTGGCTCCATAACGGTTACC |
| SEQ ID No. 1060: | 5′- GCTGGCTCCATAACGGTTAC |
| SEQ ID No. 1061: | 5′- GGCTGGCTCCATAACGGTTA |
| SEQ ID No. 1062: | 5′- GCTCCATAACGGTTACCTCA |
| SEQ ID No. 1063: | 5′- AAGTAGATGCCTACCCGTGT |
| SEQ ID No. 1064: | 5′- CTCCATAACGGTTACCTCAC |
| SEQ ID No. 1065: | 5′- TGCCTACCCGTGTATTATCC |
| SEQ ID No. 1066: | 5′- TCGGTACCGTCTCACAAGGA |
| SEQ ID No. 1067: | 5′- CTCACAAGGAGCTTTCCACT |
| SEQ ID No. 1068: | 5′- GTAGATGCCTACCCGTGTAT |
| SEQ ID No. 1069: | 5′- CCTACCCGTGTATTATCCGG |
| SEQ ID No. 1070: | 5′- CACTCGGTACCGTCTCACAA |
| SEQ ID No. 1071: | 5′- CTCAGCGATGCAGTTGCATC |
| SEQ ID No. 1072: | 5′- AGTAGATGCCTACCCGTGTA |
| SEQ ID No. 1073: | 5′- GCGGCTGGCTCCATAACGGT |
| SEQ ID No. 1074: | 5′- CCAAAGCAATCCCAAGGTTG |
| SEQ ID No. 1075: | 5′- TCCATAACGGTTACCTCACC |
| SEQ ID No. 1076: | 5′- CCCGTGTATTATCCGGCATT |
| SEQ ID No. 1077: | 5′- TCTCAGCGATGCAGTTGCAT |
| SEQ ID No. 1078: | 5′- CCATAACGGTTACCTCACCG |
| SEQ ID No. 1079: | 5′- TCAGCGATGCAGTTGCATCT |
| SEQ ID No. 1080: | 5′- GGCGGCTGGCTCCATAACGG |
| SEQ ID No. 1081: | 5′- AAGCAATCCCAAGGTTGAGC |
| SEQ ID No. 1082: | 5′- TCACTCGGTACCGTCTCACA |
| SEQ ID No. 1083: | 5′- CCGAGTGTTATTCCAGTCTG |
| SEQ ID No. 1084: | 5′- CACAAGGAGCTTTCCACTCT |
| SEQ ID No. 1085: | 5′- ACAAGGAGCTTTCCACTCTC |
| SEQ ID No. 1086: | 5′- TCACAAGGAGCTTTCCACTC |
| SEQ ID No. 1087: | 5′- CAGCGATGCAGTTGCATCTT |
| SEQ ID No. 1088: | 5′- CAAGGAGCTTTCCACTCTCC |
| SEQ ID No. 1089: | 5′- CCAGTCTGAAAGGCAGATTG |
| SEQ ID No. 1090: | 5′- CAGTCTGAAAGGCAGATTGC |
| SEQ ID No. 1091: | 5′- CGGCGGCTGGCTCCATAACG |
| SEQ ID No. 1092: | 5′- CCTCTCTCAGCGATGCAGTT |
| SEQ ID No. 1093: | 5′- CTCTCTCAGCGATGCAGTTG |
| SEQ ID No. 1094: | 5′- TCTCTCAGCGATGCAGTTGC |
| SEQ ID No. 1095: | 5′- CTCTCAGCGATGCAGTTGCA |
| SEQ ID No. 1096: | 5′- CAATCCCAAGGTTGAGCCTT |
| SEQ ID No. 1097: | 5′- AATCCCAAGGTTGAGCCTTG |
| SEQ ID No. 1098: | 5′- AGCAATCCCAAGGTTGAGCC |
| SEQ ID No. 1099: | 5′- CTCACTCGGTACCGTCTCAC |
| SEQ ID No. 1100: | 5′- GCAATCCCAAGGTTGAGCCT |
| SEQ ID No. 1101: | 5′- GCCTTGGACTTTCACTTCAG |
| SEQ ID No. 1102: | 5′- CATAACGGTTACCTCACCGA |
| SEQ ID No. 1103: | 5′- CTCCTCTCTCAGCGATGCAG |
| SEQ ID No. 1104: | 5′- TCGGCGGCTGGCTCCATAAC |
| SEQ ID No. 1105: | 5′- AGTCTGAAAGGCAGATTGCC |
| SEQ ID No. 1106: | 5′- TCCTCTCTCAGCGATGCAGT |
| SEQ ID No. 1107: | 5′- CCCAAGGTTGAGCCTTGGAC |
| SEQ ID No. 1108: | 5′- ATAACGGTTACCTCACCGAC |
| SEQ ID No. 1109: | 5′- TCCCAAGGTTGAGCCTTGGA |
| SEQ ID No. 1110: | 5′- ATTATCCGGCATTAGCACCC |
| SEQ ID No. 1111: | 5′- CTACGTGCTGGTAACACAGA |
| SEQ ID No. 1112: | 5′- GCCGCTAGCCCCGAAGGGCT |
| SEQ ID No. 1113: | 5′- CTAGCCCCGAAGGGCTCGCT |
| SEQ ID No. 1114: | 5′- CGCTAGCCCCGAAGGGCTCG |
| SEQ ID No. 1115: | 5′- AGCCCCGAAGGGCTCGCTCG |
| SEQ ID No. 1116: | 5′- CCGCTAGCCCCGAAGGGCTC |
| SEQ ID No. 1117: | 5′- TAGCCCCGAAGGGCTCGCTC |
| SEQ ID No. 1118: | 5′- GCTAGCCCCGAAGGGCTCGC |
| SEQ ID No. 1119: | 5′- GCCCCGAAGGGCTCGCTCGA |
| SEQ ID No. 1120: | 5′- ATCCCAAGGTTGAGCCTTGG |
| SEQ ID No. 1121: | 5′- GAGCCTTGGACTTTCACTTC |
| SEQ ID No. 1122: | 5′- CAAGGTTGAGCCTTGGACTT |
| SEQ ID No. 1123: | 5′- GAGCTTTCCACTCTCCTTGT |
| SEQ ID No. 1124: | 5′- CCAAGGTTGAGCCTTGGACT |
| SEQ ID No. 1125: | 5′- CGGGCTCCTCTCTCAGCGAT |
| SEQ ID No. 1126: | 5′- GGAGCTTTCCACTCTCCTTG |
| SEQ ID No. 1127: | 5′- GGGCTCCTCTCTCAGCGATG |
| SEQ ID No. 1128: | 5′- TCTCCTTGTCGCTCTCCCCG |
| SEQ ID No. 1129: | 5′- TCCTTGTCGCTCTCCCCGAG |
| SEQ ID No. 1130: | 5′- AGCTTTCCACTCTCCTTGTC |
| SEQ ID No. 1131: | 5′- CCACTCTCCTTGTCGCTCTC |
| SEQ ID No. 1132: | 5′- GGCTCCTCTCTCAGCGATGC |
| SEQ ID No. 1133: | 5′- CCTTGTCGCTCTCCCCGAGC |
| SEQ ID No. 1134: | 5′- CACTCTCCTTGTCGCTCTCC |
| SEQ ID No. 1135: | 5′- ACTCTCCTTGTCGCTCTCCC |
| SEQ ID No. 1136: | 5′- CTCTCCTTGTCGCTCTCCCC |
| SEQ ID No. 1137: | 5′- GCGGGCTCCTCTCTCAGCGA |
| SEQ ID No. 1138: | 5′- GGCTCCATCATGGTTACCTC |
| SEQ ID No. 1142: | 5′- CTTCCTCCGGCTTGCGCCGG |
| SEQ ID No. 1143: | 5′- CGCTCTTCCCGA(G/T)TGACTGA |
| SEQ ID No. 1144: | 5′- CCTCGGGCTCCTCCATC(A/T)GC |

2. The method according to claim 1, wherein drink-spoiling microorganisms belonging to the genus Zygosacchaeromyces are detected with oligonucleotide probe SEQ ID No. 1.

3. The method according to claim 1, wherein the drink-spoiling microorganism Zygosacchaeromyces bailii is detected with at least one oligonucleotide probe selected from the group consisting of SEQ ID No. 5 through SEQ ID No. 21.

4. The method according to claim 1, wherein the drink-spoiling microorganism Zygosacchaeromyces fermentati is detected with oligonucleotide probe SEQ ID No. 22.

5. The method according to claim 1, wherein the drink-spoiling microorganism Zygosacchaeromyces microellipsoides is detected with at least one oligonucleotide probe selected from the group consisting of SEQ ID No. 23 and SEQ ID No. 24.

6. The method according to claim 1, wherein the drink-spoiling microorganism Zygosacchaeromyces mellis is detected with at least one oligonucleotide probe selected from the group consisting of SEQ ID No. 25 through SEQ ID No. 75.

7. The method according to claim 1, wherein the drink-spoiling microorganism Zygosacchaeromyces rouxii is detected with at least one oligonucleotide probe selected from the group consisting of SEQ ID No. 76 through SEQ ID No. 126.

8. The method according to claim 1, wherein the drink-spoiling microorganisms Zygosacchaeromyces mellis and Zygosacchaeromyces rouxii are detected simultaneously with oligonucleotide probe SEQ ID No. 127.

9. The method according to claim 1, wherein the drink-spoiling microorganism Zygosacchaeromyces bisporus is detected with at least one oligonucleotide probe selected from the group consisting of SEQ ID No. 128 through SEQ ID No. 142.

10. The method according to claim 1, wherein the drink-spoiling microorganism Hanseniaspora uvarum is detected with at least one oligonucleotide probe selected from the group consisting of SEQ ID No. 143 and SEQ ID No. 144.

11. The method according to claim 1, wherein the drink-spoiling microorganism Candida intermedia is detected with at least one oligonucleotide probe selected from the group consisting of SEQ ID No. 145 and SEQ ID No. 146.

12. The method according to claim 1, wherein the drink-spoiling microorganism Candida parapsilosis is detected with oligonucleotide probe SEQ ID No. 148.

13. The method according to claim 1, wherein the drink-spoiling microorganism Candida crusei (Issatchenkia orientalis) is detected with oligonucleotide probe SEQ ID No. 149.

14. The method according to claim 1, wherein the drink-spoiling microorganisms Brettanomyces (Dekkera) anomala and Dekkera bruxellensis are detected simultaneously with oligonucleotide probe SEQ ID No. 150.

15. The method according to claim 1, wherein the drink-spoiling microorganism Brettanomyces (Dekkera) bruxellensis is detected with oligonucleotide probe SEQ ID No. 151.

16. The method according to claim 1, wherein the drink-spoiling microorganism Brettanomyces (Dekkera) naardenensis is detected with oligonucleotide probe SEQ ID No. 152.

17. The method according to claim 1, wherein the drink-spoiling microorganism Pichia membranaefaciens is detected with oligonucleotide probe SEQ ID No. 153.

18. The method according to claim 1, wherein the drink-spoiling microorganisms Pichia minuta and Pichia anomala are detected simultaneously with oligonucleotide probe SEQ ID No. 154.

19. The method according to claim 1, wherein the drink-spoiling microorganism Saccharomyces exiguus is detected with oligonucleotide probe SEQ ID No. 157.

20. The method according to claim 1, wherein the drink-spoiling microorganism Saccharomycodes ludwigii is detected with at least one oligonucleotide probe selected from the group consisting of SEQ ID No. 158 and SEQ ID No. 159.

21. The method according to claim 1, wherein the drink-spoiling microorganism Saccharomyces cerevisiae is detected with oligonucleotide probe SEQ ID No. 160.

22. The method according to claim 1, wherein the drink-spoiling microorganism Mucor racemosus is detected with oligonucleotide probe SEQ ID No. 163.

23. The method according to claim 1, wherein the drink-spoiling microorganism Byssochlamys nivea is detected with oligonucleotide probe SEQ ID No. 164.

24. The method according to claim 1, wherein the drink-spoiling microorganism Neosartorya fischeri is detected with oligonucleotide probe SEQ ID No. 165.

25. The method according to claim 1, wherein the drink-spoiling microorganisms Aspergillus fumigatus and A. fischeri are detected simultaneously with oligonucleotide probe SEQ ID No. 166.

26. The method according to claim 1, wherein the drink-spoiling microorganism Talaromyces flavus is detected with oligonucleotide probe SEQ ID No. 167.

27. The method according to claim 1, wherein the drink-spoiling microorganisms Talaromyces bacillisporus and T. flavus are detected simultaneously with oligonucleotide probe SEQ ID No. 168.

28. The method according to claim 1, wherein the drink-spoiling microorganism Lactobacillus collinoides is detected with at least one oligonucleotide probe selected from the group consisting of SEQ ID No. 169 through SEQ ID No. 269.

29. The method according to claim 1, wherein drink-spoiling microorganisms of the genus Leuconostoc are detected with at least one oligonucleotide probe selected from the group consisting of SEQ ID No. 270 and SEQ ID No. 271.

30. The method according to claim 1, wherein the drink-spoiling microorganisms Leuconostoc mesenteroides and L. pseudomesenteroides are detected simultaneously with at least one oligonucleotide probe selected from the group consisting of SEQ ID No. 272 through SEQ ID No. 301.

31. The method according to claim 1, wherein the drink-spoiling microorganism Leuconostoc pseudomesenteroides is detected with at least one oligonucleotide probe selected from the group consisting of SEQ ID No. 302 through SEQ ID No. 341.

32. The method according to claim 1, wherein the drink-spoiling microorganism Oenococcus oenis is detected with at least one oligonucleotide probe selected from the group consisting of SEQ ID No. 342 through SEQ ID No. 444.

33. The method according to claim 1, wherein drink-spoiling microorganisms of the genus Weissella are detected with at least one oligonucleotide probe selected from the group consisting of SEQ ID No. 445 through SEQ ID No. 495.

34. The method according to claim 1, wherein drink-spoiling microorganisms of the genus Lactococcus are detected with at least one oligonucleotide probe selected from the group consisting of SEQ ID No. 496 through SEQ ID No. 546.

35. The method according to claim 1, wherein drink-spoiling microorganisms of the genera Acelobacter and Gluconobacter are detected simultaneously with at least one oligonucleotide probe selected from the group consisting of SEQ ID No. 547 through SEQ ID No. 608.

36. The method according to claim 1, wherein drink-spoiling microorganisms of the genera Acetobacter, Gluconobacter and Gluconoacetobacter are detected simultaneously with at least one oligonucleotide probe selected from the group consisting of SEQ ID No. 609 through SEQ ID No. 842.

37. The method according to claim 1, wherein the drink-spoiling microorganism Bacillus coagulans is detected with at least one oligonucleotide probe selected from the group consisting of SEQ ID No. 843 through SEQ ID No. 932.

38. The method according to claim 1, wherein drink-spoiling microorganisms of the genus Alicyclobacilus are detected with at least one oligonucleotide probe selected from the group consisting of SEQ ID No. 933 through SEQ ID No. 1033.

39. The method according to claim 1, wherein the drink-spoiling microorganism Alicyclobacillus acidoterrestris is detected with at least one oligonucleotide probe selected from the group consisting of SEQ ID No. 1037 and SEQ ID No. 1138.

40. The method according to claim 1, wherein the drink-spoiling microorganisms Alicyclobacillus cycloheptanicus and A. herbarius are detected simultaneously with at least one oligonucleotide probe selected from the group consisting of SEQ ID No. 1142 through SEQ ID No. 1144.

41. The method according to claim 2, wherein the at least one oligonucleotide probe is used in combination with one or more competitor probes.

42. The method according to claim 41, wherein the oligonucleotide probe SEQ ID No. 1 is used in combination with one or more competitor probes selected from the group consisting of SEQ ID No. 2 through SEQ ID No. 4.

43. The method according to claim 11, wherein the at least one oligonucleotide probe is used in combination with one or more competitor probes.

44. The method according to claim 43, wherein the oligonucleotide probe SEQ ID No. 146 is used in combination with competitor probe SEQ ID No. 147.

45. The method according to claim 18, wherein the at least one oligonucleotide probe is used in combination with one or more competitor probes.

46. The method according to claim 45, wherein the oligonucleotide probe SEQ ID No. 154 is used in combination with one or more competitor probes selected from the group consisting of SEQ ID No. 155 and SEQ ID No. 156.

47. The method according to claim 21, wherein the at least one oligonucleotide probe is used in combination with one or more competitor probes.

48. The method according to claim 47, wherein the oligonucleotide probe SEQ ID No. 160 is used in combination with one or more competitor probes selected from the group consisting of SEQ ID No. 161 and SEQ ID No. 162.

49. The method according to claim 38, wherein the at least one oligonucleotide probe is used in combination with one or more competitor probes.

50. The method according to claim 49, wherein the oligonucleotide probe SEQ ID No. 933 is used in combination with one or more competitor probes selected from the group consisting of SEQ ID No. 1034 through SEQ ID No. 1036.

51. The method according to claim 39, wherein the at least one oligonucleotide probe is used in combination with one or more competitor probes.

52. The method according to claim 51, wherein the oligonucleotide probe SEQ ID No. 1044 is used in combination with the competitor probe SEQ ID No. 1139.

53. The method according to claim 51, wherein the oligonucleotide probe SEQ ID No. 1057 is used in combination with one or more competitor probes selected from the group consisting of SEQ ID No. 1140 and SEQ ID No. 1141.

54. The method according to claim 1, characterized in by comprising the following steps:
a) cultivating the drink-spoiling microorganisms contained in the sample,
b) fixing the drink-spoiling microorganisms contained in the sample,
c) incubating the fixed microorganisms with at least one oligonucleotide probe optionally in combination with a competitor probe,
d) removing non-hybridised oligonucleotide probes,
e) detecting and visualizing and optionally quantifiying the drink-spoiling microorganisms with the hybridized oligonucleotide probes.

55. The method according to claim 1, wherein the sample is a sample from a non-alcoholic beverage.

56. A kit for performing a method according to claim 1, containing at least one oligonucleotide according to claim 1.
